(19) United States
(12) Patent Application Publication
(10) Pub. No.: US 2025/0188463 A1
(43) Pub. Date: Jun. 12, 2025

(54) ANTISENSE OLIGONUCLEOTIDES TARGETING ALPHA-SYNUCLEIN AND USES THEREOF

(71) Applicants: Bristol-Myers Squibb Company, Princeton, NJ (US); Roche Innovation Center Copenhagen A/S, Hørsholm (DK)

(72) Inventors: Richard E. OLSON, Cambridge, MA (US); Angela M. CACACE, Haddam Neck, CT (US); Jere E. MEREDITH, JR., Haddam, CT (US); Nino DEVIDZE, Conshohocken, PA (US); James K. LOY, Gales Ferry, CT (US); Carl J. BALDICK, Pennington, NJ (US); Annapurna PENDRI, South Glastonbury, CT (US); Ivar M. MCDONALD, Woodstock, CT (US); Peter HAGEDORN, Hørsholm (DK); Marianne Lerbech JENSEN, Køge (DK)

(73) Assignees: Bristol-Myers Squibb Company, Princeton, NJ (US); Roche Innovation Center Copenhagen A/S, Hørsholm (DK)

(21) Appl. No.: 19/030,693

(22) Filed: Jan. 17, 2025

Related U.S. Application Data
(62) Division of application No. 16/961,578, filed on Jul. 10, 2020, now Pat. No. 12,241,065.
(60) Provisional application No. 62/616,994, filed on Jan. 12, 2018.

(51) Int. Cl. C12N 15/113; A61K 9/00; A61K 31/7088; A61K 39/395

(52) U.S. Cl. CPC C12N 15/113; A61K 9/0085; A61K 31/7088; A61K 39/3955; C12N 2310/31; C12N 2310/3231; C12N 2310/3341; C12N 2310/341; C12N 2310/351; C12N 2320/31; C12N 2320/32

(57) ABSTRACT

The present disclosure relates to antisense oligonucleotides, which target SNCA mRNA (e.g., at an intron exon junction) in a cell, leading to reduced expression of SNCA protein. Reduction of SNCA protein expression is beneficial for the treatment of certain medical disorders, e.g., a neurological disorder.

### CROSS-REFERENCE TO RELATED APPLICATIONS

This application is a divisional application of U.S. application Ser. No. 16/961,578, 371(c) date of Jul. 10, 2020 (currently allowed), which is the National Phase of International Application No. PCT/US2019/013255, filed Jan. 11, 2019, which claims the priority benefit of U.S. Provisional Application No. 62/616,994, filed Jan. 12, 2018, each of which is hereby incorporated by reference herein in its entirety.

### REFERENCE TO SEQUENCE LISTING SUBMITTED ELECTRONICALLY

The content of the electronically submitted sequence listing (Name: 3338.109PC01_st26.xml, Size: 39,912 bytes; and Date of Creation: Jan. 11, 2024) is herein incorporated by reference in its entirety.

### FIELD OF THE DISCLOSURE

The present disclosure relates to antisense oligomeric compounds (ASOs) that target an intron exon junction of alpha-synuclein (SNCA) transcript in a cell, leading to reduced expression of alpha-synuclein (SNCA) protein. Reduction of SNCA protein expression can be beneficial for a range of medical disorders, such as multiple system atrophy, Parkinson's disease, Parkinson's Disease Dementia (PDD), and dementia with Lewy bodies.

### BACKGROUND OF THE DISCLOSURE

Alpha-synuclein (SNCA), a member of the synuclein protein family, is a small soluble protein that is expressed primarily within the neural tissues. See Marques O et al., Cell Death Dis. 19: e350 (2012). It is expressed in many cell types but is predominantly localized within the presynaptic terminals of neurons. While the precise function has yet to be fully elucidated, SNCA has been suggested to play an important role in the regulation of synaptic transmission. For instance, SNCA functions as a molecular chaperone in the formation of SNARE complexes, which mediate the docking of synaptic vesicles with the presynaptic membranes of neurons. SNCA can also interact with other proteins like the microtubule-associated protein tau, which helps stabilize microtubules and regulate vesicle trafficking.

Due to SNCA's role in the regulation of synaptic transmission, alterations of SNCA expression and/or function can disrupt critical biological processes. Such disruptions have been thought to contribute to α-synucleinopathies, which are neurodegenerative diseases characterized by abnormal accumulation of SNCA protein aggregates within the brain. Accordingly, insoluble inclusions of misfolded, aggregated, and phosphorylated SNCA protein are a pathological hallmark for diseases such as Parkinson's disease (PD), Parkinson's Disease Dementia (PDD), dementia with Lewy bodies (DLB), and multiple system atrophy (MSA). See Galvin J E et al., Archives of Neurology 58: 186-190 (2001); and Valera E et al., J Neurochem 139 Suppl 1: 346-352 (Oct. 2016).

α-Synucleinopathies, such as Parkinson's disease, are highly prevalent progressive neurodegenerative brain disorders, especially among the elderly. See Recchia A et al., FASEB J. 18: 617-26 (2004). It is estimated that approximately seven to ten million people worldwide are living with such disorders, with about 60,000 new cases each year in the United States alone. Medication costs for an individual person can easily exceed $2,500 a year and therapeutic surgery can cost up to $100,000 per patient. Therefore, a more robust and cost-effective treatment options are greatly needed.

### BRIEF SUMMARY OF THE DISCLOSURE

The present disclosure is directed to an antisense oligonucleotide (ASO) comprising a contiguous nucleotide sequence of 10 to 30 nucleotides in length that are complementary to a nucleic acid sequence within an alpha-synuclein (SNCA) transcript, wherein the nucleic acid sequence comprises nucleotides 7592-7637 of SEQ ID NO: 1. In some embodiments, the nucleic acid sequence comprises nucleotides 7602-7627 of SEQ ID NO: 1. In other embodiments, the nucleic acid sequence corresponds to nucleotides 7603-7620 of SEQ ID NO: 1. In certain embodiments, the nucleic acid sequence corresponds to nucleotides 7604-7620 of SEQ ID NO: 1. In some embodiments, the SNCA transcript comprises SEQ ID NO: 1. In some embodiments, the contiguous nucleotide sequence comprises SEQ ID NO: 4 to SEQ ID NO: 21 with one, two, three, or four mismatches. In other embodiments, the contiguous nucleotide sequence comprises SEQ ID NO: 4 to SEQ ID NO: 21. In certain embodiments, the contiguous nucleotide sequence comprises SEQ ID NO: 15 or SEQ ID NO: 7.

In some embodiments, the antisense oligonucleotide of the present disclosure is capable of inhibiting the expression of the human SNCA transcript in a cell which is expressing the human SNCA transcript.

In some embodiments, the contiguous nucleotide sequence comprises at least one nucleotide analogue. In some embodiments, the antisense oligonucleotide is a gapmer. In other embodiments, the antisense oligonucleotide is an alternating flank gapmer.

In some embodiments, the antisense oligonucleotide as disclosed herein comprises the formula of 5′-A-B-C-3′, wherein, (i) region B is a contiguous sequence of at least 6 DNA units, which are capable of recruiting RNase; (ii) region A is a first wing sequence of 1 to 10 nucleotides, wherein the first wing sequence comprises one or more nucleotide analogues and optionally one or more DNA units and wherein at least one of the nucleotide analogues is located at the 3′ end of A; and (iii) region C is a second wing sequence of 1 to 10 nucleotides, wherein the second wing sequence comprises one or more nucleotide analogues and optionally one or more DNA units and wherein at least one of the nucleotide analogues is located at the 5′ end of C.

In some embodiments, region A comprises a combination of nucleotide analogues and DNA unit selected from (i) 1-10 nucleotide analogs and 0 DNA unit; (ii) 1-9 nucleotide analogues and 1 DNA unit; (iii) 1-8 nucleotide analogues and 1-2 DNA units; (iv) 1-7 nucleotide analogues and 1-3 DNA units; (v) 1-6 nucleotide analogues and 1-4 DNA units; (vi) 1-5 nucleotide analogues and 1-5 DNA units; (vii) 1-4 nucleotide analogues and 1-6 DNA units; (viii) 1-3 nucleotide analogues and 1-7 DNA units; (ix) 1-2 nucleotide analogues and 1-8 DNA units; and (x) 1 nucleotide analogue and 1-9 DNA units. In some embodiments, region C comprises a combination of nucleotide analogues and DNA unit selected from (i) 1-10 nucleotide analogs and 0 DNA unit; (ii) 1-9 nucleotide analogues and 1 DNA unit; (iii) 1-8 nucleotide analogues and 1-2 DNA units; (iv) 1-7 nucleotide analogues and 1-3 DNA units; (v) 1-6 nucleotide analogues and 1-4 DNA units; (vi) 1-5 nucleotide analogues and 1-5 DNA units; (vii) 1-4 nucleotide analogues and 1-6 DNA units; (viii) 1-3 nucleotide analogues and 1-7 DNA units; (ix) 1-2 nucleotide analogues and 1-8 DNA units; and (x) 1 nucleotide analogue and 1-9 DNA units. In some embodiments, region A is a first wing design selected from any ASOs in FIGS. 2 and 3 and/or region C is a second wing design selected from any ASOs in FIGS. 2 and 3, wherein the upper letter is a nucleoside analog and the lower letter is a DNA.

In some embodiments, the antisense oligonucleotide of the present disclosure comprises at least two, at least three, at least four, at least five, at least six, at least seven, at least eight, at least nine, or at least ten nucleotide analogues. In certain embodiments, the nucleotide analogue or analogues are one or more sugar modified nucleosides selected from the group consisting of Locked Nucleic Acid (LNA); 2′-O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleoside (cEt), 2′-O-methyl nucleic acid (2′-OMe), 2′-O-methoxyethyl nucleic acid (2′-MOE), and any combination thereof. In some embodiments, the nucleotide analogue or analogues comprise a bicyclic sugar. In certain embodiments, the bicyclic sugar comprises cEt, 2′,4′-constrained 2′-O-methoxyethyl (cMOE), LNA, α-L-LNA, β-D-LNA, 2′-0,4′-C-ethylene-bridged nucleic acids (ENA), amino-LNA, oxy-LNA, or thio-LNA. In some embodiments, the nucleotide analogue or analogues comprise an LNA.

In some embodiments, the antisense oligonucleotide comprises one or more 5′ methyl cytosine nucleobases. In some embodiments, the antisense oligonucleotide comprises two to five LNAs on the 5′ region of the antisense oligonucleotide. In some embodiments, the antisense oligonucleotide comprises two to five LNAs on the 3′ region of the antisense oligonucleotide.

In some embodiments, the antisense oligonucleotide has an in vivo tolerability less than or equal to a total score of 4, wherein the total score is the sum of a unit score of five categories, which are 1) hyperactivity; 2) decreased activity and arousal; 3) motor dysfunction and/or ataxia; 4) abnormal posture and breathing; and 5) tremor and/or convulsions, and wherein the unit score for each category is measured on a scale of 0-4. In certain embodiments, the in vivo tolerability is less than or equal to the total score of 3, the total score of 2, the total score of 1, or the total score of 0.

In some embodiments, the antisense oligonucleotide reduces expression of SNCA mRNA in a cell by at least about 20%, at least about 30%, at least about 40%, at least about 50%, at least about 60%, at least about 70%, at least about 80%, at least about 90%, or about 100% compared to a cell not exposed to the antisense oligonucleotide. In other embodiments, the antisense oligonucleotide reduces expression of SNCA protein in a cell by at least about 60%, at least about 70%, at least about 80%, at least about 90%, or at least about 95% compared to a cell not exposed to the antisense oligonucleotide.

In some embodiments, the antisense oligonucleotide comprises the nucleotides A, T, C, and G and at least one analogue of the nucleotides A, T, C, and G, and has a sequence score greater than or equal to 0.2, wherein the sequence score is calculated by formula I: # of C nucleotides and analogues thereof—# of G nucleotides and analogues thereof (I) Total nucleotide length.

In some embodiments, the antisense oligonucleotide has from 10 to 24 nucleotides in length or from 14 to 21 nucleotides in length. In other embodiments, the antisense oligonucleotide has 14, 15, 16, 17, 18, 19, 20, or 21 nucleotides in length.

In some embodiments, the nucleotide sequence of the antisense oligonucleotide comprises, consists essentially of, or consists of a sequence selected from the group consisting of SEQ ID NOs: 4 to 21 with a design selected from the group consisting of the designs in FIGS. 2 and 3, wherein the upper case letter is a sugar modified nucleoside and the lower case letter is DNA. In certain embodiments, the nucleotide sequence comprises, consists essentially of, or consists of SEQ ID NO: 15 with the design of ASO-005459 and SEQ ID NO: 7 with the design of ASO-005578 or ASO-005584. In other embodiments, the nucleotide sequence comprises, consists essentially of, or consists of a sequence selected from the group consisting of SEQ ID NOs: 4-21 with the corresponding chemical structure in FIGS. 2 and 3. In certain embodiments, the nucleotide sequence comprises, consists essentially of, or consists of ASO-005459, ASO-005578, and ASO-005584. In some embodiments, the ASO of the present disclosure comprises a molecular formula of C₁₇₁H₂₁₄N₅₆O₉₀P₁₆S₁₆. In some embodiments, the ASO comprises a molecular formula of C₁₈₂H₂₂₉N₅₈O₉₆P₁₇S₁₇. In other embodiments, the ASO comprises a molecular formula of C₁₈₁H₂₂₇N₅₈O₉₆P₁₇S₁₇.

In some embodiments, the antisense oligonucleotide of the present disclosure comprises an internucleoside linkage selected from: a phosphodiester linkage, a phosphotriester linkage, a methylphosphonate linkage, a phosphoramidate linkage, a phosphorothioate linkage, and combinations thereof. In certain embodiments, the internucleoside linkage comprises one or more stereo-defined, modified phosphate linkages.

The present disclosure also provides a conjugate comprising the antisense oligonucleotide as disclosed herein, wherein the antisense oligonucleotide is covalently attached to at least one non-nucleotide or non-polynucleotide moiety. In some embodiments, the non-nucleotide or non-polynucleotide moiety comprises a protein, a fatty acid chain, a sugar residue, a glycoprotein, a polymer, or any combinations thereof.

Also provided herein is a pharmaceutical composition comprising the antisense oligonucleotide or the conjugate as disclosed herein and a pharmaceutically acceptable carrier. In some embodiments, the composition further comprises a therapeutic agent. In certain embodiments, the therapeutic agent is an alpha-synuclein antagonist. In some embodiments, the alpha-synuclein antagonist is an anti-alpha-synuclein antibody or fragment thereof.

The present disclosure further provides a kit comprising the antisense oligonucleotide, the conjugate, or the composition as disclosed herein. Also disclosed is a diagnostic kit comprising the antisense oligonucleotide, the conjugate, or the composition of the present disclosure.

The present disclosure is also directed to method of inhibiting or reducing SNCA protein expression in a cell, the method comprising administering the antisense oligonucleotide, the conjugate, or the composition as disclosed herein to the cell expressing SNCA protein, wherein the SNCA protein expression in the cell is inhibited or reduced after the administration. In some embodiments, the antisense oligonucleotide inhibits or reduces expression of SNCA mRNA in the cell after the administration. In certain embodiments, the expression of SNCA mRNA is reduced by at least about 20%, at least about 30%, at least about 40%, at least about 50%, at least about 60%, at least about 70%, at least about 80%, at least about 90%, or about 100% after the administration compared to a cell not exposed to the antisense oligonucleotide. In other embodiments, the antisense oligonucleotide reduces expression of SNCA protein in the cell after the administration by at least about 60%, at least about 70%, at least about 80%, or at least about 90% compared to a cell not exposed to the antisense oligonucleotide. In some embodiments, the cell is a neuron.

Provided herein is a method for treating a synucleinopathy in a subject in need thereof, comprising administering an effective amount of the antisense oligonucleotide, the conjugate, or the composition of the present disclosure. In some embodiments, the synucleinopathy is selected from the group consisting of Parkinson's disease, Parkinson's Disease Dementia (PDD), multiple system atrophy, dementia with Lewy bodies, and any combinations thereof.

Also provided herein is a use of the antisense oligonucleotide, the conjugate, or the composition of the present disclosure for the manufacture of a medicament. The present disclosure also provides the use of the antisense oligonucleotide, the conjugate, or the composition for the manufacture of a medicament for the treatment of a synucleinopathy in a subject in need thereof. In some embodiments, the antisense oligonucleotide, the conjugate, or the composition of the present disclosure is for use in therapy of a synucleinopathy in a subject in need thereof. In other embodiments, the antisense oligonucleotide, the conjugate, or the composition of the present disclosure is for use in therapy.

In some embodiments, the subject is a human. In some embodiments, the antisense oligonucleotide, the conjugate, or the composition is administered orally, parenterally, intrathecally, intra-cerebroventricularly, pulmonarily, topically, or intraventricularly.

In some embodiments, the nucleotide analog comprises a sugar modified nucleoside. In some embodiments, the sugar modified nucleoside is an affinity enhancing sugar modified nucleoside.

### BRIEF DESCRIPTION OF THE FIGURES

FIGS. 1A, 1, and 1C show alpha-synuclein genomic (partial), mRNA, and protein sequences. The sequence in FIG. 1A represents a partial SNCA genomic sequence (i.e., residues 6001 to 8400 of SEQ ID NO: 1). SEQ ID NO: 1 is identical to a SNCA pre-mRNA sequence except that the nucleotide “t” in SEQ ID NO: 1 is shown as “u” in the pre-mRNA. SEQ ID NO: 2 in FIG. 1B represents a SNCA mRNA sequence except that the nucleotide “t” in SEQ ID NO: 2 is shown as “u” in the mRNA. FIG. 1C shows SNCA protein sequence (SEQ ID NO: 3).

FIG. 2 shows exemplary ASOs targeting the intron exon junction of the SNCA pre-mRNA. Each column of FIG. 2 shows the Sequence ID number (SEQ ID No.) designated for the sequence only, the target start and end positions on the SNCA pre-mRNA sequence, the target start and end positions on the SNCA mRNA sequence, the design number (DES No.), the ASO sequence with a design, the ASO number (ASO No.), and the ASO sequence with a chemical structure.

FIG. 3 shows ASOs targeting the intron exon junction of the SNCA pre-mRNA with exemplary wing design modification. Each column of FIG. 3 shows the Sequence ID number (SEQ ID No.) designated for the sequence only, the target start and end positions on the SNCA pre-mRNA sequence, the design number (DES No.), the ASO sequence with a design, the ASO number (ASO No.), and the ASO sequence with a chemical structure and wing design modification identified. DES-316392, DES-316393, DES-316394, and DES-316395 show various ASO designs possible for SEQ ID NO: 7. DES-287031, DES-287957, DES-287959, DES-287962, DES-288906, and DES-313413 show various ASO designs possible for SEQ ID NO: 12. DES-319536, DES-319537, DES-325058, DES-325059, DES-325060, DES-325061, DES-325062, DES-325063, DES-325064, DES-325065, DES-325066, DES-325067, DES-325068, and DES-325069 show various ASO designs possible for SEQ ID NO: 14. DES-324636, DES-324637, DES-324638, DES-324639, DES-324640, DES-324641, DES-324642, DES-324643, DES-324644, DES-324645, DES-324646, and DES324647 show various ASO designs possible for SEQ ID NO: 15. For the ASO designs, the upper letters indicate nucleotide analogues (e.g., LNA or 2′-O-Methyl (OMe)), and the lower letters indicate DNAs. The upper letters with or without underlines indicate the two letters can be different nucleotide analogues, e.g., LNA and 2′-O-Methyl. For example, the underlined upper letters can be 2′-O-Methyl while the upper letters without underlines are LNA. In the ASOs with chemical structure column, OMe is 2′-O-Methyl nucleotide, L is LNA, D is DNA, and the numbers followed by L or D mean the number of LNAs or DNAs

FIG. 4 shows the percent reduction of SNCA protein expression in both a human neuroblastoma cell line SK-N-BE(2) (“SK cells”) and primary neurons isolated from A53T-PAC transgenic mice (“PAC neurons”) after in vitro culture with various ASOs as described in Example 2A for PAC neurons and Example 2E for SK cells. For the SK cells, the cells were treated with 25 μM of ASO and the SNCA mRNA expression (normalized to GAPDH) is shown as a percent of the control. For the PAC neurons, the cells were treated with either 40 nM or 5 μM of ASO and the SNCA protein expression (normalized to tubulin) is shown as percent inhibition. Where no value is provided, the particular ASO was not tested under the particular conditions.

FIG. 5 shows the potency of the various ASOs in reducing SNCA protein expression in primary neurons isolated from A53T-PAC transgenic mice in vitro. As described in Example 2A, the PAC neurons were cultured in vitro with a 10-point titration of the different ASOs and the potency (IC₅₀) of the ASOs is shown as a ratio of SNCA to tubulin expression (μM).

FIG. 6 shows the tolerability score (“Tox Score”) and the percent reduction (or knockdown, “KD”) of both the SNCA mRNA and SNCA protein expression in ASO-treated A53T-PAC transgenic or WT (wild-type) mice (see Example 4). The tolerability scores are provided for days 1 (1D) and 28 (28D) post ASO administration. The percent reduction in SNCA mRNA and SNCA protein expression is shown for days 3 and 28 post ASO administration in the hippocampus (Hippo), brain stem (BS), and striatum (Str).

FIGS. 7A and 7B show the effect of ASO-005459 on SNCA and tubulin (Tub) protein expression in primary neurons isolated from A53T-PAC transgenic mice. Neurons were treated with a 10-point titration of ASO-005459 and amounts of SNCA and tubulin protein measured. Percent inhibition of α-Syn/Tub ratio (FIG. 7A) and Tub levels (FIG. 7B) are shown. Each data point represents an individual replicate.

FIG. 8 shows the effect of ASO-005459 on the expression level of SNCA (circle), Protein S (alpha) (PROS1 (square)), and Tubulin (TUBB3 (triangle)) mRNAs in human neurons. The neurons were treated with various concentrations of ASO-005459 for 6 days, and then, the mRNA levels were measured by QUANTIGENE® assay. The expression level of the mRNAs is shown as a percent of the control. Data shown represents the mean±SD from duplicate determinations.

FIG. 9 shows the SNCA mRNA expression levels in the hippocampus of A53T-PAC mice at three days post ICV administration of 100 μg of ASO-005459 (open square) or control vehicle (closed circle). SNCA mRNA expression levels were measured by qRT-PCR, normalized to GAPDH mRNA, and then expressed relative to the mean expression level of the vehicle group. The horizontal line marks the reference value of 1 (i.e., value at which the SNCA mRNA expression would be equivalent to expression level observed in the vehicle control group). Data shown represents the mean±SD from duplicate determination. Statistics shown is based on 1-way ANOVA with Dunnett's post-test. ***p<0.001.

FIGS. 10A and 10B show a comparison of the average body weight of mice treated with ASO-005459. In FIG. 10A, the A53T-PAC mice were dosed with 3.13 μg, 12.5 μg, 25 μg, or 50 μg of ASO-005459 and their bodyweights measured at weeks 0, 1, and 2 post-treatment. In FIG. 10B, C57BL/6 mice were dosed with 100 μg of ASO-005459 and the animals' body weight was measured once a week during a course of 28 days. In both FIGS. 10A and 10B, animals receiving the vehicle control were used as controls. Data shown represents the mean±SD from multiple animals (n=5). Statistics shown is based on 2-way ANOVA.

FIGS. 11A, 111B, and 11C show the SNCA mRNA expression levels in the hippocampus (FIG. 11A), brainstem (FIG. 11B), and striatum (FIG. 11C) of A53T-PAC mice at 14 days post ICV administration of ASO-005459 (3.13 μg, 12.5 μg, 25 μg, or 50 μg) or the vehicle control. SNCA mRNA levels were measured by qRT-PCR, normalized to GAPDH mRNA, and then expressed relative to the mean of the vehicle group. Data shown represents the mean±SD from multiple animals (n=5). Each circle represents an individual animal. The horizontal line marks the reference value of 1 (i.e., value at which the SNCA mRNA expression would be equivalent to expression level observed in the vehicle control group). Statistics shown is based on 1-way ANOVA with Dunnett's post-test. ***p<0.001.

FIG. 12 shows the level of ASO-005459 detected in the hippocampus (black), brainstem (light gray), and the striatum (dark gray) of A53T-PAC mice at 14 days post ASO-005459 treatment. The mice received 3.13 μg, 12.5 μg, 25 μg, or 50 μg of ASO-005459 via ICV administration. Data shown represents the mean±SD from multiple animals (n=5).

FIGS. 13A, 13B, 13C, and 13D show the relationship between ASO-005459 exposure levels and SNCA mRNA expression in the hippocampus (FIG. 13A), brainstem (FIG. 13B), and striatum (FIG. 13C) of A53T-PAC mice at fourteen days post ASO-005459 treatment. FIG. 13D shows the data from the hippocampus (circle), brainstem (square), and striatum (triangle) in combination. Each data point represents an individual animal. A four-parameter, nonlinear fit is shown for the hippocampus (FIG. 13A) and the brainstem (FIG. 13B).

FIGS. 14A and 14B show the dose response curve of the effect of ASO-005459 on SNCA mRNA expression level in A53T-PAC mice. The animals received (via ICV injection) 12.5 μg (circle), 25 μg (box), or 50 μg (triangle) of ASO-005459 and were sacrificed at 24 hours, 3 days, 4, 8, 12, 16, and 20 weeks post-dosing. SNCA mRNA expression levels both in the brain stem (FIG. 14A) and in the striatum (FIG. 14B) were assessed via qRT-PCR and then normalized to the vehicle control. The mean data is shown. The horizontal line marks the reference value of 100% (i.e., value at which the SNCA mRNA expression would be equivalent to expression level observed in the vehicle control group).

FIGS. 15A and 15B show the effect of ASO-005459 on SNCA mRNA expression level in A53T-PAC mice after 4 weeks post ASO-005459 administration. The animals received either the vehicle control or varying concentration s of ASO-005459 (12.5, 25, or 50 μg). The relative SNCA mRNA expression level (normalized to the vehicle control) are shown for both the brain stem (FIG. 15A) and the striatum (FIG. 15B). Each data point represents an individual animal. The mean±SD from multiple animals is also shown. Statistics shown is based on comparison to the vehicle group using 1-way ANOVA with Dunnett's correction for multiple comparisons. The horizontal line marks the reference value of 1 (i.e., value at which the SNCA mRNA expression would be equivalent to expression level observed in the vehicle control group). *p<0.05, **p<0.01, ***p<0.001, ****p<0.0001.

FIGS. 16A and 16B show the dose response curve of the effect of ASO-005459 on SNCA protein expression level in the brain tissues of A53T-PAC mice. The animals received (via ICV injection) 12.5 μg (circle), 25 μg (box), or 50 μg (triangle) of ASO-005459 and were sacrificed at 24 hours, 3 days, 4, 8, 12, 16, and 20 weeks post-dosing. SNCA proteins levels were measured both in the brain stem (FIG. 16A) and in the striatum (FIG. 16B) by ELISA and then normalized to the vehicle control. The mean data is shown. The horizontal line marks the reference value of 100% (i.e., value at which the SNCA mRNA expression would be equivalent to expression level observed in the vehicle control group).

FIGS. 17A and 17B show the effect of ASO-005459 on SNCA protein expression level in A53T-PAC mice at 8 weeks post ASO-005459 administration. The animals received either the vehicle control or varying concentration s of ASO-005459 (12.5, 25, or 50 μg). The relative SNCA protein expression levels (normalized to the vehicle control) are shown for both the brain stem (FIG. 17A) and the striatum (FIG. 17B). Each data point represents an individual animal. The horizontal line marks the reference value of 100% (i.e., value at which the SNCA mRNA expression would be equivalent to expression level observed in the vehicle control group). The mean±SD from multiple animals is also shown. Statistics shown is based on comparison to the vehicle group using 1-way ANOVA with Dunnett's correction for multiple comparisons.

FIGS. 18A and 18B show the effect of ASO-005578 and ASO-005584 on SNCA mRNA expression level in the brain tissues of cyno monkeys. In FIG. 18A, the animals received one of the following: vehicle control (circle), ASO-005578 (square, 4 mg), or ASO-005584 (triangle, 4 mg). Then, the animals were sacrificed at 2 weeks post-dosing. In FIG. 18B, the animals received either the vehicle control or ASO-005584 (8 mg per animal) and then were sacrificed at 2 weeks (square) and 4 weeks post dosing (triangle). In both FIGS. 18A and 18B, the SNCA mRNA expression level in the following tissues was assessed: medulla (top left panel), caudate putamen (top middle panel), pons (top right panel), cerebellum (bottom left panel), lumbar spinal cord (bottom middle panel), and frontal cortex (bottom right panel). The SNCA mRNA expression levels shown in FIGS. 18A and 18B were normalized to GAPDH and then shown relative to the mean of the control group (“vehicle”). Both the data for the individual animals and the mean are shown. The horizontal line marks the reference value of 100% (i.e., value at which the SNCA mRNA expression would be equivalent to expression level observed in the vehicle control group).

FIGS. 19A and 19B show the kinetics of SNCA mRNA and SNCA protein expression levels in cyno monkeys after ASO-005459 administration. Each of the animals received either the vehicle control or ASO-005459 (8 mg) and then were sacrificed at 24 hours, 3 days, 2, 4, 8, 13, or 20 weeks post-dosing. At each time point, the expression levels of SNCA mRNA (FIG. 19A) and SNCA protein (FIG. 19B) were assessed in the following tissues: medulla (top left panel), caudate putamen (top middle panel), pons (top right panel), cerebellum (bottom left panel), lumbar spinal cord (bottom middle panel), and frontal cortex (bottom right panel). The expression levels are shown as a percentage of the vehicle control. Both the data for the individual animals and the mean are shown. The horizontal line marks the reference value of 100% (i.e., value at which the SNCA mRNA expression would be equivalent to expression level observed in the vehicle control group).

FIGS. 20A and 20B show the relative expression level (as percentage of the vehicle control) for both the SNCA mRNA (FIG. 20A) and SNCA protein (FIG. 20B) in cyno monkeys at 2 weeks post ASO-005459 administration. The animals received either the vehicle control or varying concentrations of the ASO-005459 (2, 4, or 8 mg). The expression levels were assessed in the following tissues: medulla (top left panel), caudate putamen (top middle panel), pons (top right panel), cerebellum (bottom left panel), lumbar spinal cord (bottom middle panel), and frontal cortex (bottom right panel). The expression levels are shown as a percentage of the vehicle control. Both the data for the individual animals and the mean are shown. The horizontal line marks the reference value of 100% (i.e., value at which the SNCA mRNA expression would be equivalent to expression level observed in the vehicle control group).

FIG. 21A shows the contiguous nucleotide sequence of ASO-005459. OxyA, OxyT, and Oxy MC are adenine beta D-oxy-LNA, thymine beta D-oxy-LNA, and methyl cytosine beta D-oxy-LNA, respectively; DNAt, DNAc, and DNAa are thymine DNA, cytosine DNA, and adenine DNA, respectively; and s is a phosphorothioate linkage.

FIG. 21B shows the molecular structure of ASO-005459 as disclosed herein. The provided structure has a molecular formula of C₁₇₁H₂₁₄N₅₆O₉₀P₁₆Si₆ and each of the M+ is a pharmaceutically acceptable counterion such as H⁺, Na⁺, or NH₄⁺.

### DETAILED DESCRIPTION OF DISCLOSURE

### I. Definitions

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). For example, if it is stated that “the ASO reduces expression of SNCA protein in a cell following administration of the ASO by at least about 60%,” it is implied that the SNCA levels are reduced by a range of 50% to 70%.

The term “antisense oligonucleotide” (ASO) refers to an oligomer or polymer of nucleosides, such as naturally-occurring nucleosides or modified forms thereof, that are covalently linked to each other through internucleotide linkages. The ASO useful for the disclosure includes at least one non-naturally occurring nucleoside. An ASO is complementary to a target nucleic acid, such that the ASO hybridizes to the target nucleic acid sequence. The terms “antisense ASO,” “ASO,” and “oligomer” as used herein are interchangeable with the term “ASO.”

The term “nucleic acids” or “nucleotides” is intended to encompass plural nucleic acids. In some embodiments, the term “nucleic acids” or “nucleotides” refers to a target sequence, e.g., pre-mRNAs, mRNAs, or DNAs in vivo or in vitro. When the term refers to the nucleic acids or nucleotides in a target sequence, the nucleic acids or nucleotides can be naturally occurring sequences within a cell. In other embodiments, “nucleic acids” or “nucleotides” refer to a sequence in the ASOs of the disclosure. When the term refers to a sequence in the ASOs, the nucleic acids or nucleotides are not naturally occurring, i.e., chemically synthesized, enzymatically produced, recombinantly produced, or any combination thereof. In one embodiment, the nucleic acids or nucleotides in the ASOs are produced synthetically or recombinantly, but are not a naturally occurring sequence or a fragment thereof. In another embodiment, the nucleic acids or nucleotides in the ASOs are not naturally occurring because they contain at least one nucleotide analog that is not naturally occurring in nature. The term “nucleic acid” or “nucleoside” refers to a single nucleic acid segment, e.g., a DNA, an RNA, or an analog thereof, present in a polynucleotide. “Nucleic acid” or “nucleoside” includes naturally occurring nucleic acids or non-naturally occurring nucleic acids. In some embodiments, the terms “nucleotide”, “unit” and “monomer” are used interchangeably. It will be recognized that when referring to a sequence of nucleotides or monomers, what is referred to is the sequence of bases, such as A, T, G, C or U, and analogs thereof.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base, which are also referred to as “nucleotide analogs” herein. Herein, a single nucleotide (unit) can also be referred to as a monomer or nucleic acid unit. In certain embodiments, the term “nucleotide analogs” refers to nucleotides having modified sugar moieties. Non-limiting examples of the nucleotides having modified sugar moieties (e.g., LNA) are disclosed elsewhere herein. In other embodiments, the term “nucleotide analogs” refers to nucleotides having modified nucleobase moieties. The nucleotides having modified nucleobase moieties include, but are not limited to, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

The term “nucleoside” as used herein is used to refer to a glycoside comprising a sugar moiety and a base moiety, which can be covalently linked by the internucleotide linkages between the nucleosides of the ASO. In the field of biotechnology, the term “nucleoside” is often used to refer to a nucleic acid monomer or unit. In the context of an ASO, the term “nucleoside” can refer to the base alone, i.e., a nucleobase sequence comprising cytosine (DNA and RNA), guanine (DNA and RNA), adenine (DNA and RNA), thymine (DNA) and uracil (RNA), in which the presence of the sugar backbone and internucleotide linkages are implicit. Likewise, particularly in the case of oligonucleotides where one or more of the internucleotide linkage groups are modified, the term “nucleotide” can refer to a “nucleoside.” For example the term “nucleotide” can be used, even when specifying the presence or nature of the linkages between the nucleosides.

The term “nucleotide length” as used herein means the total number of the nucleotides (monomers) in a given sequence. For example, the sequence of AtTcctttacaccACAC (SEQ ID NO: 15) has 17 nucleotides; thus the nucleotide length of the sequence is 17. The term “nucleotide length” is therefore used herein interchangeably with “nucleotide number.”

As one of ordinary skill in the art would recognize, the 5′ terminal nucleotide of an oligonucleotide does not comprise a 5′ internucleotide linkage group, although it can comprise a 5′ terminal group.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, untranslated regions (“UTRs”), and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The term “non-coding region” as used herein means a nucleotide sequence that is not a coding region. Examples of non-coding regions include, but are not limited to, promoters, ribosome binding sites, transcriptional terminators, introns, untranslated regions (“UTRs”), non-coding exons and the like. Some of the exons can be wholly or part of the 5′ untranslated region (5′ UTR) or the 3′ untranslated region (3′ UTR) of each transcript. The untranslated regions are important for efficient translation of the transcript and for controlling the rate of translation and half-life of the transcript.

The term “region” when used in the context of a nucleotide sequence refers to a section of that sequence. For example, the phrase “region within a nucleotide sequence” or “region within the complement of a nucleotide sequence” refers to a sequence shorter than the nucleotide sequence, but longer than at least 10 nucleotides located within the particular nucleotide sequence or the complement of the nucleotides sequence, respectively. The term “sub-sequence” or “subsequence” can also refer to a region of a nucleotide sequence.

The term “downstream,” when referring to a nucleotide sequence, means that a nucleic acid or a nucleotide sequence is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence.

Unless otherwise indicated, the sequences provided herein are listed from 5′ end (left) to 3′ end (right).

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, UTRs, and stem-loop structures. If a coding region is intended for expression in a eukaryotic cell, a polyadenylation signal and transcription termination sequence will usually be located 3′ to the coding sequence.

The term “transcript” as used herein can refer to a primary transcript that is synthesized by transcription of DNA and becomes a messenger RNA (mRNA) after processing, i.e., a precursor messenger RNA (pre-mRNA), and the processed mRNA itself. The term “transcript” can be interchangeably used with “pre-mRNA” and “mRNA.” After DNA strands are transcribed to primary transcripts, the newly synthesized primary transcripts are modified in several ways to be converted to their mature, functional forms such as mRNA, tRNA, rRNA, lncRNA, miRNA and others. Thus, the term “transcript” can include exons, introns, 5′ UTRs, and 3′ UTRs.

The term “expression” as used herein refers to a process by which a polynucleotide produces a gene product, for example, a RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

The terms “identical” or percent “identity” in the context of two or more nucleic acids refer to two or more sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, California) or Megalign (DNASTAR) are additional publicly available software programs that can be used to align sequences. In certain embodiments, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. One skilled in the art can determine appropriate parameters for maximal alignment by particular alignment software. In certain embodiments, the default parameters of the alignment software are used.

In certain embodiments, the percentage identity “X” of a first nucleotide sequence to a second nucleotide sequence is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Different regions within a single polynucleotide target sequence that align with a polynucleotide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

As used herein, the terms “homologous” and “homology” are interchangeable with the terms “identity” and “identical.”

The term “naturally occurring variant thereof” refers to variants of the SNCA polypeptide sequence or SNCA nucleic acid sequence (e.g., transcript) which exist naturally within the defined taxonomic group, such as mammalian, such as mouse, monkey, and human. Typically, when referring to “naturally occurring variants” of a polynucleotide the term also can encompass any allelic variant of the SNCA-encoding genomic DNA which is found at Chromosomal position 17q21 by chromosomal translocation or duplication, and the RNA, such as mRNA derived therefrom. “Naturally occurring variants” can also include variants derived from alternative splicing of the SNCA mRNA. When referenced to a specific polypeptide sequence, e.g., the term also includes naturally occurring forms of the protein, which can therefore be processed, e.g., by co- or post-translational modifications, such as signal peptide cleavage, proteolytic cleavage, glycosylation, etc.

In determining the degree of “complementarity” between ASOs of the disclosure (or regions thereof) and the target region of the nucleic acid which encodes mammalian SNCA protein (e.g., the SNCA gene), such as those disclosed herein, the degree of “complementarity” (also, “homology” or “identity”) is expressed as the percentage identity (or percentage homology) between the sequence of the ASO (or region thereof) and the sequence of the target region (or the reverse complement of the target region) that best aligns therewith. The percentage is calculated by counting the number of aligned bases that are identical between the two sequences, dividing by the total number of contiguous monomers in the ASO, and multiplying by 100. In such a comparison, if gaps exist, it is preferable that such gaps are merely mismatches rather than areas where the number of monomers within the gap differs between the ASO of the disclosure and the target region.

The term “complement” as used herein indicates a sequence that is complementary to a reference sequence. It is well known that complementarity is the base principle of DNA replication and transcription as it is a property shared between two DNA or RNA sequences, such that when they are aligned antiparallel to each other, the nucleotide bases at each position in the sequences will be complementary, much like looking in the mirror and seeing the reverse of things. Therefore, for example, the complement of a sequence of 5′ “ATGC”3′ can be written as 3′ “TACG”5′ or 5′ “GCAT”3′. The terms “reverse complement”, “reverse complementary” and “reverse complementarity” as used herein are interchangeable with the terms “complement”, “complementary” and “complementarity.”

The terms “corresponding to” and “corresponds to,” when referencing two separate nucleic acid or nucleotide sequences can be used to clarify regions of the sequences that correspond or are similar to each other based on homology and/or functionality, although the nucleotides of the specific sequences can be numbered differently. For example, different isoforms of a gene transcript can have similar or conserved portions of nucleotide sequences whose numbering can differ in the respective isoforms based on alternative splicing and/or other modifications. In addition, it is recognized that different numbering systems can be employed when characterizing a nucleic acid or nucleotide sequence (e.g., a gene transcript and whether to begin numbering the sequence from the translation start codon or to include the 5′UTR). Further, it is recognized that the nucleic acid or nucleotide sequence of different variants of a gene or gene transcript can vary. As used herein, however, the regions of the variants that share nucleic acid or nucleotide sequence homology and/or functionality are deemed to “correspond” to one another. For example, a nucleotide sequence of a SNCA transcript corresponding to nucleotides X to Y of SEQ ID NO: 1 (“reference sequence”) refers to an SNCA transcript sequence (e.g., SNCA pre-mRNA or mRNA) that has an identical sequence or a similar sequence to nucleotides X to Y of SEQ ID NO: 1. A person of ordinary skill in the art can identify the corresponding X and Y residues in the SNCA transcript sequence by aligning the SNCA transcript sequence with SEQ ID NO: 1.

The terms “corresponding nucleotide analog” and “corresponding nucleotide” are intended to indicate that the nucleobase in the nucleotide analog and the naturally occurring nucleotide have the same pairing, or hybridizing, ability. For example, when the 2-deoxyribose unit of the nucleotide is linked to an adenine, the “corresponding nucleotide analog” contains a pentose unit (different from 2-deoxyribose) linked to an adenine.

The term “DES Number” or “DES No.” as used herein refers to a unique number given to a nucleotide sequence having a specific pattern of nucleosides (e.g., DNA) and nucleoside analogs (e.g., LNA). As used herein, the design of an ASO is shown by a combination of upper case letters and lower case letters. For example, DES-005459 refers to an ASO sequence of AtTcctttacaccACAC (SEQ ID NO: 15) with an ASO design of LDLDDDDDDDDDDLLLL (i.e., AtTcctttacaccACAC), wherein the L (i.e., upper case letter) indicates a nucleoside analog (e.g., LNA) and the D (i.e., lower case letter) indicates a nucleoside (e.g., DNA).

The term “ASO Number” or “ASO No.” as used herein refers to a unique number given to a nucleotide sequence having the detailed chemical structure of the components, e.g., nucleosides (e.g., DNA), nucleoside analogs (e.g., beta-D-oxy-LNA), nucleobase (e.g., A, T, G, C, U, or MC), and backbone structure (e.g., phosphorothioate or phosphodiester). For example, ASO-005459 refers to OxyAs DNAts OxyTs DNAcs DNAcs DNAts DNAts DNAts DNAas DNAcs DNAas DNAcs DNAcs OxyAs OxyMCs OxyAs OxyMC.

“Potency” is normally expressed as an IC₅₀ or EC₅₀ value, in μM, nM or pM unless otherwise stated. Potency can also be expressed in terms of percent inhibition. IC₅₀ is the median inhibitory concentration of a therapeutic molecule. EC₅₀ is the median effective concentration of a therapeutic molecule relative to a vehicle or control (e.g., saline). In functional assays, IC₅₀ is the concentration of a therapeutic molecule that reduces a biological response, e.g., transcription of mRNA or protein expression, by 50% of the biological response that is achieved by the therapeutic molecule. In functional assays, EC₅₀ is the concentration of a therapeutic molecule that produces 50% of the biological response, e.g., transcription of mRNA or protein expression. IC₅₀ or EC₅₀ can be calculated by any number of means known in the art.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

An “effective amount” of an ASO as disclosed herein is an amount sufficient to carry out a specifically stated purpose. An “effective amount” can be determined empirically and in a routine manner, in relation to the stated purpose.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for a disease or condition disclosed elsewhere herein according to the methods provided herein if the patient shows, e.g., total, partial, or transient alleviation or elimination of symptoms associated with the disease or disorder.

### II. Antisense Oligonucleotides

The present disclosure employs antisense oligonucleotides for use in modulating the function of nucleic acid molecules encoding mammalian α-Syn, such as the SNCA nucleic acid, e.g., SNCA transcript, including SNCA pre-mRNA, and SNCA mRNA, or naturally occurring variants of such nucleic acid molecules encoding mammalian α-Syn. The term “ASO” in the context of the present disclosure, refers to a molecule formed by covalent linkage of two or more nucleotides (i.e., an oligonucleotide).

The ASO comprises a contiguous nucleotide sequence of from about 10 to about 30, such as 10-20, 16-20, or 15-25 nucleotides in length. The terms “antisense ASO,” “antisense oligonucleotide,” and “oligomer” as used herein are interchangeable with the term “ASO.”

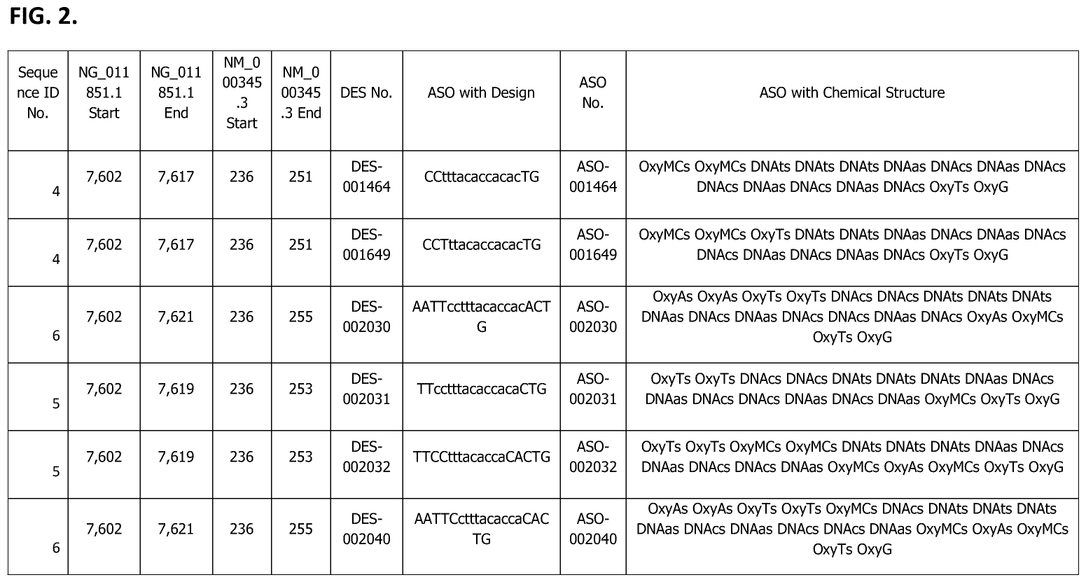

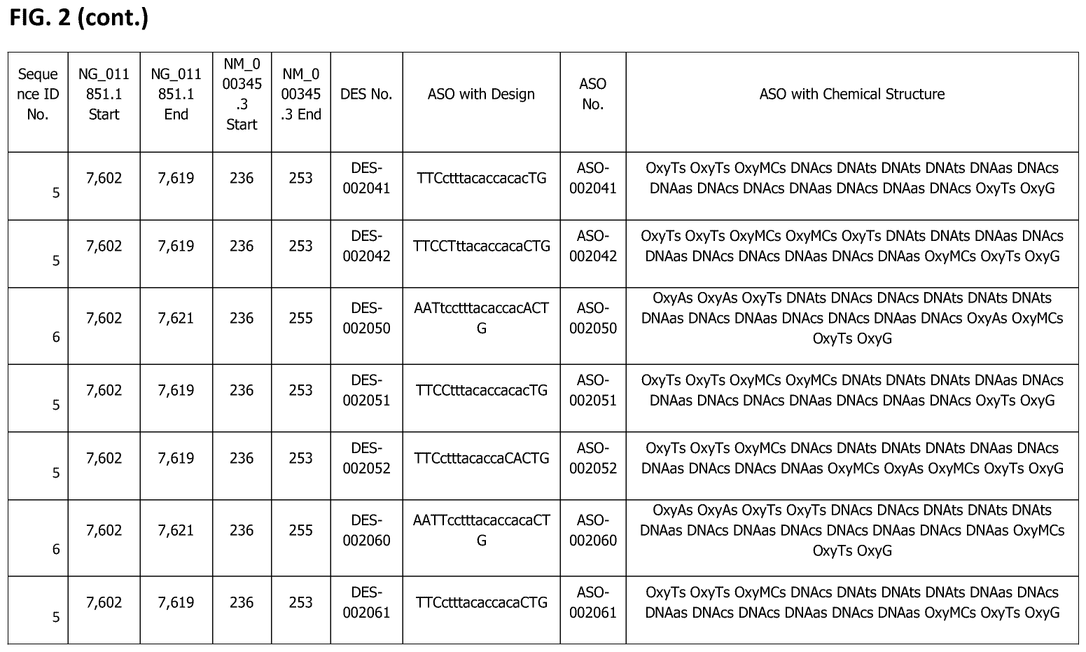

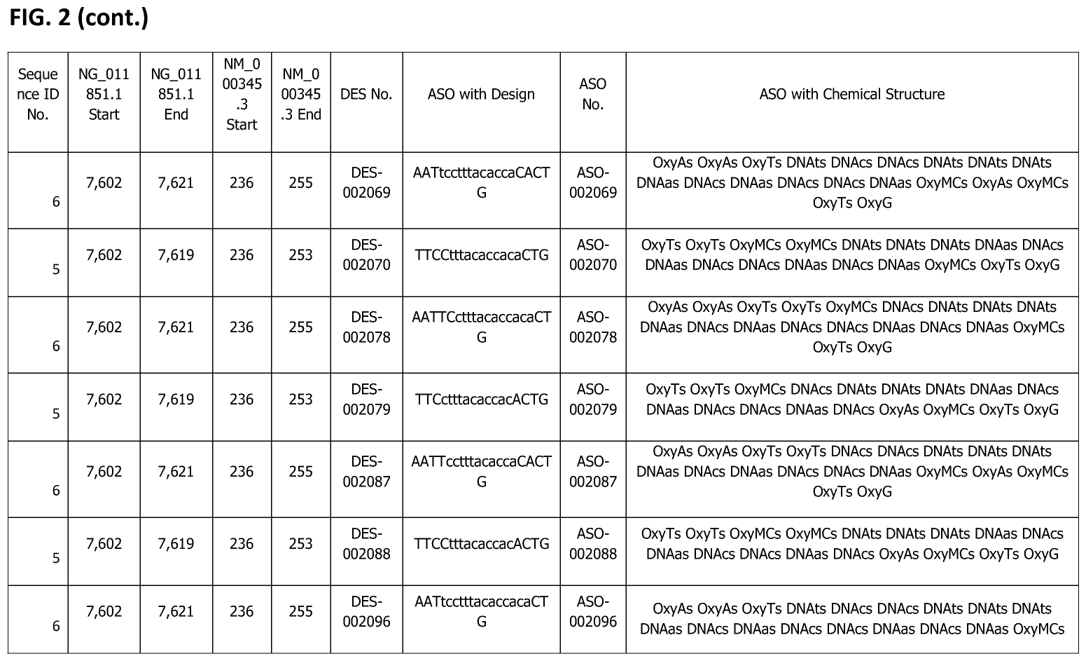

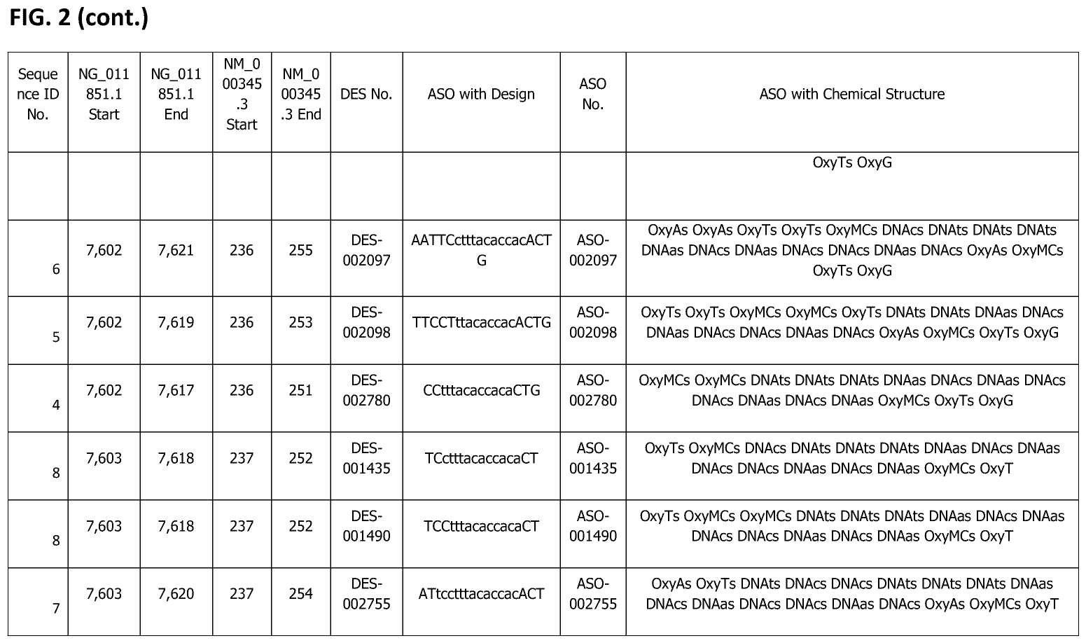

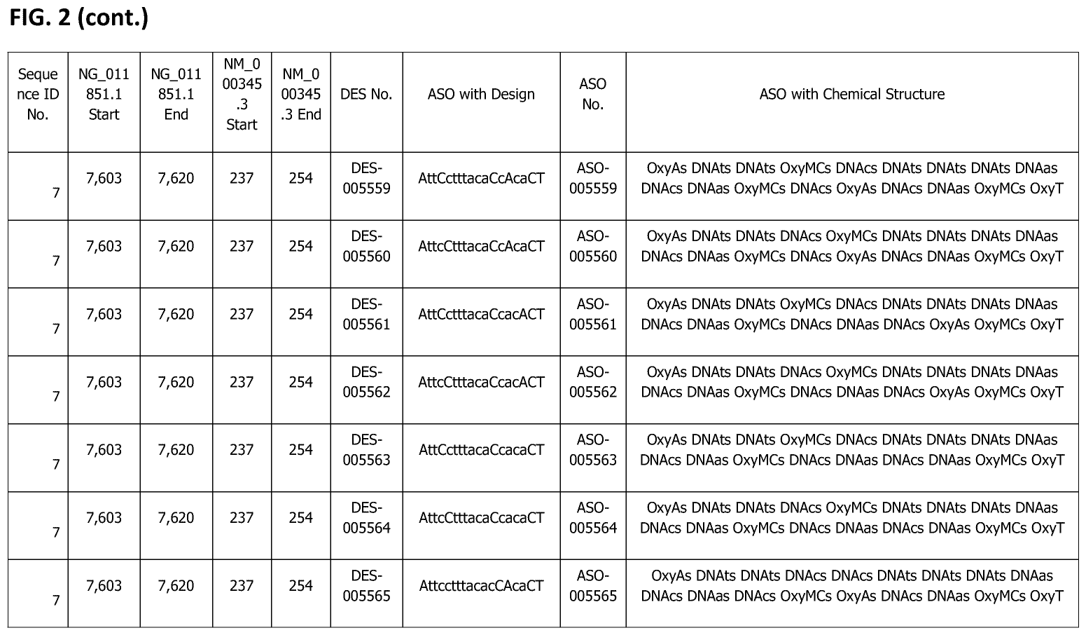

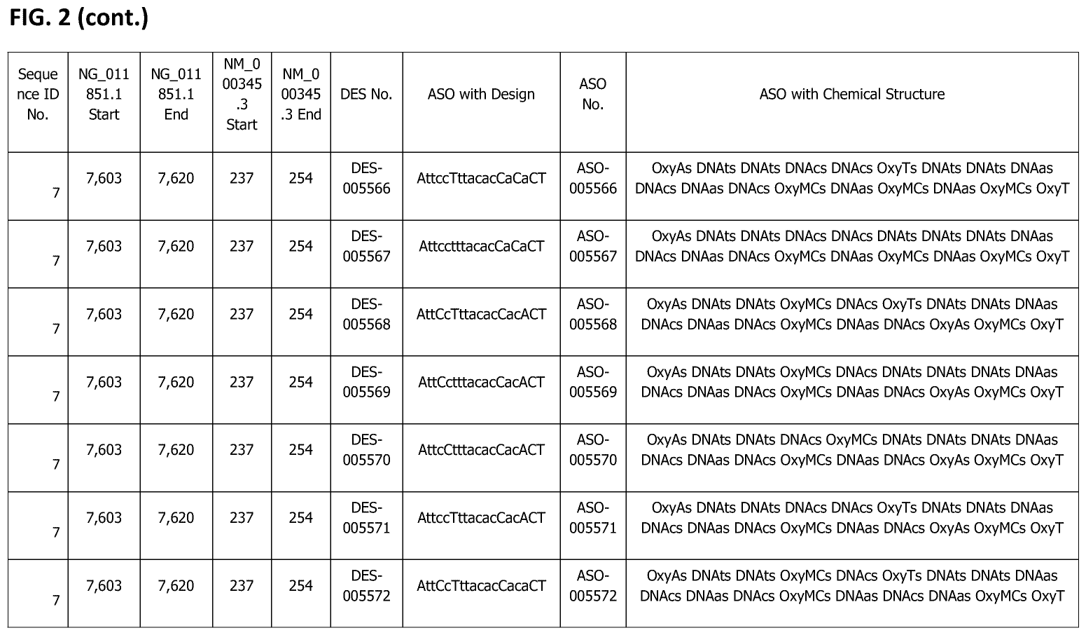

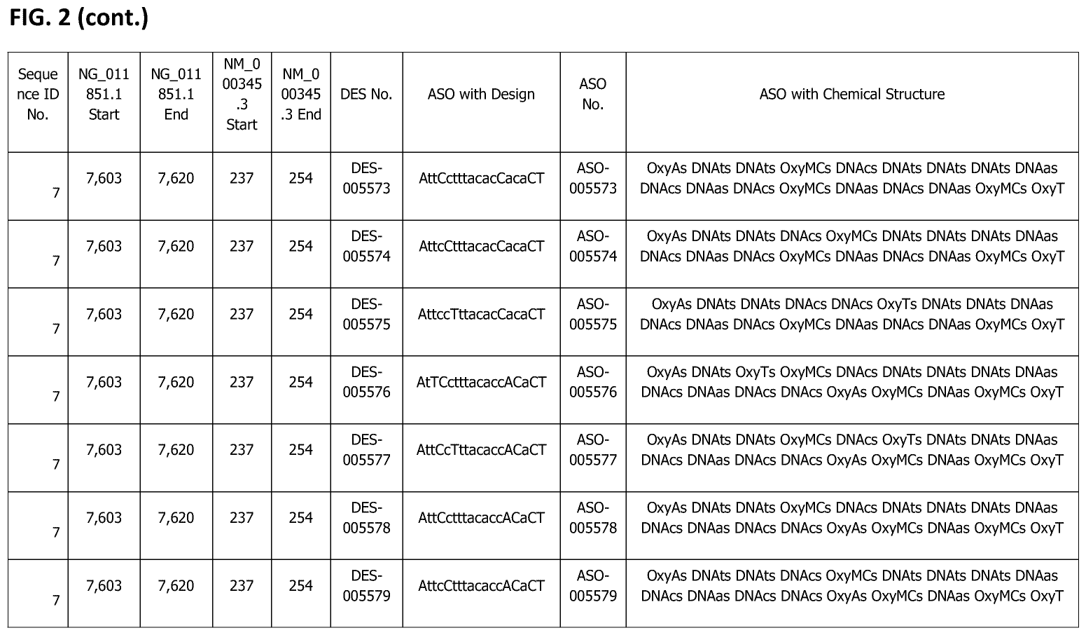

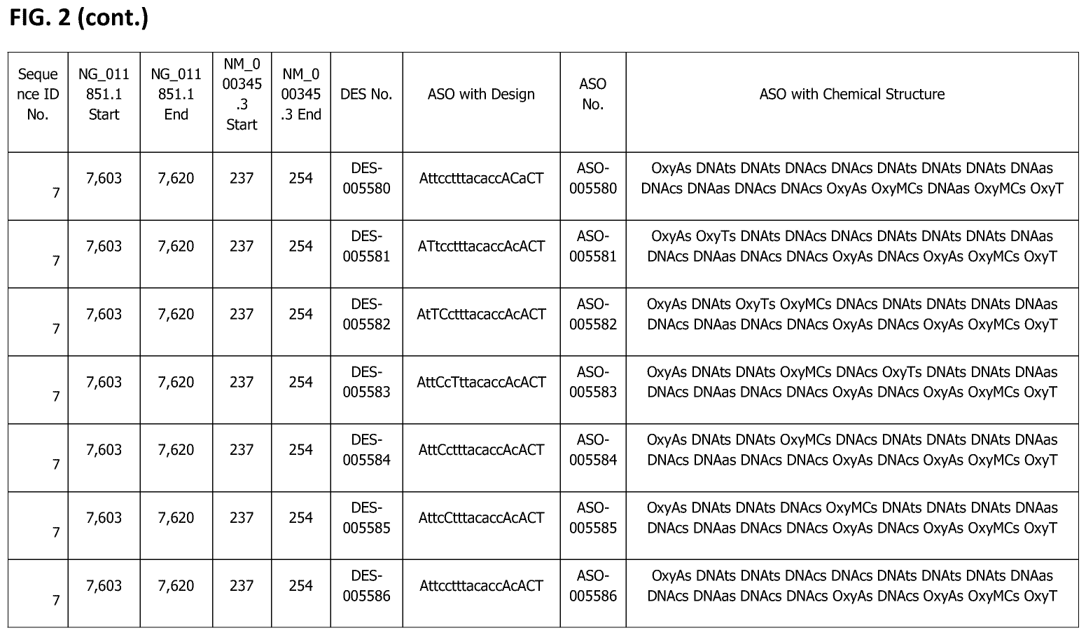

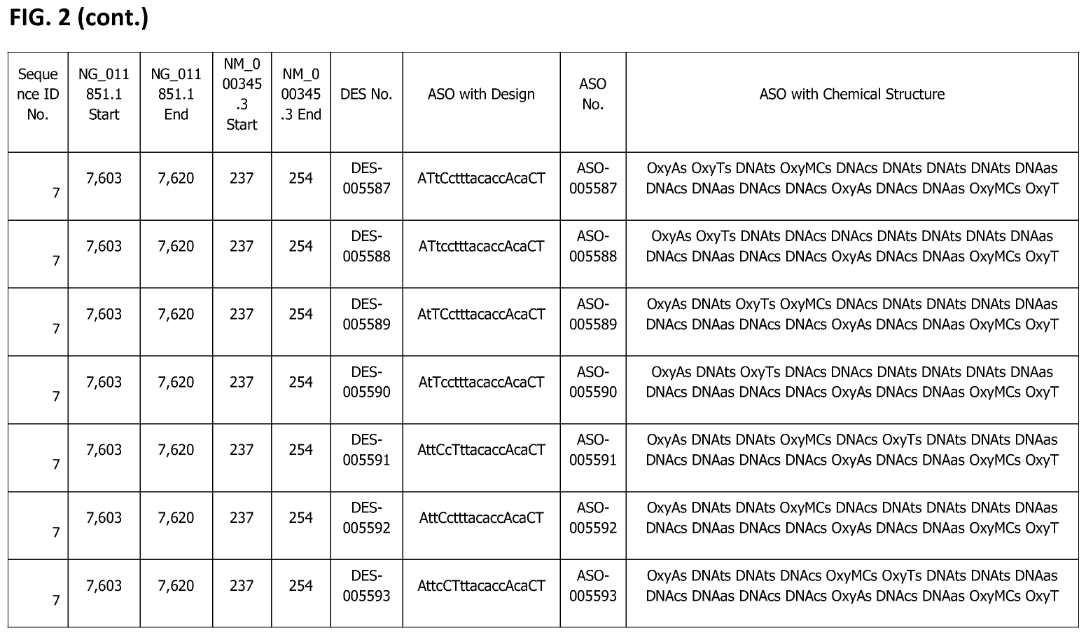

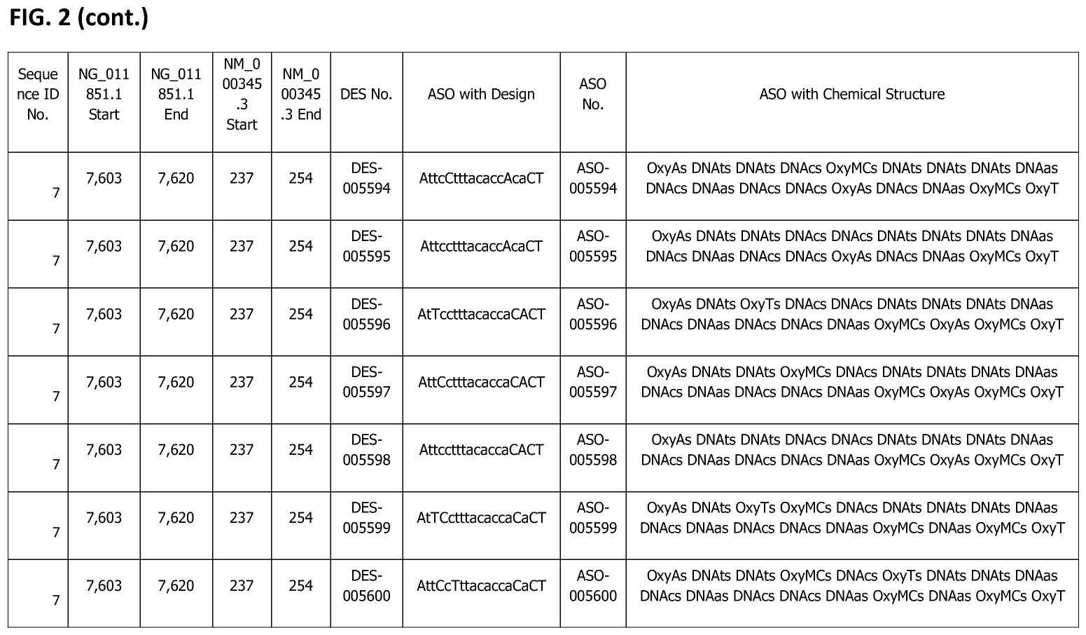

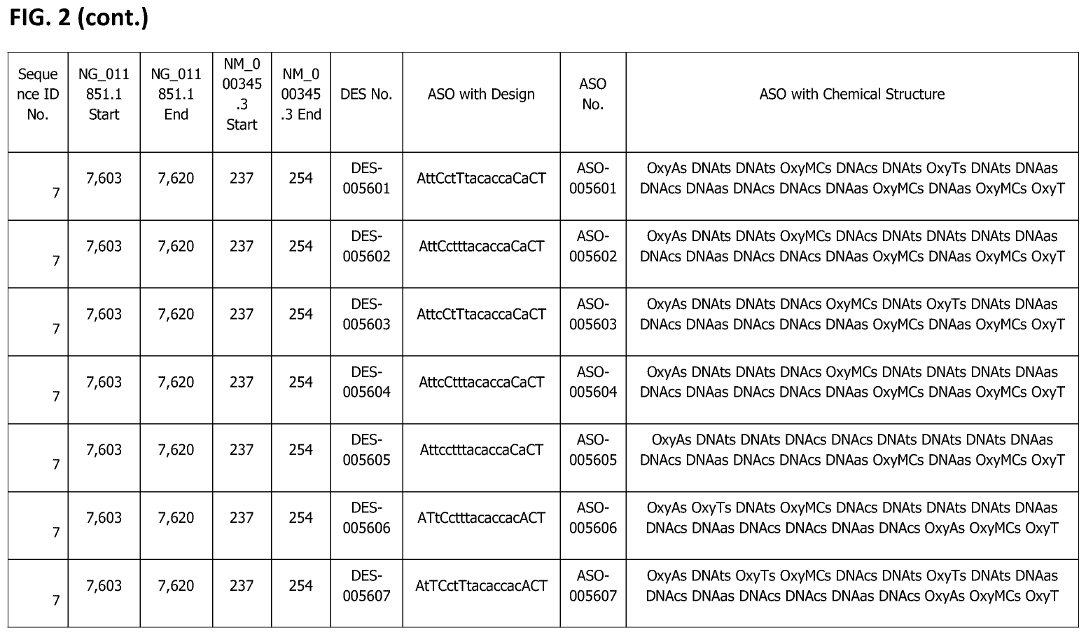

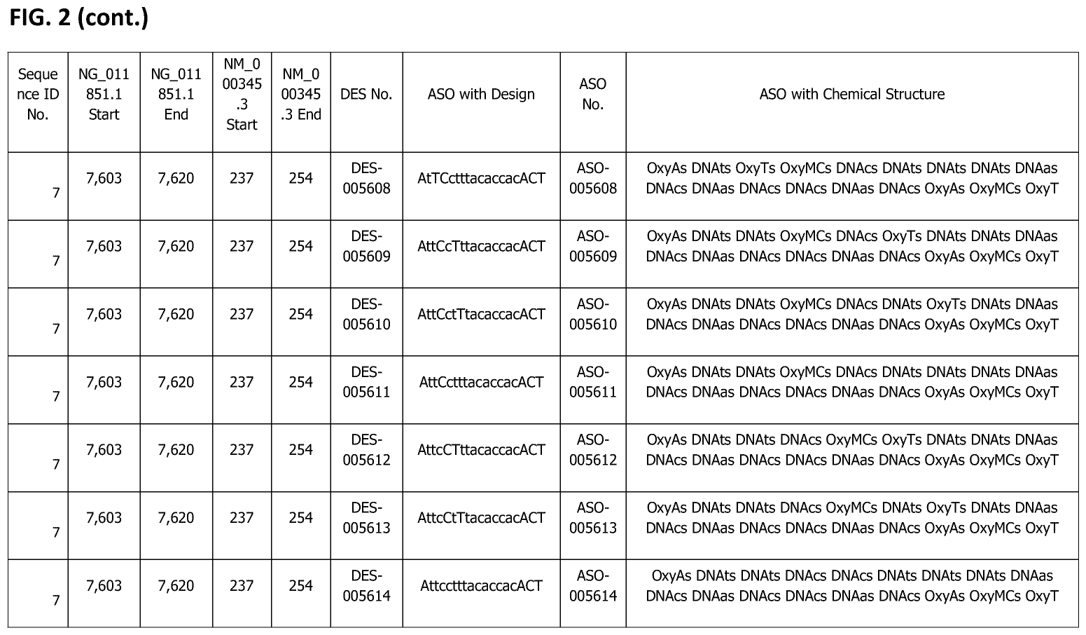

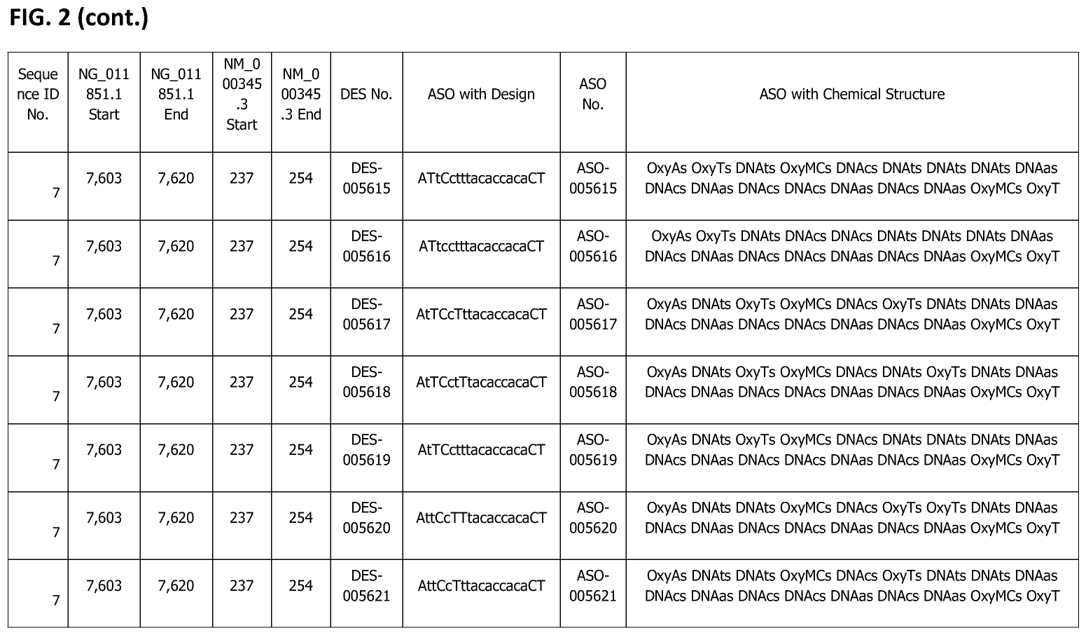

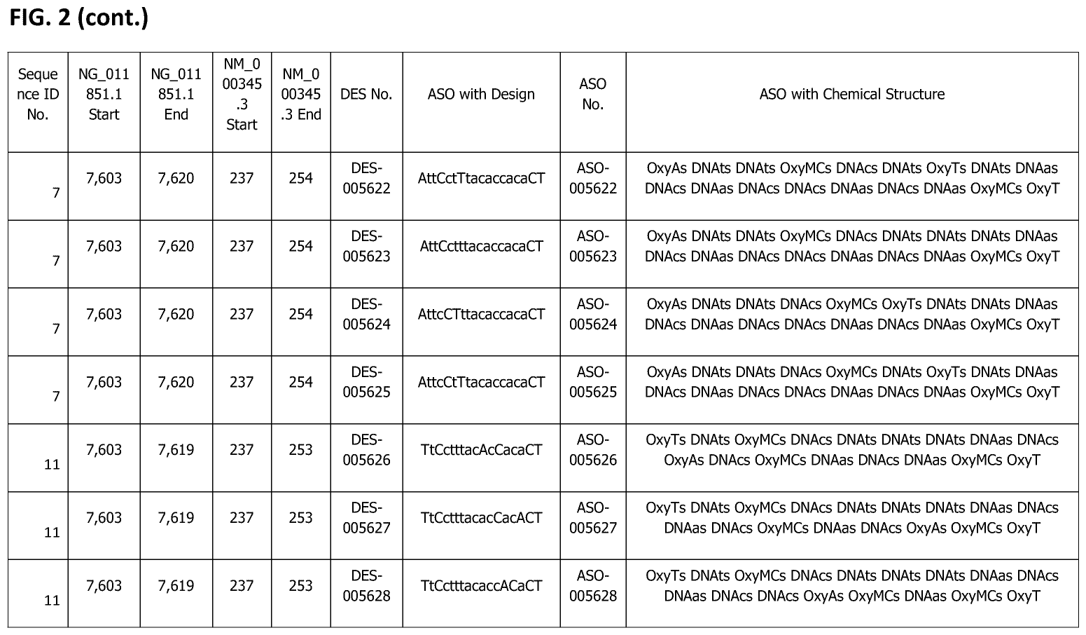

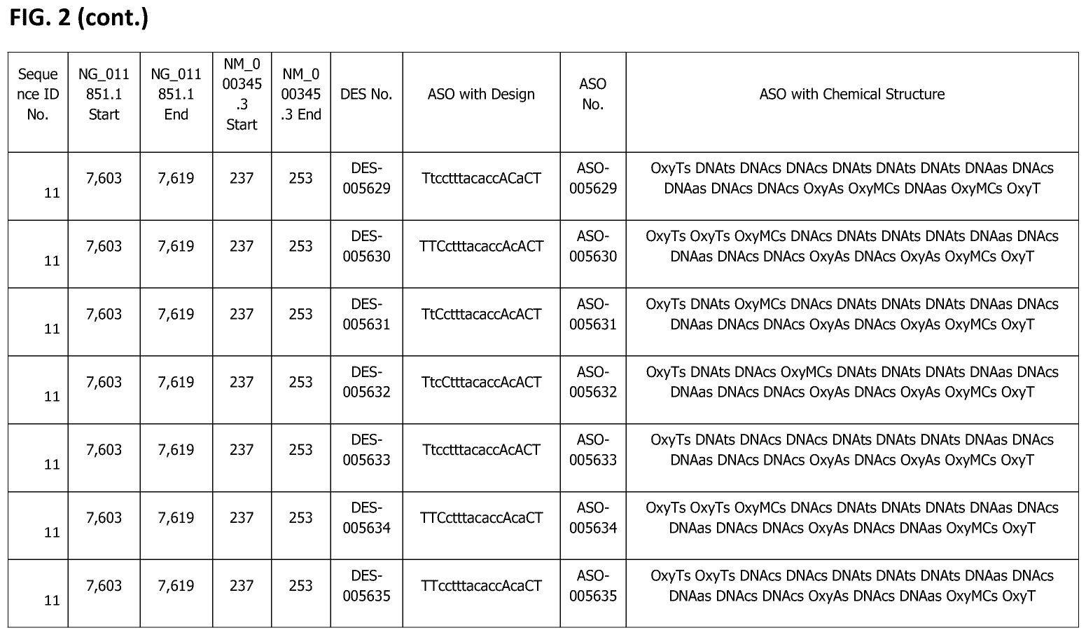

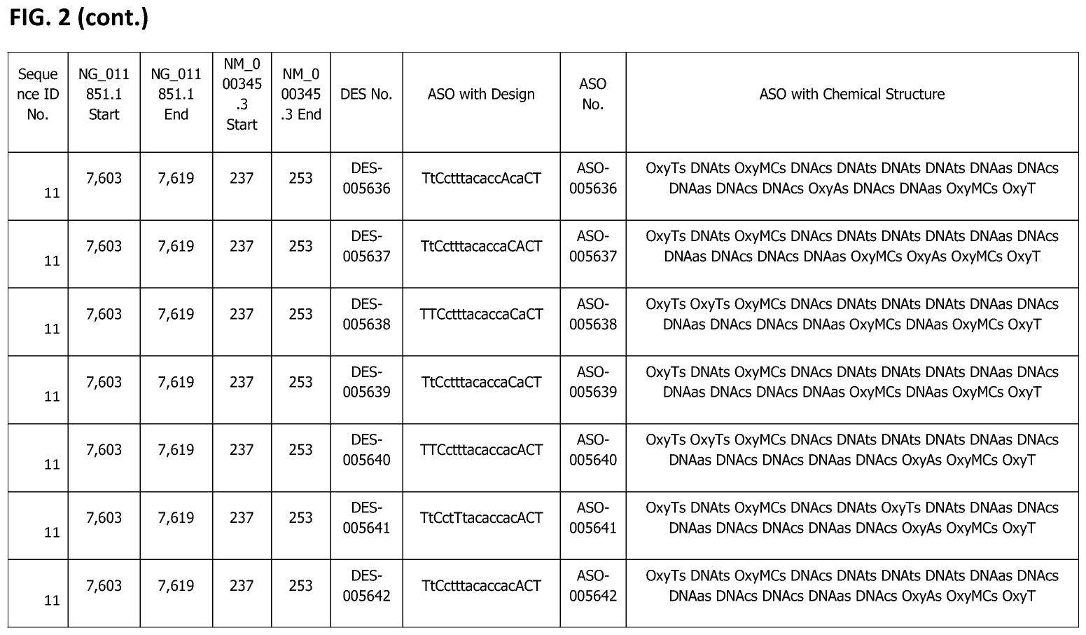

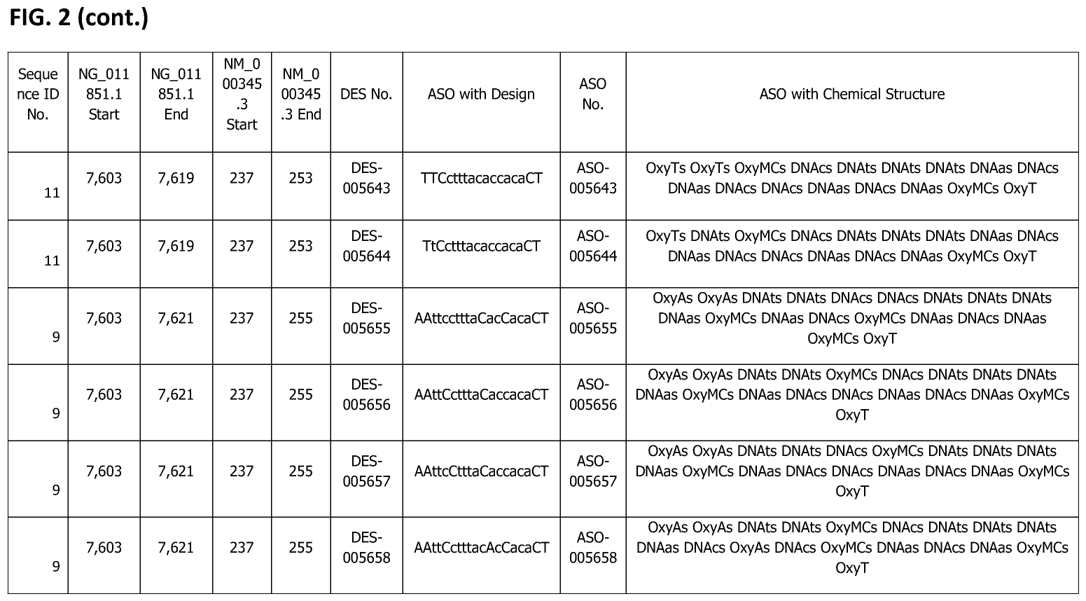

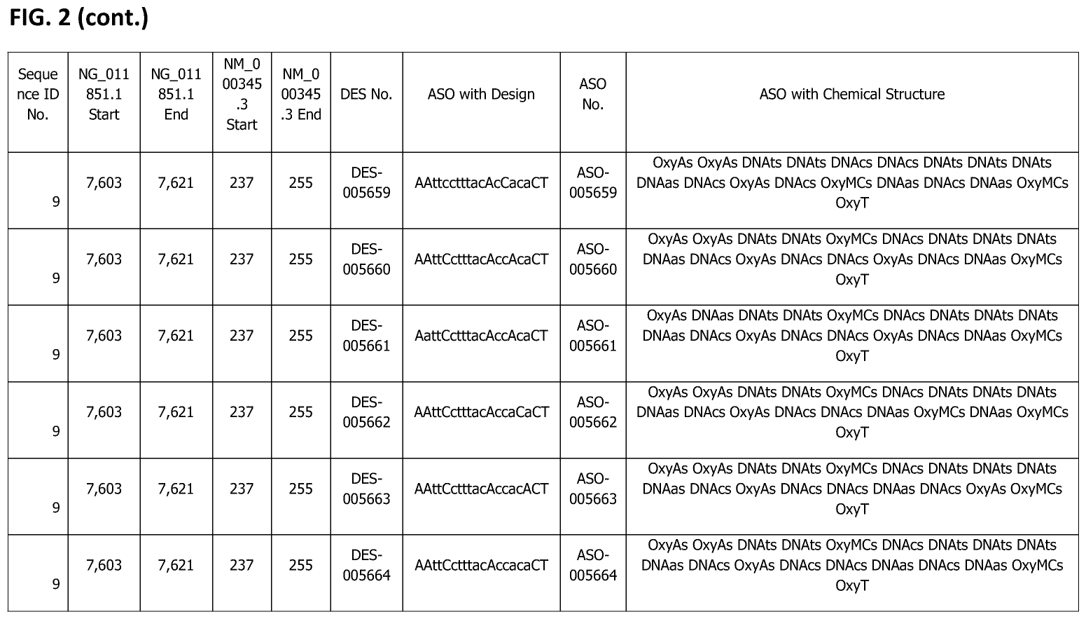

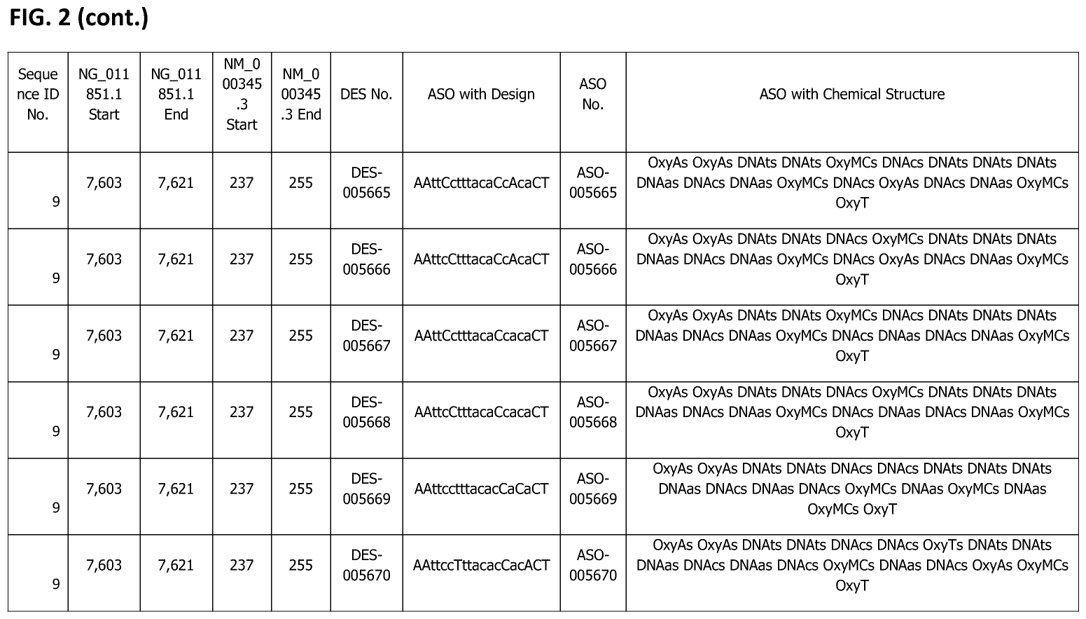

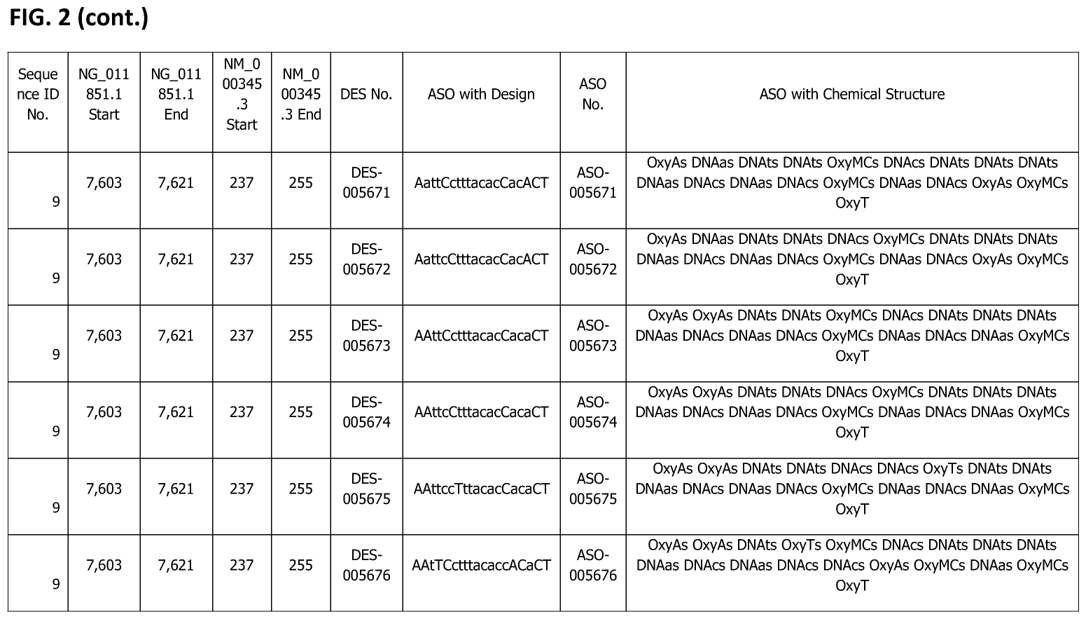

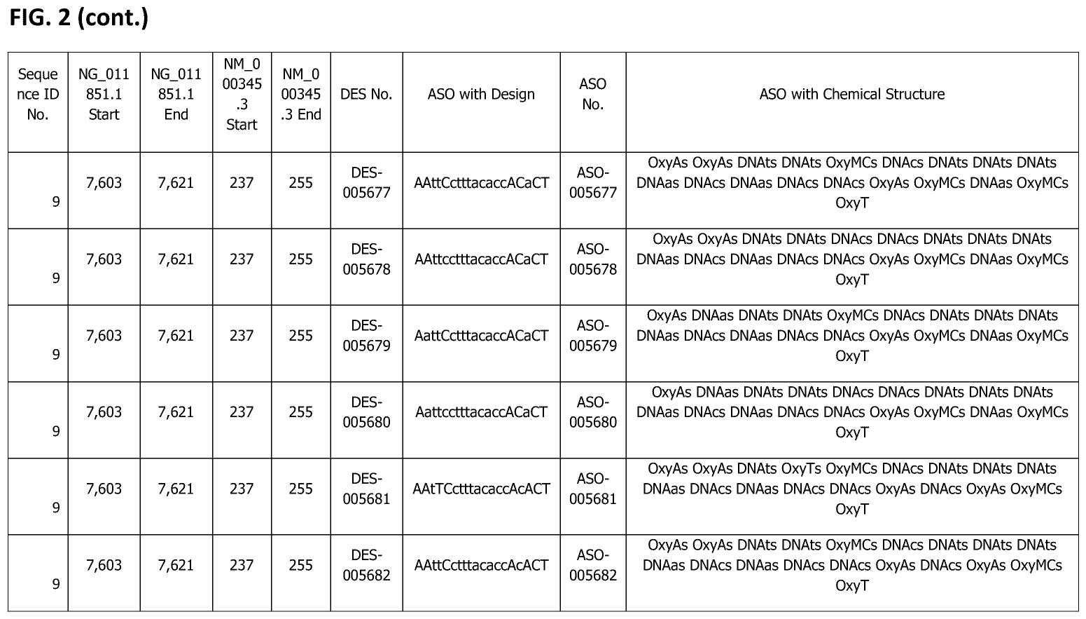

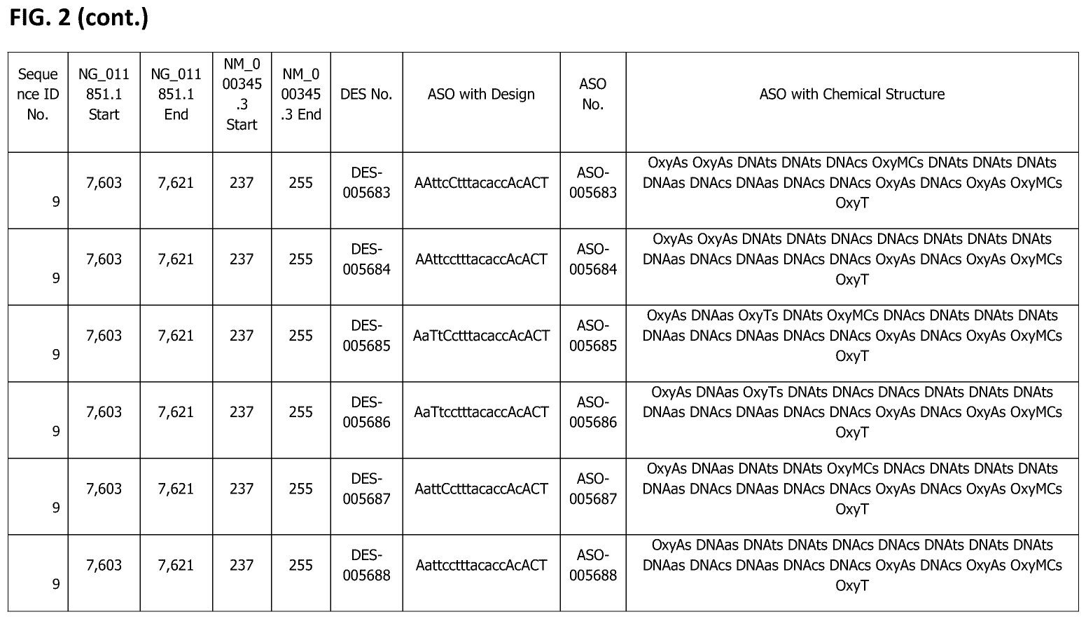

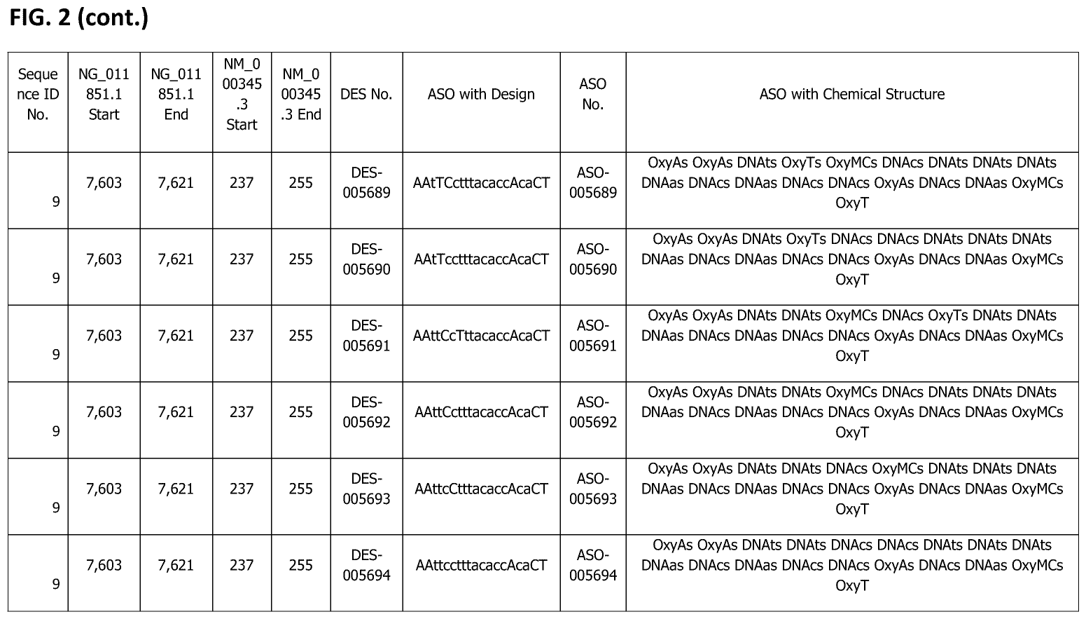

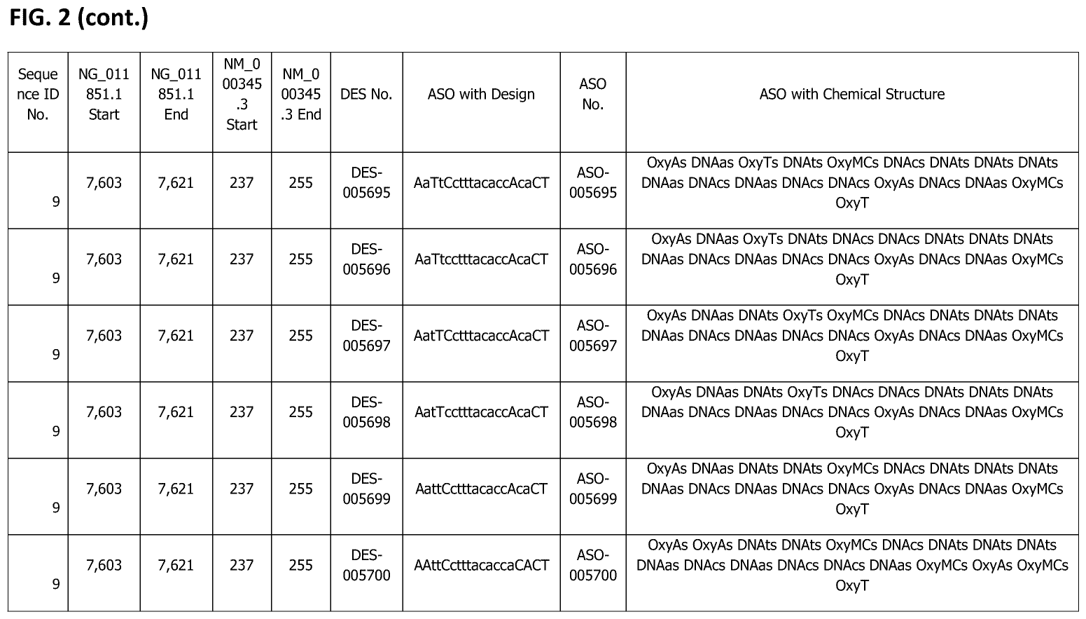

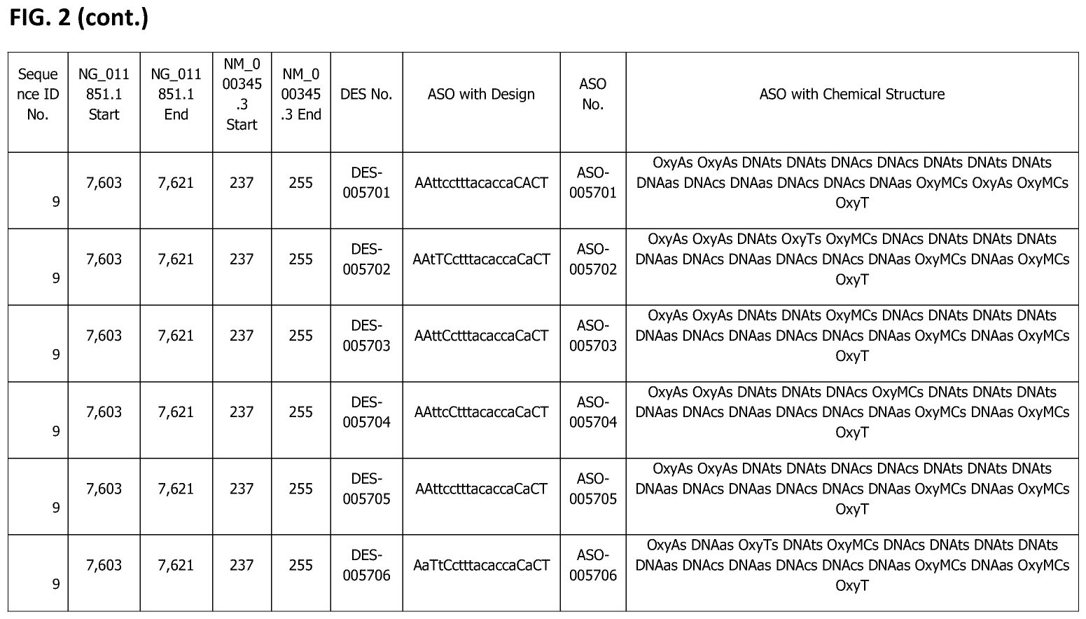

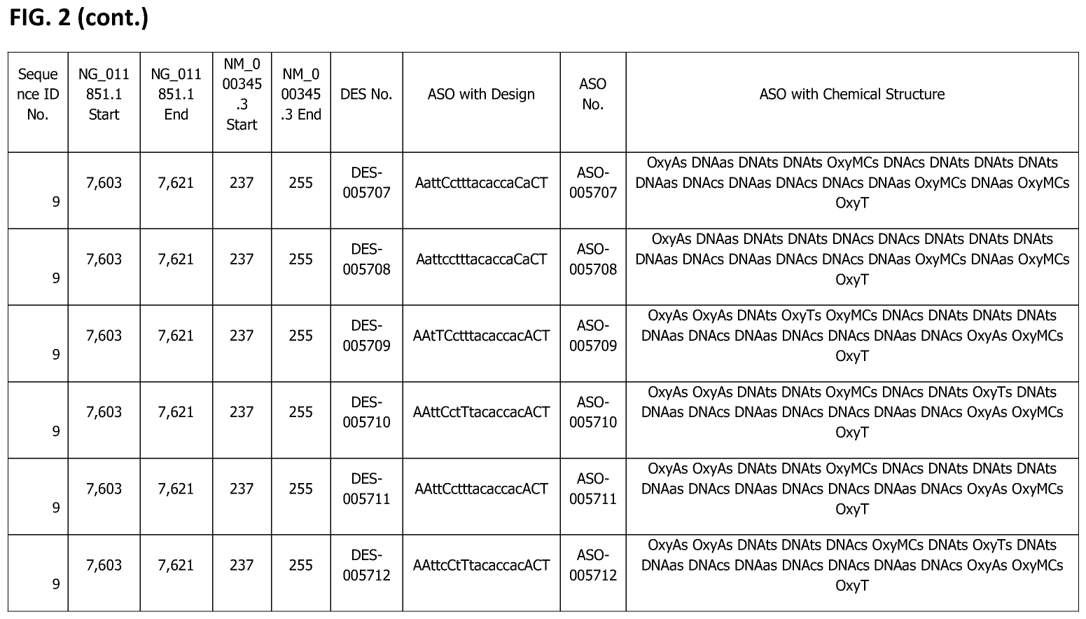

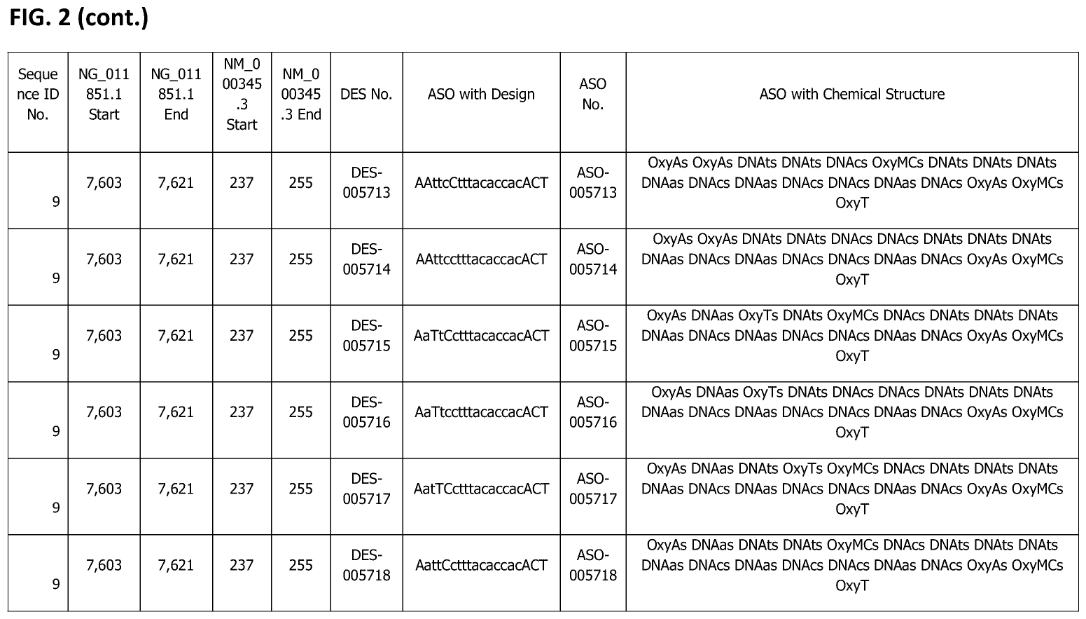

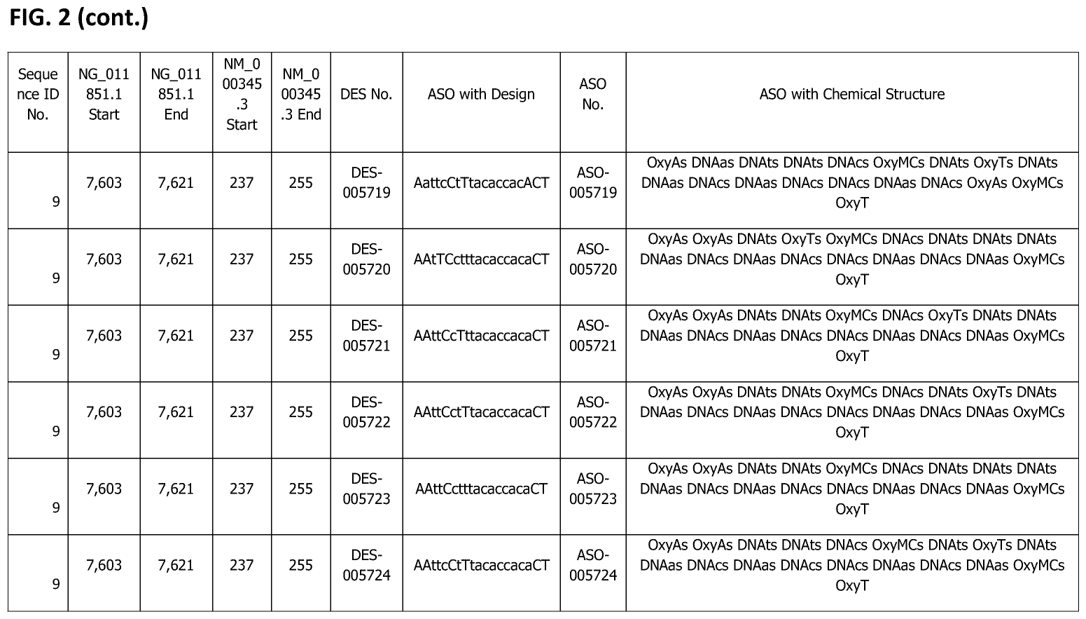

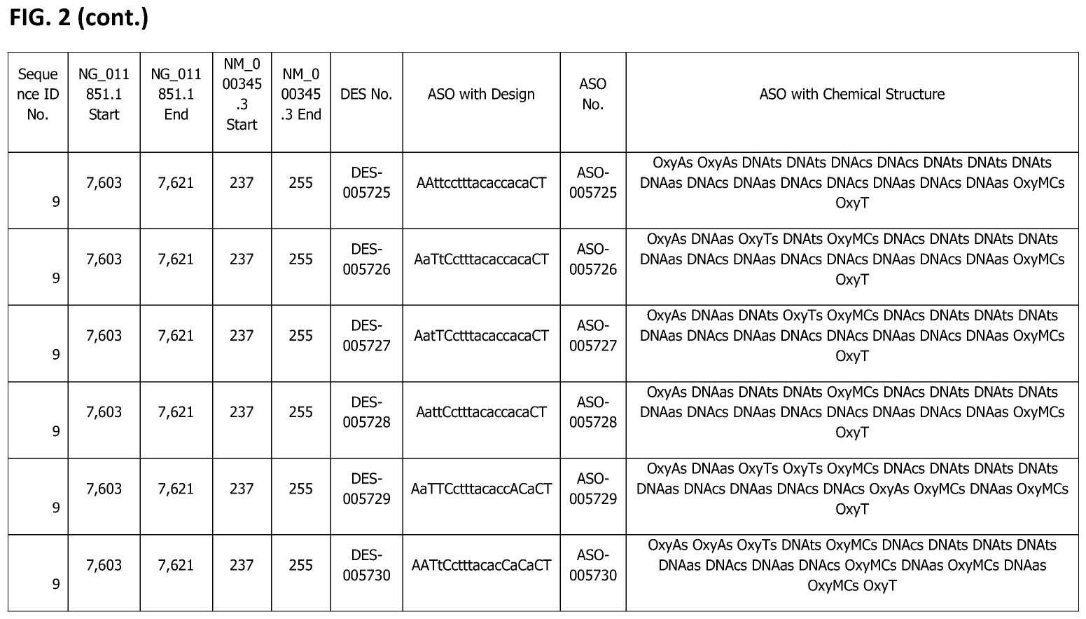

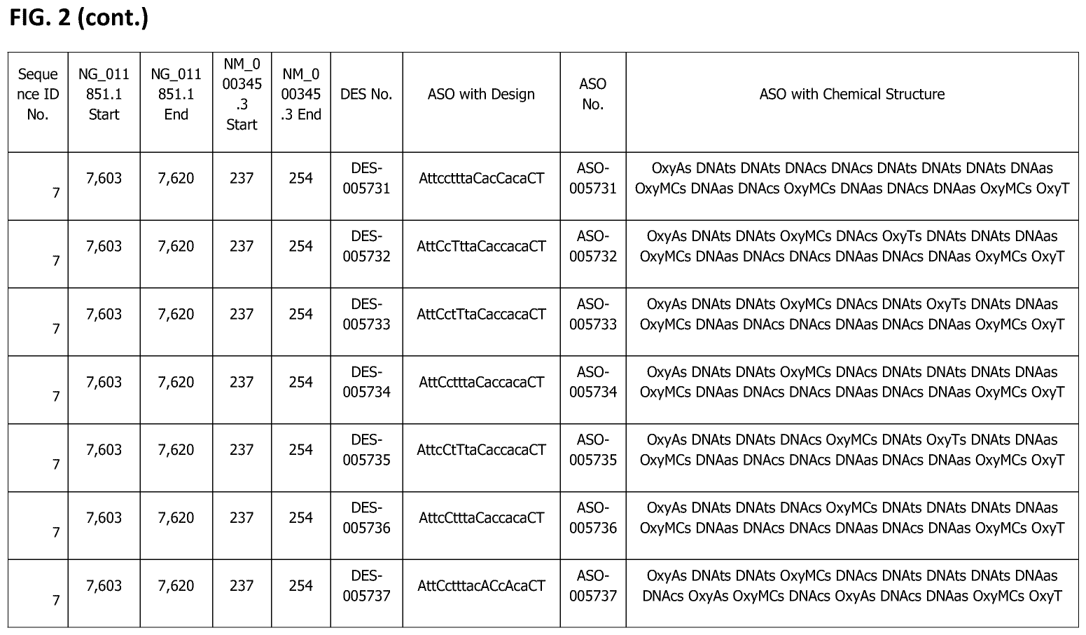

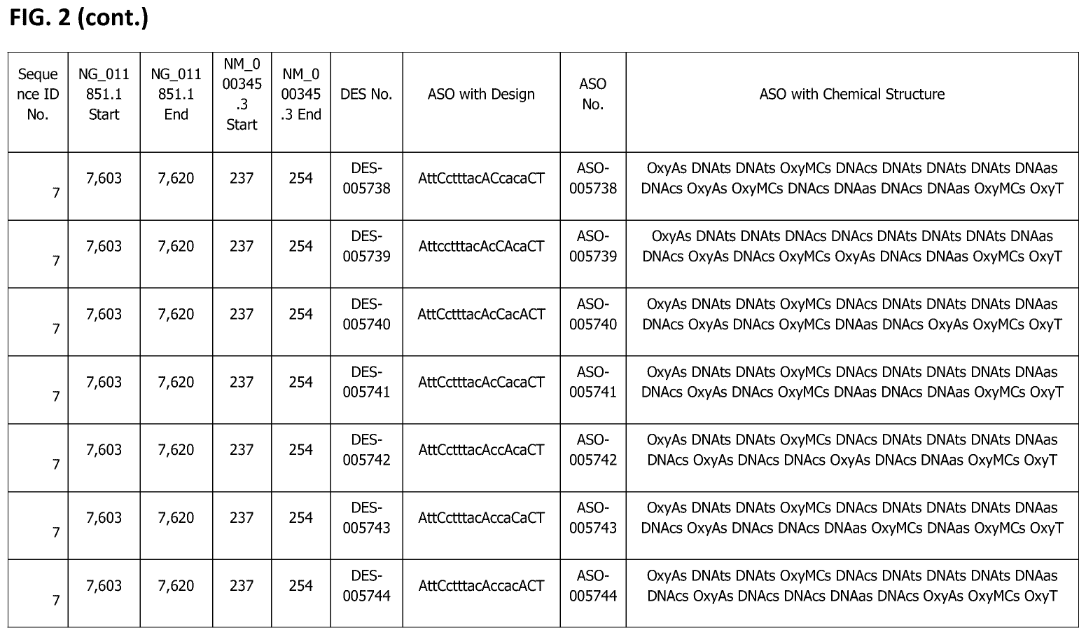

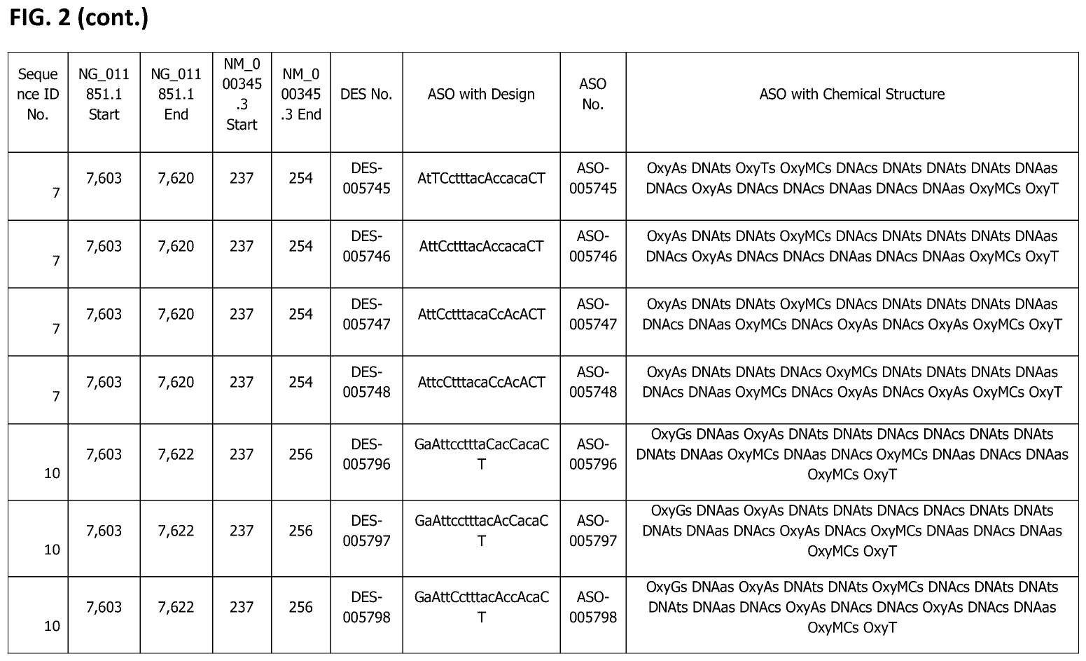

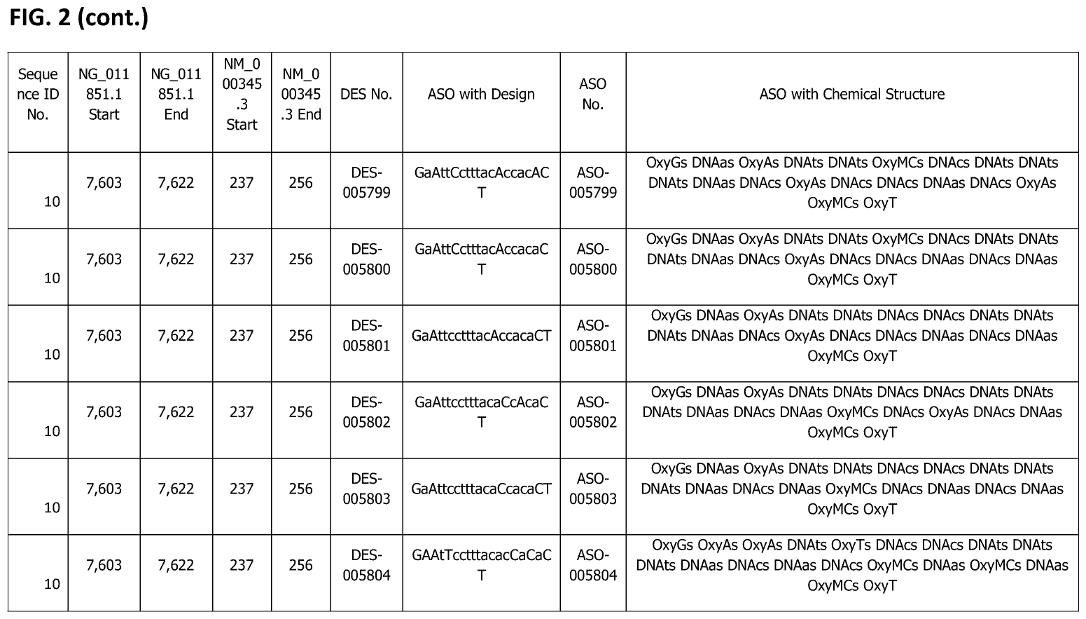

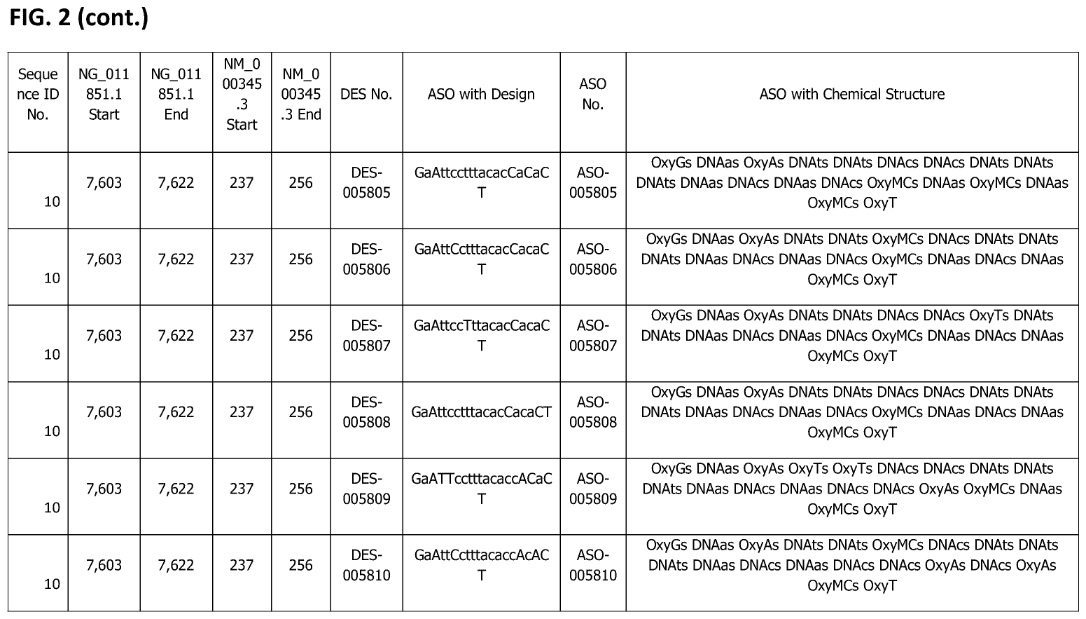

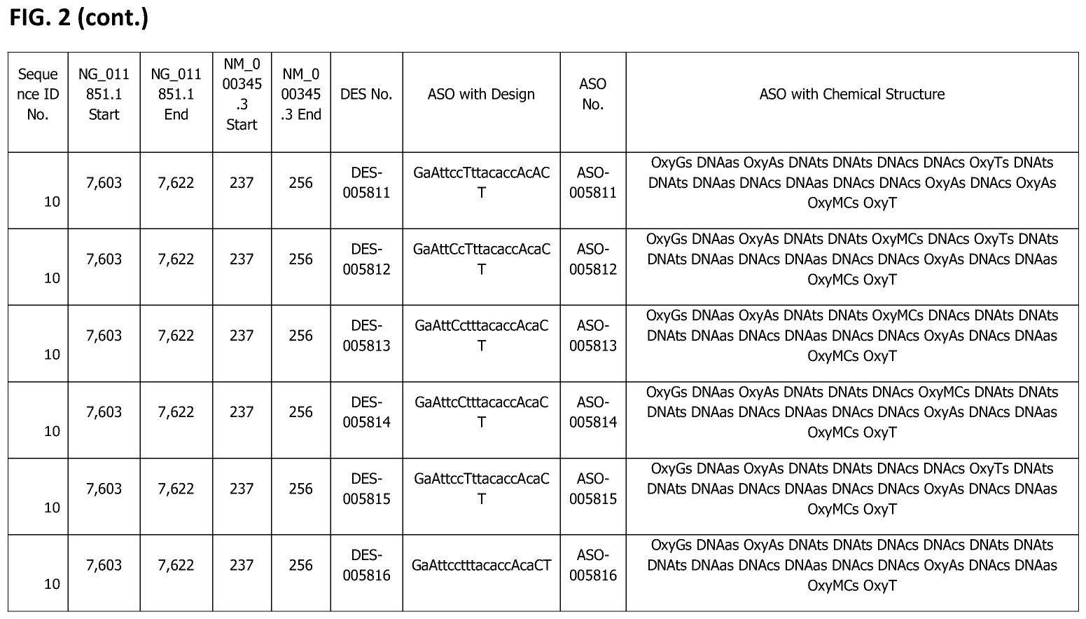

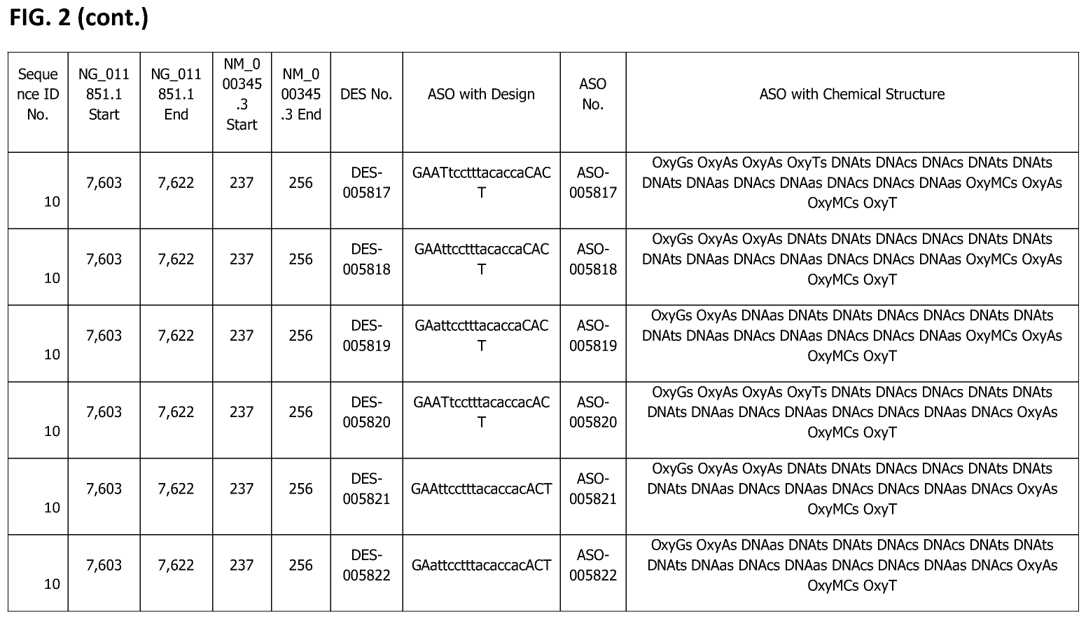

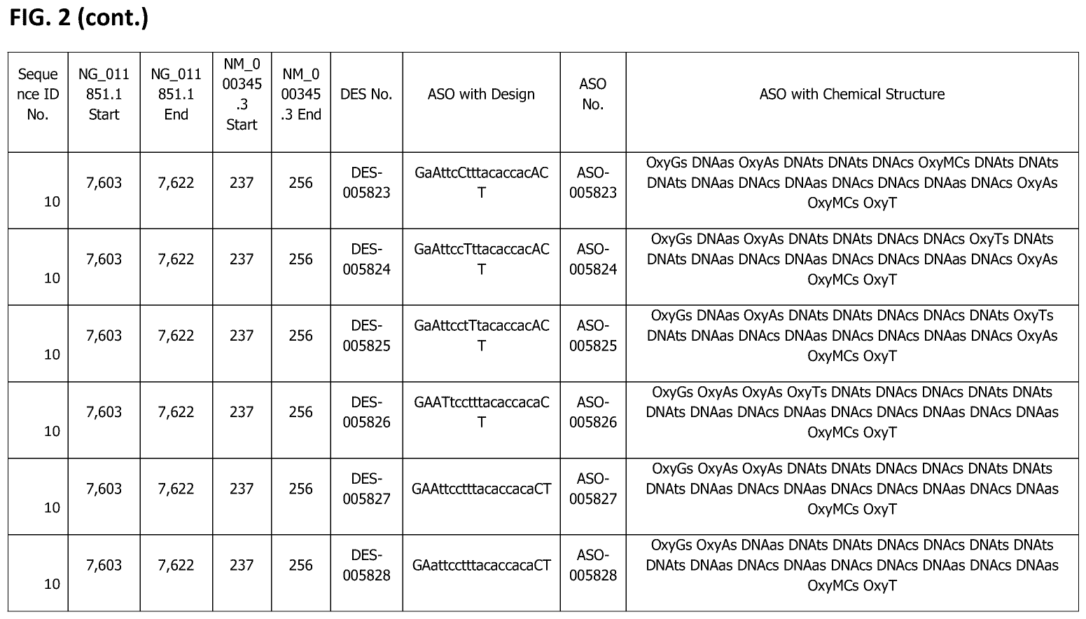

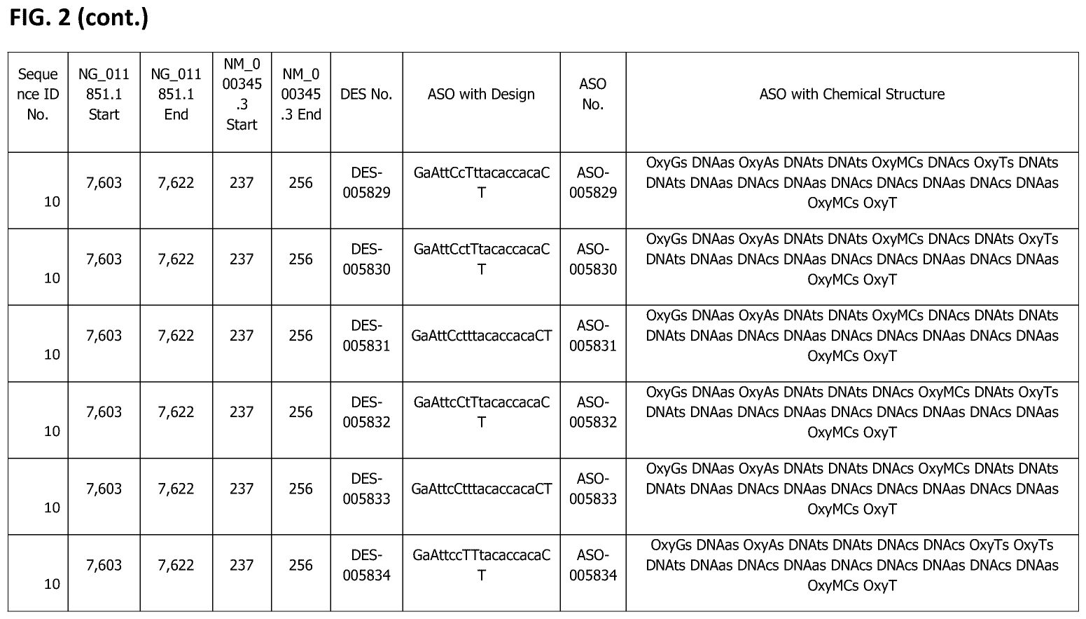

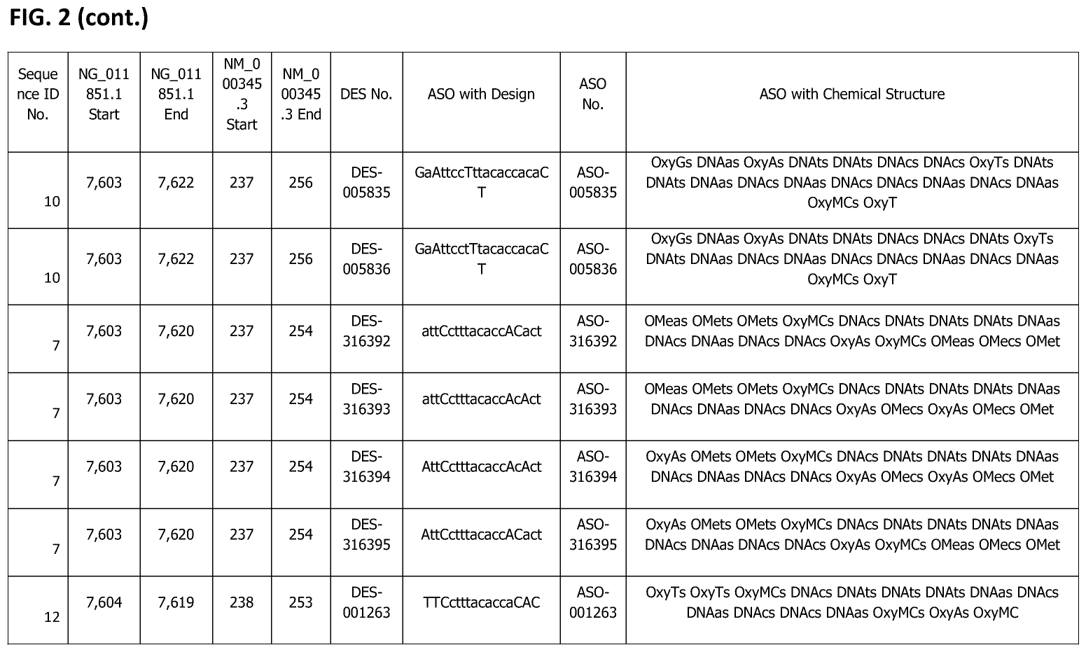

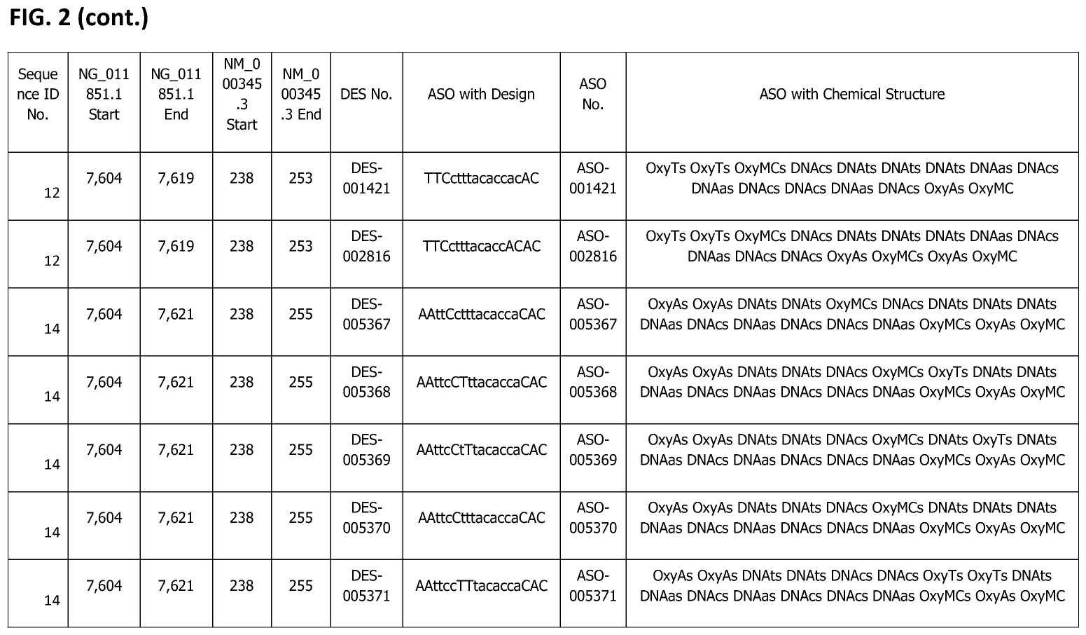

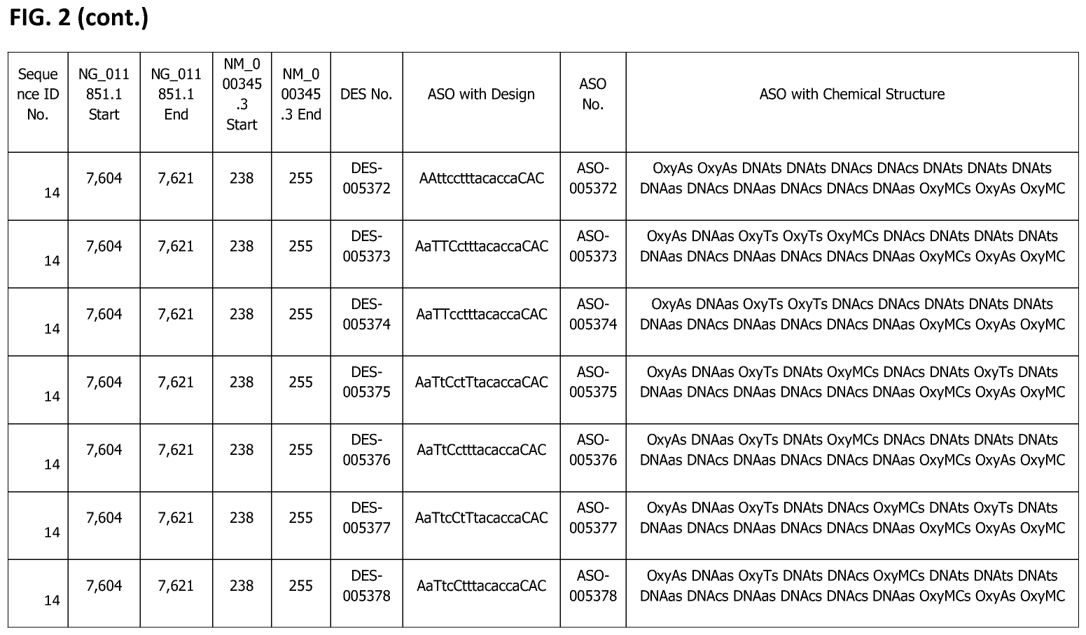

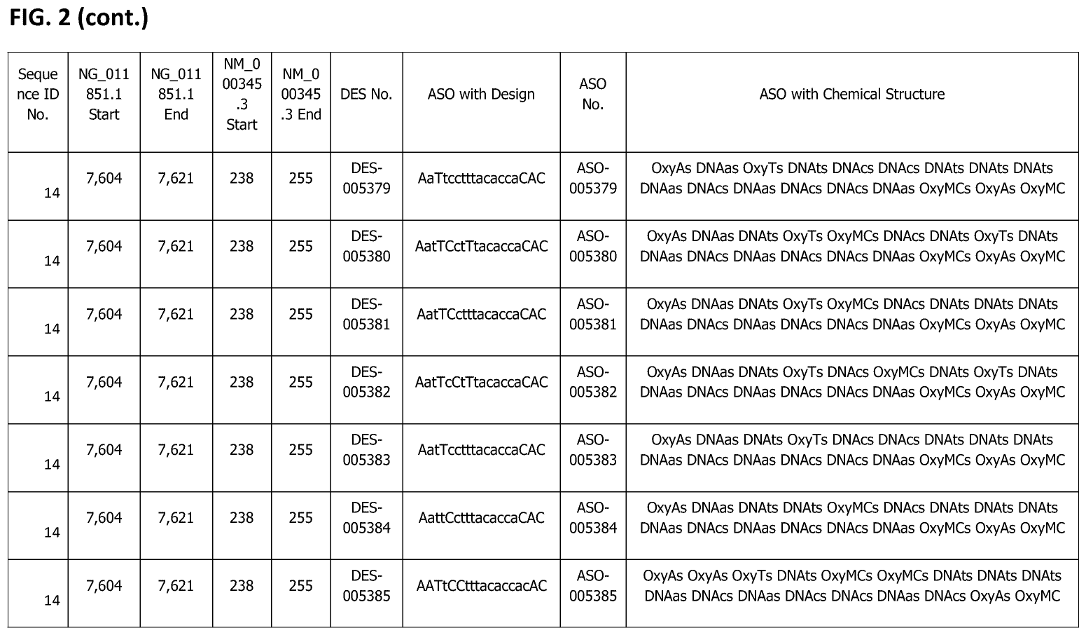

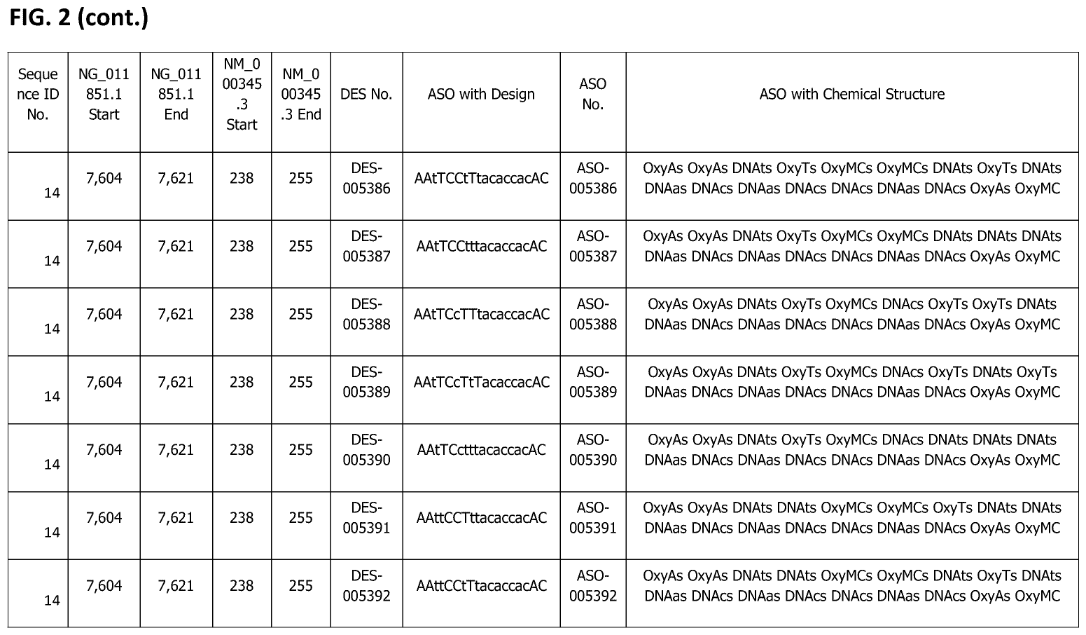

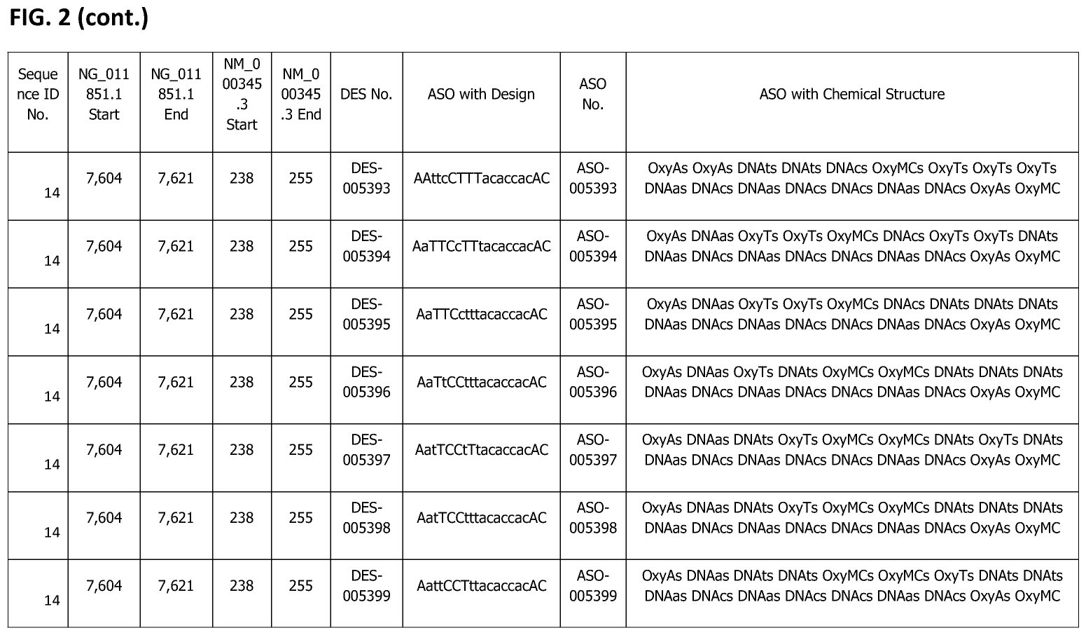

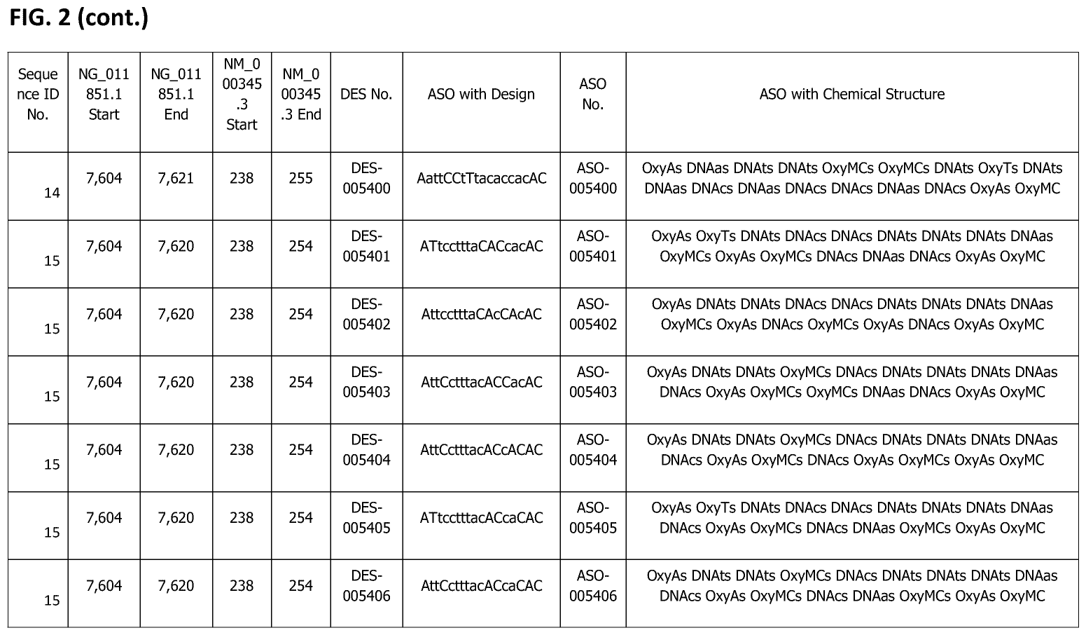

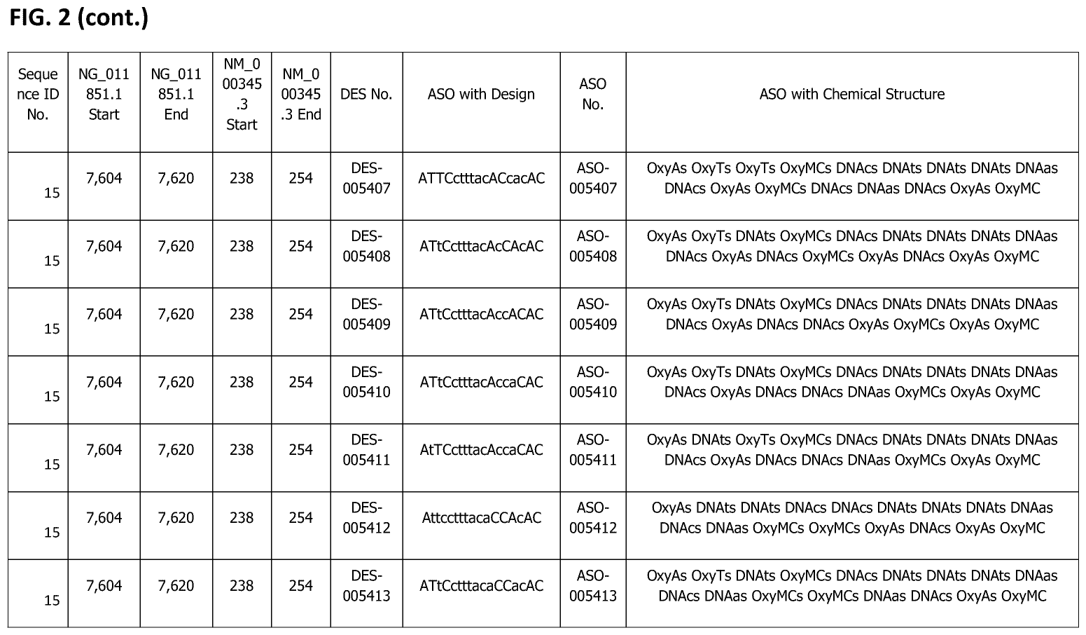

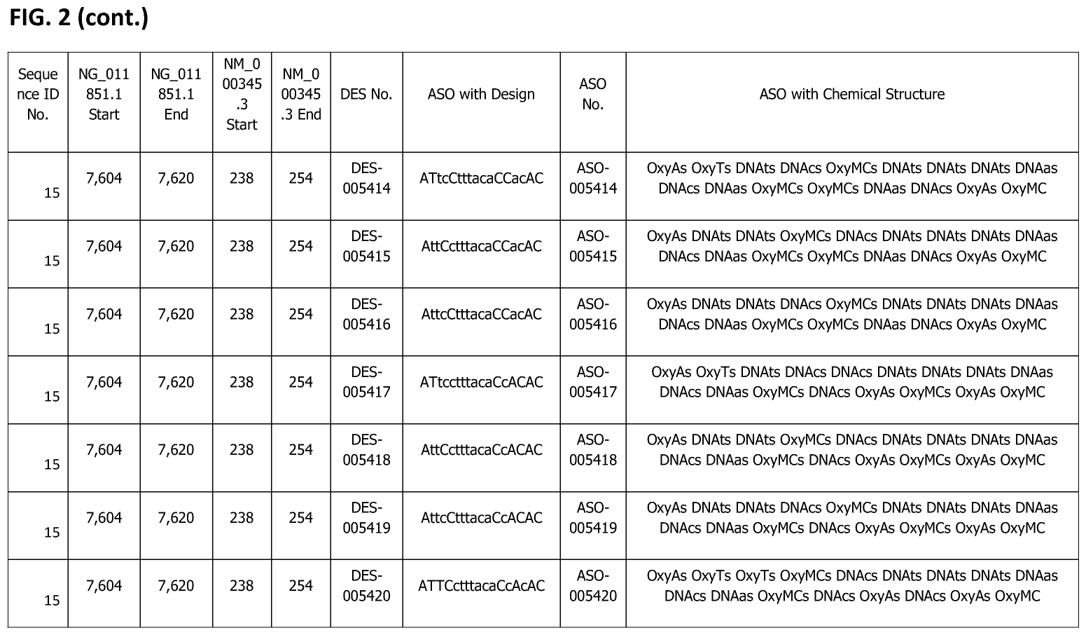

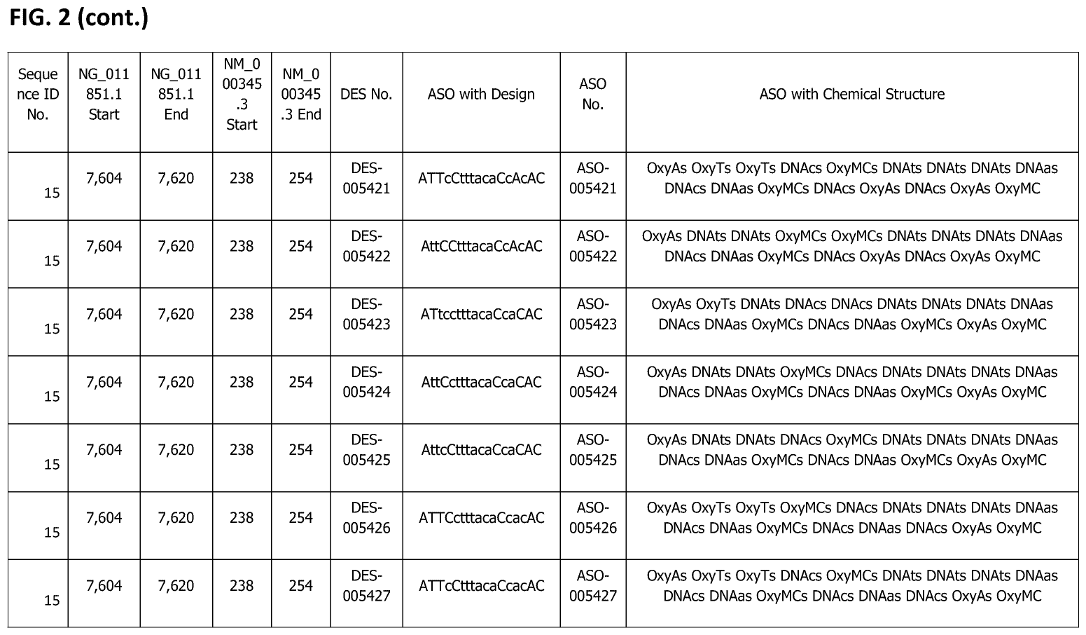

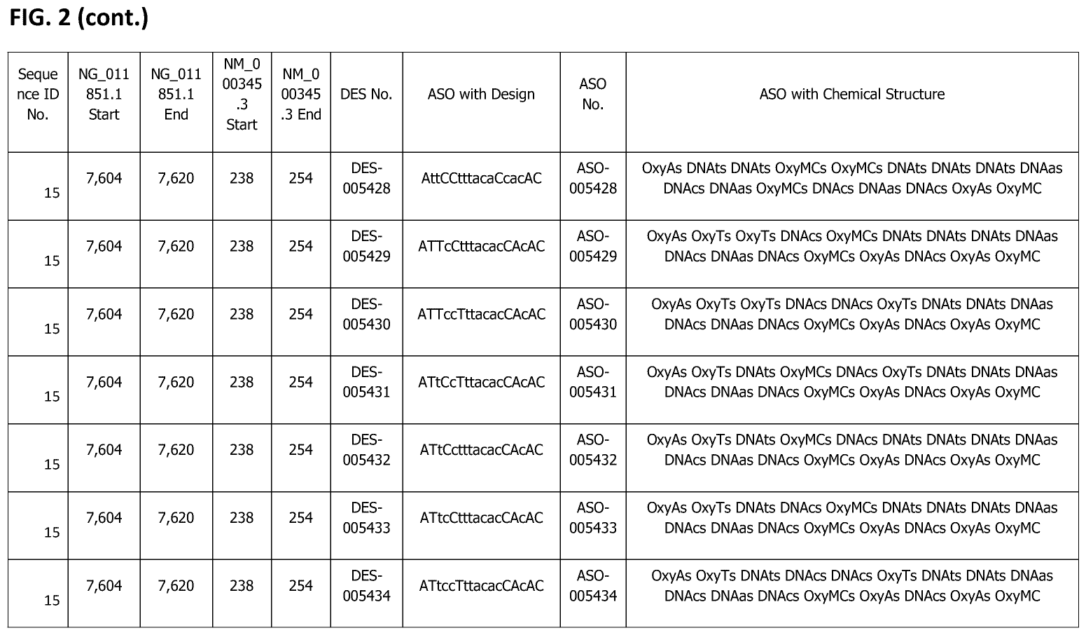

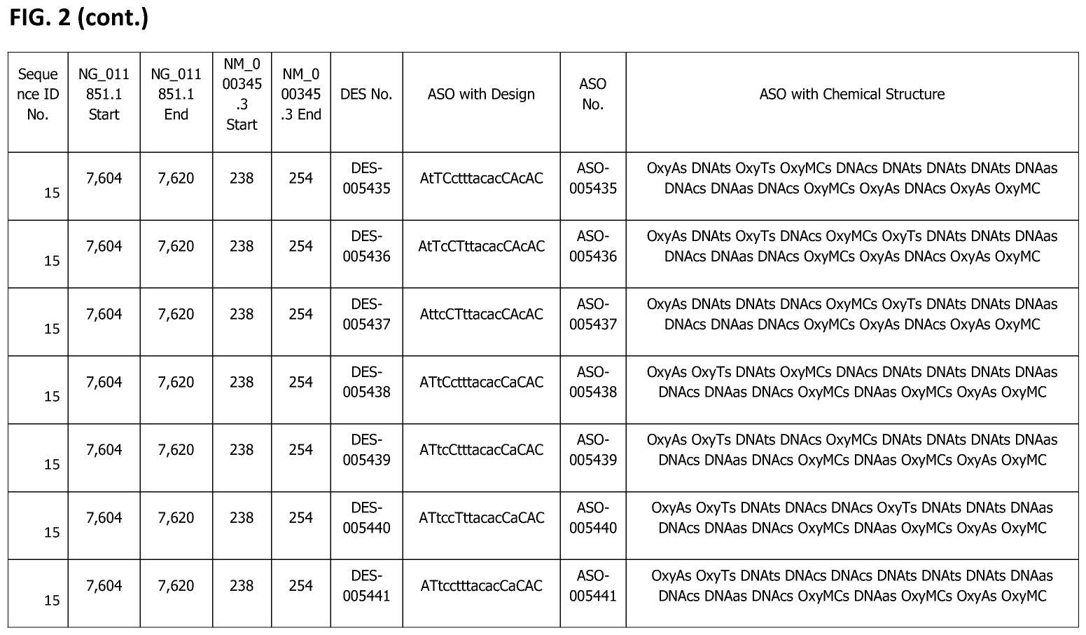

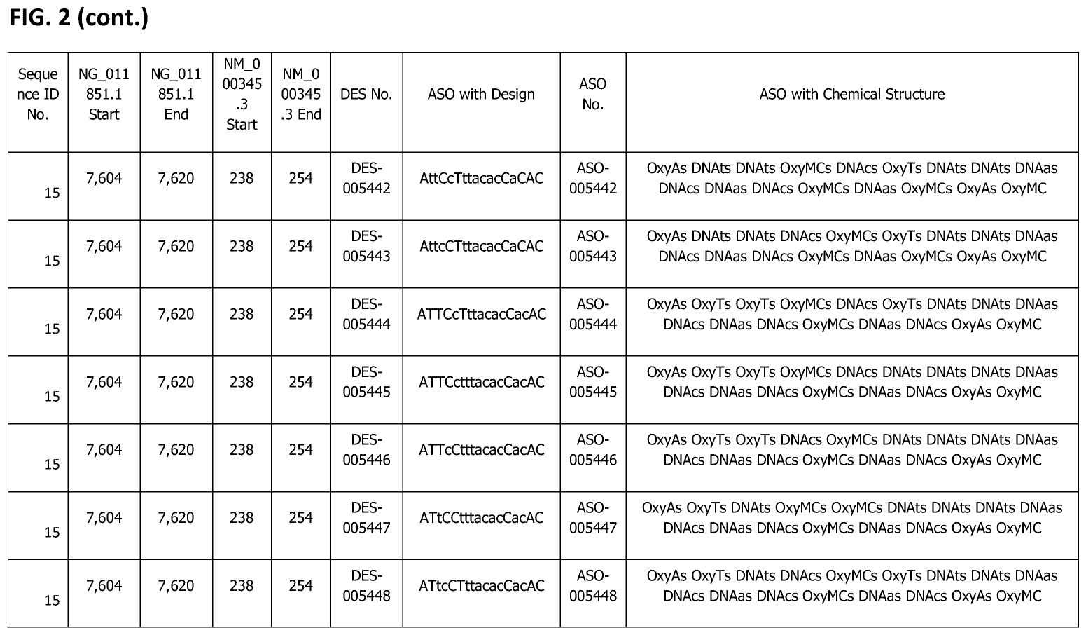

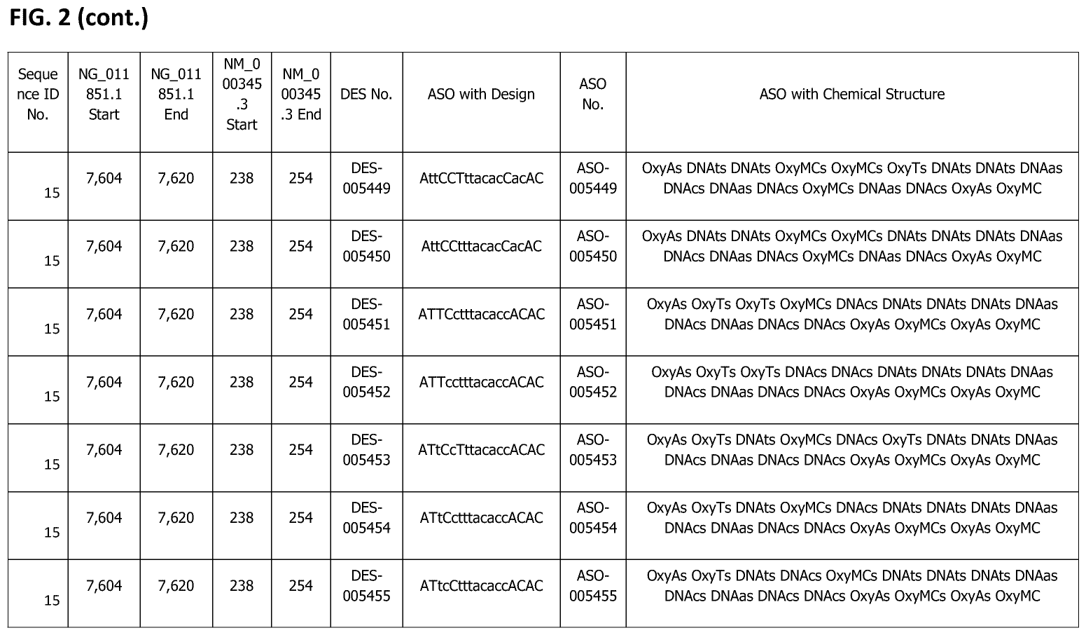

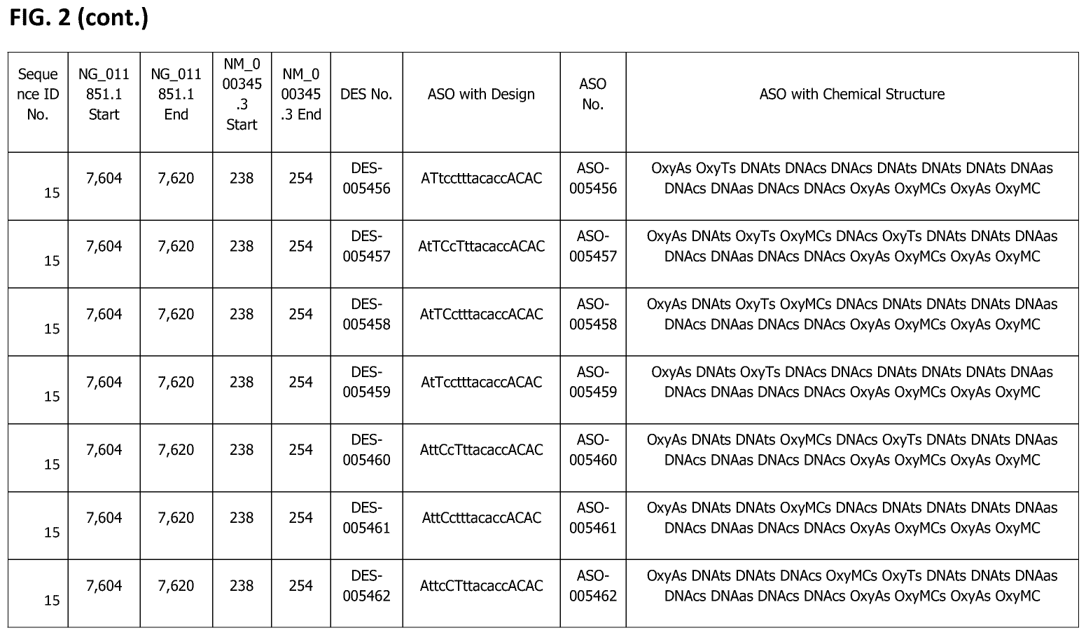

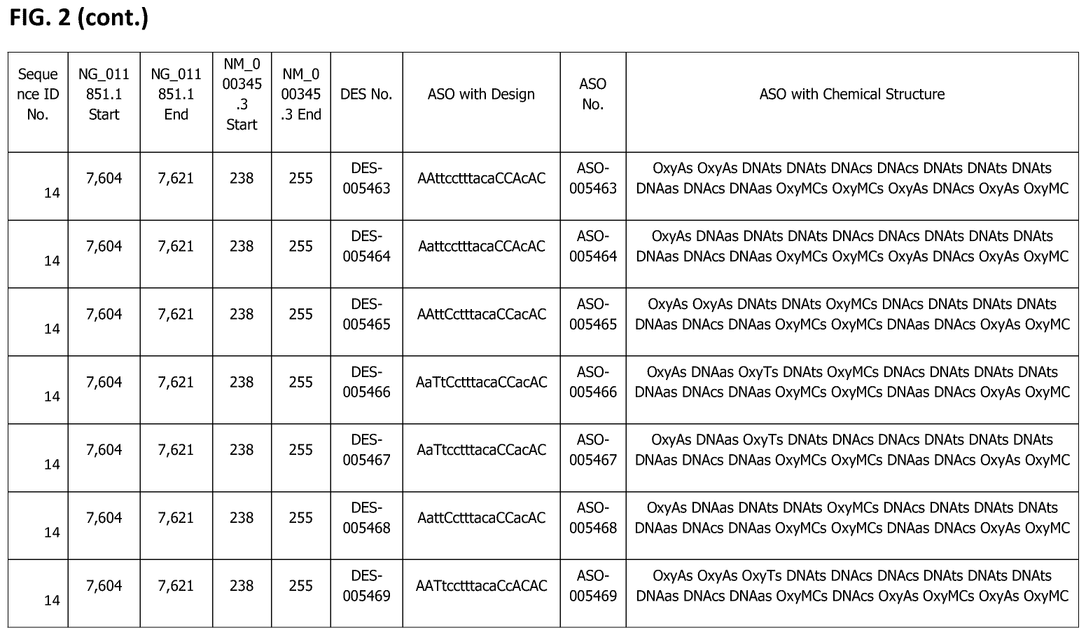

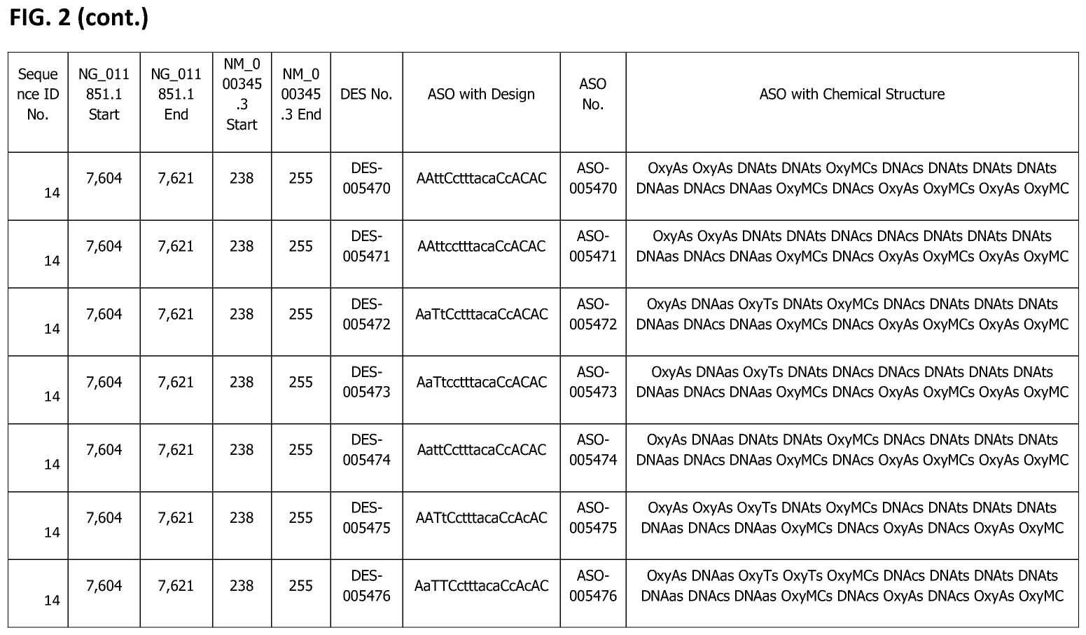

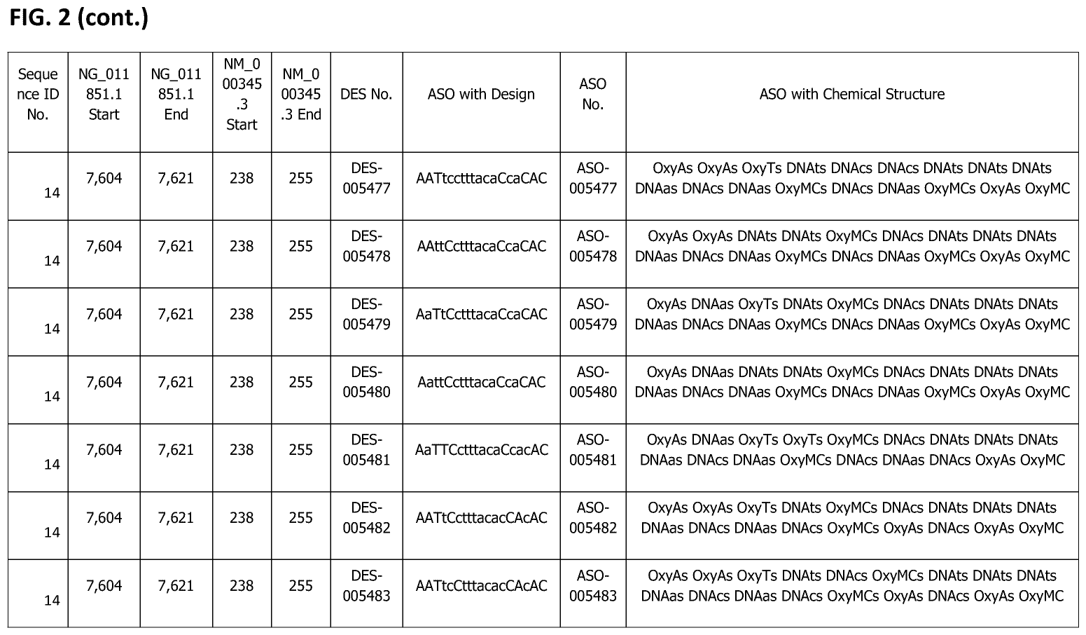

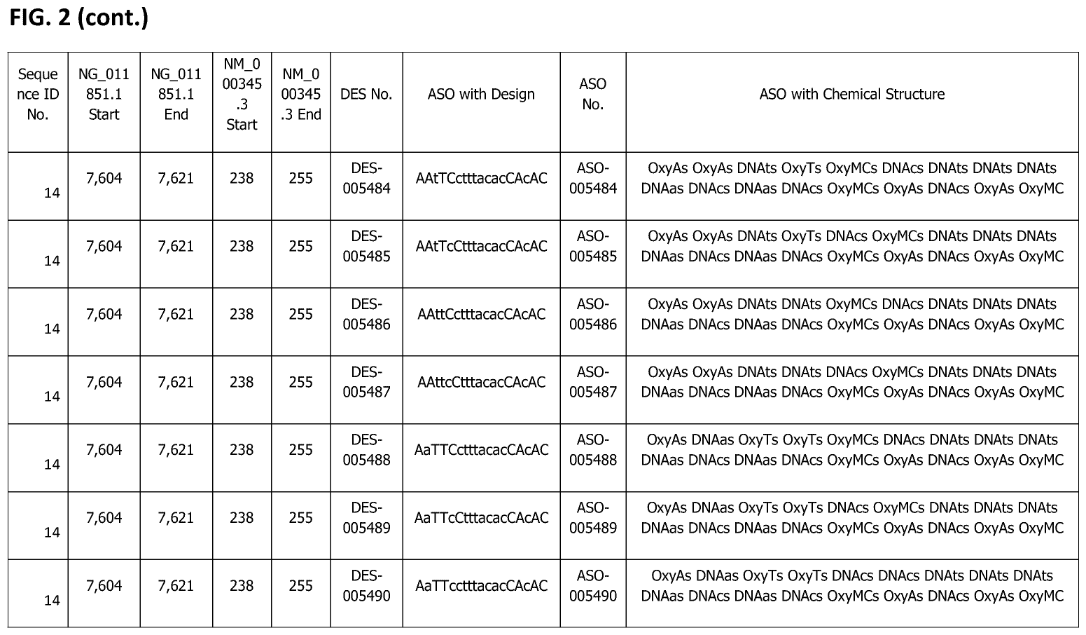

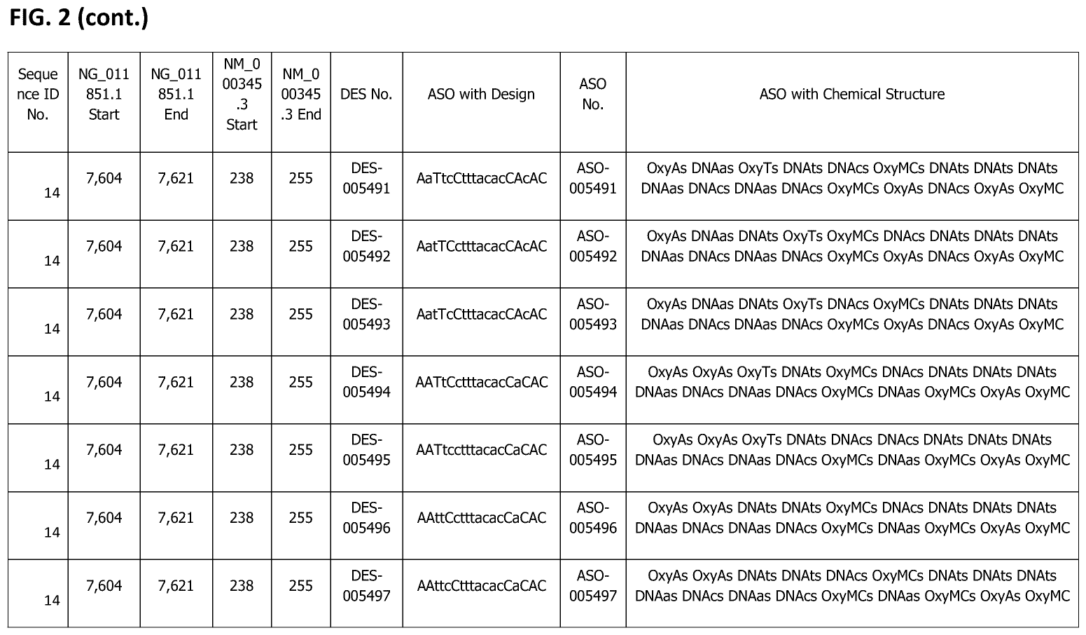

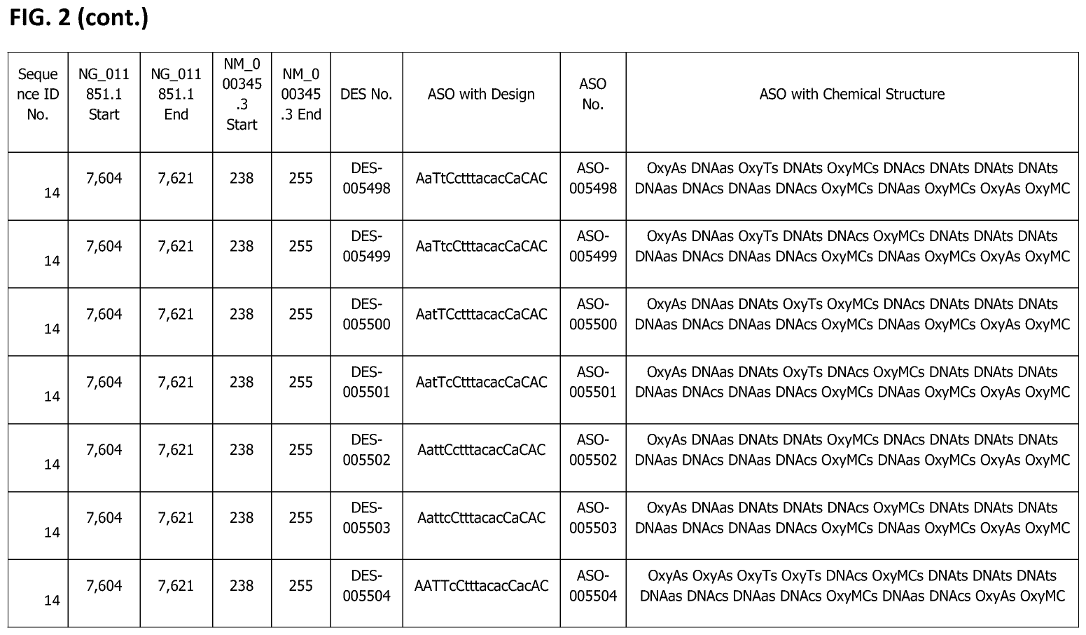

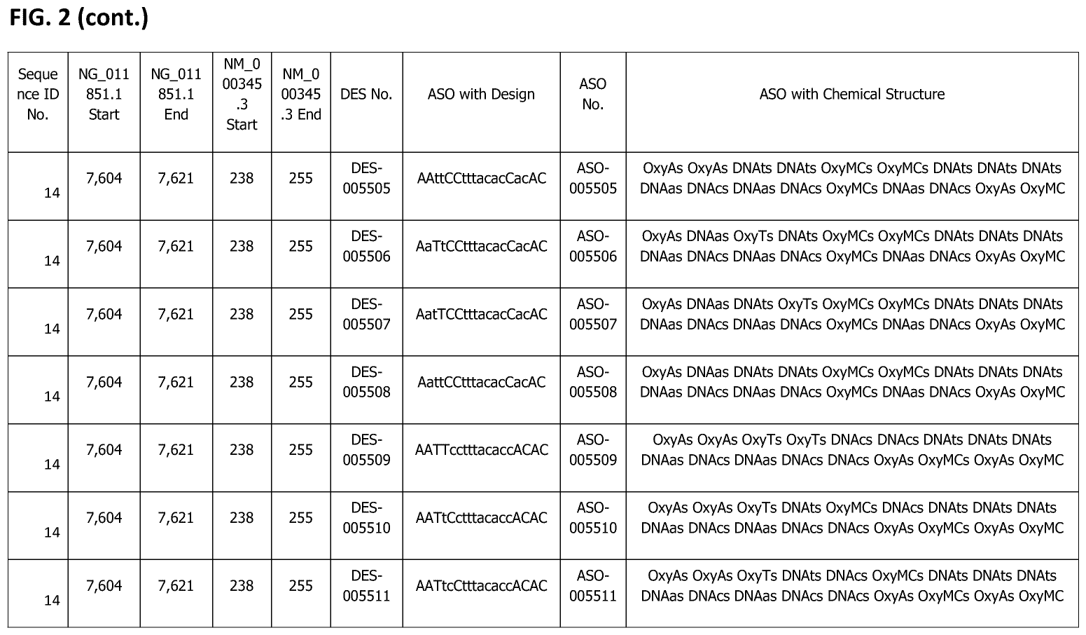

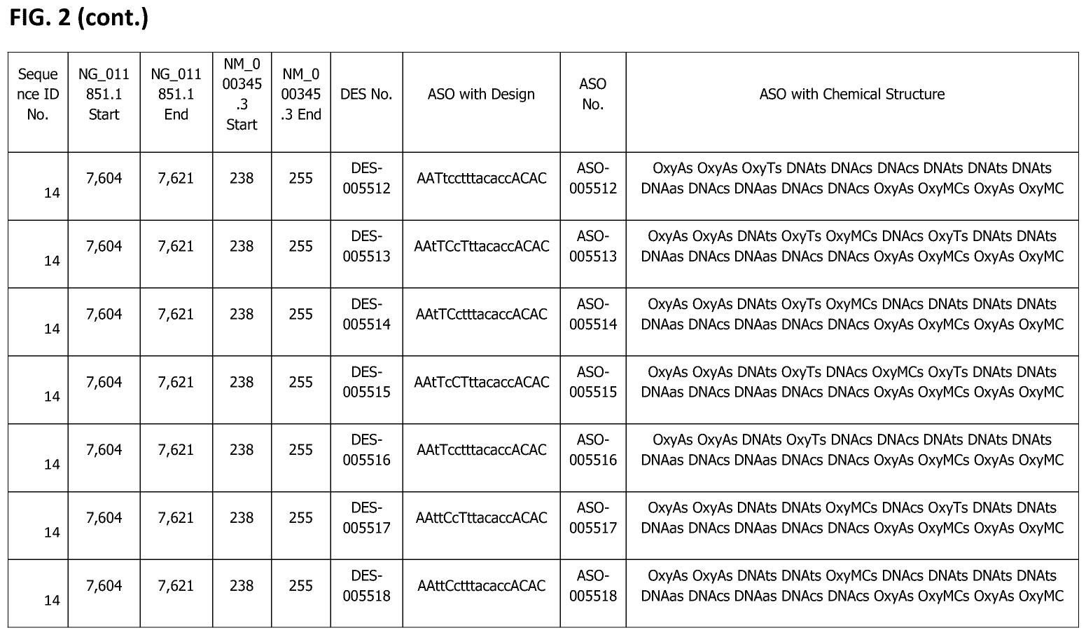

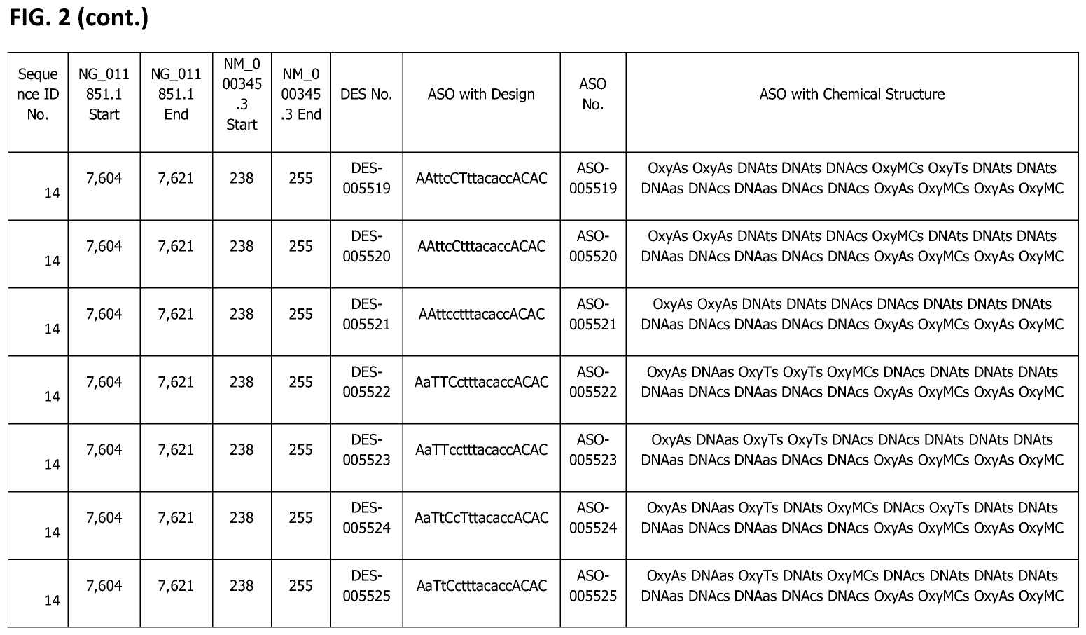

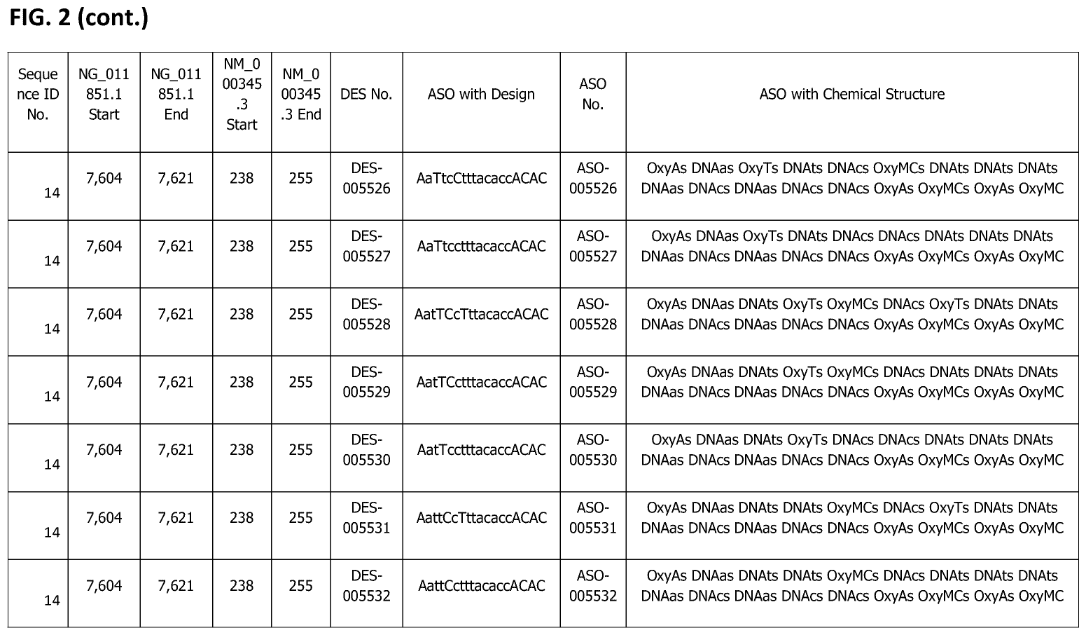

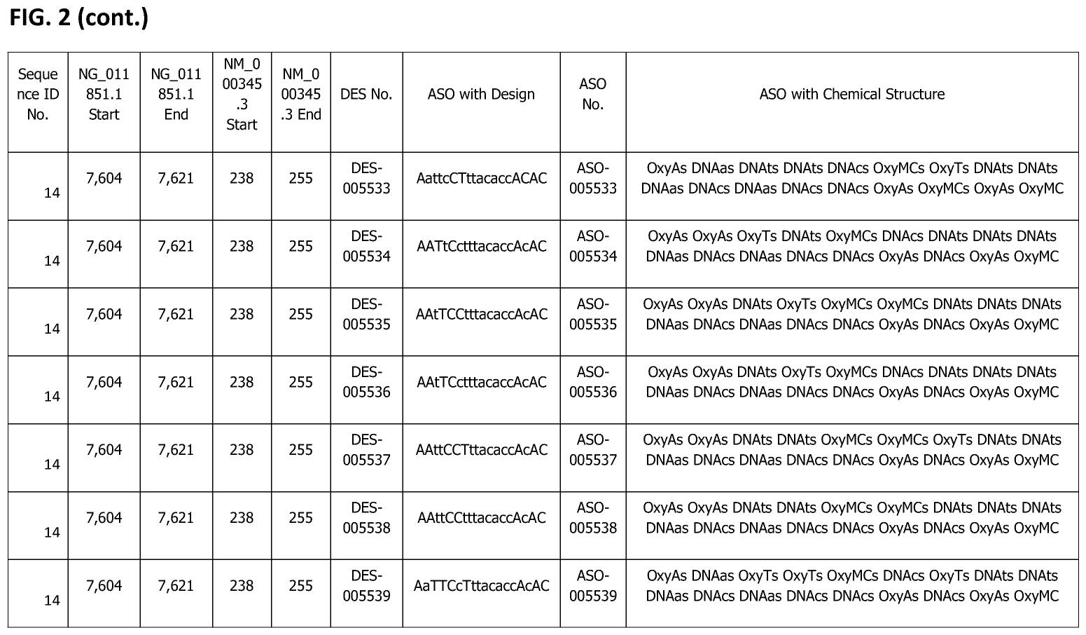

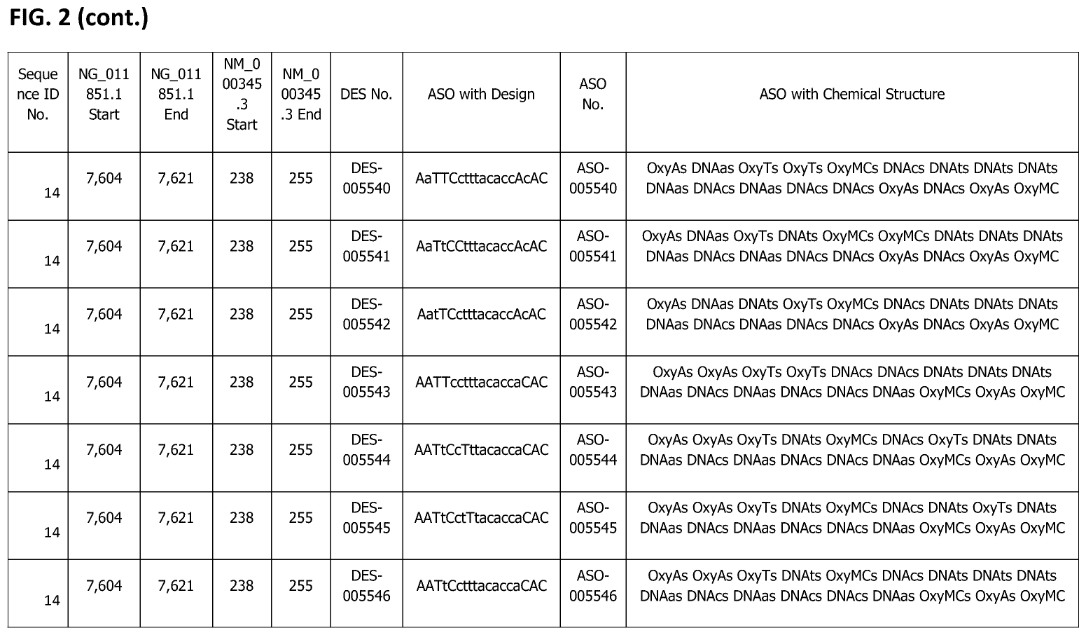

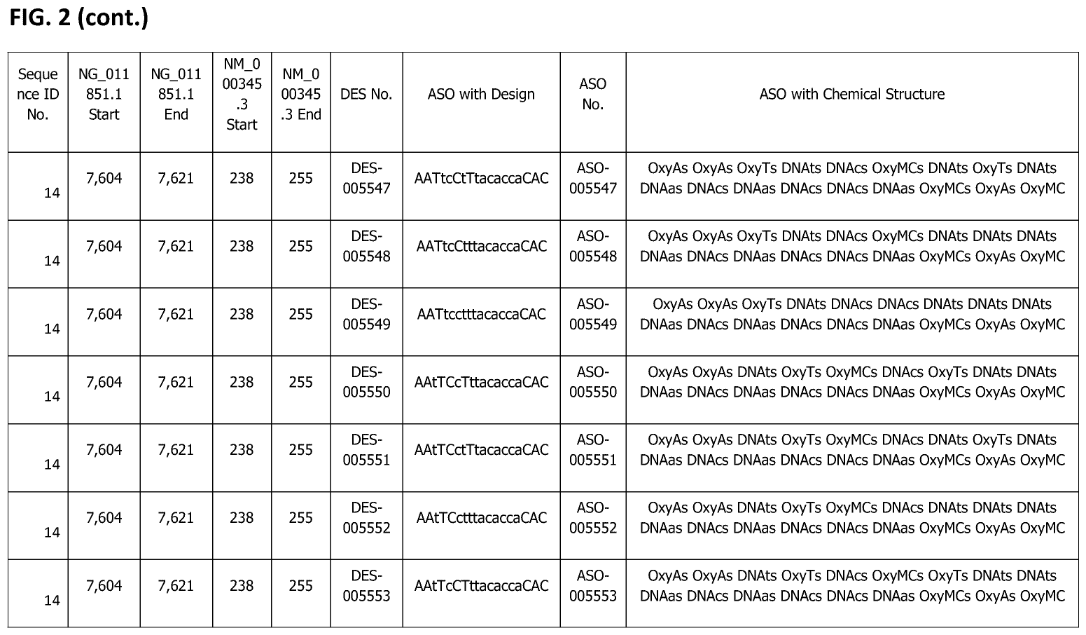

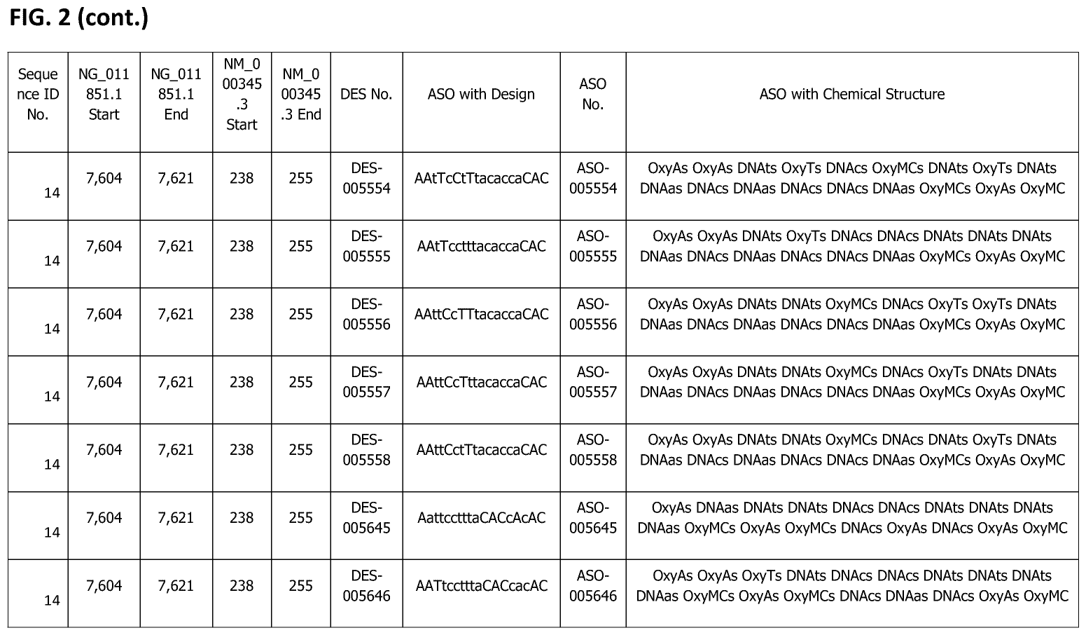

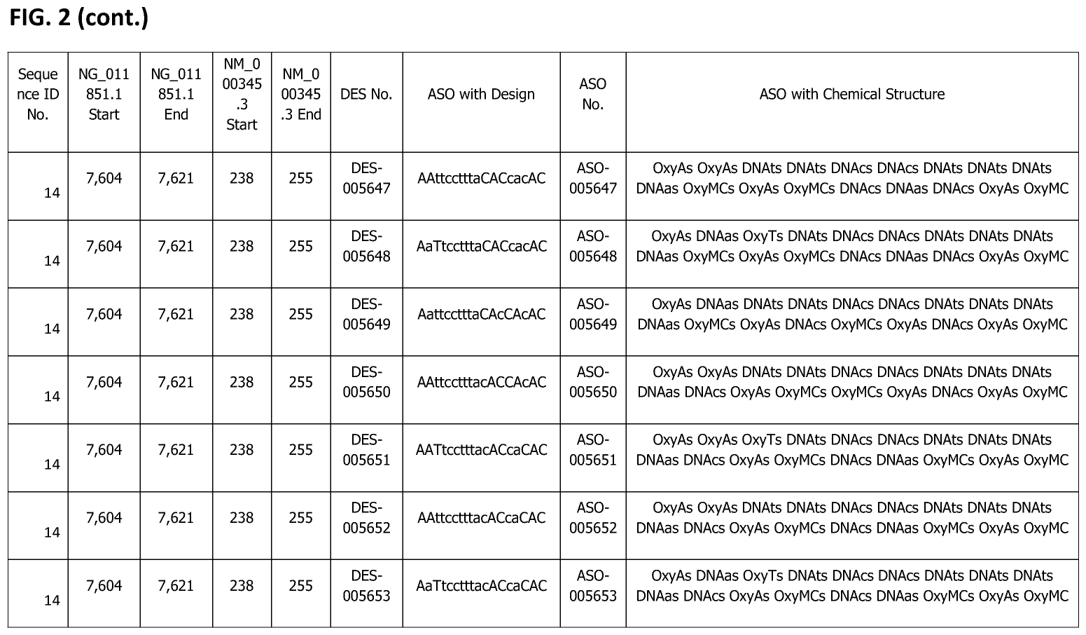

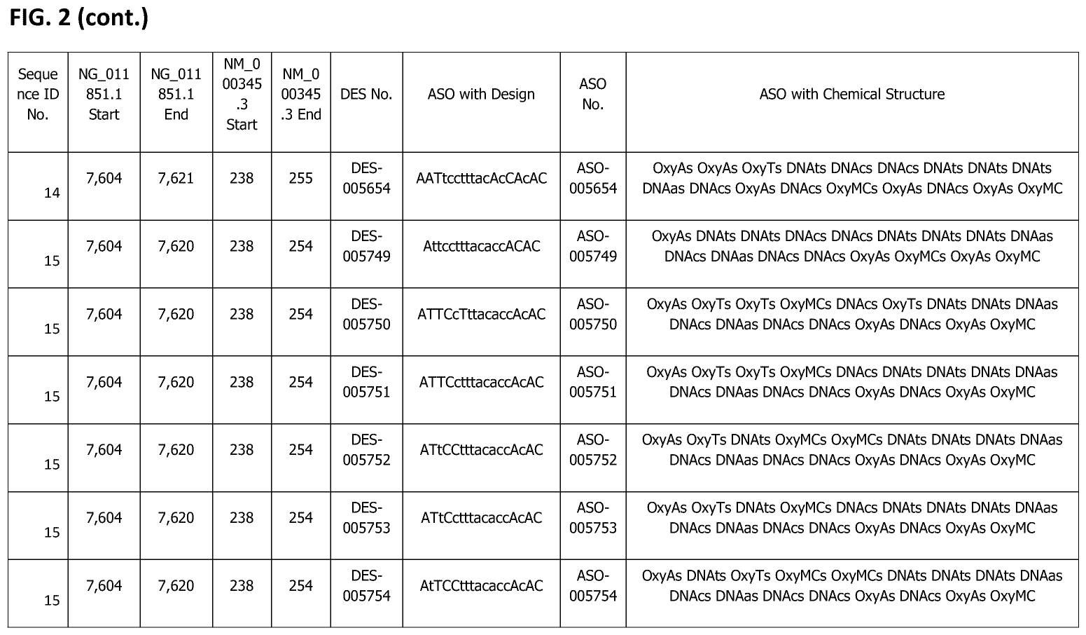

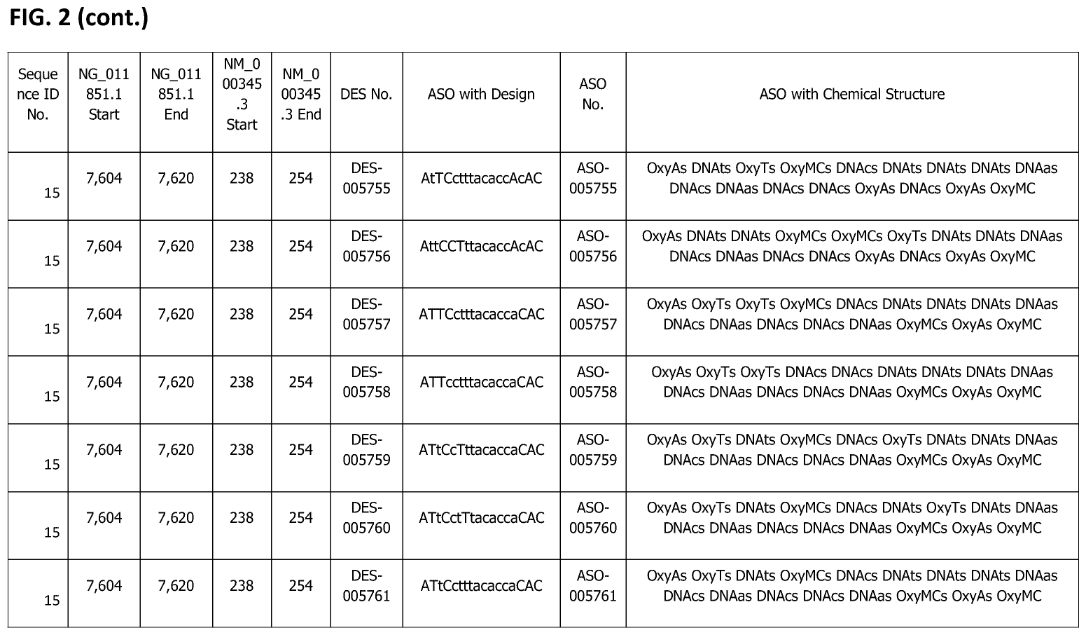

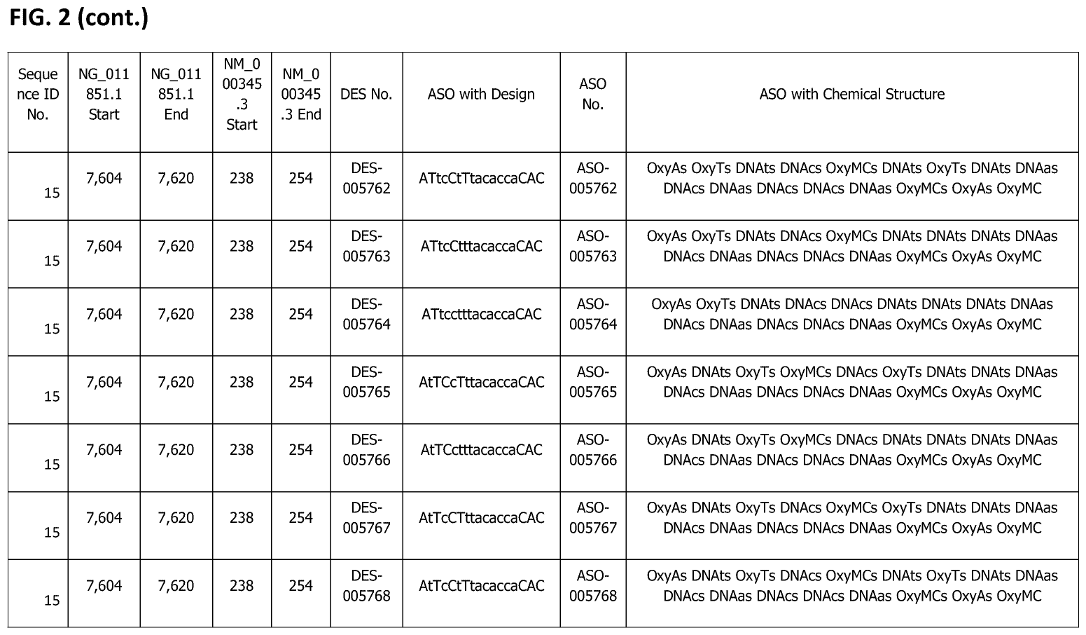

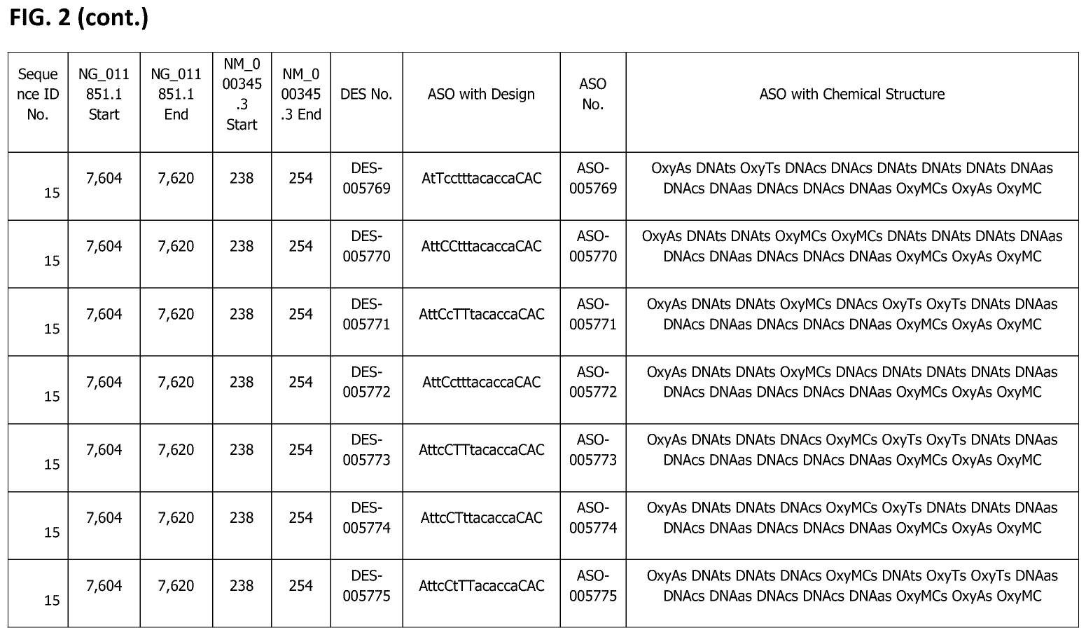

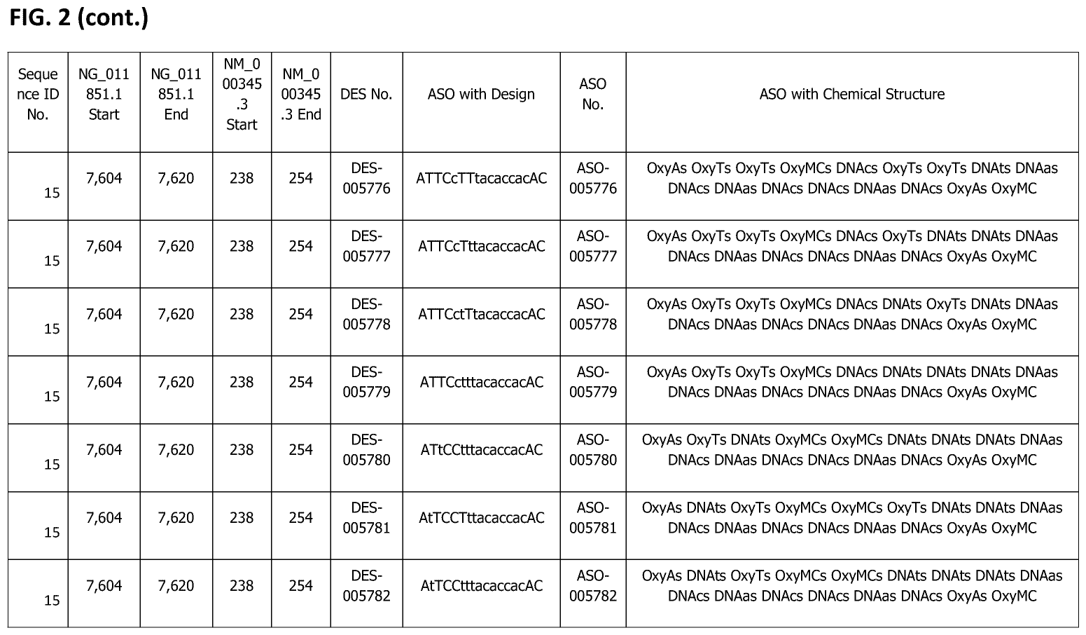

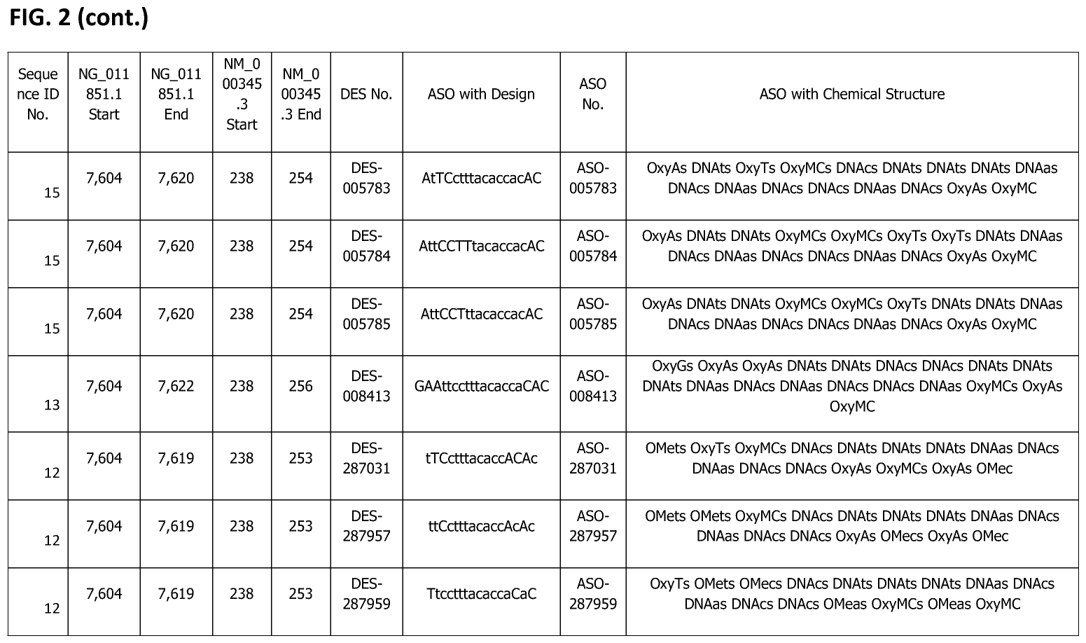

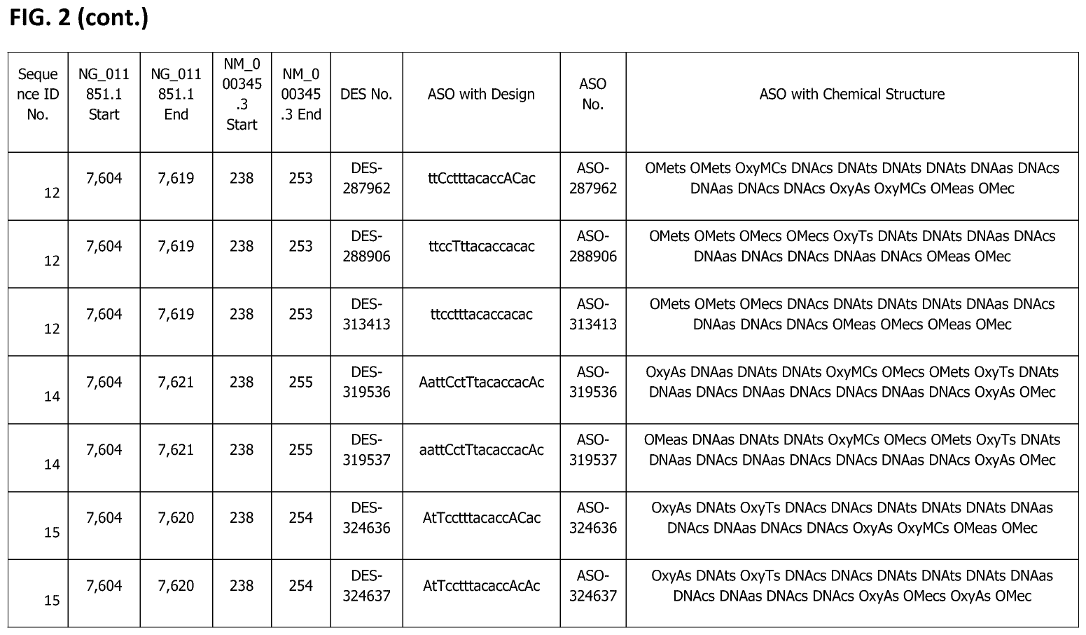

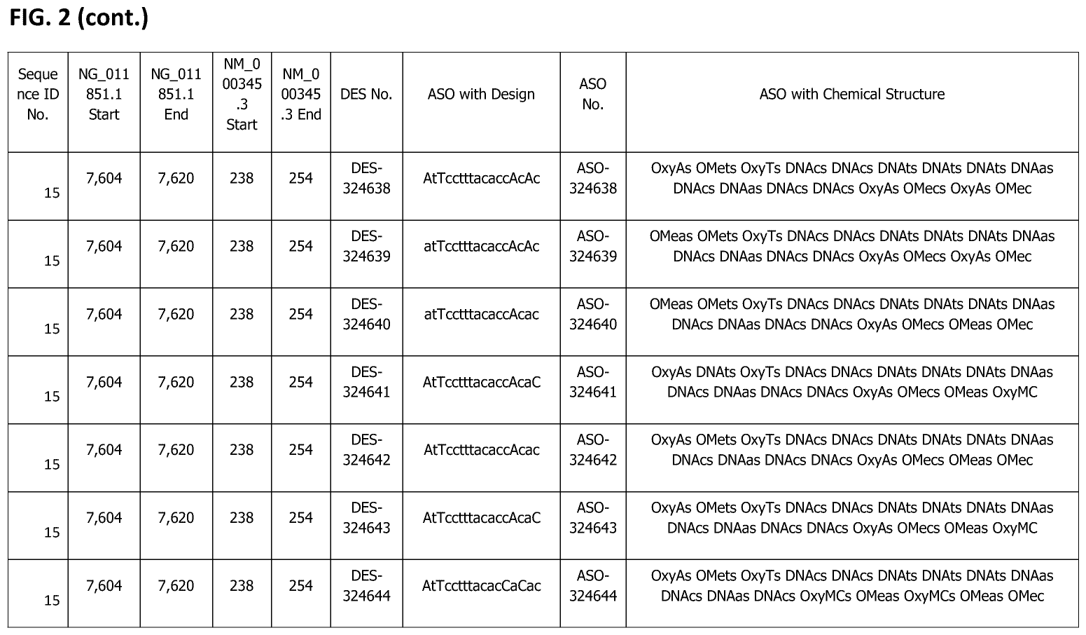

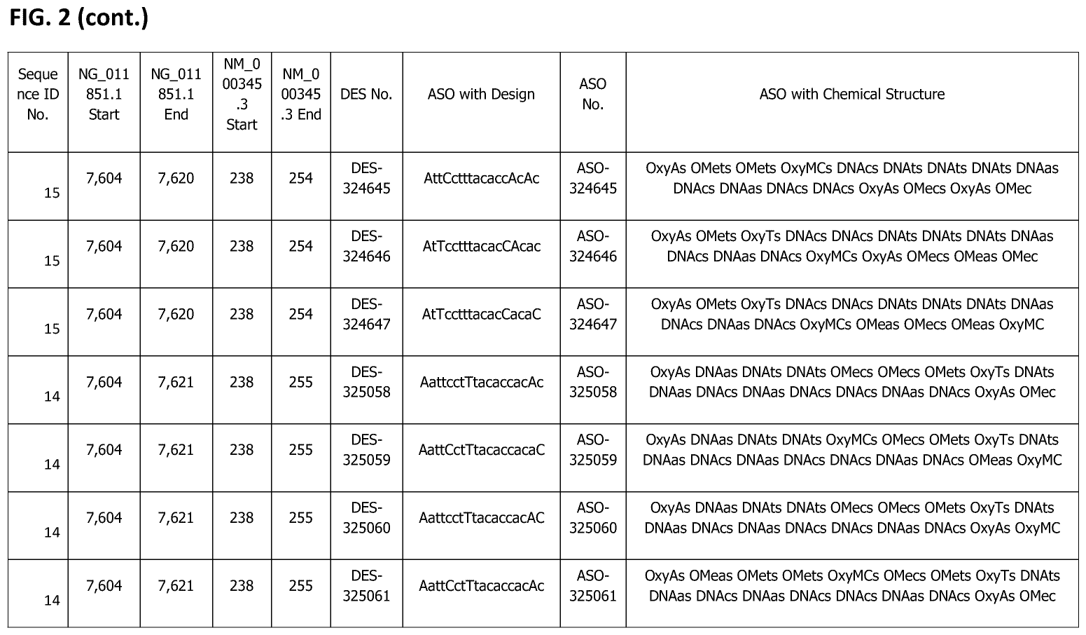

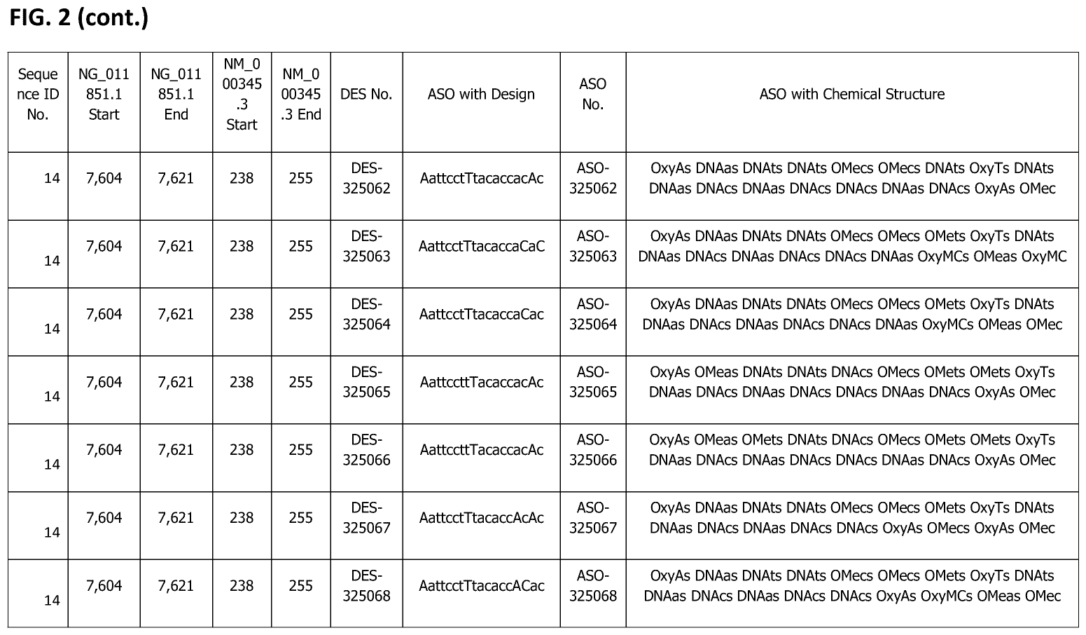

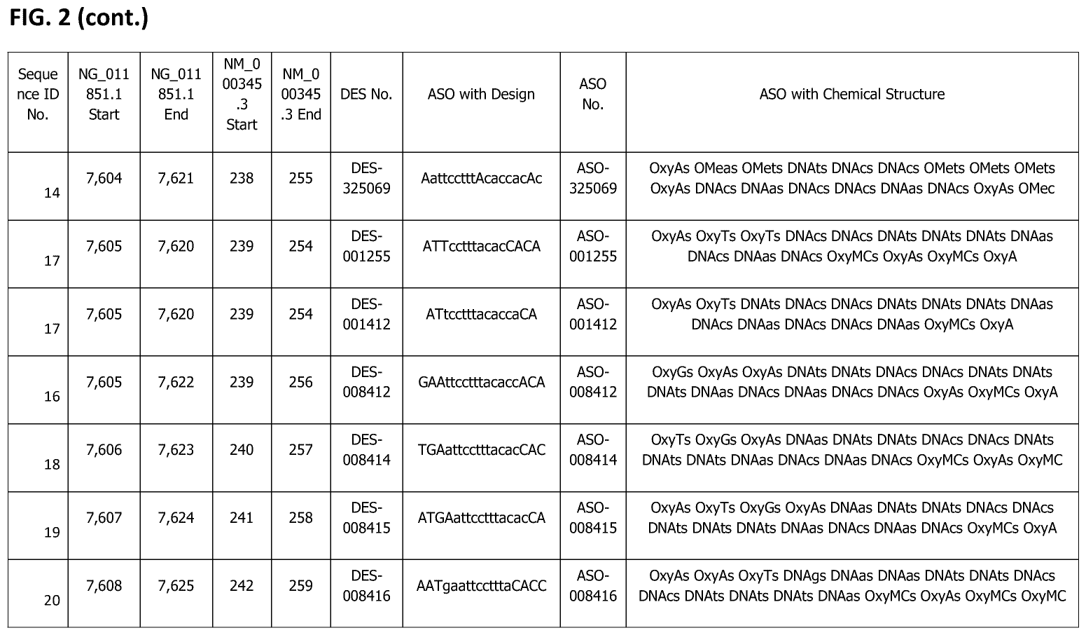

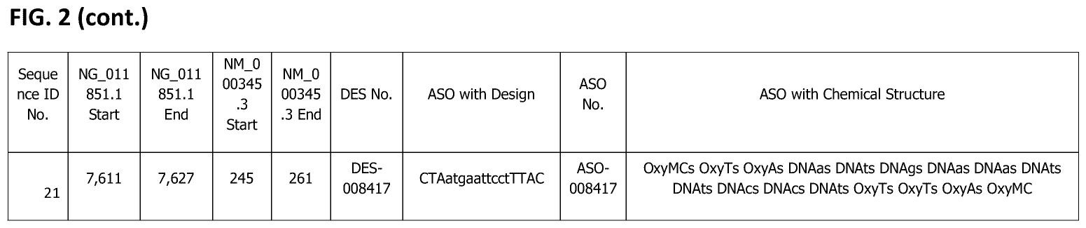

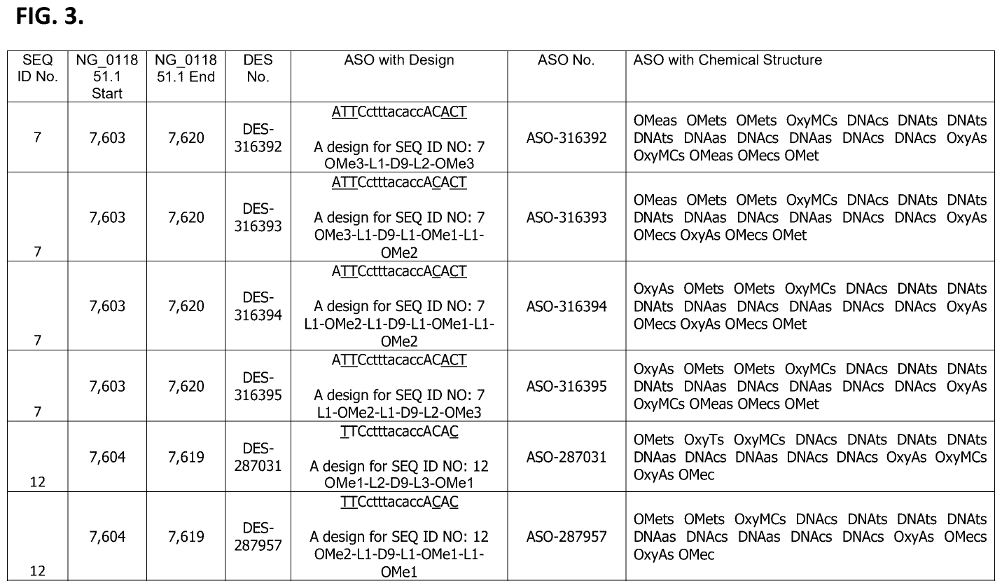

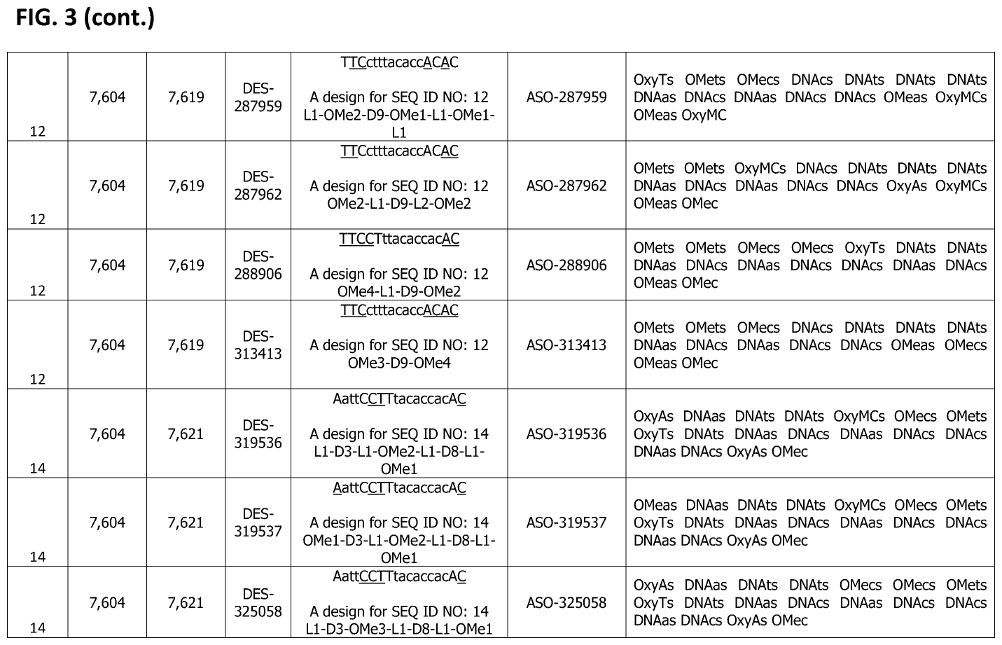

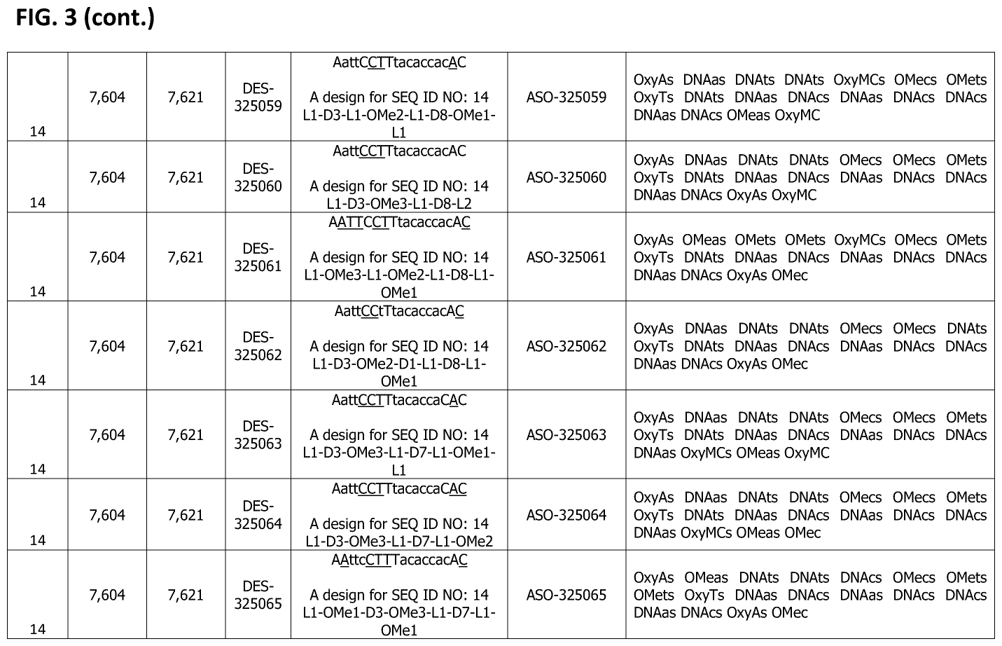

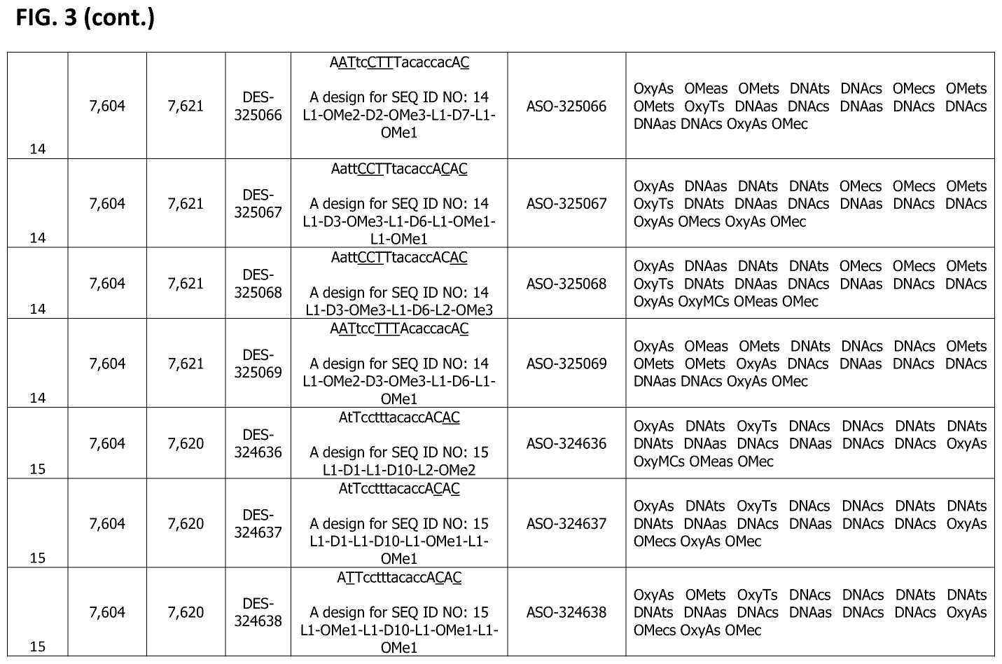

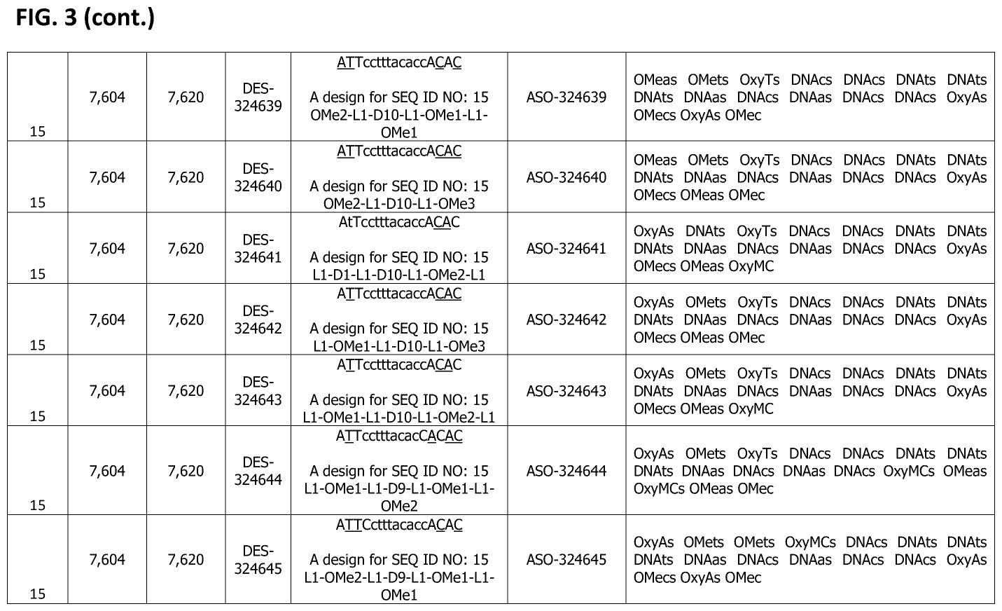

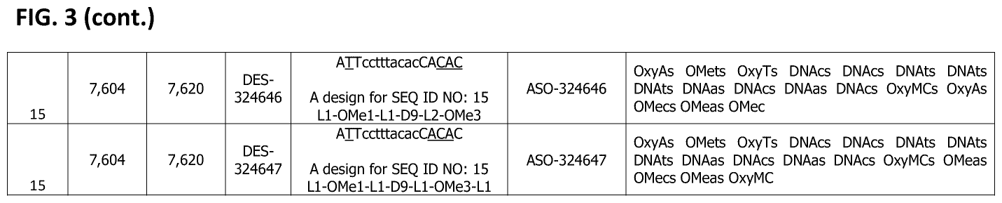

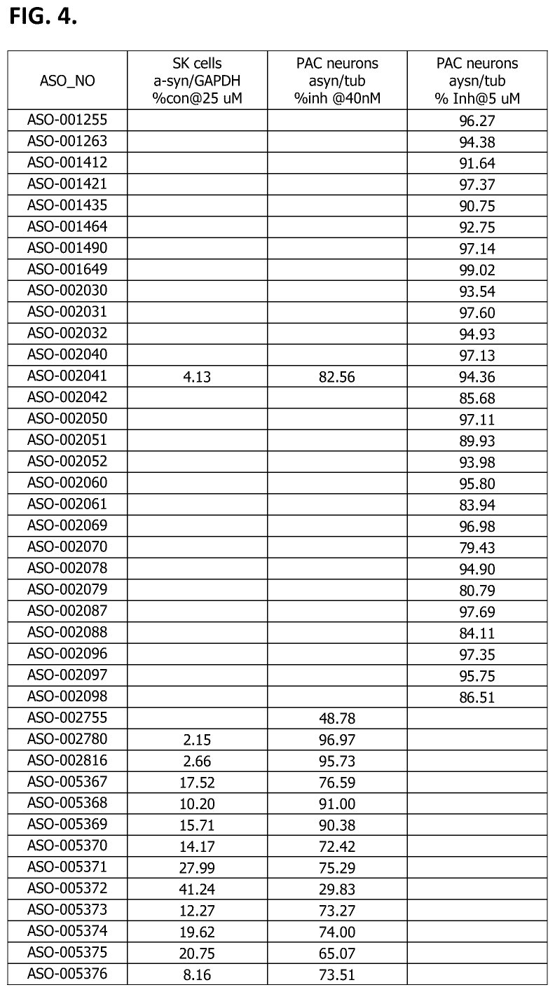

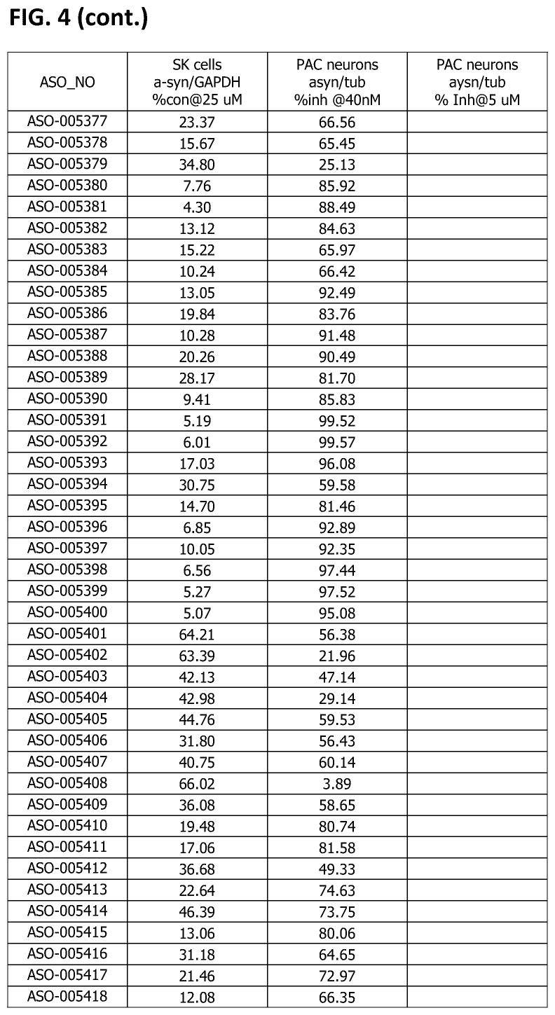

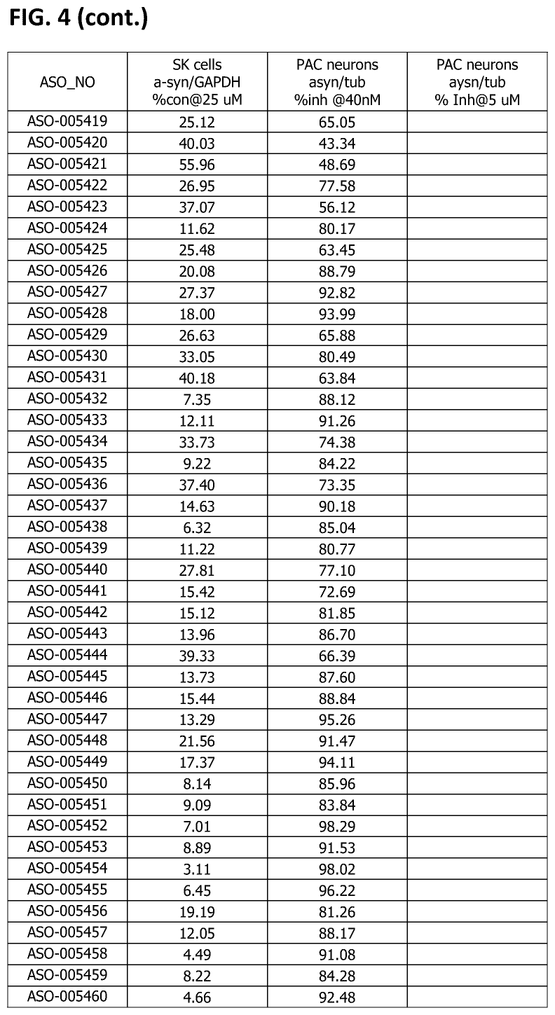

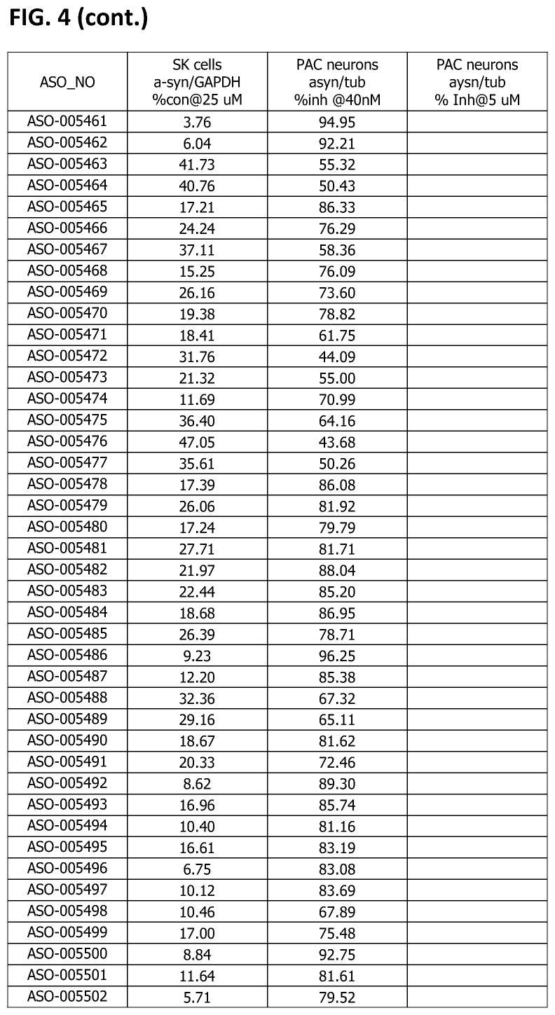

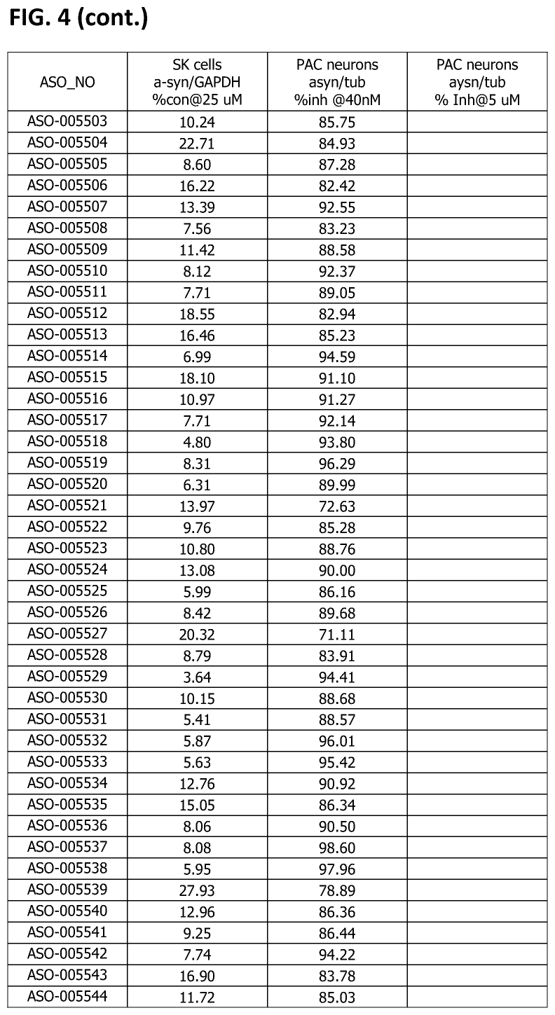

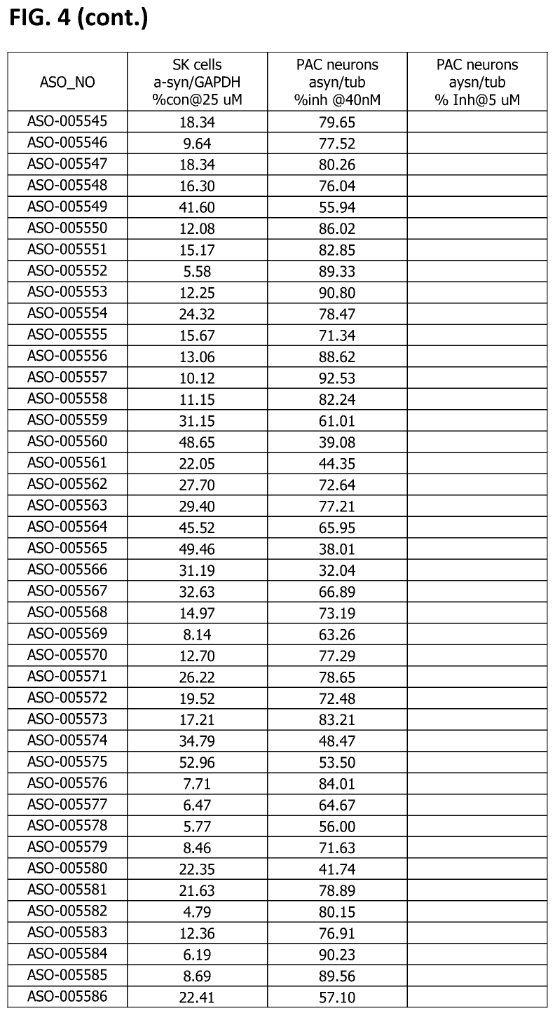

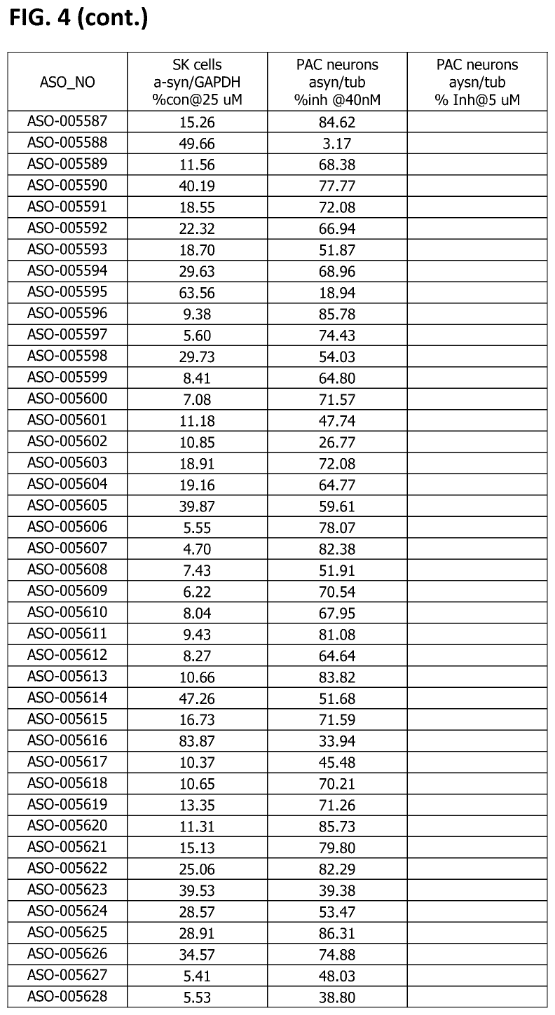

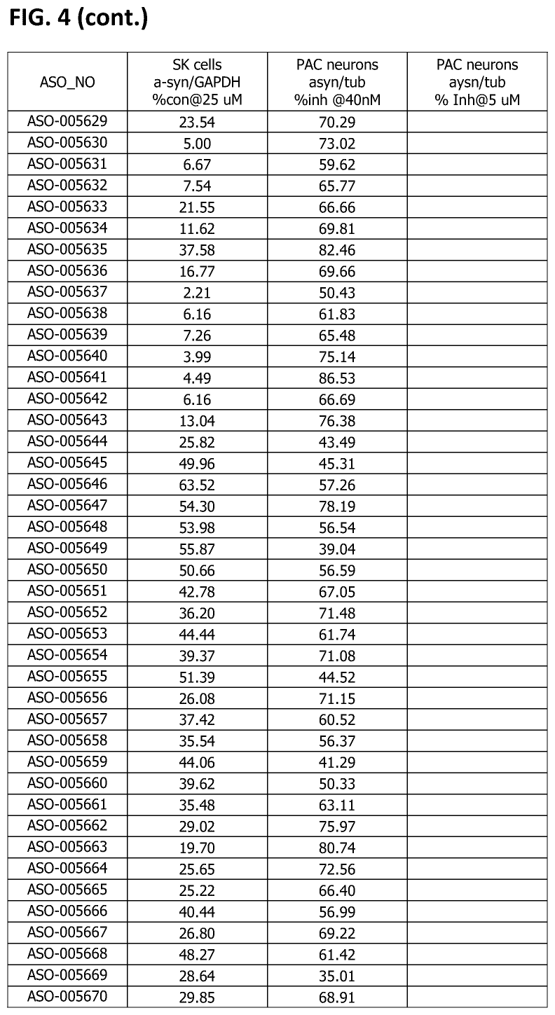

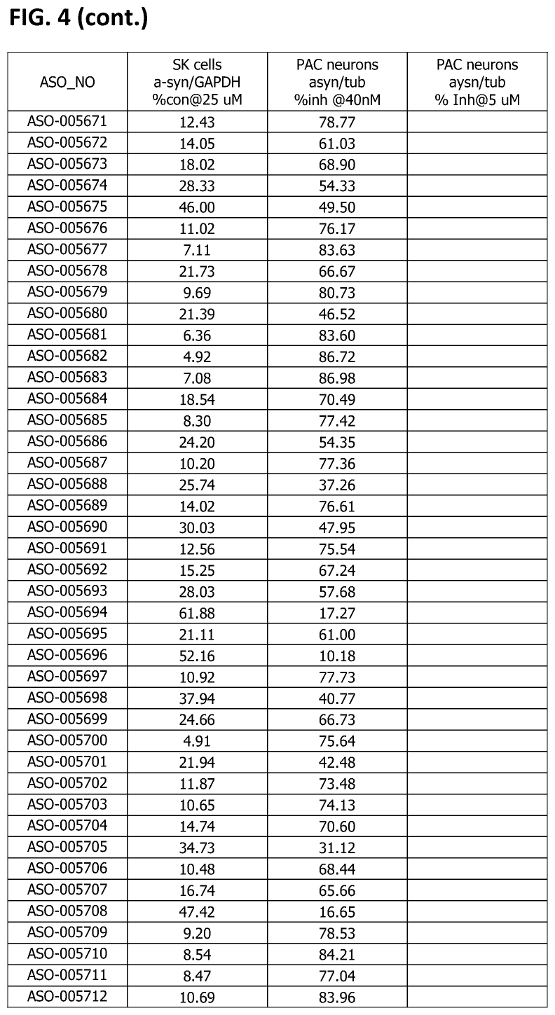

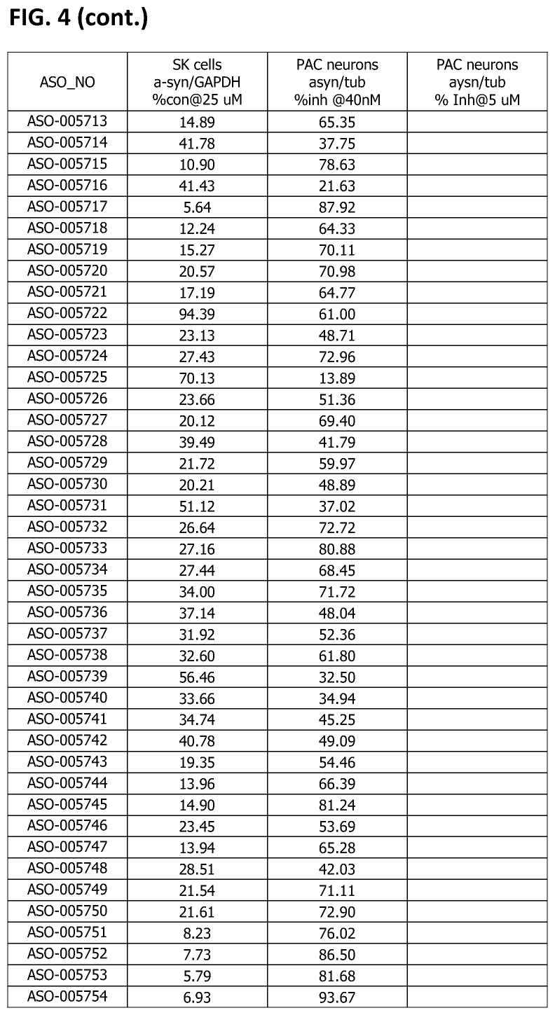

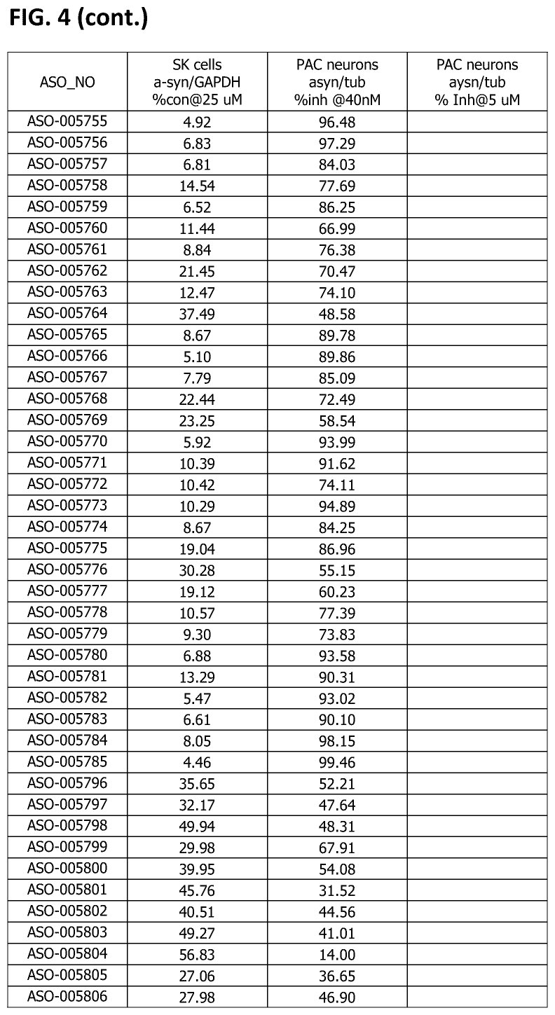

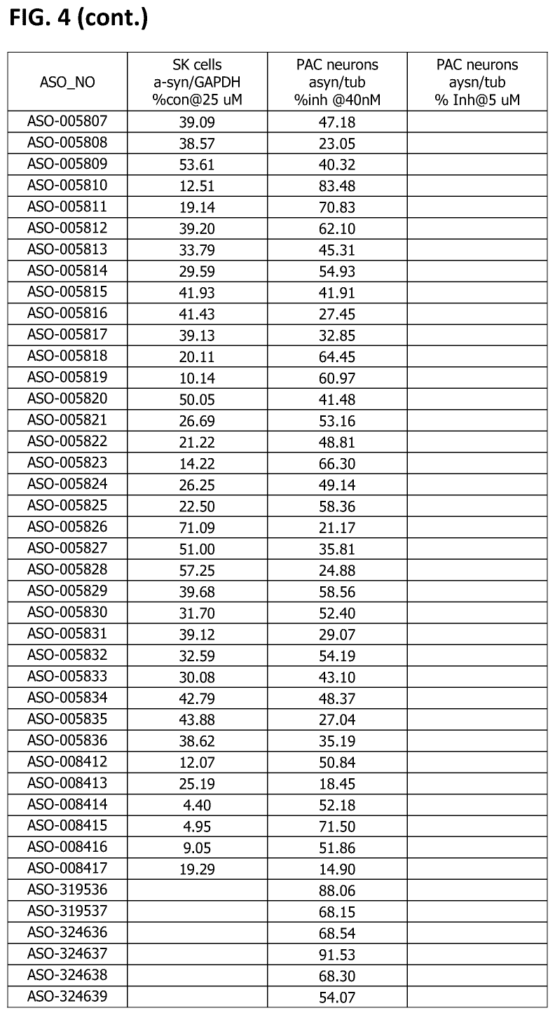

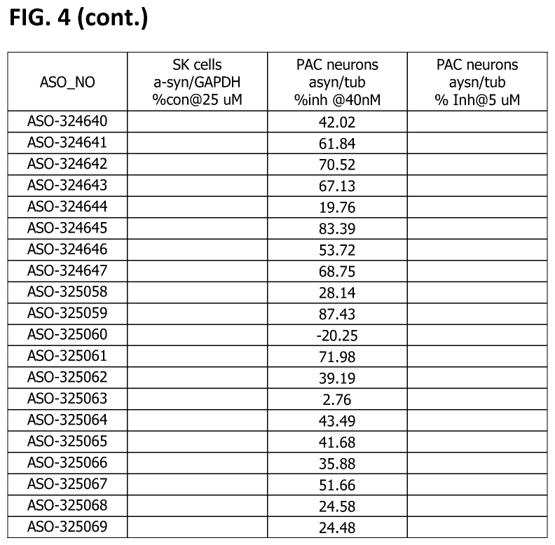

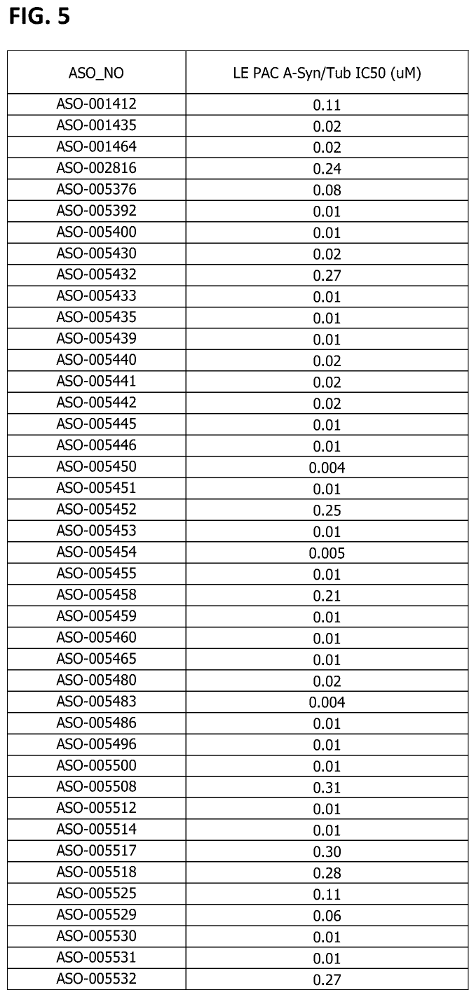

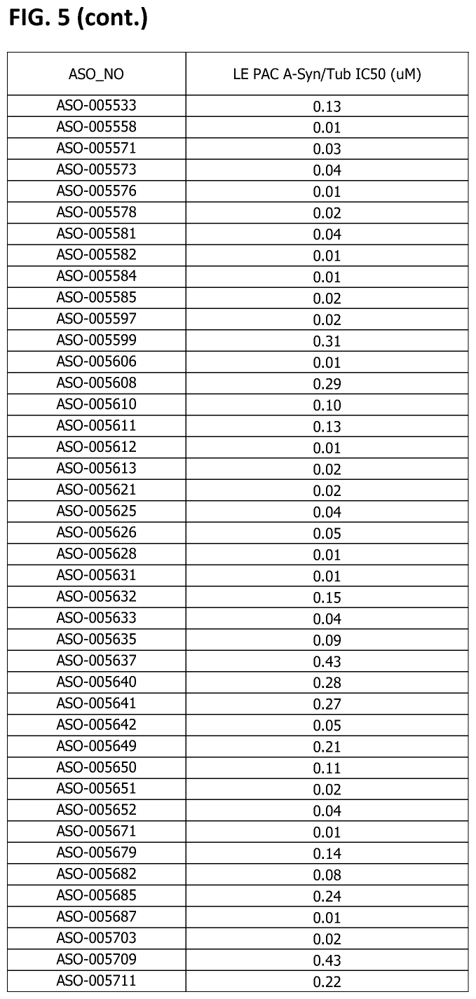

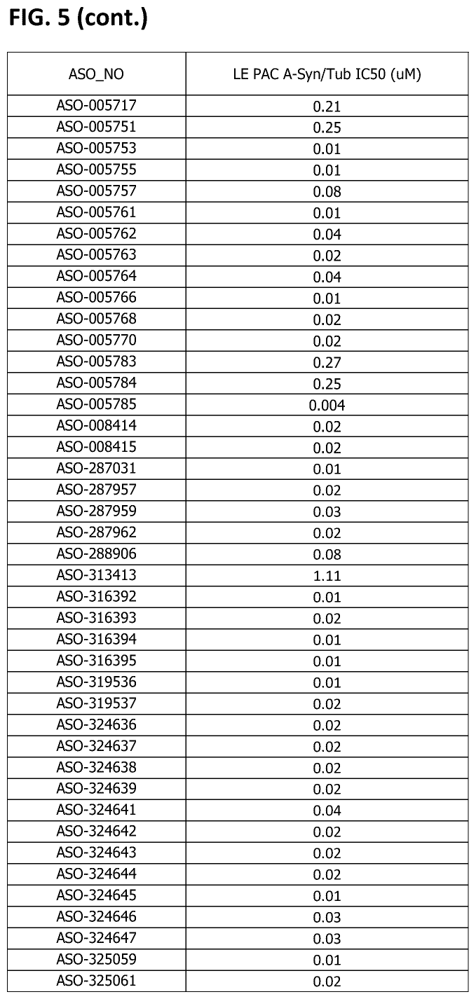

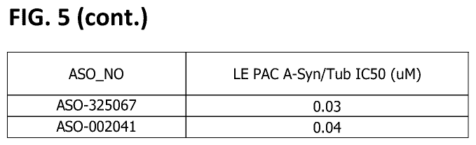

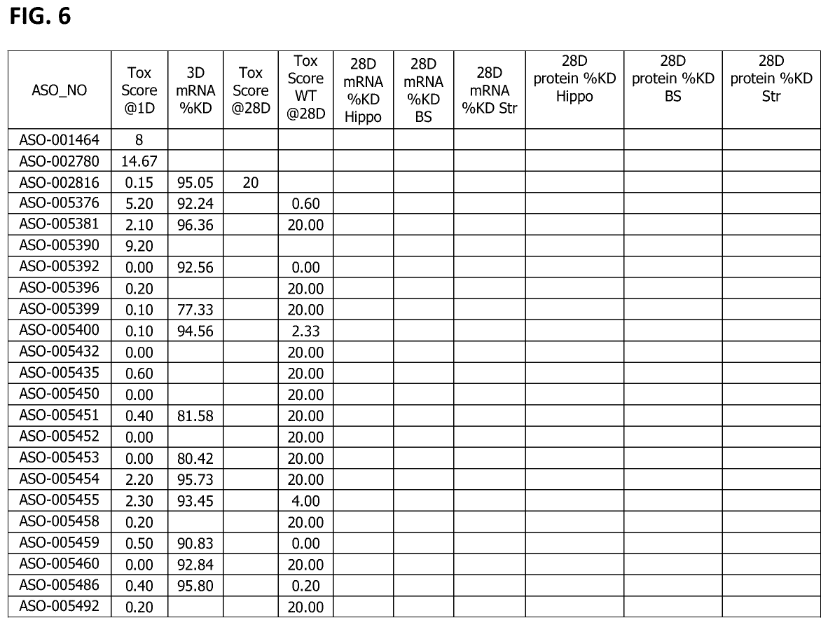

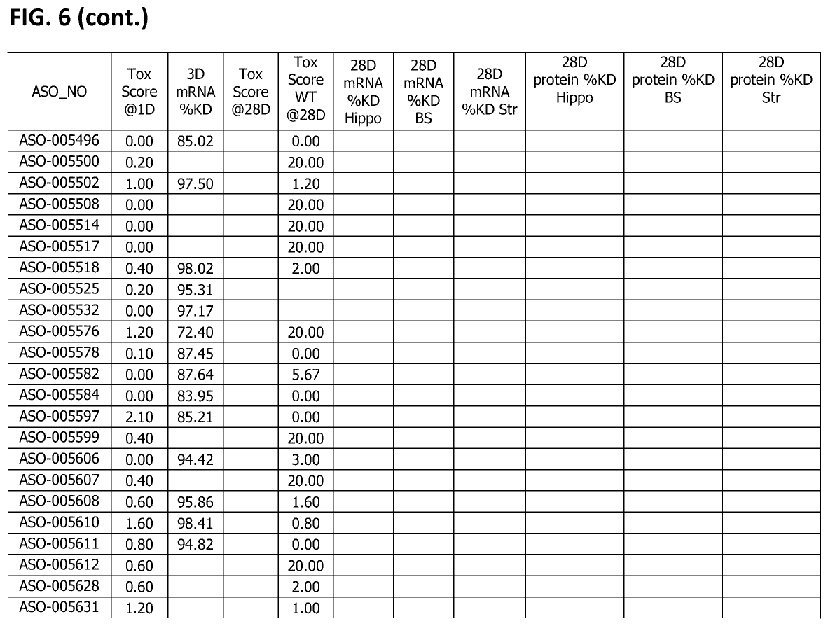

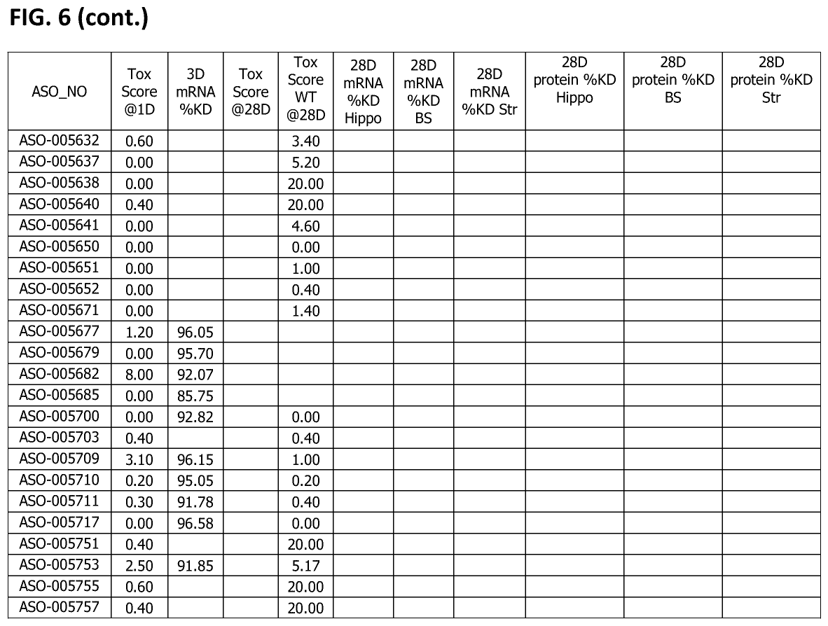

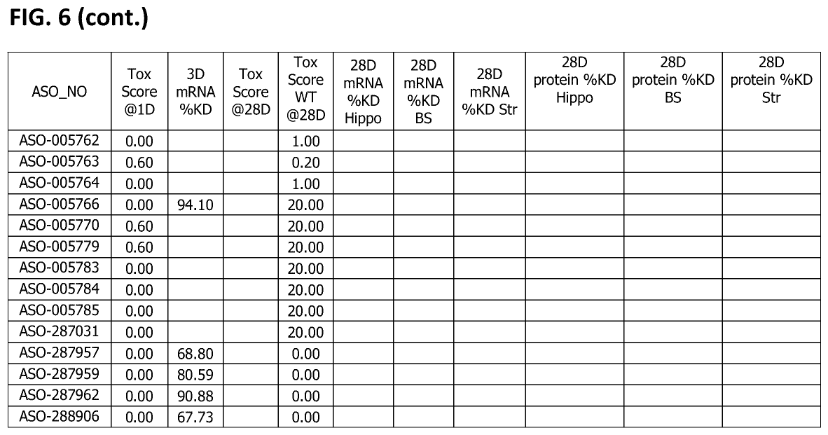

A reference to a SEQ ID number includes a particular nucleobase sequence, but does not include any design or full chemical structure shown in FIG. 2 or 3. Furthermore, the ASOs disclosed in the FIGS. herein show a representative design, but are not limited to the specific design shown in the FIGS. unless otherwise indicated. Herein, a single nucleotide (unit) can also be referred to as a monomer or unit. When this specification refers to a specific ASO number, the reference includes the sequence, the specific ASO design, and the chemical structure. When this specification refers to a specific DES number, the reference includes the sequence and the specific ASO design. For example, when a claim (or this specification) refers to SEQ ID NO: 15, it includes the nucleotide sequence of attcctttacaccacac only. When a claim (or the specification) refers to DES-005459, it includes the nucleotide sequence of attcctttacaccacac with the ASO design shown in the FIGS. (i.e., AtTcctttacaccACAC). Alternatively, the design of ASO-005459 can also be written as SEQ ID NO: 15, wherein each of the first nucleotide, the third nucleotide, and the 14^{th}-17^{th} nucleotides from the 5′ end is a modified nucleotide, e.g., LNA, and each of the other nucleotides is a non-modified nucleotide (e.g., DNA). The ASO number includes the sequence and the ASO design as well as the specific details of the ASO. Therefore, ASO-005459 referred in this application indicates OxyAs DNAts OxyTs DNAcs DNAcs DNAts DNAts DNAts DNAas DNAcs DNAas DNAcs DNAcs OxyAs OxyMCs OxyAs OxyMC, wherein “s” indicates a phosphorothioate linkage.

In various embodiments, the ASO of the disclosure does not comprise RNA (units). In some embodiments, the ASO comprises one or more DNA units. In one embodiment, the ASO according to the disclosure is a linear molecule or is synthesized as a linear molecule. In some embodiments, the ASO is a single stranded molecule, and does not comprise short regions of, for example, at least 3, 4 or 5 contiguous nucleotides, which are complementary to equivalent regions within the same ASO (i.e. duplexes)—in this regard, the ASO is not (essentially) double stranded. In some embodiments, the ASO is essentially not double stranded. In some embodiments, the ASO is not a siRNA. In various embodiments, the ASO of the disclosure can consist entirely of the contiguous nucleotide region. Thus, in some embodiments the ASO is not substantially self-complementary.

In one embodiment, the ASO of the disclosure can be in the form of any pharmaceutically acceptable salts. The term “pharmaceutically acceptable salts” as used herein refers to derivatives of the ASOs of the disclosure wherein the ASO is modified (e.g., addition of a cation) by making salts thereof. Such salts retain the desired biological activity of the ASOs without imparting undesired toxicological effects. In some embodiments, the ASO of the disclosure is in the form of a sodium salt. In other embodiments, the ASO is in the form of a potassium salt.

### II.A. The Target

Suitably the ASO of the disclosure is capable of down-regulating (e.g., reducing or removing) expression of the SNCA mRNA or protein. In this regard, the ASO of the disclosure can affect indirect inhibition of SNCA protein through the reduction in SNCA mRNA levels, typically in a mammalian cell, such as a human cell, such as a neuronal cell. In particular, the present disclosure is directed to ASOs that target one or more regions of the SNCA pre-mRNA.

Synonyms of SNCA are known and include NACP, non A-beta component of AD amyloid, PARK1, PARK4, and PD1. The sequence for the SNCA gene can be found under publicly available Accession Number NC_000004.12 and the partial sequence for the SNCA pre-mRNA transcript (residues 6001 to 8400) can be found under publicly available Accession Number NG_011851.1 (SEQ ID NO: 1). The sequence for SNCA protein can be found under publicly available Accession Numbers: P37840, A8K2A4, Q13701, Q4JHI3, and Q6IAU6, each of which is incorporated by reference herein in its entirety. Natural variants of the SNCA gene product are known. For example, natural variants of SNCA protein can contain one or more amino acid substitutions selected from: A30P, E46K, H50Q, A53T, and any combinations thereof. Therefore, the ASOs of the present disclosure can be designed to reduce or inhibit expression of the natural variants of the SNCA protein.

Mutations in SNCA are known to cause one or more pathological conditions. The ASOs of the disclosure can be used to reduce or inhibit the expression of a SNP or alternatively spliced SNCA transcript containing one or more mutations and consequently reduce the formation of a mutated SNCA protein. Examples of SNCA protein mutants include, but are not limited to a SNCA protein comprising one or more mutations selected from: D2A, E35K, Y39F, H50A, E57K, G67_V71del, V71_V82del, A76_V77del, A76del, V77del, A78del, A85_F94del, Y125F, Y133F, Y136F, and any combination thereof. The ASO of the disclosure can be designed to reduce or inhibit expression of any mutants of SNCA proteins.

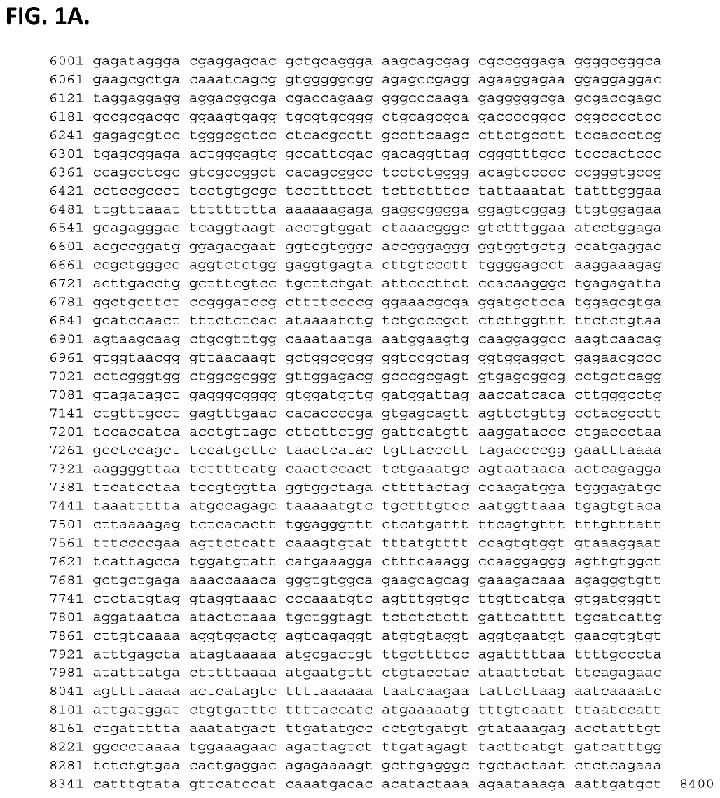

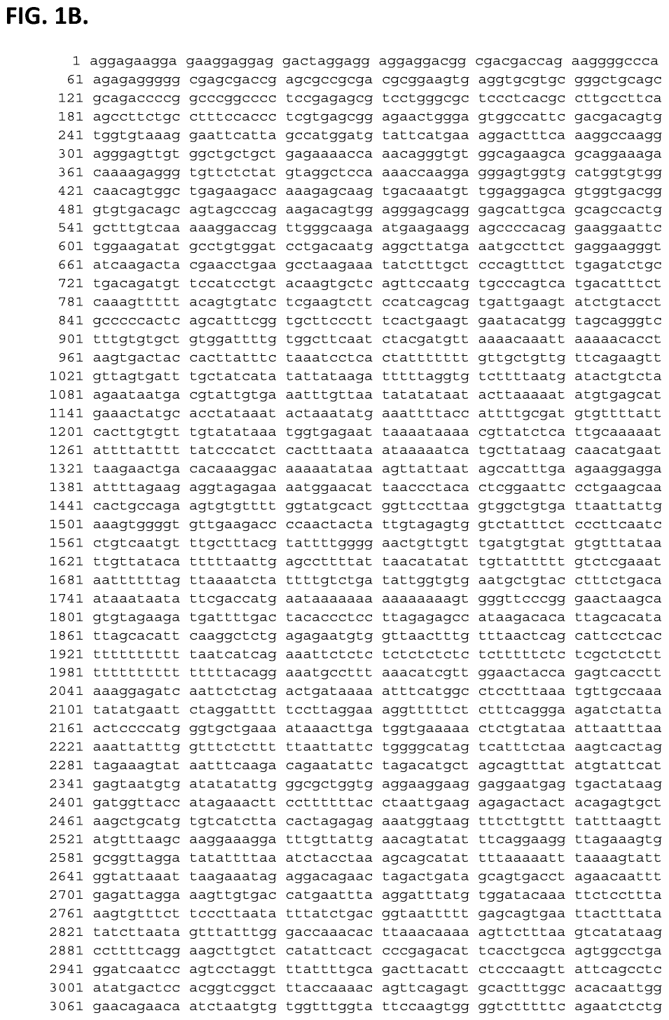

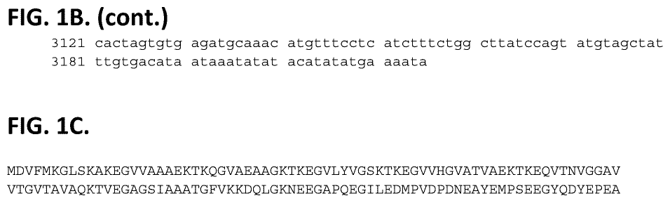

An example of a target nucleic acid sequence of the ASOs is SNCA pre-mRNA. SEQ ID NO: 1 in FIG. 1A represents a partial SNCA genomic sequence (residues 6001 to 8400). SEQ ID NO: 1 is identical to a SNCA pre-mRNA sequence except that the nucleotide “t” in SEQ ID NO: 1 is shown as “u” in the pre-mRNA. In certain embodiments, the “target nucleic acid” comprises an intron region of an SNCA protein-encoding nucleic acids or naturally occurring variants thereof, and RNA nucleic acids derived therefrom, e.g., pre-mRNA. In other embodiments, the “target nucleic acid” comprises an exon region of an SNCA protein-encoding nucleic acids or naturally occurring variants thereof, and RNA nucleic acids derived therefrom. In some embodiments, for example when used in research or diagnostics the “target nucleic acid” can be a cDNA or a synthetic oligonucleotide derived from the above DNA or RNA nucleic acid targets. In one embodiment, the partial SNCA genomic sequence is shown as GenBank Accession No. NG_011851.1 (residues 6001 to 8400) (SEQ ID NO: 1). The mRNA encoding SNCA protein is shown as SEQ ID NO: 2. See FIG. 1B. The SNCA protein sequence encoded by the SNCA mRNA is shown as SEQ ID NO: 3. See FIG. 1C.

In one embodiment, the ASO according to the disclosure comprises a contiguous nucleotide sequence of 10 to 30 nucleotides in length that are complementary to a nucleic acid sequence within a SNCA transcript, e.g., a region corresponding to an intron exon junction of SEQ ID NO: 1, wherein the nucleic acid sequence corresponds to nucleotides 7,602-7,627 of SEQ ID NO: 1

In certain embodiments, the ASOs hybridize to or are complementary to a region within a SNCA transcript, e.g., nucleotides 7,602-7,627 of SEQ ID NO: 1, and have a sequence score equal to or greater than about 0.1, 0.2, 0.3, 0.4, 0.5, 0.6, 0.7, 0.8, 0.9, or 1.0. Calculation methods of the sequence score are disclosed elsewhere herein.

In some embodiments, the ASOs of the present disclosure hybridize to an intron exon junction of a SNCA transcript, e.g., a region corresponding to a junction between intron 1 and exon 2 of SEQ ID NO: 1. In some embodiments, the ASO of the disclosure comprises a contiguous nucleotide sequence that hybridizes to a nucleic acid sequence, or a region within the sequence, of a SNCA transcript (“target region”), wherein the nucleic acid sequence corresponds to nucleotides 7,602-7,627 of SEQ ID NO: 1. In another embodiment, the ASO of the disclosure comprises a contiguous nucleotide sequence that hybridizes to a nucleic acid sequence, or a region within the sequence, of a SNCA transcript, wherein the nucleic acid sequence corresponds to nucleotides 7,602-7,627 of SEQ ID NO: 1, and wherein the ASO has one of the designs described herein (e.g., Section II.H. e.g., a gapmer design, e.g., an alternating flank gapmer design) or a chemical structure shown elsewhere herein (e.g., FIGS. 2 and 3).

In some embodiments, the target region corresponds to nucleotides 7,604-7,620 of SEQ ID NO: 1. In other embodiments, the target region corresponds to nucleotides 7,603-7,620 of SEQ ID NO: 1. In other embodiments, the target region corresponds to nucleotides 7,602-7,619 of SEQ ID NO: 1. In certain embodiments, the ASOs hybridize to a region within an exon of a SNCA transcript, e.g., SEQ ID NO: 1, and have a sequence score equal to or greater than about 0.1, 0.2, 0.3, 0.4, 0.5, 0.6, 0.7, 0.8, 0.9, or 1.0. Calculation methods of the sequence score are disclosed elsewhere herein.

In certain embodiments, the target region corresponds to nucleotides 7,602-7,627 of SEQ ID NO: 1±10 nucleotides at the 3′ end, the 5′ end, or both. In other embodiments, the target region corresponds to nucleotides 7,604-7,620 of SEQ ID NO: 1±1, ±2, ±3, ±4, ±5, ±6, ±7, ±8, ±9, or ±10 nucleotides at the 3′ end, the 5′ end, or both.

In certain embodiments, the ASO of the disclosure is capable of hybridizing to the target nucleic acid (e.g., SNCA transcript) under physiological condition, i.e., in vivo condition. In some embodiments, the ASO of the disclosure is capable of hybridizing to the target nucleic acid (e.g., SNCA transcript) in vitro. In some embodiments, the ASO of the disclosure is capable of hybridizing to the target nucleic acid (e.g., SNCA transcript) in vitro under stringent conditions. Stringency conditions for hybridization in vitro are dependent on, inter alia, productive cell uptake, RNA accessibility, temperature, free energy of association, salt concentration, and time (see, e.g., Stanley T Crooks, Antisense Drug Technology: Principles, Strategies and Applications, 2^{nd} Edition, CRC Press (2007)). Generally, conditions of high to moderate stringency are used for in vitro hybridization to enable hybridization between substantially similar nucleic acids, but not between dissimilar nucleic acids. An example of stringent hybridization conditions include hybridization in 5× saline-sodium citrate (SSC) buffer (0.75 M sodium chloride/0.075 M sodium citrate) for 1 hour at 40° C., followed by washing the sample 10 times in 1×SSC at 40° C. and 5 times in 1×SSC buffer at room temperature. In vivo hybridization conditions consist of intracellular conditions (e.g., physiological pH and intracellular ionic conditions) that govern the hybridization of antisense oligonucleotides with target sequences. In vivo conditions can be mimicked in vitro by relatively low stringency conditions. For example, hybridization can be carried out in vitro in 2×SSC (0.3 M sodium chloride/0.03 M sodium citrate), 0.1% SDS at 37° C. A wash solution containing 4×SSC, 0.1% SDS can be used at 37° C., with a final wash in 1×SSC at 45° C.

### II.B. ASO Sequences

The ASOs of the disclosure comprise a contiguous nucleotide sequence which corresponds to the complement of a region of SNCA transcript, e.g., a nucleotide sequence corresponding to SEQ ID NO: 1.

In certain embodiments, the disclosure provides an ASO which comprises a contiguous nucleotide sequence of a total of from 10-30 nucleotides, such as 10-15 nucleotides, 10-20 nucleotides, or 10-25 nucleotides in length wherein the contiguous nucleotide sequence has at least about 85%, at least about 90%, at least about 95%, at least about 98%, or at least about 99% sequence identity to a region within the complement of a mammalian SNCA transcript, such as SEQ ID NO: 1 or SEQ ID NO: 2.

The ASO can comprise a contiguous nucleotide sequence which is fully complementary (perfectly complementary) to the equivalent region of a nucleic acid which encodes a mammalian SNCA protein (e.g., SEQ ID NOs: 1 and 2). The ASO can comprise a contiguous nucleotide sequence which is fully complementary (perfectly complementary) to a nucleic acid sequence, or a region within the sequence, corresponding to nucleotides X-Y of SEQ ID NO: 1, wherein X and Y are the pre-mRNA start site and the pre-mRNA end site of NG_011851.1, respectively, as shown in FIG. 2. Furthermore, the ASO can have a design described elsewhere herein (e.g., Section II.G. e.g., a gapmer design, e.g., an alternating flank gapmer design) or a chemical structure shown elsewhere herein (e.g., FIGS. 2 and 3). In some embodiments, the ASO comprises a contiguous nucleotide sequence which is fully complementary (perfectly complementary) to a nucleic acid sequence, or a region within the sequence, corresponding to nucleotides X-Y of SEQ ID NO: 2, wherein X and Y are the mRNA start site and the mRNA end site, respectively.

In certain embodiments, the nucleotide sequence of the ASOs of the disclosure or the contiguous nucleotide sequence has at least about 80% sequence identity to a sequence selected from SEQ ID NOs: 4 to 21 (i.e., the sequences in FIGS. 2 and 3), such as at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some embodiments, the ASO has a design described elsewhere herein (e.g., Section II.G.I, e.g., a gapmer design, e.g., an alternating flank gapmer design) or a nucleoside chemical structure shown elsewhere herein (e.g., FIGS. 2 and 3).

In some embodiments, the ASO of the disclosure comprises at least one ASO with the design (e.g., DES number) disclosed in FIGS. 2 and 3. In some embodiments, the ASO of the disclosure comprises at least one ASO with the design (e.g., DES number) disclosed in FIGS. 2 and 3, wherein the ASO is one nucleotide, two nucleotides, three nucleotides, or four nucleotides shorter at the 3′ end than the ASOs disclosed in FIGS. 2 and 3. In other embodiments, the ASO of the disclosure comprises at least one ASO with the design (e.g., DES number) disclosed in FIGS. 2 and 3, wherein the ASO is one nucleotide, two nucleotides, three nucleotides, or four nucleotides shorter at the 5′ end than the ASOs disclosed in FIGS. 2 and 3. In yet other embodiments, the ASO of the disclosure comprises at least one ASO with the design (e.g., DES number) disclosed in FIGS. 2 and 3, wherein the ASO is one nucleotide, two nucleotides, three nucleotides, or four nucleotides shorter at the 5′ end and/or the 3′ end than the ASOs disclosed in FIGS. 2 and 3.

In other embodiments, the ASO of the disclosure comprises at least one ASO with the chemical structure (e.g., ASO number) disclosed in FIGS. 2 and 3. In some embodiments, the ASO of the disclosure comprises at least one ASO with the chemical structure (e.g., ASO number) disclosed in FIGS. 2 and 3, wherein the ASO is one nucleotide, two nucleotides, three nucleotides, or four nucleotides shorter at the 3′ end than the ASOs disclosed in FIGS. 2 and 3. In other embodiments, the ASO of the disclosure comprises at least one ASO with the chemical structure (e.g., ASO number) disclosed in FIGS. 2 and 3, wherein the ASO is one nucleotide, two nucleotides, three nucleotides, or four nucleotides shorter at the 5′ end than the ASOs disclosed in FIGS. 2 and 3. In yet other embodiments, the ASO of the disclosure comprises at least one ASO with the chemical structure (e.g., ASO number) disclosed in FIGS. 2 and 3, wherein the ASO is one nucleotide, two nucleotides, three nucleotides, or four nucleotides shorter at the 5′ end and/or the 3′ end than the ASOs disclosed in FIGS. 2 and 3.

In some embodiments the ASO (or contiguous nucleotide portion thereof) is selected from, or comprises, one of the sequences selected from the group consisting of SEQ ID NOs: 4 to 21 and a region of at least 10 contiguous nucleotides thereof, wherein the ASO (or contiguous nucleotide portion thereof) can optionally comprise one, two, three, or four mismatches when compared to the corresponding SNCA transcript.

In one embodiment, the ASO comprises a sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21. In some embodiments, the ASO comprises a sequence selected from the group consisting of SEQ ID NO: 7 (e.g., the sequence of ASO-005578 and ASO-005584) and SEQ ID NO: 15 (e.g., the sequence of ASO-005459).

In some embodiments, the ASOs of the disclosure bind to the target nucleic acid sequence (e.g., SNCA transcript) and are capable of inhibiting or reducing expression of the SNCA transcript by at least about 10%, at least about 20%, at least about 30%, at least about 40%, at least about 50%, at least about 60%, at least about 70%, at least about 80%, at least about 90%, or about 100% in a tissue (e.g., a brain region) of a mouse expressing a human SNCA gene (e.g., A53T-PAC) when administered in vivo at doses of 3.13 μg, 12.5 μg, 25 μg, 50 μg, or 100 μg compared to the control (e.g., an internal control such as GADPH or tubulin, or a mouse administered with vehicle control alone), as measured by an assay, e.g., quantitative PCR or QUANTIGENE® analysis disclosed herein.

In some embodiments, the ASOs are capable of reducing expression of SNCA protein by at least about 10%, at least about 20%, at least about 30%, at least about 40%, at least about 50%, at least about 60%, at least about 70%, at least about 80%, at least about 90%, or about 100% in a tissue (e.g., a brain region) of a mouse expressing a human SNCA gene (e.g., A53T-PAC) when administered in vivo at doses of 3.13 μg, 12.5 μg, 25 μg, 50 μg, or 100 μg compared to the control (e.g., an internal control such as GADPH or tubulin, or a mouse administered with vehicle control alone), as measured by an assay, e.g., High Content Assay disclosed herein (see Example 2A).

In some embodiments, the ASOs of the disclosure bind to the target nucleic acid sequence (e.g., SNCA transcript) and are capable of inhibiting or reducing expression of the SNCA transcript by at least about 10%, at least about 20%, at least about 30%, at least about 40%, at least about 50%, at least about 60%, at least about 70%, at least about 80%, at least about 90%, or about 100% in a tissue (e.g., a brain region) of a cyno expressing the wild-type SNCA gene when administered once or twice in vivo at doses of 2 mg, 4 mg, or, 8 mg, compared to the control (e.g., an internal control such as GADPH or tubulin, or a cyno administered with vehicle control alone), as measured by an assay, e.g., quantitative PCR or QUANTIGENE® analysis disclosed herein.

In some embodiments, the ASOs are capable of reducing expression of SNCA protein by at least about 10%, at least about 20%, at least about 30%, at least about 40%, at least about 50%, at least about 60%, at least about 70%, at least about 80%, at least about 90%, or about 100% in a tissue (e.g., a brain region) of a cyno expressing the wild-type SNCA gene when administered once or twice in vivo at doses of 2 mg, 4 mg, or 8 mg, compared to the control (e.g., an internal control such as GADPH or tubulin, or a cyno administered with vehicle control alone), as measured by an assay, e.g., High Content Assay disclosed herein (see Example 2A).

In other embodiments, the ASOs of the disclosure are capable of reducing expression of SNCA mRNA in vitro by at least about 20%, at least about 30%, at least about 40%, at least about 50%, at least about 60%, at least about 70%, at least about 80%, at least about 90%, or about 100% in mouse primary neurons expressing a full-length human SNCA gene (e.g., PAC neurons) when the neurons are in contact with 5 μM, 3.3 μM, 1 μM, 4 nM, 40 nM, or 200 nM of the antisense oligonucleotide compared to a control (e.g., an internal control such as GADPH or tubulin, or a mouse primary neurons expressing a full-length human SNCA gene in contact with saline alone), as measured by an assay, e.g., QUANTIGENE® analysis disclosed herein.

In yet other embodiments, the ASOs are capable of reducing expression of SNCA protein in vitro by at least about 60%, at least about 70%, at least about 80%, at least about 90%, or at least about 95% in mouse primary neurons expressing a full-length human SNCA gene (e.g., PAC neurons) when the neurons are in contact with 5 μM, 3.3 μM, 1 μM, 4 nM, 40 nM, or 200 nM of the antisense oligonucleotide compared to control (e.g., an internal control such as GADPH or tubulin, or a mouse primary neurons expressing a full-length human SNCA gene (in contact with saline alone), as measured by an assay, e.g., High Content Assay disclosed herein (see Example 2A).

In some embodiments, the ASOs are capable of reducing expression of SNCA mRNA in vitro by at least about 10%, at least about 20%, at least about 30%, at least about 40%, at least about 50%, at least about 60%, at least about 70%, at least about 80%, at least about 90%, or about 100% in human neuroblastoma cell line (e.g., SK-N-BE(2)) expressing a full-length human SNCA gene when the neuroblastoma cells are in contact with 25 μM of the antisense oligonucleotide compared to control (e.g., an internal control such as GADPH or tubulin, or neuroblastoma cells expressing a full-length human SNCA gene in contact with saline alone), as measured by an assay, e.g., quantitative PCR disclosed herein.

In some embodiments, the ASOs disclosed herein are capable of reducing expression of SNCA protein in vitro by at least about 10%, at least about 20%, at least about 30%, at least about 40%, at least about 50%, at least about 60%, at least about 70%, at least about 80%, at least about 90%, or about 100% in human neuroblastoma cell line (e.g., SK-N-BE(2)) expressing a full-length human SNCA gene when the neuroblastoma cells are in contact with 25 μM of the antisense oligonucleotide compared to control (e.g., an internal control such as GADPH or tubulin, or neuroblastoma cells expressing a full-length human SNCA gene in contact with saline alone), as measured by an assay, e.g., High Content Assay analysis disclosed herein (see Example 2A).

In certain embodiments, the ASOs of the disclosure bind to the SNCA transcript and inhibit or reduce expression of the SNCA mRNA by at least about 10% or about 20% compared to the normal (i.e. control) expression level in the cell, e.g., at least about 30%, about 40%, about 50%, about 60%, about 70%, about 80%, about 90% or about 95% compared to the normal expression level (such as the expression level in the absence of the ASO(s) or conjugate(s)) in the cell. In certain embodiments, the ASO reduces expression of SNCA protein in a cell following administration of the ASO by at least 60%, at least 70%, at least 80%, or at least 90% compared to a cell not exposed to the ASO (i.e., control). In some embodiments, the ASO reduces expression of SNCA protein in a cell following administration of the ASO by at least about 60%, at least about 70%, at least about 80%, or at least about 90% compared to a cell not exposed to the ASO (i.e., control).

In certain embodiments, the ASO of the disclosure has at least one property selected from: (1) reduces expression of SNCA mRNA in a cell, compared to a control cell that has not been exposed to the ASO; (2) does not significantly reduce calcium oscillations in a cell; (3) does not significantly reduce tubulin intensity in a cell; (4) reduces expression of α-Syn protein in a cell; and (5) any combinations thereof compared to a control cell that has not been exposed to the ASO.

In some embodiments, the ASO of the disclosure does not significantly reduce calcium oscillations in a cell, e.g., neuronal cells. If the ASO does not significantly reduce calcium oscillations in a cell, this property of the ASO corresponds with a reduced neurotoxicity of the ASO. In some embodiments, calcium oscillations are greater than or equal to 95%, greater than or equal to 90%, greater than or equal to 85%, greater than or equal to 80%, greater than or equal to 75%, greater than or equal to 70%, greater than or equal to 65%, greater than or equal to 60%, greater than or equal to 55%, or greater than or equal to 50% of oscillations in a cell not exposed to the ASO.

Calcium oscillations are important for the proper functions of neuronal cells. Networks of cortical neurons have been shown to undergo spontaneous calcium oscillations resulting in the release of the neurotransmitter glutamate. Calcium oscillations can also regulate interactions of neurons with associated glia, in addition to other associated neurons in the network, to release other neurotransmitters in addition to glutamate. Regulated calcium oscillations are required for homeostasis of neuronal networks for normal brain function. (See, Shashank et al., Brain Research, 1006(1): 8-17 (2004); Rose et al., Nature Neurosci., 4:773-774 (2001); Zonta et al., JPhysiolParis., 96(3-4):193-8 (2002); Pasti et al., J. Neurosci., 21(2): 477-484 (2001).) Glutamate also activates two distinct ion channels, α-amino-3-hydroxy-5-methyl-4-isoxazolepropionic acid (AMPA) receptors and N-methyl-D-aspartate (NMDA) receptors.

In some embodiments, the calcium oscillations measured in the present methods are AMPA-dependent calcium oscillations. In some embodiments, the calcium oscillations are NMDA-dependent calcium oscillations. In some embodiments, the calcium oscillations are gamma-aminobutyric acid (GABA)-dependent calcium oscillations. In some embodiments, the calcium oscillations can be a combination of two or more of AMPA-dependent, NMDA-dependent or GABA-dependent calcium oscillations.

In certain embodiments, the calcium oscillations measured in the present methods are AMPA-dependent calcium oscillations. In order to measure AMPA-dependent calcium oscillations, the calcium oscillations can be measured in the presence of Mg²⁺ ions (e.g., MgCl₂). In certain embodiments, the method further comprises adding Mg²⁺ ions (e.g., MgCl₂) at an amount that allows for detection of AMPA-dependent calcium oscillations. In some embodiments, the effective ion concentration allowing for detection of AMPA-dependent calcium oscillations is at least about 0.5 mM. In other embodiments, the effective ion concentration to induce AMPA-dependent calcium oscillations is at least about 0.6 mM, at least about 0.7 mM, at least about 0.8 mM, at least about 0.9 mM, at least about 1 mM, at least about 1.5 mM, at least about 2.0 mM, at least about 2.5 mM, at least about 3.0 mM, at least about 4 mM, at least about 5 mM, at least about 6 mM, at least about 7 mM, at least about 8 mM, at least about 9 mM, or at least about 10 mM. In a particular embodiment, the concentration of Mg²⁺ ions (e.g., MgCl₂) useful for the methods is 1 mM. In certain embodiments, the concentration of Mg²⁺ ions (e.g., MgCl₂) useful for the present methods is about 1 mM to about 10 mM, about 1 mM to about 15 mM, about 1 mM to about 20 mM, or about 1 mM to about 25 mM. Mg²⁺ ions can be added by the addition of magnesium salts, such as magnesium carbonate, magnesium chloride, magnesium citrate, magnesium hydroxide, magnesium oxide, magnesium sulfate, and magnesium sulfate heptahydrate.

In some embodiments, calcium oscillations are measured in the present method through the use of fluorescent probes which detect the fluctuations of intracellular calcium levels. For example, detection of intracellular calcium flux can be achieved by staining the cells with fluorescent dyes which bind to calcium ions (known as fluorescent calcium indicators) with a resultant, detectable change in fluorescence (e.g., Fluo-4 AM and Fura Red AM dyes available from Molecular Probes. Eugene, OR, United States of America).

In other embodiments, the ASOs of the disclosure do not significantly reduce the tubulin intensity in a cell. In some embodiments, tubulin intensity is greater than or equal to 95%, greater than or equal to 90%, greater than or equal to 85%, greater than or equal to 80%, greater than or equal to 75%, greater than or equal to 70%, greater than or equal to 65%, greater than or equal to 60%, greater than or equal to 55%, or greater than or equal to 50% of tubulin intensity in a cell not exposed to the ASO (or exposed to saline).

In some embodiments, such property is observed when using from 0.04 nM to 400 μM concentration of the ASO of the disclosure. In the same or a different embodiment, the inhibition or reduction of expression of SNCA mRNA and/or SNCA protein in the cell results in less than 100%, such as less than 98%, less than 95%, less than 90%, less than 80%, such as less than 70%, mRNA or protein levels compared to cells not exposed to the ASO. Modulation of expression level can be determined by measuring SNCA protein levels, e.g., by methods such as SDS-PAGE followed by western blotting using suitable antibodies raised against the target protein. Alternatively, modulation of expression levels can be determined by measuring levels of SNCA mRNA, e.g., by northern blot or quantitative RT-PCR. When measuring inhibition via mRNA levels, the level of down-regulation when using an appropriate dosage, such as from about 0.04 nM to about 400 μM concentration, is, in some embodiments typically to a level of from about 10-20% the normal levels in the cell in the absence of the ASO.

In certain embodiments, the ASO of the disclosure has an in vivo tolerability less than or equal to a total score of 4, wherein the total score is the sum of a unit score of five categories, which are 1) hyperactivity; 2) decreased activity and arousal; 3) motor dysfunction and/or ataxia; 4) abnormal posture and breathing; and 5) tremor and/or convulsions, and wherein the unit score for each category is measured on a scale of 0-4. In certain embodiments, the in vivo tolerability is less than or equal to the total score of 3, the total score of 2, the total score of 1, or the total score of 0. In some embodiment, the assessment for in vivo tolerability is determined as described in the examples below.

In some embodiments, the ASO can tolerate 1, 2, 3, or 4 (or more) mismatches, when hybridizing to the target sequence and still sufficiently bind to the target to show the desired effect, i.e., down-regulation of the target mRNA and/or protein. Mismatches can, for example, be compensated by increased length of the ASO nucleotide sequence and/or an increased number of nucleotide analogs, which are disclosed elsewhere herein.

In some embodiments, the ASO of the disclosure comprises no more than 3 mismatches when hybridizing to the target sequence. In other embodiments, the contiguous nucleotide sequence comprises no more than 2 mismatches when hybridizing to the target sequence. In other embodiments, the contiguous nucleotide sequence comprises no more than 1 mismatch when hybridizing to the target sequence.

In some embodiments the ASO according to the disclosure comprises a nucleotide sequence, or a region within the sequence, according to any one of SEQ ID NOs: 4 to 21, the ASO sequences with the design as described in FIGS. 2 and 3, and the ASO sequence with the chemical structure as described in FIGS. 2 and 3.

However, it is recognized that, in some embodiments, the nucleotide sequence of the ASO can comprise additional 5′ or 3′ nucleotides, such as, independently, 1, 2, 3, 4 or 5 additional nucleotides 5′ and/or 3′, which are non-complementary to the target sequence. In this respect the ASO of the disclosure, can, in some embodiments, comprise a contiguous nucleotide sequence which is flanked 5′ and/or 3′ by additional nucleotides. In some embodiments the additional 5′ and/or 3′ nucleotides are naturally occurring nucleotides, such as DNA or RNA.

In some embodiments, the ASO of the disclosure has a sequence score greater than or equal to 0.2, wherein the sequence score is calculated by formula I:

# of C nucleotides and analogs thereof—# of G nucleotides and analogs thereof (I)

Total nucleotide length.

In other embodiments, the ASO of the disclosure has a sequence score greater than or equal to 0.2, wherein the sequence score is calculated by formula IA:

# of C nucleotides and 5-methylcytosine nucleotides—# of G nucleotides (IA)

Total nucleotide length.

In these embodiments, a sequence score of greater than or equal to a cut off value corresponds to a reduced neurotoxicity of the ASO.

In certain embodiments, the ASO of the disclosure has a sequence score greater than or equal to about 0.1, 0.2, 0.25, 0.3, 0.35, 0.4, 0.45, 0.5, 0.55, 0.6, 0.65, 0.7, 0.75, 0.8, 0.85, 0.9, 0.95, or 1.0. In one embodiment, the ASO of the disclosure comprises a contiguous nucleotide sequence hybridizing to a non-coding region of a SNCA transcript, wherein the sequence score of the ASO is greater than or equal to about 0.1, 0.2, 0.25, 0.3, 0.35, 0.4, 0.45, 0.5, 0.55, 0.6, 0.65, 0.7, 0.75, 0.8, 0.85, 0.9, 0.95, or 1.0. In another embodiment, the ASO of the disclosure comprises a contiguous nucleotide sequence hybridizing to an intron region of a SNCA transcript, wherein the sequence score of the ASO is greater than or equal to about 0.1, 0.2, 0.25, 0.3, 0.35, 0.4, 0.45, 0.5, 0.55, 0.6, 0.65, 0.7, 0.75, 0.8, 0.85, 0.9, 0.95, or 1.0. In another embodiment, the ASO of the disclosure comprises a contiguous nucleotide sequence hybridizing to an intron exon junction of a SNCA transcript, wherein the sequence score of the ASO is greater than or equal to about 0.1, 0.2, 0.25, 0.3, 0.35, 0.4, 0.45, 0.5, 0.55, 0.6, 0.65, 0.7, 0.75, 0.8, 0.85, 0.9, 0.95, or 1.0. In all of these embodiments, when the sequence score is greater than or equal to the cut off value, the ASO is considered to have reduced neurotoxicity.

### II.C. ASO Length

The ASOs can comprise a contiguous nucleotide sequence of a total of 10, 11, 12, 13, 14, 15, 16, 17, 18, 19, 20, 21, 22, 23, 24, 25, 26, 27, 28, 29, or 30 contiguous nucleotides in length.

In some embodiments, the ASOs comprise a contiguous nucleotide sequence of a total of about 10-22, such as 10-21 or 12-18, such as 13-17 or 12-16, such as 13, 14, 15, 16, 17, 18, 19, 20, or 21 contiguous nucleotides in length.

In some embodiments, the ASOs comprise a contiguous nucleotide sequence of a total of 16, 17, 18, 19, or 20 (16 to 20) contiguous nucleotides in length.

In some embodiments, the ASO according to the disclosure consists of no more than 22 nucleotides, such as no more than 21 or 20 nucleotides, such as no more than 18 nucleotides, such as 15, 16 or 17 nucleotides. In some embodiments the ASO of the disclosure comprises less than 22 nucleotides. It should be understood that when a range is given for an ASO, or contiguous nucleotide sequence length, the range includes the lower and upper lengths provided in the range, for example from (or between) 10-30, includes both 10 and 30.

II.D. Nucleosides and Nucleoside analogs

In one aspect of the disclosure, the ASOs comprise one or more non-naturally occurring nucleotide analogs. “Nucleotide analogs” as used herein are variants of natural nucleotides, such as DNA or RNA nucleotides, by virtue of modifications in the sugar and/or base moieties. Analogs could in principle be merely “silent” or “equivalent” to the natural nucleotides in the context of the oligonucleotide, i.e. have no functional effect on the way the oligonucleotide works to inhibit target gene expression. Such “equivalent” analogs can nevertheless be useful if, for example, they are easier or cheaper to manufacture, or are more stable to storage or manufacturing conditions, or represent a tag or label. In some embodiments, however, the analogs will have a functional effect on the way in which the ASO works to inhibit expression; for example by producing increased binding affinity to the target and/or increased resistance to intracellular nucleases and/or increased ease of transport into the cell. Specific examples of nucleoside analogs are described by e.g. Freier & Altmann; Nucl. Acid Res., 1997, 25, 4429-4443 and Uhlmann; Curr. Opinion in Drug Development, 2000, 3(2), 293-213, and in Scheme 1.

### II.D.1. Nucleobase

The term nucleobase includes the purine (e.g., adenine and guanine) and pyrimidine (e.g., uracil, thymine and cytosine) moiety present in nucleosides and nucleotides which form hydrogen bonds in nucleic acid hybridization. In the context of the present disclosure the term nucleobase also encompasses modified nucleobases which may differ from naturally occurring nucleobases, but are functional during nucleic acid hybridization. In some embodiments the nucleobase moiety is modified by modifying or replacing the nucleobase. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al., (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g., A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### II.D.2. Sugar Modification

The ASO of the disclosure can comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2′ and C4′ carbons on the ribose ring (LNA), or an unlinked ribose ring which typically lacks a bond between the C2′ and C3′ carbons (e.g., UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

In some embodiments, the sugar modification comprises an affinity enhancing sugar modification, e.g., LNA. An affinity enhancing sugar modification increases the binding affinity of the ASOs to the target RNA sequence (e.g., intron1/exon2 junction of SNCA mRNA). In some embodiments, an ASO comprising a sugar modification disclosed herein has a binding affinity to a target RNA sequence that is enhanced by at least 5%, at least 10%, at least 20%, at least 30%, at least 40%, at least 50%, at least 60%, at least 70%, at least 80%, at least 90%, or at least 100% compared to a control (e.g., an ASO without such sugar modification).

### II.D.2.a 2′ Modified Nucleosides

A 2′ sugar modified nucleoside is a nucleoside which has a substituent other than H or —OH at the 2′ position (2′ substituted nucleoside) or comprises a 2′ linked biradical, and includes 2′ substituted nucleosides and LNA (2′-4′ biradical bridged) nucleosides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

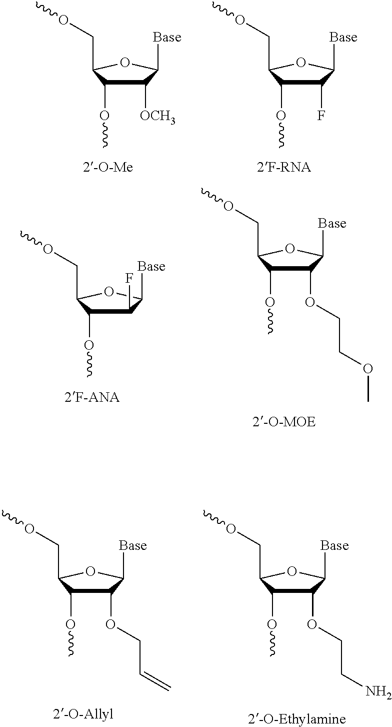

### IL.D.2.b Locked Nucleic Acid Nucleosides (LNA).

LNA nucleosides are modified nucleosides which comprise a linker group (referred to as a biradical or a bridge) between C2′ and C4′ of the ribose sugar ring of a nucleotide. These nucleosides are also termed bridged nucleic acid or bicyclic nucleic acid (BNA) in the literature.

In some embodiments, the modified nucleoside or the LNA nucleosides of the ASO of the disclosure has a general structure of the formula II or III:

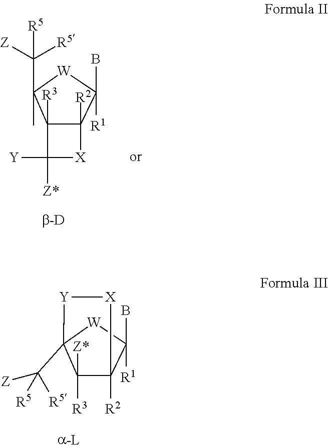

wherein W is selected from —O—, —S—, —N(R^{a})—, —C(R^{a}R^{b})—, such as, in some embodiments —O—; B designates a nucleobase or modified nucleobase moiety; Z designates an internucleoside linkage to an adjacent nucleoside, or a 5′-terminal group; Z* designates an internucleoside linkage to an adjacent nucleoside, or a 3′-terminal group; and X designates a group selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z.

In some embodiments, X is selected from the group consisting of: —O—, —S—, NH—, NR^{a}R^{b}, —CH₂—, CR^{a}R^{b}, —C(═CH₂)—, and —C(═CR^{a}R^{b})—. In some embodiments, X is —O—.

In some embodiments, Y designates a group selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z. In some embodiments, Y is selected from the group consisting of: —CH₂—, —C(R^{a}R^{b})—, —CH₂CH₂—, —C(R^{a}R^{b})—C(R^{a}R^{b})—, —CH₂CH₂CH₂—, —C(R^{a}R^{b})C(R^{a}R^{b})C(R^{a}R^{b})—, —C(R^{a})═C(R^{b})—, and —C(R^{a})═N—.

In some embodiments, Y is selected from the group consisting of: —CH₂—, —CHR^{a}—, —CHCH₃—, CR^{a}R^{b}—, and —X—Y— together designate a bivalent linker group (also referred to as a radicle) together designate a bivalent linker group consisting of 1, 2, 3 or 4 groups/atoms selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z.

In some embodiments, —X—Y designates a biradical selected from the groups consisting of: —X—CH₂—, —X—CR^{a}R^{b}—, —X—CHR^{a}—, —X—C(HCH₃)—, —O—Y—, —O—CH₂—, —S—CH₂—, —NH—CH₂—, —O—CHCH₃—, —CH₂—O—CH₂, —O—CH(CH₃CH₃)—, —O—CH₂—CH₂—, OCH₂—CH₂—CH₂—, —O—CH₂OCH₂—, —O—NCH₂—, —C(═CH₂)—CH₂—, —NR^{a}—CH₂—, N—O—CH₂, —S—CR^{a}R^{b}— and —S—CHR^{a}—.

In some embodiments —X—Y— designates —O—CH₂— or —O—CH(CH₃)—.

In certain embodiments, Z is selected from —O—, —S—, and —N(R^{a})—, and R^{a} and, when present R^{b}, each is independently selected from hydrogen, optionally substituted C₁₋₆-alkyl, optionally substituted C₂₋₆-alkenyl, optionally substituted C₂₋₆-alkynyl, hydroxy, optionally substituted C₁₋₃-alkoxy, C₂₋₆-alkoxyalkyl, C₂₋₆-alkenyloxy, carboxy, C₁₋₃-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₃-alkyl-carbonylamino, carbamido, C₁₋₆-alkanoyloxy, sulphono, C₁₋₃-alkylsulphonyloxy, nitro, azido, sulphanyl, C₁₋₆-alkylthio, halogen, where aryl and heteroaryl may be optionally substituted and where two geminal substituents R^{a} and R^{b}together may designate optionally substituted methylene (═CH₂), wherein for all chiral centers, asymmetric groups may be found in either R or S orientation.

In some embodiments, R¹, R², R³, R⁵ and R⁵* are independently selected from the group consisting of: hydrogen, optionally substituted C₁₋₃-alkyl, optionally substituted C₂₋₆-alkenyl, optionally substituted C₂₋₆-alkynyl, hydroxy, C₁₋₃-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₃-alkyl-aminocarbonyl, mono- and di(C₁₋₆-alkyl)amino-C₁₋₆-alkyl-aminocarbonyl, C₁₋₃-alkyl-carbonylamino, carbamido, C₁₋₃-alkanoyloxy, sulphono, C₁₋₆-alkylsulphonyloxy, nitro, azido, sulphanyl, C₁₋₆-alkylthio, and halogen, where aryl and heteroaryl may be optionally substituted, and where two geminal substituents together may designate oxo, thioxo, imino, or optionally substituted methylene.

In some embodiments R¹, R², R³, R⁵ and R⁵* are independently selected from C₁₋₆-alkyl, such as methyl, and hydrogen.

In some embodiments R¹, R², R³, R⁵ and R⁵* are all hydrogen.

In some embodiments R¹, R², R³, are all hydrogen, and either R⁵ and R⁵* is also hydrogen and the other of R⁵ and R⁵*is other than hydrogen, such as C₁₋₆ alkyl such as methyl.

In some embodiments, R^{a} is either hydrogen or methyl. In some embodiments, when present, R^{b} is either hydrogen or methyl.

In some embodiments, one or both of R^{a} and R^{b} is hydrogen.

In some embodiments, one of R^{a} and R^{b} is hydrogen and the other is other than hydrogen.

In some embodiments, one of R^{a} and R^{b} is methyl and the other is hydrogen.

In some embodiments, both of R^{a} and R^{b} are methyl.

In some embodiments, the biradical —X—Y— is —O—CH₂—, W is O, and all of R¹, R², R³, R⁵ and R⁵* are all hydrogen. Such LNA nucleosides are disclosed in WO99/014226, WO00/66604, WO98/039352 and WO2004/046160 which are all hereby incorporated by reference, and include what are commonly known as beta-D-oxy LNA and alpha-L-oxy LNA nucleosides.

In some embodiments, the biradical —X—Y— is —S—CH₂—, W is O, and all of R¹, R², R³, R⁵ and R⁵* are all hydrogen. Such thio LNA nucleosides are disclosed in WO99/014226 and WO2004/046160.

In some embodiments, the biradical —X—Y— is —NH—CH₂—, W is O, and all of R¹, R², R³, R⁵ and R⁵* are all hydrogen. Such amino LNA nucleosides are disclosed in WO99/014226 and WO2004/046160.

In some embodiments, the biradical —X—Y— is —O—CH₂—CH₂— or —O—CH₂—CH₂—CH₂—, W is O, and all of R¹, R², R³, R⁵ and R⁵* are all hydrogen. Such LNA nucleosides are disclosed in WO00/047599 and Morita et al., Bioorganic & Med. Chem. Lett. 12 73-76, which are hereby incorporated by reference, and include what are commonly known as 2′-O- 4′C-ethylene bridged nucleic acids (ENA).

In some embodiments, the biradical —X—Y— is —O—CH₂—, W is O, and all of R¹, R², R³, and one of R⁵ and R⁵* are hydrogen, and the other of R⁵ and R⁵* is other than hydrogen such as C₁₋₆alkyl, such as methyl. Such 5′ substituted LNA nucleosides are disclosed in WO2007/134181.

In some embodiments, the biradical —X—Y— is —O—CR^{a}R^{b}—, wherein one or both of R^{a} and R^{b} are other than hydrogen, such as methyl, W is O, and all of R¹, R², R³, and one of R⁵ and R⁵* are hydrogen, and the other of R⁵ and R⁵* is other than hydrogen such as C₁₋₆ alkyl, such as methyl. Such bis modified LNA nucleosides are disclosed in WO2010/077578.

In some embodiments, the biradical —X—Y— designate the bivalent linker group —O—CH(CH₂OCH₃)— (2′ O-methoxyethyl bicyclic nucleic acid —Seth at al., 2010, J. Org. Chem. Vol 75(5) pp. 1569-81). In some embodiments, the biradical —X—Y— designate the bivalent linker group —O—CH(CH₂CH₃)— (2′O-ethyl bicyclic nucleic acid —Seth at al., 2010, J. Org. Chem. Vol 75(5) pp. 1569-81). In some embodiments, the biradical —X—Y— is —O—CHR^{a}—, W is O, and all of R¹, R², R³, R⁵ and R⁵* are all hydrogen. Such 6′ substituted LNA nucleosides are disclosed in WO10036698 and WO07090071.

In some embodiments, the biradical —X—Y— is —O—CH(CH₂OCH₃)—, W is O, and all of R¹, R², R³, R⁵ and R⁵* are all hydrogen. Such LNA nucleosides are also known as cyclic MOEs in the art (cMOE) and are disclosed in WO07090071.

In some embodiments, the biradical —X—Y— designates the bivalent linker group —O—CH(CH₃)—. - in either the R- or S-configuration. In some embodiments, the biradical —X—Y— together designate the bivalent linker group —O—CH₂—O—CH₂— (Seth et al., 2010, J. Org. Chem). In some embodiments, the biradical —X—Y— is —O—CH(CH₃)—, W is O, and all of R¹, R², R³, R⁵ and R⁵* are all hydrogen. Such 6′ methyl LNA nucleosides are also known as cET nucleosides in the art, and may be either (S)cET or (R)cET stereoisomers, as disclosed in WO07090071 (beta-D) and WO2010/036698 (alpha-L)).

In some embodiments, the biradical —X—Y— is —O—CR^{a}R^{b}—, wherein in neither R^{a} or R^{b} is hydrogen, W is O, and all of R¹, R², R³, R⁵ and R⁵* are all hydrogen. In some embodiments, R^{a} and R^{b} are both methyl. Such 6′ di-substituted LNA nucleosides are disclosed in WO 2009006478.

In some embodiments, the biradical —X—Y— is —S—CHR^{a}—, W is O, and all of R¹, R², R³, R⁵ and R⁵* are all hydrogen. Such 6′ substituted thio LNA nucleosides are disclosed in WO 11156202. In some 6′ substituted thio LNA embodiments R^{a} is methyl.

In some embodiments, the biradical —X—Y— is —C(═CH₂)—C(R^{a}R^{b})—, such as —C(═CH₂)—CH₂—, or —C(═CH₂)—CH(CH₃)—W is O, and all of R¹, R², R³, R⁵ and R⁵* are all hydrogen. Such vinyl carbo LNA nucleosides are disclosed in WO08154401 and WO09067647.

In some embodiments the biradical —X—Y— is —N(—OR^{a})—, W is O, and all of R¹, R², R³, R⁵ and R⁵* are all hydrogen. In some embodiments R^{a} is C₁₋₆ alkyl such as methyl. Such LNA nucleosides are also known as N substituted LNAs and are disclosed in WO2008/150729. In some embodiments, the biradical —X—Y— together designate the bivalent linker group —O—NR^{a}—CH₃— (Seth et al., 2010, J. Org. Chem). In some embodiments the biradical —X—Y— is —N(R^{a})—, W is O, and all of R¹, R², R³, R⁵ and R⁵* are all hydrogen. In some embodiments R^{a} is C₁₋₆ alkyl such as methyl.

In some embodiments, one or both of R⁵ and R⁵* is hydrogen and, when substituted the other of R⁵ and R⁵* is C₁₋₆ alkyl such as methyl. In such an embodiment, R¹, R², R³, may all be hydrogen, and the biradical —X—Y— may be selected from —O—CH₂— or —O—CH(CR^{a})—, such as —O—CH(CH₃)—.

In some embodiments, the biradical is —CR^{a}R^{b}—O—CR^{a}R^{b}—, such as CH₂—O—CH₂—, W is O and all of R¹, R², R³, R⁵ and R⁵* are all hydrogen. In some embodiments R^{a} is C₁₋₆ alkyl such as methyl. Such LNA nucleosides are also known as conformationally restricted nucleotides (CRNs) and are disclosed in WO2013036868.

In some embodiments, the biradical is —O—CR^{a}R^{b}—O—CR^{a}R^{b}—, such as O—CH₂—O—CH₂—, W is O and all of R¹, R², R³, R⁵ and R⁵* are all hydrogen. In some embodiments R^{a} is C₁₋₆ alkyl such as methyl. Such LNA nucleosides are also known as COC nucleotides and are disclosed in Mitsuoka et al., Nucleic Acids Research 2009 37(4), 1225-1238.

It will be recognized than, unless specified, the LNA nucleosides may be in the beta-D or alpha-L stereoisoform.

Certain examples of LNA nucleosides are presented in Scheme 1.

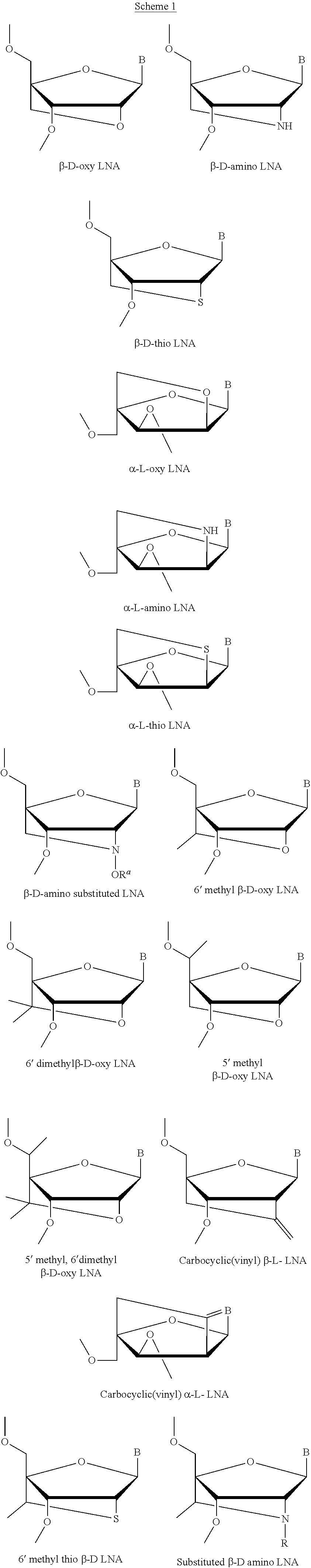

As illustrated in the examples, in some embodiments of the disclosure the LNA nucleosides in the oligonucleotides are beta-D-oxy-LNA nucleosides.

### II.E. Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the disclosure are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example gapmers.

### II.F. RNase H Activity and Recruitment

The RNase H activity of an antisense oligonucleotide refers to its ability to recruit RNase H when in a duplex with a complementary RNA molecule and induce cleavage and subsequent degradation of the complementary RNA molecule. WO01/23613 provides in vitro methods for determining RNase H activity, which may be used to determine the ability to recruit RNase H. Typically an oligonucleotide is deemed capable of recruiting RNase H if it, when provided with a complementary target nucleic acid sequence, has an initial rate, as measured in pmol/1/min, of at least 5%, such as at least 10% or more than 20% of the of the initial rate determined when using a oligonucleotide having the same base sequence as the modified oligonucleotide being tested, but containing only DNA monomers, with phosphorothioate linkages between all monomers in the oligonucleotide, and using the methodology provided by Example 91-95 of WO01/23613.

In some embodiments, an oligonucleotide is deemed essentially incapable of recruiting RNaseH if, when provided with the complementary target nucleic acid, the RNaseH initial rate, as measured in pmol/l/min, is less than 20%, such as less than 10%, such as less than 5% of the initial rate determined when using a oligonucleotide having the same base sequence as the oligonucleotide being tested, but containing only DNA monomers, with no 2′ substitutions, with phosphorothioate linkages between all monomers in the oligonucleotide, and using the methodology provided by Example 91-95 of WO01/23613.

### II.G. ASO Design

The ASO of the disclosure can comprise a nucleotide sequence which comprises both nucleotides and nucleotide analogs, and can be in the form of a gapmer. Examples of configurations of a gapmer that can be used with the ASO of the disclosure are described in U.S. Patent Appl. Publ. No. 2012/0322851.

The term gapmer as used herein refers to an antisense oligonucleotide which comprises a region of RNase H recruiting oligonucleotides (gap) which is flanked 5′ and 3′ by one or more affinity enhancing modified nucleosides (flanks). Various gapmer designs are described herein. The term LNA gapmer is a gapmer oligonucleotide wherein at least one of the affinity enhancing modified nucleosides is an LNA nucleoside. The term mixed wing gapmer refers to a LNA gapmer wherein the flank regions comprise at least one LNA nucleoside and at least one DNA nucleoside or non-LNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA and 2′-F-ANA nucleoside(s). In some embodiments the mixed wing gapmer has one flank which comprises LNA nucleosides (e.g., 5′ or 3′) and the other flank (3′ or 5′ respectfully) comprises 2′ substituted modified nucleoside(s).

In some embodiments, in addition to enhancing affinity of the ASO for the target region, some nucleoside analogs also mediate RNase (e.g., RNaseH) binding and cleavage.

### II.G.1. Gapmer Design

In one embodiment, the ASO of the disclosure is a gapmer. A gapmer ASO is an ASO which comprises a contiguous stretch of nucleotides which is capable of recruiting an RNase, such as RNaseH, such as a region of at least 6 DNA nucleotides, referred to herein in as region B (B), wherein region B is flanked both 5′ and 3′ by regions of affinity enhancing nucleotide analogs, such as from 1-10 nucleotide analogs 5′ and 3′ to the contiguous stretch of nucleotides which is capable of recruiting RNase—these regions are referred to as regions A (A) and C (C) respectively.

In certain embodiments, the gapmer is an alternating flank gapmer, examples of which are discussed below. In certain embodiments, the alternating flank gapmer exhibits less off target binding than a traditional gapmer. In certain embodiments, the alternating flank gapmer has better long term tolerability than a traditional gapmer.

An alternating flank gapmer can comprise a (poly)nucleotide sequence of formula (5′ to 3′), A-B-C, wherein: region A (A) (5′ region or a first wing sequence) comprises at least one nucleotide analog, such as at least one LNA unit, such as from 1-10 nucleotide analogs, such as LNA units, and; region B (B) comprises at least six consecutive nucleotides which are capable of recruiting RNase (when formed in a duplex with a complementary RNA molecule, such as the pre-mRNA or mRNA target), such as DNA nucleotides, and; region C (C) (3′region or a second wing sequence) comprises at least one nucleotide analog, such as at least one LNA unit, such as from 1-10 nucleotide analogs, such as LNA units; wherein regions A and C can include at any position in A and C 1-3 insertions of DNA nucleotide regions (e.g., DNA Insertions), in which these DNA insertions can each be 1-6 DNA units long.

In certain other embodiments, the gapmer, e.g., an alternating flank gapmer, comprises a (poly)nucleotide sequence of formula (5′ to 3′), A-B-C, or optionally A-B-C-D or D-A-B-C, wherein: region A (A) (5′ region) comprises at least one nucleotide analog, such as at least one LNA unit, such as from 1-10 nucleotide analogs, such as LNA units, and; region B (B) comprises at least five consecutive nucleotides which are capable of recruiting RNase (when formed in a duplex with a complementary RNA molecule, such as the mRNA target), such as DNA nucleotides, and; region C (C) (3′region) comprises at least one nucleotide analog, such as at least one LNA unit, such as from 1-10 nucleotide analogs, such as LNA units, and; region D (D), when present comprises 1, 2 or 3 nucleotide units, such as DNA nucleotides.

In some embodiments, region A comprises 1, 2, 3, 4, 5, 6, 7, 8, 9, or 10 nucleotide analogs, such as LNA units, such as from 2-5 nucleotide analogs, such as 2-5 LNA units, such as 2-5 nucleotide analogs, such as 3-5 LNA units; and/or region C consists of 1, 2, 3, 4, 5, 6, 7, 8, 9, or 10 nucleotide analogs, such as LNA units, such as from 2-5 nucleotide analogs, such as 2-5 LNA units, such as 2-5 nucleotide analogs, such as 3-5 LNA units.

In some embodiments B comprises 5, 6, 7, 8, 9, 10, 11, 12, 13, 14, 15, 16, 17, 18, 19, 20, 21, 22, or 23 consecutive nucleotides which are capable of recruiting RNase, or from 6-14, 7-14, 8-14, or from 7-10, or from 7-9, such as 8, such as 9, such as 10, or such as 14 consecutive nucleotides which are capable of recruiting RNase. In some embodiments region B comprises at least five DNA nucleotide unit, such as 5-23 DNA units, such as from 5-20 DNA units, such as from 5-18 DNA units, such as from 6-14 DNA units, such as from 8-14 DNA units, such as 5, 6, 7, 8, 9, 10, 11, 12, 13, or 14 DNA units.

In some embodiments region A comprises 3, 4, or 5 nucleotide analogs, such as LNA, region B consists of 5, 6, 7, 8, 9, 10, 11, 12, 13, or 14 DNA units, and region C consists of 3, 4, or 5 nucleotide analogs, such as LNA. Such designs include (A-B-C) 5-10-5, 3-14-3, 3-10-3, 3-10-4, 4-10-3, 3-9-3, 3-9-4, 4-9-3, 3-8-3, 3-8-4, 4-8-3, 3-7-3, 3-7-4, and 4-7-3, and can further include region D, which can have one to 3 nucleotide units, such as DNA units.

In some embodiments, the ASO of the disclosure, e.g., an alternating flank gapmer, comprises the formula of 5′-A-B-C-3′, wherein
(i) region B is a contiguous sequence of at least 5, 6, 7, 8, 9, or 10, e.g., 5 to 18 DNA units, which are capable of recruiting RNase;
(ii) region A is a first wing sequence of 1 to 10 nucleotides, wherein the first wing sequence comprises one or more nucleotide analogs and optionally one or more DNA units (e.g., DNA insertion) and wherein at least one of the nucleotide analogs is located at the 3′ end of A; and
(iii) region C is a second wing sequence of 1 to 10 nucleotides, wherein the second wing sequence comprises one or more nucleotide analogs and optionally one or more DNA units (e.g., DNA insertion) and wherein at least one of the nucleotide analogs is located at the 5′ end of C.

In some embodiments, the first wing sequence (region A in the formula) comprises a combination of nucleotide analogs and DNA units selected from (i) 1-10 nucleotide analogs and 0 DNA unit; (ii) 1-9 nucleotide analogs and 1 DNA unit; (iii) 1-8 nucleotide analogs and 1-2 DNA units; (iv) 1-7 nucleotide analogs and 1-3 DNA units; (v) 1-6 nucleotide analogs and 1-4 DNA units; (vi) 1-5 nucleotide analogs and 1-5 DNA units; (vii) 1-4 nucleotide analogs and 1-6 DNA units; (viii) 1-3 nucleotide analogs and 1-7 DNA units; (ix) 1-2 nucleotide analogs and 1-8 DNA units; (x) 1 nucleotide analog and 1-9 DNA units.

In certain embodiments, the second wing sequence (region C in the formula) comprises a combination of nucleotide analogs and DNA unit selected from (i) 1-10 nucleotide analogs and 0 DNA unit; (ii) 1-9 nucleotide analogs and 1 DNA unit; (iii) 1-8 nucleotide analogs and 1-2 DNA units; (iv) 1-7 nucleotide analogs and 1-3 DNA units; (v) 1-6 nucleotide analogs and 1-4 DNA units; (vi) 1-5 nucleotide analogs and 1-5 DNA units; (vii) 1-4 nucleotide analogs and 1-6 DNA units; (viii) 1-3 nucleotide analogs and 1-7 DNA units; (ix) 1-2 nucleotide analogs and 1-8 DNA units; (x) 1 nucleotide analog and 1-9 DNA units.

In some embodiments, region A in the ASO formula has a sub-formula selected from the first wing design of any ASOs in FIGS. 2 and 3, and/or region C in the ASO formula has a sub-formula selected from the second wing design of any ASOs in FIGS. 2 and 3, wherein the upper letter is a nucleotide analog (e.g., sugar modified analog, which can also be written as L) and the lower letter is DNA (which can also be written as D).

In certain embodiments, the ASO, e.g., an alternating flank gapmer, has the formula of 5′ A-B-C 3′, wherein region B is a contiguous sequence of 5 to 18 DNA units, region A has a formula of LLDLL, LDLLL, or LLLDL and region C has a formula of LLDLL or LDLDLL, and wherein L is an LNA unit and D is a DNA unit.

In some embodiments, the ASO has the formula of 5′ A-B-C 3′, wherein region B is a contiguous sequence of 10 DNA units, region A has the formula of LDL, and region C has the formula of LLLL, wherein L is an LNA unit and D is a DNA unit.

Further gapmer designs are disclosed in WO2004/046160, which is hereby incorporated by reference in its entirety. WO2008/113832 hereby incorporated by reference in its entirety, refers to ‘shortmer’ gapmer ASOs. In some embodiments, ASOs presented herein can be such shortmer gapmers.

In some embodiments the ASO, e.g., an alternating flank gapmer, comprises a contiguous nucleotide sequence of a total of 10, 11, 12, 13, 14, 15, 16, 17, 18, 19, or 20 nucleotide units, wherein the contiguous nucleotide sequence is of formula (5′-3′), A-B-C, or optionally A-B-C-D or D-A-B-C, wherein; region A consists of 1, 2, 3, 4, or 5 nucleotide analog units, such as LNA units; region B consists of 5, 6, 7, 8, 9, 10, 11, 12, 13, or 14 contiguous nucleotide units which are capable of recruiting RNase when formed in a duplex with a complementary RNA molecule (such as a mRNA target); and region C consists of 1, 2, 3, 4, or 5 nucleotide analog units, such as LNA units. When present, region D consists of a single DNA unit.

In some embodiments A comprises 1 LNA unit. In some embodiments region A comprises 2 LNA units. In some embodiments region A comprises 3 LNA units. In some embodiments region A comprises 4 LNA units. In some embodiments region A comprises 5 LNA units. In some embodiments region C comprises 1 LNA unit. In some embodiments C comprises 2 LNA units. In some embodiments region C comprises 3 LNA units. In some embodiments region C comprises 4 LNA units. In some embodiments region C comprises 5 LNA units. In some embodiments region B comprises 6 nucleotide units. In some embodiments region B comprises 7 nucleotide units. In some embodiments region B comprises 8 nucleotide units. In some embodiments region B comprises 9 nucleotide units. In certain embodiments, region B comprises 10 nucleoside units. In certain embodiments, region B comprises 11 nucleoside units. In certain embodiments, region B comprises 12 nucleoside units. In certain embodiments, region B comprises 13 nucleoside units. In certain embodiments, region B comprises 14 nucleoside units. In certain embodiments, region B comprises 7-23 DNA monomers or 5-18 DNA monomers. In some embodiments region B comprises from 6-23 DNA units, such as 6, 7, 8, 9, 10, 11, 12, 13, 14, 15, 16, 17, 18, 19, 20, 21, 22, or 23 DNA units. In some embodiments region B consists of DNA units. In some embodiments region B comprises at least one LNA unit which is in the alpha-L configuration, such as 2, 3, 4, 5, 6, 7, 8, 9, 10, 11, 12, 13, 14, 15, 16, 17, 18, 19, 20, 21, 22, or 23 LNA units in the alpha-L-configuration. In some embodiments region B comprises at least one alpha-L-oxy LNA unit or wherein all the LNA units in the alpha-L-configuration are alpha-L-oxy LNA units. In some embodiments the number of nucleotides present in A-B-C are selected from (nucleotide analog units—region B—nucleotide analog units): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, and 3-10- 1. In some embodiments the number of nucleotides in A-B-C is selected from: 2-7-1, 1-7-2, 2-7-2, 3-7-3, 2-7-3, 3-7-2, 3-7-4, and 4-7-3. In other embodiments, the ASO contains 10 DNA units in B, LDLLL in A (first wing) and LLDLL in C (second wing). In yet other embodiments, the ASO contains 9 DNA units in B, LDDLL in A, and LDLDLL in C. In still other embodiments, the ASO contains 10 DNA units in B, LLDLL in A, and LLDLL in C. In further embodiments, the ASO contains 9 DNA units in B, LLLLL in A, and LDDLL in C. In certain embodiments, each of regions A and C comprises three LNA monomers, and region B consists of 7, 8, 9, 10, 11, 12, 13, or 14 nucleoside monomers, for example, DNA monomers. In some embodiments both A and C consist of two LNA units each, and B consists of 7, 8, or 9 nucleotide units, for example DNA units. In various embodiments, other gapmer designs include those where regions A and/or C consists of 3, 4, 5 or 6 nucleoside analogs, such as monomers containing a 2′-O-methoxyethyl-ribose sugar (2′-MOE) or monomers containing a 2′-fluoro-deoxyribose sugar, and region B consists of 7, 8, 9, 10, 11, 12, 13, 14, 15, 16, 17, 18, 19, 20, 21, 22, or 23 nucleosides, such as DNA monomers, where regions A-B-C have 3-8-3, 3-9-3, 3-10-3, 5-10-5 or 4-12-4 monomers. Further gapmer designs are disclosed in WO 2007/146511A2, hereby incorporated by reference in its entirety.

In some embodiments, the alternating flank ASO has at least 10 contiguous nucleotides, comprising region A, region B, and region C (A-B-C), wherein region B comprises at least 5 consecutive nucleoside units and is flanked at 5′ by region A of 1-8 contiguous nucleoside units and at 3′ by region C of 1-8 contiguous nucleoside units, wherein region B, when formed in a duplex with a complementary RNA, is capable of recruiting RNaseH, and wherein region A and region C are selected from the group consisting of:
(i) region A comprises a 5′ LNA nucleoside unit and a 3′ LNA nucleoside unit, and at least one DNA nucleoside unit between the 5′ LNA nucleoside unit and the 3′ LNA nucleoside unit, and, region C comprises at least two 3′ LNA nucleosides;
(ii) region A comprises at least one 5′ LNA nucleoside and region C comprises a 5′ LNA nucleoside unit, at least two terminal 3′ LNA nucleoside units, and at least one DNA nucleoside unit between the 5′ LNA nucleoside unit and the 3′ LNA nucleoside units, and
(iii) region A comprises a 5′ LNA nucleoside unit and a 3′ LNA nucleoside unit, and at least one DNA nucleoside unit between the 5′ LNA nucleoside unit and the 3′ LNA nucleoside unit; and region C comprises a 5′ LNA nucleoside unit, at least two terminal 3′ LNA nucleoside units, and at least one DNA nucleoside unit between the 5′ LNA nucleoside unit and the 3′ LNA nucleoside units.

In some embodiments, region A or region C comprises 1, 2, or 3 DNA nucleoside units. In other embodiments, region A and region C comprise 1, 2, or 3 DNA nucleoside units. In yet other embodiments, region B comprises at least five consecutive DNA nucleoside units. In certain embodiments, region B comprises 6, 7, 8, 9, 10, 11, 12, 13 or 14 consecutive DNA nucleoside units. In some embodiments, region B is 8, 9 10, 11, or 12 nucleotides in length. In other embodiments, region A comprises two 5′ terminal LNA nucleoside units. In some embodiments, region A has formula 5′[LNA]₁₋₃[DNA]₁₋₃[LNA]₁₋₃, or 5′[LNA]₁₋₂ [DNA]₁₋₂ [LNA]₁₋₂[DNA]₁₋₂ [LNA]₁₋₂. In other embodiments, region C has formula [LNA]₁₋₃[DNA]₁₋₃[LNA]₂₋₃3′, or [LNA]₁₋₂[DNA]₁₋₂[LNA]₁₋₂[DNA]₁₋₂[LNA]₂₋₃3′. In yet other embodiments, region A has formula 5′[LNA]₁₋₃[DNA]₁₋₃[LNA]₁₋₃, or 5′[LNA]₁₋₂ [DNA]₁₋₂[LNA]₁₋₂[DNA]₁₋₂ [LNA]₁₋₂, and region C comprises 2, 3, 4 or 5 consecutive LNA nucleoside units. In some embodiments, region C has formula [LNA]₁₋₃[DNA]₁₋₃[LNA]₂₋₃3′ or [LNA]₁₋₂ [DNA]₁₋₂ [LNA]₁₋₂ [DNA]₁₋₂ [LNA]₂₋₃3′, and region A comprises 1, 2, 3, 4 or 5 consecutive LNA nucleoside units. In still other embodiments, region A has a sequence of LNA and DNA nucleosides, 5′-3′ selected from the group consisting of L, LL, LDL, LLL, LLDL, LDLL, LDDL, LLLL, LLLLL, LLLDL, LLDLL, LDLLL, LLDDL, LDDLL, LLDLD, LDLLD, LDDDL, LLLLLL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, wherein L represents a LNA nucleoside, and D represents a DNA nucleoside. In yet other embodiments, region C has a sequence of LNA and DNA nucleosides, 5′-3′ selected from the group consisting of LL, LLL, LLLL, LDLL, LLLLL, LLDLL, LDLLL, LDDLL, LDDLLL, LLDDLL, LDLDLL, LDDDLL, LDLDDLL, LDDLDLL, LDDDLLL, and LLDLDLL. In a further embodiment, region A has a sequence of LNA and DNA nucleosides, 5′-3′ selected from the group consisting of LDL, LLDL, LDLL, LDDL, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, and region C has a sequence of LNA and DNA nucleosides, 5′-3′ selected from the group consisting of LDLL, LLDL, LLLLL, LLDLL, LDLLL, LDDLL, LDDLLL, LLDDLL, LDLDLL, LDDDLL, LDLDDLL, LDDLDLL, LDDDLLL, and LLDLDLL.

In certain embodiments, the alternating flank ASO has contiguous nucleotides comprising a sequence of nucleosides, 5′-3′, selected from the group consisting of LDLDDDDDDDDDDLLLL, LLDDDLLDDDDDDDDLL, LDLLDLDDDDDDDDDLL, LLLDDDDDDDDDDLDLL, LLLDDDDDDDDDLDDLL, LLLDDDDDDDDLDDDLL, LLLDDDDDDDDLDLDLL, LLLDLDDDDDDDDDLLL, LLLDLDDDDDDDDLDLL, LLLLDDDDDDDDDLDLL, LLLLDDDDDDDDLDDLL, LLLDDDLDDDDDDDDLL, LLLDDLDDDDDDDDDLL, LLLDDLLDDDDDDDDLL, LLLDDLLDDDDDDDLLL, LLLLLDDDDDDDLDDLL, LDLLLDDDDDDDDDDLL, LDLLLDDDDDDDLDDLL, LDLLLLDDDDDDDDDLL, LLDLLLDDDDDDDDDLL, LLLDLDDDDDDDDDDLL, LLLDLDDDDDDDLDDLL, LLLDLLDDDDDDDDDLL, LLLLDDDDDDDLDDDLL, LDDLDDDDDDDDDLDLLL, LDDLDDDDDDDDDLLDLL, LLLLLDDDDDDDDDLDLL, LLLLDDDDDDDDDDLDLL, LLLDDDDDDDDDDDLDLL, LLDLDDDDDDDDDDLDLL, LDLLLDDDDDDDDDLDLL, LLLDDDDDDDDDDLDDLL, LLLDDDDDDDDDLDDDLL, LLLDDDDDDDDLDLDDLL, LLLLDDDDDDDDDLDDLL, LLLLDDDDDDDDDLDLLL, LLLLDDDDDDDDLDDDLL, LLLLDDDDDDDDLDDLLL, LLLLDDDDDDDDLDLDLL, LLLLDDDDDDDLDDLDLL, LLLLDDDDDDDLDLDDLL, LLDLLDDDDDDDDDDDLL, LLDLLLDDDDDDDDLDLL, LLLDLDDDDDDDDDDDLL, LLLDLDDDDDDDDDLDLL, LLLDLDDDDDDDDLDDLL, LLLDLDDDDDDDLDLDLL, LLLLDDDDDDDDDLLDLL, LLLLLDDDDDDDDDLDLLL, LLLLLDDDDDDDDDLDDLL, LLLLDDDDDDDDDDLLDLL, LLLLDDDDDDDDDDLDLLL, LLLLDDDDDDDDDDLDDLL, LLLDDDDDDDDDDDLLDLL, LLLDDDDDDDDDDDLDLLL, LLLLLDDDDDDDDDLLDLL, LLLDDDDDDDDDDDLDDLL, LLDLLDDDDDDDDDLDDLL, LLLDLDDDDDDDDDDLDLL, LLLDLDDDDDDDDDLDDLL, LLLLDDDDDDDDDLDLDLL, LLLLDDDDDDDDLLDLDLL, LDLLLDDDDDDDDDDLLDLL, LLDLLDDDDDDDDDDLLDLL, LLDLDDDDDDDDDDDDLLLL, LLDDLDDDDDDDDDDDLLLL, LLLDLDDDDDDDDDDDLLLL, LLDLDDDDDDDDDDDDDLLL, LLDLLDDDDDDDDDDDLLLL, LLDDLDDDDDDDDDDDDLLL, LLLDDDDDDDDDDDLDDLLL, LLLDLDDDDDDDDDDDDLLL, LLDLLDDDDDDDDDDDDLLL, LLLLDDDDDDDDDDDLLDLL, LLLLDDDDDDDDDDLLDDLL, LLLDDLDDDDDDDDDLDLLL, LLDDLDLDDDDDDDDDLLLL, LLDDLLDDDDDDDDDLDLLL, LLLDLDDDDDDDDDLDLDLL, LLDLLDDDDDDDDDLDDLLL, LLLDLDDDDDDDDDDLDLLL, LLDLDDLDDDDDDDDDLLLL, LLLLDDDDDDDDDLDLDDLL, LLLDLDDDDDDDDDLDDLLL, LLDLDLDDDDDDDDDDLLLL, LLDLLDDDDDDDDDDLDLLL, LLDLDLDDDDDDDDDLLDLL, LLDDLLDDDDDDDDDLLDLL, LLLLDDDDDDDDDLDDLDLL, LLLDDLDDDDDDDDDLLDLL, LLDLLDDDDDDDDDLLDDLL, LLDLDLDDDDDDDDDLDLLL, LLLDLDDDDDDDDDLLDDLL, LLDDLLDDDDDDDDDDLLLL, LLDLLDDDDDDDDDLDLDLL, LLLLDDDDDDDDDDLDDLLL, LLLDDLDDDDDDDDDDLLLL, LLLDLDDDDDDDDDDLLDLL, LLLLDDDDDDDDDDLDLDLL, LLLLDDDDDDDDDDDLDLLL, and LLDDLLDDDDDDDDDDLDLL; wherein L represents a LNA nucleoside, and D represents a DNA nucleoside. In other embodiments, the LNA nucleoside is beta-D-oxy LNA.

In yet other embodiments, an alternating flank ASO has contiguous nucleotides comprising an alternating sequence of LNA and DNA nucleoside units, 5′-3′, selected from the group consisting of: 1-1-1-10-4, 1-2-1-9-2-1-2, 1-2-1-9-1-1-3, 2-3-2-8-2, 1-1-2-1-1-9-2, 3-10-1-1-2, 3-9-1-2-2, 3-8-1-3-2, 3-8-1-1-1-1-2, 3-1-1-9-3, 3-1-1-8-1-1-2, 4-9-1-1-2, 4-8-1-2-2, 3-3-1-8-2, 3-2-1-9-2, 3-2-2-8-2, 3-2-2-7-3, 5-7-1-2-2, 1-1-3-10-2, 1-1-3-7-1-2-2, 1-1-4-9-2, 2-1-3-9-2, 3-1-1-10-2, 3-1-1-7-1-2-2, 3-1-2-9-2, 4-7-1-3-2, 5-9-1-1-2, 4-10-1-1-2, 3-11-1-1-2, 2-1-1-10-1-1-2, 1-1-3-9-1-1-2, 3-10-1-2-2, 3-9-1-3-2, 3-8-1-1-1-2-2, 4-9-1-2-2, 4-9-1-1-3, 4-8-1-3-2, 4-8-1-2-3, 4-8-1-1-1-1-2, 4-7-1-2-1-1-2, 4-7-1-1-1-2-2, 2-1-2-11-2, 2-1-3-8-1-1-2, 3-1-1-11-2, 3-1-1-9-1-1-2, 3-1-1-8-1-2-2, 3-1-1-7-1-1-1-1-2, 4-9-2-1-2, 4-7-1-3-3, 5-9-1-1-3, 5-9-1-2-2, 4-10-2-1-2, 4-10-1-1-3, 4-10-1-2-2, 3-11-2-1-2, 3-11-1-1-3, 5-9-2-1-2, 3-11-1-2-2, 2-1-2-9-1-2-2, 3-1-1-10-1-1-2, 3-1-1-9-1-2-2, 4-9-1-1-1-1-2, 4-8-2-1-1-1-2, 1-1-3-10-2-1-2, 2-1-2-10-2-1-2, 2-1-1-12-4, 2-2-1-11-4, 3-1-1-11-4, 2-1-1-13-3, 2-1-2-11-4, 2-2-1-12-3, 3-11-1-2-3, 3-1-1-12-3, 2-1-2-12-3, 4-11-2-1-2, 4-10-2-2-2, 3-2-1-9-1-1-3, 2-2-1-1-1-9-4, 2-2-2-9-1-1-3, 3-1-1-9-1-1-1-1-2, 2-1-2-9-1-2-3, 3-1-1-10-1-1-3, 2-1-1-2-1-9-4, 4-9-1-1-1-2-2, 3-1-1-9-1-2-3, 2-1-1-1-1-10-4, 2-1-2-10-1-1-3, 2-1-1-1-1-9-2-1-2, 2-2-2-9-2-1-2, 4-9-1-2-1-1-2, 3-2-1-9-2-1-2, 2-1-2-9-2-2-2, 2-1-1-1-1-9-1-1-3, 3-1-1-9-2-2-2, 2-2-2-10-4, 2-1-2-9-1-1-1-1-2, 4-10-1-2-3, 3-2-1-10-4, 3-1-1-10-2-1-2, 4-10-1-1-1-1-2, 4-11-1-1-3, and 2-2-2-10-1-1-2; wherein the first numeral represents an number of LNA units, the next a number of DNA units, and alternating LNA and DNA regions thereafter.

In other embodiments, the ASOs of the disclosure are represented as any one of ASO numbers selected from FIGS. 2 and 3.

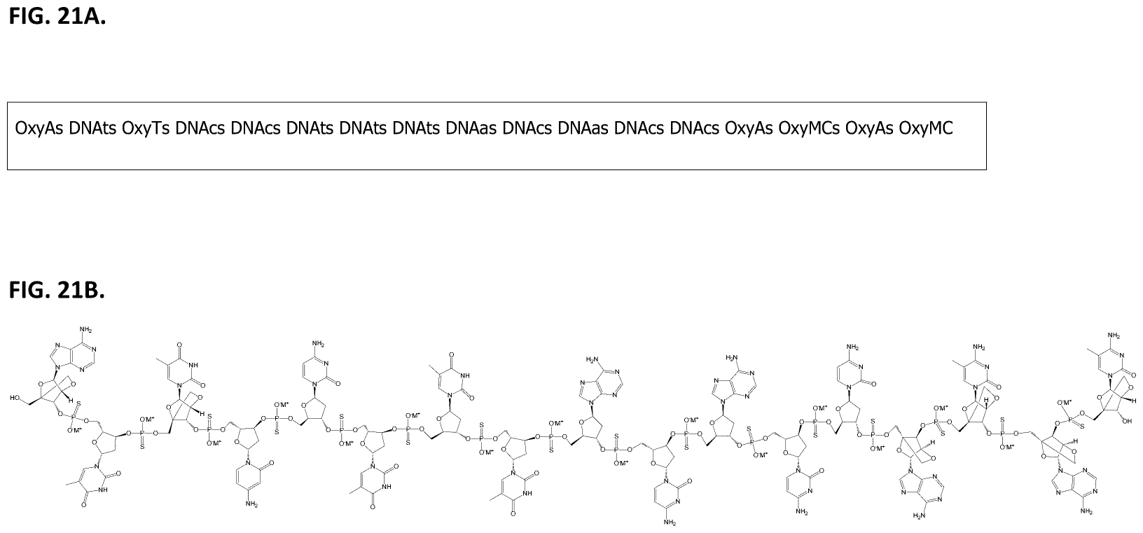

In some embodiments, the ASO of the disclosure (i.e., ASO-005459) comprises a contiguous nucleotide sequence of 17 nucleotides in length, which corresponds to the complement of a region (i.e., junction between intron 1 and exon 2) of SNCA transcript, i.e., nucleotides 7,604-7,620 of SEQ ID NO: 1. In some embodiments, the ASO of the disclosure has the nucleotide sequence as set forth in SEQ ID NO: 15 (i.e., attcctttacaccacac) with an ASO design of LDLDDDDDDDDDDLLLL (i.e., AtTcctttacaccACAC), wherein the L indicates a locked nucleic acid nucleoside (i.e., LNA, e.g., beta-D-oxy-LNA) and the D indicates a deoxyribonucleic acid (DNA). Accordingly, the 1^{st}, 3^{rd} and the 14^{th}-17^{th} nucleotides from the 5′ end of ASO-005459 is beta-D-oxy-LNA and each of the other nucleotides is, DNA. In other embodiments, the ASO disclosed herein also has the following chemical structure: OxyAs DNAts OxyTs DNAcs DNAcs DNAts DNAts DNAts DNAas DNAcs DNAas DNAcs DNAcs OxyAs OxyMCs OxyAs OxyMC, wherein “s” indicates a phosphorothioate linkage. The structural formula for ASO-005459 is provided in FIG. 21B, wherein M+ is a pharmaceutically acceptable counterion. The term “pharmaceutically acceptable counterion,” as used herein,” refers to an ion that accompanies an ionic species in order to maintain electric neutrality that is not biologically or otherwise undesirable and thereby, allowing for the production of a pharmaceutically acceptable salt form. Accordingly, in some embodiments, pharmaceutically acceptable counterion can be H⁺, Na⁺, K⁺, NH₄⁺, Li⁺, or any other cation, e.g., a cation with a charge of 1+. In some embodiments, the pharmaceutically acceptable counterion is H⁺, Na⁺, NI₄⁺, or combinations thereof.

### II.H. Internucleotide Linkages

The monomers of the ASOs described herein are coupled together via linkage groups. Suitably, each monomer is linked to the 3′ adjacent monomer via a linkage group.

The person having ordinary skill in the art would understand that, in the context of the present disclosure, the 5′ monomer at the end of an ASO does not comprise a 5′ linkage group, although it may or may not comprise a 5′ terminal group.

The terms “linkage group” and “internucleotide linkage” are intended to mean a group capable of covalently coupling together two nucleotides. Specific and preferred examples include phosphate groups and phosphorothioate groups.

The nucleotides of the ASO of the disclosure or contiguous nucleotides sequence thereof are coupled together via linkage groups. Suitably each nucleotide is linked to the 3′ adjacent nucleotide via a linkage group.

Suitable internucleotide linkages include those listed within WO2007/031091, for example the internucleotide linkages listed on the first paragraph of page 34 of WO2007/031091 (hereby incorporated by reference in its entirety).

Examples of suitable internucleotide linkages that can be used with the disclosure include phosphodiester linkage, a phosphotriester linkage, a methylphosphonate linkage, a phosphoramidate linkage, a phosphorothioate linkage, and combinations thereof.

It is, in some embodiments, preferred to modify the internucleotide linkage from its normal phosphodiester to one that is more resistant to nuclease attack, such as phosphorothioate or boranophosphate—these two, being cleavable by RNaseH, also allow that route of antisense inhibition in reducing the expression of the target gene.

Suitable sulphur (S) containing internucleotide linkages as provided herein may be preferred. Phosphorothioate internucleotide linkages are also preferred, particularly for the gap region (B) of gapmers. Phosphorothioate linkages can also be used for the flanking regions (A and C, and for linking A or C to D, and within region D, as appropriate).

Regions A, B and C, can, however, comprise internucleotide linkages other than phosphorothioate, such as phosphodiester linkages, particularly, for instance when the use of nucleotide analogs protects the internucleotide linkages within regions A and C from endo-nuclease degradation—such as when regions A and C comprise LNA nucleotides.

The internucleotide linkages in the ASO can be phosphodiester, phosphorothioate or boranophosphate so as to allow RNaseH cleavage of targeted RNA. Phosphorothioate is preferred for improved nuclease resistance and other reasons, such as ease of manufacture. In some embodiments, the internucleotide linkages comprise one or more stereo-defined internucleotide linkages (e.g., such as stereo-defined modified phosphate linkages, e.g., phosphodiester, phosphorothioate, or boranophosphate linkages with a defined stereochemical structure). The term “stereo-defined internucleotide linkage” is used interchangeably with “chirally controlled internucleotide linkage” and refers to a internucleotide linkage in which the stereochemical designation of the phosphorus atom is controlled such that a specific amount of Rₚ or Sₚ of the internucleotide linkage is present within an ASO strand. The stereochemical designation of a chiral linkage can be defined (controlled) by, for example, asymmetric synthesis. An ASO having at least one stereo-defined internucleotide linkage can be called as a stereo-defined ASO, which includes both a fully stereo-defined ASO and a partially stereo-defined ASO.

In some embodiments, an ASO is fully stereo-defined. A fully stereo-defined ASO refers to an ASO sequence having a defined chiral center (Rₚ or Sₚ) in each internucleotide linkage in the ASO. In some embodiments, an ASO is partially stereo-defined. A partially stereo-defined ASO refers to an ASO sequence having a defined chiral center (Rₚ or Sₚ) in at least one internucleotide linkage, but not in all of the internucleotide linkages. Therefore, a partially stereo-defined ASO can include linkages that are achiral or stereo-nondefined in addition to the at least one stereo-defined linkage. When an internucleotide linkage in an ASO is stereo-defined, the desired configuration, either Rₚ or Sₚ, is present in at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or essentially 100% of the ASO.

In one aspect of the ASO of the disclosure, the nucleotides and/or nucleotide analogs are linked to each other by means of phosphorothioate groups.

It is recognized that the inclusion of phosphodiester linkages, such as one or two linkages, into an otherwise phosphorothioate ASO, particularly between or adjacent to nucleotide analog units (typically in region A and or C) can modify the bioavailability and/or bio-distribution of an ASO—see WO2008/113832, hereby incorporated by reference.

In some embodiments, such as the embodiments referred to above, where suitable and not specifically indicated, all remaining linkage groups are either phosphodiester or phosphorothioate, or a mixture thereof.

In some embodiments all the internucleotide linkage groups are phosphorothioate.

When referring to specific gapmer oligonucleotide sequences, such as those provided herein it will be understood that, in various embodiments, when the linkages are phosphorothioate linkages, alternative linkages, such as those disclosed herein can be used, for example phosphate (phosphodiester) linkages can be used, particularly for linkages between nucleotide analogs, such as LNA, units. Likewise, when referring to specific gapmer oligonucleotide sequences, such as those provided herein, when the C residues are annotated as 5-′methyl modified cytosine, in various embodiments, one or more of the Cs present in the ASO can be unmodified C residues.

US Publication No. 2011/0130441, which was published Jun. 2, 2011 and is incorporated by reference herein in its entirety, refers to ASO compounds having at least one bicyclic nucleoside attached to the 3′ or 5′ termini by a neutral internucleoside linkage. The ASOs of the disclosure can therefore have at least one bicyclic nucleoside attached to the 3′ or 5′ termini by a neutral internucleoside linkage, such as one or more phosphotriester, methylphosphonate, MMI (3′-CH₂—N(CH₃) O—5′), amide-3 (3′-CH₂—C(═O)—N(H)-5′), formacetal (3′-O—CH₂—O—5′) or thioformacetal (3′-S—CH₂—O-5′). The remaining linkages can be phosphorothioate.

In some embodiments, the ASOs of the disclosure have internucleotide linkages described in FIGS. 2 and 3. As used herein, e.g., FIGS. 2 and 3, phosphorothioate linkages are indicated as “s”, and phosphodiester linkages are indicated by the absence of “s.”

### II.I. Conjugates

The term conjugate as used herein refers to an oligonucleotide which is covalently linked to a non-nucleotide moiety (conjugate moiety or region C or third region).

Conjugation of the oligonucleotide of the disclosure to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. At the same time the conjugate may serve to reduce activity of the oligonucleotide in non-target cell types, tissues or organs, e.g., off target activity or activity in non-target cell types, tissues or organs. WO 93/07883 and WO2013/033230 provides suitable conjugate moieties. Further suitable conjugate moieties are those capable of binding to the asialoglycoprotein receptor (ASGPr). In particular tri-valent N-acetylgalactosamine conjugate moieties are suitable for binding to the ASGPr, see for example WO 2014/076196, WO 2014/207232 and WO 2014/179620.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids), and combinations thereof.

### II.J. Activated ASOs

The term “activated ASO,” as used herein, refers to an ASO of the disclosure that is covalently linked (i.e., functionalized) to at least one functional moiety that permits covalent linkage of the ASO to one or more conjugated moieties, i.e., moieties that are not themselves nucleic acids or monomers, to form the conjugates herein described. Typically, a functional moiety will comprise a chemical group that is capable of covalently bonding to the ASO via, e.g., a 3′-hydroxyl group or the exocyclic NH₂ group of the adenine base, a spacer that can be hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999).

In some embodiments, ASOs of the disclosure are functionalized at the 5′ end in order to allow covalent attachment of the conjugated moiety to the 5′ end of the ASO. In other embodiments, ASOs of the disclosure can be functionalized at the 3′ end. In still other embodiments, ASOs of the disclosure can be functionalized along the backbone or on the heterocyclic base moiety. In yet other embodiments, ASOs of the disclosure can be functionalized at more than one position independently selected from the 5′ end, the 3′ end, the backbone and the base.

In some embodiments, activated ASOs of the disclosure are synthesized by incorporating during the synthesis one or more monomers that is covalently attached to a functional moiety. In other embodiments, activated ASOs of the disclosure are synthesized with monomers that have not been functionalized, and the ASO is functionalized upon completion of synthesis.

### III. Pharmaceutical Compositions and Administration Routes

The ASO of the disclosure can be used in pharmaceutical formulations and compositions. Suitably, such compositions comprise a pharmaceutically acceptable diluent, carrier, salt or adjuvant.

The ASO of the disclosure can be included in a unit formulation such as in a pharmaceutically acceptable carrier or diluent in an amount sufficient to deliver to a patient a therapeutically effective amount without causing serious side effects in the treated patient. However, in some forms of therapy, serious side effects may be acceptable in terms of ensuring a positive outcome to the therapeutic treatment.

The formulated drug may comprise pharmaceutically acceptable binding agents and adjuvants. Capsules, tablets, or pills can contain for example the following compounds: microcrystalline cellulose, gum or gelatin as binders; starch or lactose as excipients; stearates as lubricants; various sweetening or flavoring agents. For capsules the dosage unit may contain a liquid carrier like fatty oils. Likewise coatings of sugar or enteric agents may be part of the dosage unit. The oligonucleotide formulations can also be emulsions of the active pharmaceutical ingredients and a lipid forming a micellular emulsion.

The pharmaceutical compositions of the present disclosure can be administered in a number of ways depending upon whether local or systemic treatment is desired and upon the area to be treated. Administration can be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal, intra-cerebroventricular, or intraventricular, administration. In one embodiment the ASO is administered IV, IP, orally, topically or as a bolus injection or administered directly in to the target organ. In another embodiment, the ASO is administered intrathecal or intra-cerebroventricular as a bolus injection.

Pharmaceutical compositions and formulations for topical administration can include transdermal patches, ointments, lotions, creams, gels, drops, sprays, suppositories, liquids and powders. Conventional pharmaceutical carriers, aqueous, powder or oily bases, thickeners and the like may be necessary or desirable. Examples of topical formulations include those in which the ASO of the disclosure are in admixture with a topical delivery agent such as lipids, liposomes, fatty acids, fatty acid esters, steroids, chelating agents and surfactants. Compositions and formulations for oral administration include but are not limited to powders or granules, microparticulates, nanoparticulates, suspensions or solutions in water or non-aqueous media, capsules, gel capsules, sachets, tablets or minitablets. Compositions and formulations for parenteral, intrathecal, intra-cerebroventricular, or intraventricular administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids. Delivery of drug to the target tissue can be enhanced by carrier-mediated delivery including, but not limited to, cationic liposomes, cyclodextrins, porphyrin derivatives, branched chain dendrimers, polyethylenimine polymers, nanoparticles and microspheres (Dass C R. J Pharm Pharmacol 2002; 54(1):3-27).

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

For parenteral, subcutaneous, intradermal or topical administration the formulation can include a sterile diluent, buffers, regulators of tonicity and antibacterials. The active ASOs can be prepared with carriers that protect against degradation or immediate elimination from the body, including implants or microcapsules with controlled release properties. For intravenous administration the carriers can be physiological saline or phosphate buffered saline. International Publication No. WO2007/031091 (A2), published Mar. 22, 2007, further provides suitable pharmaceutically acceptable diluent, carrier and adjuvants—which are hereby incorporated by reference.

### IV. Diagnostics

This disclosure further provides a diagnostic method useful during diagnosis of SNCA related diseases, e.g., a synucleinopathy. Non-limiting examples of synucleinopathy include, but are not limited to, Parkinson's disease, Parkinson's Disease Dementia (PDD), dementia with Lewy bodies, and multiple system atrophy.

The ASOs of the disclosure can be used to measure expression of SNCA transcript in a tissue or body fluid from an individual and comparing the measured expression level with a standard SNCA transcript expression level in normal tissue or body fluid, whereby an increase in the expression level compared to the standard is indicative of a disorder treatable by an ASO of the disclosure.

The ASOs of the disclosure can be used to assay SNCA transcript levels in a biological sample using any methods known to those of skill in the art. (Touboul et. al., Anticancer Res. (2002) 22 (6A): 3349-56; Verjout et. al., Mutat. Res. (2000) 640: 127-38); Stowe et. al., J. Virol. Methods (1998) 75 (1): 93-91).

By “biological sample” is intended any biological sample obtained from an individual, cell line, tissue culture, or other source of cells potentially expressing SNCA transcript. Methods for obtaining tissue biopsies and body fluids from mammals are well known in the art.

### V. Kits Comprising ASOs

This disclosure further provides kits that comprise an ASO of the disclosure described herein and that can be used to perform the methods described herein. In certain embodiments, a kit comprises at least one ASO in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed ASO can be readily incorporated into one of the established kit formats which are well known in the art.

### VI. Methods of Using

The ASOs of the disclosure can be utilized for therapeutics and prophylaxis.

SNCA is a 140 amino acid protein preferentially expressed in neurons at presynaptic terminals where it is thought to play a role in regulating synaptic transmission. It has been proposed to exist natively as both an unfolded monomer and a stable tetramer of α-helices and has been shown to undergo several posttranslational modifications. One modification that has been extensively studied is phosphorylation of SNCA at amino acid serine 129 (S129). Normally, only a small percentage of SNCA is constitutively phosphorylated at S129 (pS129), whereas the vast majority of SNCA found in pathological intracellular inclusions is pS129 SNCA. These pathological inclusions consist of aggregated, insoluble accumulations of misfolded SNCA proteins and are a characteristic feature of a group of neurodegenerative diseases collectively known as synucleinopathies.

In synucleinopathies, SNCA can form pathological aggregates in neurons known as Lewy bodies, which are characteristic of both Parkinson's Disease (PD), Parkinson's Disease Dementia (PDD), and dementia with Lewy bodies (DLB). The present ASOs therefore can reduce the number of the SNCA pathological aggregates or prevent formation of the SNCA pathological aggregates. Additionally, abnormal SNCA-rich lesions called glial cytoplasmic inclusions (GCIs) are found in oligodendrocytes, and represent the hallmark of a rapidly progressing, fatal synucleinopathy known as multiple systems atrophy (MSA). In some embodiments, the ASOs of the disclosure reduce the number of GCIs or prevent formation of GCIs. Reports of either undetectable or low levels of SNCA mRNA expression in oligodendrocytes suggest that some pathological form of SNCA is propagated from neurons, where it is highly expressed, to oligodendrocytes. In certain embodiments, the ASOs of the disclosure reduce or prevent propagation of SNCA, e.g., pathological form of SNCA, from neurons.

The ASOs can be used in research, e.g., to specifically inhibit the synthesis of SNCA protein (typically by degrading or inhibiting the mRNA and thereby prevent protein formation) in cells and experimental animals thereby facilitating functional analysis of the target or an appraisal of its usefulness as a target for therapeutic intervention. Further provided are methods of down-regulating the expression of SNCA mRNA and/or SNCA protein in cells or tissues comprising contacting the cells or tissues, in vitro or in vivo, with an effective amount of one or more of the ASOs, conjugates or compositions of the disclosure.

For therapeutics, an animal or a human, suspected of having a disease or disorder, which can be treated by modulating the expression of SNCA transcript and/or SNCA protein is treated by administering ASO compounds in accordance with this disclosure. Further provided are methods of treating a mammal, such as treating a human, suspected of having or being prone to a disease or condition, associated with expression of SNCA transcript and/or SNCA protein by administering a therapeutically or prophylactically effective amount of one or more of the ASOs or compositions of the disclosure. The ASO, a conjugate or a pharmaceutical composition according to the disclosure is typically administered in an effective amount. In some embodiments, the ASO or conjugate of the disclosure is used in therapy.

The disclosure further provides for an ASO according to the disclosure, for use in treating one or more of the diseases referred to herein, such as a disease selected from the group consisting of Parkinson's disease, Parkinson's Disease Dementia (PDD), dementia with Lewy bodies, multiple system atrophy, and any combinations thereof.

The disclosure further provides for a method for treating α-synucleinopathies, the method comprising administering an effective amount of one or more ASOs, conjugates, or pharmaceutical compositions thereof to an animal in need thereof (such as a patient in need thereof).

In certain embodiments, the disease, disorder, or condition is associated with overexpression of SNCA gene transcript and/or SNCA protein.

The disclosure also provides for methods of inhibiting (e.g., by reducing) the expression of SNCA gene transcript and/or SNCA protein in a cell or a tissue, the method comprising contacting the cell or tissue, in vitro or in vivo, with an effective amount of one or more ASOs, conjugates, or pharmaceutical compositions thereof, of the disclosure to affect degradation of expression of SNCA gene transcript thereby reducing SNCA protein.

In certain embodiments, the ASOs are used to reduce the expression of SNCA mRNA in one or more sections of brain, e.g., hippocampus, brainstem, striatum, or any combinations thereof. In other embodiments, the ASOs reduce the expression of SNCA mRNA, e.g., in brain stem and/or striatum, less than 70%, less than 60%, less than 50%, less than 40%, less than 30%, less than 20%, less than 10%, or less than 5% compared to the SNCA mRNA expression after administration of or exposure to a vehicle (no ASO), at day 3, day 5, day 7, day 10, day 14, day 15, day 20, day 21, or day 25. In some embodiments, the expression of SNCA mRNA is maintained below 70%, below 60%, below 50%, below 40%, below 30%, below 20%, below 10%, or below 5% compared to the SNCA mRNA expression after administration of or exposure to a vehicle (no ASO) until day 28, day 30, day 32, day 35, day 40, day 42, day 45, day 49, day 50, day 56, day 60, day 63, day 70, or day 75.

In other embodiments, the ASOs of the present disclosure reduces SNCA mRNA and/or SNCA protein expression in medulla, caudate putamen, pons cerebellum, lumbar spinal cord, frontal cortex, and/or any combinations thereof.

The disclosure also provides for the use of the ASO or conjugate of the disclosure as described for the manufacture of a medicament. The disclosure also provides for a composition comprising the ASO or conjugate thereof for use in treating a disorder as referred to herein, or for a method of the treatment of as a disorder as referred to herein. The present disclosure also provides ASOs or conjugates for use in therapy. The present disclosure additionally provides ASOs or conjugates for use in the treatment of synucleinopathy.

The disclosure further provides for a method for inhibiting SNCA protein in a cell which is expressing SNCA comprising administering an ASO or a conjugate according to the disclosure to the cell so as to affect the inhibition of SNCA protein in the cell.

The disclosure includes a method of reducing, ameliorating, preventing, or treating neuronal hyperexcitability in a subject in need thereof comprising administering an ASO or a conjugate according to the disclosure.

The disclosure also provides for a method for treating a disorder as referred to herein the method comprising administering an ASO or a conjugate according to the disclosure as herein described and/or a pharmaceutical composition according to the disclosure to a patient in need thereof.

The ASOs and other compositions according to the disclosure can be used for the treatment of conditions associated with over expression or expression of mutated version of SNCA protein.

The disclosure provides for the ASO or the conjugate according to disclosure, for use as a medicament, such as for the treatment of α-Synucleinopathies. In some embodiments the α-Synucleinopathy is a disease selected from the group consisting of Parkinson's disease, Parkinson's Disease Dementia (PDD), dementia with Lewy bodies, multiple system atrophy, and any combinations thereof.

The disclosure further provides use of an ASO of the disclosure in the manufacture of a medicament for the treatment of a disease, disorder or condition as referred to herein. In some embodiments, the ASO or conjugate of the disclosure is used for the manufacture of a medicament for the treatment of a α-Synucleinopathy, a seizure disorder, or a combination thereof.

Generally stated, one aspect of the disclosure is directed to a method of treating a mammal suffering from or susceptible to conditions associated with abnormal levels of SNCA i.e., a α-synucleinopathy), comprising administering to the mammal and therapeutically effective amount of an ASO targeted to SNCA transcript that comprises one or more LNA units. The ASO, a conjugate or a pharmaceutical composition according to the disclosure is typically administered in an effective amount.

The disease or disorder, as referred to herein, can, in some embodiments be associated with a mutation in the SNCA gene or a gene whose protein product is associated with or interacts with SNCA protein. Therefore, in some embodiments, the target mRNA is a mutated form of the SNCA sequence.

An interesting aspect of the disclosure is directed to the use of an ASO (compound) as defined herein or a conjugate as defined herein for the preparation of a medicament for the treatment of a disease, disorder or condition as referred to herein.

The methods of the disclosure can be employed for treatment or prophylaxis against diseases caused by abnormal levels of SNCA protein. In some embodiments, diseases caused by abnormal levels of SNCA protein are α-synucleinopathies. In certain embodiments, α-synucleinopathies include Parkinson's disease, Parkinson's Disease Dementia (PDD), dementia with Lewy bodies, and multiple system atrophy.

Alternatively stated, in some embodiments, the disclosure is furthermore directed to a method for treating abnormal levels of SNCA protein, the method comprising administering a ASO of the disclosure, or a conjugate of the disclosure or a pharmaceutical composition of the disclosure to a patient in need thereof.

The disclosure also relates to an ASO, a composition or a conjugate as defined herein for use as a medicament.

The disclosure further relates to use of a compound, composition, or a conjugate as defined herein for the manufacture of a medicament for the treatment of abnormal levels of SNCA protein or expression of mutant forms of SNCA protein (such as allelic variants, such as those associated with one of the diseases referred to herein).

A patient who is in need of treatment is a patient suffering from or likely to suffer from the disease or disorder.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1: Construction of ASOs

Antisense oligonucleotides described herein were designed to target various regions in the SNCA pre-mRNA. See FIG. 1A for genomic SNCA sequence and FIG. 1B for SNCA cDNA. For example, the ASOs were constructed to target the regions denoted using the pre-mRNA start site and pre-mRNA end site of NG_011851.1 as shown in FIG. 2 and/or mRNA start site and end site of its mRNAs. The exemplary sequences of the ASOs (e.g., SEQ ID Numbers) are described in FIGS. 2 and 3. In some embodiments, the ASOs were designed to be gapmers (e.g., alternating gapmers). See DES Numbers.

FIGS. 2 and 3 show non-limiting examples of the ASO design for selected sequences. The same methods can be applied to any other sequences disclosed herein. The gapmers were constructed to contain locked nucleic acids—LNAs (upper case letters). For example, a gapmer can have Beta-deoxy LNA at the 5′ end and the 3′ end and have a phosphorothioate backbone. But the LNAs can also be substituted with any other nucleotide analogs and the backbone can be other types of backbones (e.g., a phosphodiester linkage, a phosphotriester linkage, a methylphosphonate linkage, a phosphoramidate linkage, or combinations thereof).

The ASOs were synthesized using methods well known in the art. Exemplary methods of preparing such ASOs are described in Barciszewski et al., Chapter 10-“Locked Nucleic Acid Aptamers” in Nucleic Acid and Peptide Aptamers: Methods and Protocols, vol. 535, Gunter Mayer (ed.) (2009), the entire contents of which is hereby expressly incorporated by reference herein.

### Example 2A: High Content Assay to Measure Reduction of SNCA Protein in Primary Neurons

ASOs targeting SNCA were tested for their ability to reduce SNCA protein expression in primary mouse neurons. The primary neuronal cultures were established from the forebrain of PAC-Tg(SNCA^{A53T})^{+/+};SNCA^{-/-} (“PAC-A53T”) mice carrying the entire human SNCA gene with a A53T mutation on a mouse SNCA knockout background. See Kuo Y et al., Hum Mol Genet., 19: 1633-50 (2010). All procedures involving mice were conducted according to Animal Test Methods (ATM) approved by the Bristol-Myers Squibb Animal Care and Use Committee (ACUC). Primary neurons were generated by papain digestion according to manufacturer's protocol (Worthington Biochemical Corporation, LK0031050). Isolated neurons were washed and resuspended in Neurobasal medium (NBM, Invitrogen) supplemented with B27 (Gibco), 1.25 M Glutamax (Gibco), 100 unit/ml penicillin, 100 μg/ml streptomycin, and 25 μg/ml Amphotericin B.

Cells were plated on multi-well poly D-Lysine coated plates at 5,400 cells/cm² (for example in 384 well plates 6,000 cells/well in 25 μl NBM). ASOs were diluted in water and added to the cells at DIV01 (i.e., 1 day post plating). ASOs were added to 2× final concentration in medium then delivered to cells manually. Alternatively, ASOs in water were dispensed using a Labcyte ECHO acoustic dispenser. For ECHO dispense, 250 nl of ASO in water was added to cells in medium followed by the addition of an equal volume aliquot of fresh aliquot of NBM. For primary screening, the ASOs were added to final concentrations of 5 μM, 3.3 μM, 1 μM, 200 nM, or 40 nM. For potency determination, 8-10 point titrations of the ASOs were prepared from 0.75 mM stock then delivered to cultured cells for a final concentration range of 2.7-4000 nM or 4.5-10,000 nM. ASO-000010 (TCTgtcttggctTTG, SEQ ID NO: 22) and ASO-000838 (AGAaataagtggtAGT, SEQ ID NO: 23) (5 μM) were included in each plate as reference control inhibitors for tubulin and SNCA, respectively. The cells were incubated with the ASOs for 14 days to achieve steady state reduction of mRNA.

After the 14-day incubation, the cells were fixed by the addition of fixative to final concentrations of 4% formaldehyde (J. T. Baker) and 4% sucrose (Sigma) in the wells. The cells were fixed for 15 minutes, and then, the fixative aspirated from the wells. Then, the cells were permeabilized for 20 minutes with a phosphate buffered saline (PBS) solution containing 0.3% Triton-X 100 and 3% bovine serum albumin (BSA) or 3% Normal goat serum. Afterwards, the permeabilization buffer was aspirated from the wells, and the cells were washed once with PBS. The primary antibodies were then diluted in PBS containing 0.1% Triton X-100 and 3% BSA. Dilutions of 1:1000 of rabbit anti-SNCA (Abcam) and 1:500 of chicken anti-tubulin (Abcam) were used. Cells were incubated with the primary antibodies between 2 hours to overnight. Following the incubation, the primary antibody staining solution was aspirated, and the cells were washed 2-times with PBS. A secondary staining solution containing 1:500 dilution of goat-anti-chicken Alexa 567 antibody, goat anti-rabbit-Alexa 488 antibody, and Hoechst (10 μg/ml) in PBS containing 0.1% Triton X-100 with 3% BSA was added to the wells, and the plates were incubated for 1 hour. Afterwards, the secondary staining solution was aspirated from the wells, and the cells were washed 3-times with PBS. After washing the cells, 60 μl of PBS was added to each well. Plates were then stored in the PBS until imaging.

For imaging, the plates were scanned on a Thermo-Fisher (Cellomics) CX5 imager using the Spot Detector bio-application (Cellomics) to quantify nuclei (Hoechst stain, Channel 1), tubulin extensions (Alexa 567, channel 2) and SNCA (Alexa 488, channel 3). Object count (nuclei) was monitored but not published to the database. The total area covered by tubulin was quantified as the feature SpotTotalAreaCh2 and total intensity of staining for SNCA quantified as SpotTotalIntenCh3. The tubulin measure was included to monitor toxicity. To determine the reduction of SNCA protein, the ratio of SNCA intensity to the tubulin staining area was calculated and results normalized as % inhibition median using the median of vehicle treated wells as total and ASO-000010 or ASO-000838 wells as maximally inhibited wells for tubulin or SNCA, respectively.

### Example 2B: Spontaneous Calcium Oscillation Measurement

Reduced oscillations in intracellular free calcium concentration (calcium oscillation) corresponds to increased neurotoxicity and therefore, can indicate reduced tolerability in vivo. To measure primary cortical neuron spontaneous calcium oscillation, rat primary cortical neurons were prepared from Sprague-Dawley rat embryos (E19). Briefly, the brain cortex was dissected and incubated at 37° C. for 30-45 minutes in papain/DNase/Earle's balanced salt solution (EBSS) solution. After trituration and centrifugation of the cell pellet, the reaction was stopped by incubation with EBSS containing protease inhibitors, bovine serum albumin (BSA), and DNase. The cells were then triturated and washed with Neurobasal (NB, Invitrogen) supplemented with 2% B-27, 100 μg/ml penicillin, 85 μg/ml streptomycin, and 0.5 mM glutamine.

The cells were plated at a concentration of 25,000 cells/well onto 384-well poly-D-lysine coated fluorescent imaging plates (BD Biosciences) in 25 μl/well supplemented Neurobasal (NB) media (containing B27 supplement and 2 mM glutamine). The cells were grown for 12 days at 37° C. in 5% CO₂ and fed with 25 μl of additional media on DIV04 (i.e., 4 days after plating) and DIV08 (i.e., 8 days after plating) for use on DIV12 (i.e., 12 days after plating).

On the day of the experiment, the NB media was removed from the plate and the cells were washed once with 50 μl/well of 37° C. assay buffer (Hank's Balanced Salt Solution, containing 2 mM CaCl₂) and 10 mM Hopes pH 7.4). Oscillations were tested both in the presence and in the absence of 1 mM MgCl₂. The cells were loaded with a cell permanent fluorescent calcium dye, Fluo-4-AM (Invitrogen, Molecular Probes F14201). Fluo-4-AM was prepared at 2.5 mM in DMSO containing 10% pluronic F-127 and then diluted 1:1000 in the assay buffer for a final concentration of 2.5 μM. The cells were incubated for 1 hr with 20 μl of 2.5 μM Fluo-4-AM at 37° C. in 5% CO₂. After the incubation, an additional 20 μl of room temperature assay buffer was added, and the cells were allowed to equilibrate to room temperature in the dark for 10 minutes.

The plates were read on a FDSS 7000 fluorescent plate reader (Hamamatsu) at an excitation wavelength of 485 nm and emission wavelength of 525 nm. The total fluorescence recording time was 600 seconds at 1 Hz acquisition rate for all 384 wells. An initial baseline signal (measurement of intracellular calcium) was established for 99 seconds before the addition of the ASOs. ASOs were added with a 384 well head in the FLIPR in 20 μl of assay buffer at 75 μM for a final concentration of 25 μM. In some instances an ASO targeting tau such as ASO-000013 (OxyAs OxyTs OxyTs DNAts DNAcs DNAcs DNAas DNAas DNAas DNAts DNAts DNAcs DNAas OxyMCs OxyTs OxyT; ATTtccaaattcaCTT, SEQ ID NO: 24) or ASO-000010 (TCTgtcttggctTTG, SEQ ID NO: 22) was included as controls.

Fluorescence time sequence intensity measurements (described above) were exported from the Hamamatsu reader, and transferred to an in-house proprietary application in IDBS E-Workbook suite for data reduction and normalization. In each 384 well screening plate, up to a maximum of 48 individual ASOs were tested in quadruplicate wells. 12 wells were exposed to a positive control (ASO-000010), which significantly inhibits the calcium oscillations counted during the 300 sec acquisition time frame and 12 wells were exposed to an negative control inactive ASO (ASO-000013) which does not inhibit the observation of calcium oscillations. Finally, 24 wells were dedicated to a vehicle control consisting of RNase-DNase-free water at the same concentration used to dilute the test ASOs. The effects of test ASOs in individual wells on calcium oscillation frequency (over the 300 sec period) were expressed as a % control of the median number of calcium oscillations counted in the 24 vehicle control wells. Individual 384 well assay plates passed QC standards if the positive and negative ASO controls (ASO-000010 and ASO-000013) exhibited well characterized pharmacology in the Ca assay, and if the vehicle and pharmacological control wells generated a minimum of ˜20 calcium oscillations over the 300 sec experimental time period.

### Example 2C: QUANTIGENE® Analysis (96-Well Assay) to Measure mRNA Reduction in Human Neurons

The ability of ASOs to reduce human SNCA mRNA and/or possible human off target mRNA species was measured in vitro by QUANTIGENE® analysis. Human neurons (Cellular Dynamics Inc., “iNeurons”), were thawed, plated, and cultured per manufacturer's specifications. These iNeurons are highly pure population of human neurons derived from induced pluripotent stem (iPS) cells using Cellular Dynamic's proprietary differentiation and purification protocols.

Lysis: Cells were plated on poly-L-ornithine/laminin coated 96-well plates at 50,000 to 100,000 cells per well (dependent on the expression of the off target being investigated) and maintained in Neurobasal media supplemented with B27, glutamax, and Penicillin-Streptomycin. The ASOs were diluted in water and added to cells at DIV01 (i.e., 1 day post plating). For single point measurements, a final ASO concentration of 0.5 μM was typically used. For IC₅₀ determinations, the neurons were treated with a seven-point concentration response dilution of 1:4, with the highest concentration as 5 μM to define the IC₅₀. The cells were then incubated at 37° C. and 5% CO₂ for 6 days to achieve steady state reduction of mRNA.

After the incubation, the media was removed and cells were washed 1× in DPBS and lysed as follows. Measurement of lysate messenger RNA was performed using the QUANTIGENE® 2.0 Reagent System (AFFYMETRIX®), which quantitates RNA using a branched DNA-signal amplification method reliant on the specifically designed RNA capture probe set. The working cell lysis buffer solution was made by adding 50 μl proteinase K to 5 ml of pre-warmed (37° C.) Lysis mix and diluted in dH₂O to a 1:4 final dilution. The working lysis buffer was added to the plates (100 to 150 μl/well, depending on the expression of the off target being investigated), triturated 10 times, sealed and incubated for 30 min at 55° C. Following the lysis, the wells were triturated 10 more times, and the plates were stored at −80° C. or assayed immediately.

Assay: Depending on the specific capture probe used (i.e., SNCA, PROS1, or tubulin), the lysates were diluted (or not diluted) in the lysis mix. Then, the lysates were added to the capture plates (96-well polystyrene plate coated with capture probes) at a total volume of 80 μl/well. Working probe sets reagents were generated by combining nuclease-free water (12.1 μl), lysis mixture (6.6 μl), blocking reagent (1 μl), and specific 2.0 probe set (0.3 μl) (human SNCA catalogue #SA-50528, human PROS1 catalogue #SA-10542, or human beta 3 tubulin catalogue #SA-15628) per manufacturer's instructions (QUANTIGENE® 2.0 AFFYMETRIX®). Next, 20 μl working probe set reagents were added to 80 μl lysate dilution (or 80 μl lysis mix for background samples) on the capture plate. Plates were centrifuged at 240 g for 20 seconds and then incubated for 16-20 hours at 55° C. to hybridize (target RNA capture).

Signal amplification and detection of target RNA began by washing plates with buffer 3 times (300 μl/well) to remove any unbound material. Next, the 2.0 Pre-Amplifier hybridization reagent (100 μl/well) was added, incubated at 55° C. for 1 hour, then aspirated, and wash buffer was added and aspirated 3 times. The 2.0 Amplifier hybridization reagent was then added as described (100 μl/well), incubated for 1 hour at 55° C. and the wash step repeated as described previously. The 2.0 Label Probe hybridization reagent was added next (100 μl/well), incubated for 1 hour at 50° C. and the wash step was repeated as described previously. The plates were again centrifuged at 240 g for 20 seconds to remove any excess wash buffer and then, the 2.0 Substrate was added (100 μl/well) to the plates. Plates were incubated for 5 minutes at room temperature and then, the plates were imaged on a PerkinElmer Envision multilabel reader in luminometer mode within 15 minutes.

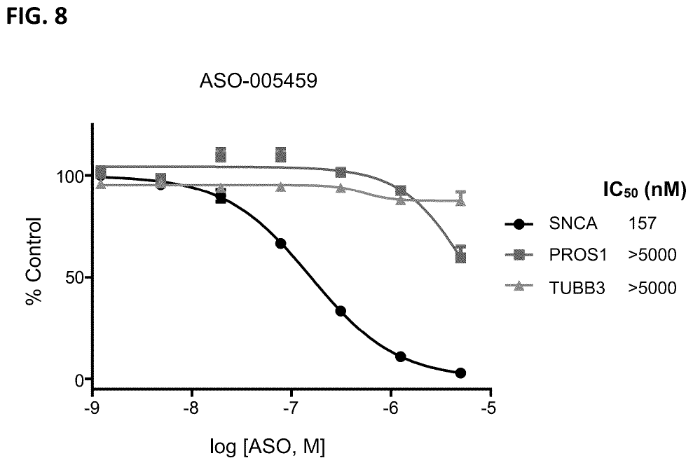

Data determination: For the gene of interest, the average assay background signal was subtracted from the average signal of each technical replicate. The background-subtracted, average signals for the gene of interest were then normalized to the background-subtracted average signal for the housekeeping tubulin RNA. The percent inhibition for the treated sample was calculated relative to the control treated sample lysate. Results of QUANTIGENE® assays for cells treated with the ASOs are shown in e.g., FIG. 8.

### Example 2D: QUANTIGENE® Analysis (96-Well Assay) to Measure mRNA Reduction in Ramos Cells

To measure possible human off target IKZF3 (IKAROS family zinc finger 3) mRNA reduction, Ramos cells (a human lymphocytic cell line) were used. Since Ramos cells do not express SNCA, RB1 (RB transcriptional corepressor 1), which is expressed in Ramos cells, was used as a positive control for assessing ASO-mediated knockdown IKZF3 mRNA expression. Two ASOs were synthesized to bind to and knockdown human RB1 mRNA expression. Beta-2 microglobulin (β2M) was used as a housekeeping gene control. The Ramos cells were grown in suspension in RPMI media supplemented with FBS, glutamine, and Pen/Strep.

Lysis: Cells were plated on poly-L-ornithine/laminin coated 96 well plates at 20,000 cells per well and maintained in Neurobasal media containing B27, glutamax and Penicillin-Streptomycin. ASOs were diluted in water and added to cells at 1 day post plating (DIV01) to a final concentration of 1 μM. Following ASO treatment, the cells were incubated at 37° C. for 4 days to achieve steady state reduction of mRNA. After the incubation, the media was removed and cells lysed as follows. Measurement of lysate messenger RNA was performed using the QUANTIGENE® 2.0 Reagent System (AFFYMETRIX®), which quantitated RNA using a branched DNA-signal amplification method reliant on the specifically designed RNA capture probe set. Lysis mix (QUANTIGENE®2.0 AFFYMETRIX®) was pre-warmed in an incubator at 37° C. for 30 minutes. For lysing cells in suspension, 100 μl of 3× Lysis Buffer (with 10 μl/ml proteinase K) was added to 200 μl of cells in suspension. The cells were then triturated 10 times to lyse, and the plate sealed and incubated for 30 min at 55° C. Afterwards, the lysates were stored at −80° C. or assayed immediately.

Assay: Depending on the specific capture probe used (i.e., IKZF3, RB1, and 02M), the lysates were diluted (or not diluted) in the lysis mix. Then, the lysates were added to the capture plate (96 well polystyrene plate coated with capture probes) at a total volume of 80 μl/well. Working probe sets reagents were generated by combining nuclease-free water 12.1 μl, lysis mixture 6.6 μl, blocking reagent 1 μl, specific 2.0 probe set 0.3 μl (human IKZF3 catalogue #SA-17027, human RB1 catalogue #SA-10550, or human beta-2 microglobulin catalogue #SA-10012) per manufacturer instructions (QUANTIGENE® 2.0 AFFYMETRIX®). Then 20 μl working probe set reagents were added to 80 μl lysate dilution (or 80 μl lysis mix for background samples) on the capture plate. Plates were then incubated for 16-20 hours at 55° C. to hybridize (target RNA capture). Signal amplification and detection of target RNA was begun by washing plates with buffer 3 times (300 μl/well) to remove any unbound material. Next, the 2.0 Pre-Amplifier hybridization reagent (100 μl/well) was added, incubated at 55° C. for 1 hour then aspirated and wash buffer was added and aspirated 3 times. The 2.0 Amplifier hybridization reagent was then added as described (100 μl/well), incubated for 1 hour at 55° C. and the wash step was repeated as described previously. The 2.0 Label Probe hybridization reagent was added next (100 μl/well), incubated for 1 hour at 50° C. and the wash step again was repeated as described previously. The plates were again centrifuged at 240 g for 20 seconds to remove any excess wash buffer and then, the 2.0 Substrate was added (100 μl/well) to the plates. Plates were incubated for 5 minutes at room temperature, and then, the plates were imaged on a PerkinElmer Envision multilabel reader in luminometer mode within 15 minutes.

Data determination: For the gene of interest, the average assay background signal (i.e., no lysate, just 1× lysis buffer) was subtracted from the average signal of each technical replicate. The background-subtracted, average signals for the gene of interest were then normalized to the background-subtracted average signal for the housekeeping mRNA (for Ramos cells, it was beta-2-microglobulin). The percent inhibition for the treated sample was calculated relative to the average of the untreated sample lysate. Results of QUANTIGENE® assays for cells treated with the ASOs are shown in Table 4.

### Example 2E: qPCR Assay to Measure Reduction of SNCA mRNA in SK-N-BE(2) Cells

ASO SAN-005459 targeting SNCA was tested for its ability to reduce SNCA mRNA expression in human SK-N-BE(2) neuroblastoma cell acquired from ATCC (CRL-2271).

SK-N-BE(2) cells were grown in cell culturing media (MEM [Sigma, cat.no M2279] supplemented with 10% Fetal Bovine Serum [Sigma, cat.no F7524], 1× GLUTAMAX™ [Sigma, cat.no 3050-038]1×MEM Non-essential amino acid solution [Sigma, cat.no M7145] and 0.025 mg/ml Gentamycin [Sigma, cat.no G1397]). Cells were trypsinized every 5 days, by washing with Phosphate Buffered Saline (PBS), [Sigma cat.no 14190-094] followed by addition of 0.25% Trypsin-EDTA solution (Sigma, T3924), 2-3 minutes incubation at 37° C., and trituration before cell seeding. Cells were maintained in culture for up to 15 passages.

For experimental use, 12,500 cells per well were seeded in 96 well plates (Nunc cat.no 167008) in 100 μL growth media. Oligonucleotides were prepared from a 750 μM stock. ASO dissolved in PBS was added approximately 24 hours after the cells were seeded to a final concentration of 25 μM for single point studies. Cells were incubated for 4 days without any media change. For potency determination, 8 concentrations of ASO were prepared for a final concentration range of 16-50,000 nM. ASO-004316 (CcAAAtcttataataACtAC, SEQ ID NO: 25) and ASO-002816 (TTCctttacaccACAC, SEQ ID NO: 12) were included as controls.

After incubation, cells were harvested by removal of media followed by addition of 125 μL PURELINK® Pro 96 Lysis buffer (Invitrogen 12173.001A) and 125 μL 70% ethanol. RNA was purified according to the manufacture's instruction and eluted in a final volume of 50 μL water resulting in an RNA concentration of 10-20 ng/μl. RNA was diluted 10 fold in water prior to the one-step qPCR reaction. For one-step qPCR reaction qPCR-mix (qScript TMXLE 1-step RT-qPCR TOUGHMIX® Low ROX from QauntaBio, cat.no 95134-500) was mixed with two Tagman probes in a ratio 10:1:1 (qPCR mix: probe1:probe2) to generate the mastermix. Taqman probes were acquired from LifeTechnologies: SNCA: Hs01103383_ml; PROS1: Hs00165590_ml: TBP: 4325803; GAPDH 4325792. Mastermix (6 μL) and RNA (4 μL, 1-2 ng/μL) were then mixed in a qPCR plate (MICROAMP® optical 384 well, 4309849). After sealing, the plate was given a quick spin, 1000 g for 1 minute at RT, and transferred to a Viia™ 7 system (Applied Biosystems, Thermo), and the following PCR conditions used: 50° C. for 15 minutes; 95° C. for 3 minutes; 40 cycles of: 95° C. for 5 sec followed by a temperature decrease of 1.6° C./sec followed by 60° C. for 45 sec. The data was analyzed using the QUANTSTUDIO™ Real-Time PCR Software.

### Example 3: In Vitro Analysis of ASO-005459 on the Reduction of Human SNCA mRNA

ASO-005459 is a 17-base, LNA-modified ASO that targets the intron1/exon2 boundary of human SNCA pre-mRNA (see FIG. 2, SEQ ID NO: 15).

### Potency of ASO-005459 in Mouse Neurons

Using the methods described above in Example 2A, ASO-005459 was tested for its ability to reduce SNCA protein expression as a downstream result of reduction in SNCA mRNA. Briefly, primary neurons derived from PAC-A53T mice were treated with ASO-005459 or control ASOs for 14 days. Cells were then fixed and the levels of SNCA protein and tubulin protein were measured by high content imaging. Tubulin levels were measured to monitor toxicity and to normalize SNCA protein reduction.

As shown in Table 1 below, incubation of cells with 4 nM of ASO-005459 resulted in a 21% reduction in the SNCA protein expression. With 40 nM and 5 μM of ASO-005459, the SNCA protein expression was reduced by 84% and 86%, respectively. In contrast, ASO-005459 had minimal to no effect on the level of tubulin protein

TABLE 1
ASO-005459 activity in A53T-PAC neurons

| ASO-005459 | aSyn/tub | | | Tub | | |
|---|---|---|---|---|---|---|
| concentration | % inh | SD | N | % inh | SD | N |
| 4 nM | 21.11 | 33.71 | 3 | −8.60 | 12.07 | 4 |
| 40 nM | 84.28 | 13.21 | 8 | 4.32 | 28.65 | 7 |
| 5 μM | 86.45 | 7.91 | 2 | 29.01 | 18.06 | 2 |

SD = standard deviation
N = number of tests

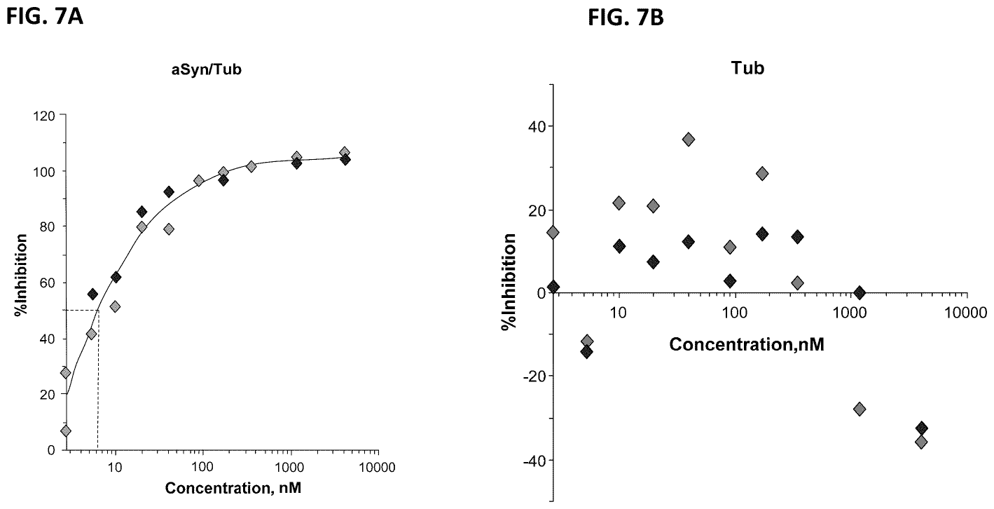

To further evaluate potency, A53T-PAC neurons were treated with a 10-point titration of ASO-005459, as described above in Example 2A, and the IC₅₀ s for the effect on SNCA and tubulin proteins determined. As shown in FIGS. 7A and 7B, and Table 2 (below), a concentration-dependent reduction in α-Syn/tubulin ratio was observed, with an average IC₅₀ of 7.4 nM. This observation was consistent with the single point activity data shown in Table 1 (above). Moreover, ASO-005459 produced negligible effects on Tub levels. Taken together these results indicate that ASO-005459 potently reduces SNCA protein levels with minimal effect on overall cell viability.

TABLE 2
Potency and selectivity estimate for ASO-005459 for
SNCA and tubulin protein in A53T-PAC neurons

| aSyn/tub IC₅₀ | | | | | |
|---|---|---|---|---|---|
| (nM) | SD | N | tub IC₅₀ (nM) | SD | N |
| 7.42 | 2.73 | 6 | >4000 | NA | 5 |

SD = standard deviation
N = number of tests

### Efficacy of ASO-005459 in SK-N-BE(2) Cells

Using the method described in Example 2E, ASO-005459 was tested for its ability to reduce SNCA mRNA in SK-N-BE(2) after 4 days treatment.

Incubation of cells with 25 μM ASO-005459 resulted in 92% reduction in SNCA mRNA in SK-N-BE(2) cells.

### Potency of ASO-005459 in Human Neurons

ASO-005459 potency for SNCA was confirmed using primary human neurons as described above in Example 2C. Briefly, human neurons were derived from induced pluripotent stem (iPS) cells. Cells were treated with ASO-005459 or control ASOs for 6 days and then, mRNA levels were measured by QUANTIGENE® Assay. Because human neurons also express PROS1, a potential off-target for ASO-005459, the PROS1 mRNA level was also measured to assess the effect of ASO-005459 on off-target genes. In addition, tubulin (TUBB) mRNA was measured to monitor toxicity.

As shown in FIG. 8 and Table 3 (below), ASO-005459 induced a concentration-dependent reduction in SNCA expressed in human neurons, with an average IC₅₀ of 100 nM. This IC₅₀ is ˜13-fold weaker than the IC₅₀ for reducing SNCA protein in PAC-A53T neurons (Table 2, above). The reason for this potency shift is not clear but could be due to differences in ASO uptake, metabolism, or kinetics of SNCA knock-down. While ASO-005459 also exhibited concentration-dependent reductions of PROS1 mRNA in the human neurons; the IC₅₀ was 30-50 fold weaker than the IC₅₀ for SNCA. These results confirm ASO-005459 activity for SNCA in human neurons and indicate that ASO-005459 is 30-50-fold more selective for SNCA compared to PROS1.

TABLE 3
Potency and Selectivity of ASO-005459 on SNCA
and PROS1 mRNA in human neurons

| | | SNCA | PROS1 | TUBB3 | PROS1/ |
|---|---|---|---|---|---|
| | | IC₅₀ | IC₅₀ | IC₅₀ | SNCA |
| Assay Date | Batch | (nM) | (nM) | (nM) | IC₅₀ ratio |
| Aug. 26, 2016 | 01-001 | 42 | 2127 | >5000 | 51 |
| Dec. 8, 2016 | 01-004 | 157 | >5000 | >5000 | >32 |

Batch = particular lot-number of ASO-005459 used

### Potency of ASO-005459 in Ramos Cells

IKZF3 is another potential off-target for ASO-005459, but, unlike PROS1, it is not expressed in human neurons. Instead, IKZF3 is robustly expressed in Ramos cells, a human lymphocytic cell line. Ramos cells were treated with 1 μM ASO-005459 and the effect on IKZF3 mRNA expression was measured. To monitor toxicity, beta-2 microglobulin mRNA was also measured. Since Ramos cells do not express SNCA, another gene, RB1 (RB transcriptional corepressor 1), was used as a positive control for ASO-mediated knockdown. ASO-006754 (GGTgaggtttggtaGA, SEQ ID NO: 26) and ASO-006755 (GGTgaggtttggtagaAG, SEQ ID NO: 27) target RB1 and were included the study. As shown in Table 4 (below), at 1 μM concentration, ASO-005459 did not affect IKZF3 mRNA expression, demonstrating ASO-005459's specificity for SNCA. ASO-005459 also did not affect the expression of RB1 and 02M mRNAs, indicating that ASO-005459 is not toxic to Ramos cells. In contrast, ASO-006754 and ASO-006755 (the control ASOs) reduced RB1 levels by 87% and 83%, respectively, confirming the ASO activity in Ramos cells.

TABLE 4
Effect of 1 μM ASO-005459 on IKZF3 in Ramos cells

| | | | IKZ F3 | RB1 | β2M |
|---|---|---|---|---|---|
| ASO | Target | Batch | (% con) | (% con) | (% con) |
| ASO-005459 | SNCA | 01-004 | 102 | 99 | 94 |
| ASO-006754 | RB1 | 01-001 | 104 | 13 | 87 |
| ASO-006755 | RB1 | 01-001 | 116 | 17 | 83 |

Batch = particular lot-number of the ASO used

Collectively, the results presented here demonstrate that ASO-005459 is potent and highly selective for reducing SNCA mRNA, which in turn mediates the reduction of SNCA protein levels. The results also show ASO-005459 is well tolerated both in mouse and in human neurons. These findings support the continued development of ASO-005459 as a disease-modifying therapeutic for the treatment of synucleinopathies.

### Example 4: In Vivo Tolerability and In Vivo SNCA mRNA Reduction

The in vivo tolerability of selected ASOs was tested to see how the ASOs were tolerated when injected into different animal models (i.e., mice and cynomolgus monkeys):

### Mice

Subjects: Male and female (2-3 months old) PAC-Tg(SNCA^{A53T})^{+/+};SNCA^{-/-} (“PAC-A53T”) mice carrying the entire human SNCA gene with a A53T mutation on a mouse SNCA knockout background were used for acute, long term, and PK/PD in vivo efficacy studies. In some cases wild-type (WT) C57B/6 mice were used for long term (i.e., 4 weeks) health assessment. Mice were housed in groups of 4 or 5 in a temperature controlled housing room with food and water available ad libitum. All procedures involving mice were conducted according to Animal Test Methods (ATM) approved by the Bristol-Myers Squibb Animal Care and Use Committee (ACUC).

ASO Dosing Solution Preparation: Sterile saline (1 mL) syringes fitted with 0.2 m Whatman filters and nuclease free centrifuge tubes were used to prepare dosing solutions. Indicated volume of water or saline was added to an ASO powder and was vortexed (˜1 min) to dissolve the ASO powder. The solution was then allowed to sit for 10 min and was vortexed again for ˜1 min. The tubes were briefly centrifuged to return all of the liquid to the bottom of the tube, and then, the solution was filtered through a 0.2 m sterile filter into a 2nd RNase free tube. A small aliquot of the primary stock was diluted to 1 mg/ml for analysis of the concentration using Nanodrop. The analytical sample was vortexed three times with manual inversion to mix thoroughly. Then, the UV absorbance of the sample was measured twice at 260 nm with Nanodrop (the pedestal was rinsed and wiped three times before applying the sample). The test sample was discarded once the analysis was complete. The sample was considered ready for dosing if UV absorbance was between 90 and 110% of the sample. If UV absorbance exceeded 110% of the sample, a secondary dilution was prepared; if the absorbance was <90%, the sample was prepared at a higher initial concentration and similar steps were followed as described above. Samples were stored at 4° C. until use.

Freehand Intracerebroventricular (ICV) Injection: ICV injections were performed using a Hamilton micro syringe fitted with a 27 or 30-gauge needle, according to the method of Haley and McCormick. The needle was equipped with a polyethylene guard at 2.5-3 mm from the tip in order to limit its penetration into the brain. Mice were anesthetized using isoflurane anesthetic (1-4%). Once sufficiently anesthetized, the mice were held by the loose skin at the back of the neck with the thumb and first fingers of one hand. Applying gentle but firm pressure, the head of the animal was then immobilized by pressing against a firm flat level surface. Dosing was conducted using 10 μL Hamilton syringes fitted with a 27½g needle. The needle tip was then inserted through the scalp and the skull, about 1 mm lateral and 1 mm caudal to bregma (i.e., right of the midline, about 3 mm back as measured from the eye line). Once the needle was positioned, the ASO was given in a volume of 5 μl in saline vehicle and injected over ˜30 seconds. The needle was left in place for 5-10 seconds before removal. The mice were returned to their home cage and allowed to recover for ˜2-4 min. Mice were observed continuously for 30 minutes immediately after dosing for adverse behavioral effects of drug and/or dosing. During this time, any mouse that convulsed more than 3 separate times was immediately euthanized and given an automatic score of 20. Drug tolerability was scored 1 hr±15 min post dosing. Animals dosed with non-tolerated compounds (tolerability score >4) were euthanized immediately following the 1 hr evaluation.

ASO Tolerability Assessment: Animals dosed with the ASOs were evaluated right after the dosing and monitored for 2 hours for any adverse effects. For acute tolerability (AT) studies, mice were evaluated at the time of dosing and again at the takedown, i.e., 3 days post ASO injection. For long term health assessment, the mice were weighed weekly and monitored for any health and behavioral issues until the completion of the experiment. Mice that had weight losses of greater than 15% of their initial body weight or exhibited tolerability issues were removed from the studies and euthanized. Health and tolerability assessments were conducted according to the following chart:

TABLE 5
Tolerability scoring system^{a}

| Category | Score 1 | Score 2 | Score 3 | Score 4 |
|---|---|---|---|---|
| Hyperactivity, | Very slightly | Increased home | Moderately | Marked |
| stereotypies, | increased home cage | cage exploration | increased home cage | hyperactivity |
| home cage | exploration or | (e.g. digging, | activity | Marked |
| behavior | rearing compared to | burying, etc.) | Detectable | stereotypies |
| controls | | Increased | stereotypies (e.g. | |
| | | grooming | circling, repetitive | |
| | | | behaviors, etc.) | |
| Decreased | Some reduction in | Drowsiness | Stupor (reduced | Coma (does not |
| vigilance, | exploratory activity | Slightly reduced | responsiveness, | respond to |
| exploration and | Responds normally | response to touch | decreased corneal | stimulation, e.g. |
| responsiveness | to stimulation | or handling | reflex) | pinch), no corneal |
| | | | | reflex |
| Motor | Mild change to gait | Reduced grip | Highly reduced grip | Severe ataxia (e.g. |
| coordination and | or grip strength | strength (falls in | strength (falls in less | crawling, fails to |
| strength | (falls between 5- | less than 5 sec) | than 2 sec) | grip bar) |
| | 10 sec) | Mild ataxia (e.g. | Ataxia | No ability |
| | No falls, | slow righting | (e.g. staggering, | to right |
| | normal righting | response, swaying) | falling impaired | |
| | response | | walking) | |
| Posture, | Very slight | Slight abnormal | Moderately | Markedly abnormal |
| appearance, | abnormal posture | posture (e.g. | abnormal posture | posture (e.g. lateral |
| breathing | (subtle) | hunched, extended, | (e.g. ventral | recumbency) |
| | | low posture, tail | recumbency) | Facial |
| | | position, straub | Shallow breathing | paralysis (e.g. |
| | | tail) | | drooling, protruding |
| | | Piloerection | | tongue) |
| | | or ptosis | | Labored |
| | | unkempt | | breathing |
| | | coat | | |
| Tremor, hyper- | Detectable tremor | Hyper-responsive | Few or partial | Repeated or |
| activity, | | to stimuli (e.g. | seizures, rearing and | continuous seizure |
| convulsion | | noise) | falling as part of | (miming, bouncing, |
| | | Marked | convulsing | clonic and/or tonic) |
| | | tremors | | |

^{a}Normal is scored as “0”. Animals are scored on an individual basis at successive time points post dosing. Observations are rated at 1 h ± 15 min, then 24 h ± 2 h, then 7 days (if appropriate). Convulsions count for the 1 hr timepoint, even if they occur prior to the observation window. A total tolerability score is calculated based on the sum of the individual category scores, with a maximum possible score of 20.

Tissue Collection: Following final behavioral and health assessments, mice were decapitated on a guillotine and the brains were quickly removed. Each brain was split into two hemispheres and a) hippocampus was dissected for mRNA measurements in the 3-day acute tolerability studies; b) hippocampus, brain stem, and striatum from one hemisphere were dissected for mRNA measurements, whereas the same regions were dissected from the second hemisphere for protein/PK measurements in the dose-response time course PK/PD studies.

In some of the studies, the blood and the cerebrospinal fluid (CSF) were also collected for PK (blood) and PK/protein (CSF) measurements. To collect the blood and the CSF, the mice were deeply anesthetized with Isoflurane (4%). Blood was collected via cardiac puncture using 23 G needle. Once removed, the blood was transferred into 2 ml BD Microtainer (K2EDTA BD #365974) tubes and placed on ice until processing. To process the blood, the tubes were centrifuged at 4500×g for 10 min at 4° C. Then, the plasma was removed and placed into 0.5 ml Eppendorf tubes and stored at −80° C. until use. To collect the CSF, the thoracic cavity was opened exposing the heart, and as much of the blood was drained to avoid contamination of the CSF. The CSF samples were collected via Cisterna magna using micropipettes and placed into lo-bind protein Eppendorf tubes. Then, the tubes were centrifuged at 4500×g for 15 min at 4° C. The CSF was carefully transferred to clean lo-bind 0.5 ml Eppendorf tubes and stored at −80° C. until further use.

### Cyno Data

Subject: Male cynomolgus monkeys weighing 3.5-10.0 kg at the start of the study were used. Each was implanted with an intrathecal cerebrospinal fluid (CSF) catheter entering at the L3 or L4 vertebrae. The distal tip of the polyurethane catheter extended within the intrathecal space to approximately the L1 vertebrae. The proximal end was connected to a subcutaneous access port located on the animal's lower back. Animals were allowed to heal for at least two weeks prior to the start of the study. Laboratory animal care was according to Public Health Service Policy on the Humane Care and Use of Laboratory Animals and the Guide for the Care and use of Laboratory Animals NRC (2011) (National Research Council: Guide for the Care and Use of Laboratory Animals (The National Academies Collection: Reports funded by National Institutes of Health). National Academies Press (US), Washington (DC)). The protocol was approved by the Wallingford Animal Care and Use Committee of the Bristol-Myers Squibb Company.

CSF & BloodSampling: The CSF port was accessed subcutaneously using aseptic techniques, and CSF was sampled from awake animals sitting upright in a primate restraint chair. Approximately 0.1 ml of CSF was discarded at the start of collection to clear dead space in the catheter and port. CSF was collected by gravity flow to a maximum of 0.5 ml CSF per sample. CSF was spun at 2,000 g at 4° C. for 10 min. The supernatant was frozen on dry ice or in liquid nitrogen and kept at −90° C. until analyzed.

Blood was sampled from an available vein, typically the saphenous vein. Blood samples were prepared in a number of procedures depending upon the particular measure in question. For plasma, blood was collected into EDTA-treated tubes. For serum, blood was collected into serum-separator tubes and allowed to clot for at least 30 min prior to centrifugation. For measures of clotting and clotting factors, blood was collected into citrated tubes, and for analysis of RNA, blood was collected into tubes containing RNA later. After processing, samples were frozen on dry ice or in liquid nitrogen and kept frozen until analyzed.

IntrathecalDosing: Animals were trained to be dosed while awake and using modified commercially-available restraint chairs, animals were maintained in a prone position. SNCA-targeted anti-sense oligonucleotides (ASOs) were dissolved in saline, sterilized by filtration, and administered at 0.33 ml/min in a 1.0 ml volume followed by a 0.5 ml sterile water flush. Total infusion time was 4.5 min. Animals remained in the prone position for 30 min post infusion.

Necropsy: Cynomolgus monkeys were administered the appropriate volume of a commercially available euthanasia solution while anesthetized with ketamine and/or isoflurane. Necropsy tissues were obtained immediately thereafter and the brain was transferred to wet ice for dissection. Areas of interest were dissected using 4-6 mm slices in an ASI Cyno Brain Matrix as well as free handed techniques. Samples were placed fresh in RNAlater, or frozen on dry ice for later analysis. CNS tissue was rapidly dissected form cynomolgus monkeys and pieces no longer than 4 mm on any axis were collected and placed in 5 mLs of RNA later. Samples were stored at 4° C. overnight then transferred to −20° C. for storage until analyzed.

Brain regions analyzed included medulla, pons, midbrain, cerebellum, caudate-putamen (left and right), hippocampus (left and right), frontal cortex (left and right), temporal cortex (left and right), parietal cortex (left and right), occipital cortex (left and right) and cortical white matter. Additionally, spinal cord was sampled at the cervical, thoracic and lumbar regions. Samples were also collected from liver, kidney and heart. On some occasions, samples of trigeminal nuclei, tibial nerve and the aorta were collected to examine off-target pharmacology in those areas.

### ELISA Quantitation of ASO Concentration in Mouse or Monkey Tissue, Plasma, and CSF:

Tissue was homogenized with plasma and water in a 1:1 ratio. Standard curve was generated by 2-fold serial dilution from 5000 to 4.9 nM in plasma (for plasma and CSF) and in plasma:water (for tissues samples) and then further diluted to 5000-fold total with 5×SSCT (750 mM NaCl, and 75 mM sodium citrate, pH 7.0, containing 0.05% (v/v) Tween-20) alone and in 5×SSCT containing 35 nM capture and 35 nM detection reagents to obtain a standard range of 1-1000 pM. The dilution factor used varied depending on the expected sample concentration range. The capture probe was AAAGGAA with a 3′ Biotin (Exiqon) and the detection probe was 5′ DigN-isopropyl 18 linker-GTGTGGT (Exiqon).

Experimental samples and standards were added to Clarity lysis buffer (Phenomenex, cat #ALO-8579) in a 1:1 ratio prior to dilution with capture and detection buffer and before transferring to the ELISA plate. CSF samples were diluted with plasma (2-fold) prior to addition of lysis buffer. A streptavidin-coated plate (Thermo 15119) was washed 3 times with 5×SSCT buffer. 100 μl samples were added and incubated for 60 min at room temperature. The detection probe, 100 μl anti-Dig-AP Fab fragment diluted 1:4000 in PBS containing 0.05% Tween-20 (Roche Applied Science, Cat. No. 11 093 274 910), was added and incubated for 60 min at room temperature. After washing the plate with 2×SSCT buffer, 100 μl Tropix CDP-star Sapphire II substrate (Applied Biosystems) was added for 30 min at room temperature. Antisense oligonucleotide concentrations were measured by luminescence (Enspire-PerkinElmer).

### Alpha-Synuclein Protein Measurements:

Brain tissue samples were homogenized at 10 ml/g tissue in RIPA buffer (50 mM Tris HCl, 150 mM NaCl, 1% NP-40, 0.5% sodium deoxycholate, 0.1% sodium dodecyl sulfate) using bead homogenizer Qiagen Tissuelyser II for 25 cycles/sec, with a 5 mm stainless steel bead for 2 min total. Homogenized samples were incubated 30 min on ice. 50 μL aliquot of each sample was retained for PK analysis. The remaining samples were centrifuged 20,800 g, for 60 min, 4° C. The supernatant was retained and used for analysis. Total protein was measured using Pierce BCA protein assay kit (23227).

Brain tissue extracts: SNCA protein was measured using the MJFR1+4B12 ELISA. Briefly, ELISA plates (Costar) were coated with 100 μl of the anti-SNCA antibody MJFR1 (Abcam) at a concentration of 0.1 μg/ml diluted in BupH carbonate-bicarbonate buffer, pH 9.4 (Thermo Scientific) overnight (O/N) at 4° C. The next day plates were washed 4-times with Dulbecco's PBS (Life Technologies) and blocked with 3% BSA (bovine serum albumin, protease free, Fraction V, Roche Diagnostic) in PBS for 2˜3 h at room temperature (RT) or overnight at 4° C. Both the standards and the brain samples were diluted with 1% BSA/0.05% Tween/PBS containing Roche protease inhibitor (Roche 11836145001, 1 pellet/25 ml) and Phosphatase Inhibitor 2&3 (Sigma, 1:100). SNCA wild-type (rPeptide) was used as a standard. Samples were loaded in duplicate (50 μl/well) and incubated for O/N at 4° C. After plates were equilibrated to RT, 50 μl of the detection antibody 4B12 (Biolegend) (diluted 1:4000 in 1% BSA/0.1% Tween/DPBS) was added to each well and co-incubated with the samples at RT for ˜2 hours. Detection antibody was pre-conjugated with alkaline phosphatase (AP kit from Novus Biologicals). Plates were then washed 4-times with 0.05% Tween/PBS and developed with 100 μl of alkaline phosphatase substrate (Tropix CDP Star Ready-to-Use with Sapphire II, T-2214, Life Technologies) for 30 minutes. Luminescence counts were measured with Perkin Elmer EnVision (2102 Multilabel Reader). The plates were kept constant shaking (Titer plate shaker, speed 3) during the assay. Data was analyzed using GraphPad Prism. Total protein in brain tissue was measured using a Micro protein assay kit (Thermofisher #23235) according to manufacturer's instructions.

Cerebral spinalfluid (CSF): SNCA protein was measured using the U-PLEX Human SNCA Kit: (cat #K151WKK-2, Meso Scale Discovery) according to manufacturer's instructions. CSF samples were diluted 10-fold. Hemoglobin was measured in CSF samples using the Abcam mouse Hemoglobin ELISA kit (ab157715). CSF samples were diluted 40-fold for the hemoglobin measurements.

mRNA measurements by qRT-PCR

Brain regions were harvested and placed in 1.5 ml RNA-later Tissue Protect tubes (Qiagen cat #76514) that were prefilled with RNA-later, a RNA stabilization solution. Tissue in RNA-later solution can be stored at 4° C. for 1 month, or at −20° C. or −80° C. indefinitely.

RNA Isolation: RNeasy Plus Mini Kit: RNA from mouse hippocampus and cortex and was isolated using the RNeasy Plus Mini Kit (Qiagen cat #74134). Tissue samples were homogenized in a volume of 600 μL or 1200 1 RLT Plus buffer containing 10 μl/ml of 2-mercaptoethanol and 0.5% Reagent Dx. 600 μL lysis buffer was used if the tissue sample was <20 mg, 1200 μl lysis buffer was used for tissue samples >20 mg. For homogenization, tissue sample was transferred to a 2.0 mL round-bottom Eppendorf Safe-Lock tube (Eppendorf cat #022600044) containing 600 μL RLT Plus Buffer (plus 10 ul/ml of 2-mercaptoethanol and 0.5% Reagent Dx), and a 5 mm stainless steel Bead (Qiagen cat #69989) Samples were homogenized, using a Qiagen's TissueLyser II instrument. Samples were processed for 2.0 min at 20 Hz, samples rotated 180° and processed for another 2.0 min at 20 Hz. Samples were then processed 2.0 min at 30 Hz, samples rotated 1800 and processed for another 2.0 min at 30 Hz. Longer and/or at higher frequency homogenization used if processing not complete. A 600 μL of the tissue lysate was then transferred into a gDNA Eliminator spin column in a 2.0 mL collection tube and samples centrifuged for 30 secs at 10,000 g. All centrifugation steps were performed at RT. The flow-through was collected and an equal volume of 70% ethanol added and mixed. 600 μL was transferred to RNeasy spin column placed in a 2.0 mL collection tube and samples centrifuged for 15 secs at 10,000 g. The flow-through was discarded and the remaining 600 μL sample added to the spin column. The spin columns were centrifuged and the flow-through discarded. Columns were washed with 700 μL of Wash Buffer RW1, centrifuged for 15 secs at 10,000 g, and the flow-through discarded The columns were then washed 2-times with 500 μL of Buffer RPE containing 4 volumes of ethanol as described in kit protocol. Columns were first centrifuged for 15 secs at 10,000 g for first wash and then for 2.0 min at 10,000 g for the second wash. After second wash, columns were centrifuged once for 1.0 min at 10,000 g to dry the membranes. Columns were then transferred to a new 1.5 mL collection tube and 30 μL of RNase-free water was added directly to the center of the membrane. The membranes were allowed to incubate for 10 min at RT. Then, the columns were centrifuged for 1.0 min at 10,000 g to elute the RNA. The elution, containing the RNA, was collected and stored on ice until the RNA concentrations could be determined by UV absorbance using a NanoDrop Spectrophotometer (Thermo). RNA samples were stored at −80° C.

RNA Isolation: RNEASY® Plus Universal Mini Kit: RNA from all other Cyno, Mouse, and Rat tissue samples was isolated using RNEASY® Plus Universal Mini Kit (Qiagen cat #73404). For homogenization, 50 μg or less of tissue sample was transferred to a 2.0 mL round-bottom Eppendorf Safe-Lock tube (Eppendorf cat #022600044) containing 900 μL QIAZOL® Lysis Reagent, and a 5 mm stainless steel Bead (Qiagen cat #69989) Samples were homogenized, using a Qiagen's TissueLyser II instrument. Samples were processed for 2.0 min at 20 Hz, samples rotated 180° and processed for another 2.0 min at 20 Hz. Samples were then processed 2.0 min at 30 Hz, samples rotated 180° and processed for another 2.0 min at 30 Hz. Longer and/or at higher frequency homogenization used if processing not complete. Homogenized tissue lysate was then transferred into a new 2.0 mL round-bottom Eppendorf Safe-Lock tube and left at RT for 5.0 min. 100 μL of gDNA Eliminator Solution was added to each tube and tubes were vigorously shaken for 30 secs. 180 μL of Chloroform (Sigma cat #496189) was added to each tube and tubes were vigorously shaken for 30 secs. Tubes were left at RT for 3 min. Centrifuge tubes at 12,000 g for 15 min at 4° C. After centrifugation the upper aqueous phase was transferred to a new 2.0 mL round-bottom Eppendorf Safe-Lock tube ˜500 μL. An equal volume of 70% ethanol added and mixed. All future centrifugation steps were performed at RT. 500 μL was transferred to RNeasy spin column placed in a 2.0 mL collection tube and samples centrifuged for 15 secs at 10,000 g. The flow-through was discarded and the remaining 500 μL sample added to the spin column. The spin columns were centrifuged and the flow-through discarded and the columns washed with 700 μL of Wash Buffer RWT containing 2 volumes of ethanol. Columns were centrifuged for 15 secs at 10,000 g, the flow-through discarded. The columns were then washed twice with 500 μL of Buffer RPE containing 4-volumes of ethanol as described in kit protocol. Columns were first centrifuged for 15 secs at 10,000 g for first wash and then for 2.0 min at 10,000 g for the second wash. After second wash, columns were centrifuged once for 1.0 min at 10,000 g to dry the membranes. Columns were then transferred to a new 1.5 mL collection tube and 30 μL of RNase-free water added directly to the center of the membrane. Membranes were allowed to incubate for 10 min at RT. Columns were centrifuged for 1.0 min at 10,000 g to elute the RNA. The elutions, containing the RNA, were collected and stored on ice until RNA concentration determined by UV absorbance using a NanoDrop Spectrophotometer (Thermo). RNA samples were stored at −80° C.

cDNA Synthesis by Reverse Transcription: 300 ng of RNA was diluted to a final volume of 10.8 μL using nuclease-free water (Invitrogen cat #10977-015) in a PCR-96-AB-C microplate (Axygen cat #321-65-051). Added 6.0 μL to each well of reaction mix 1 containing the following: 2.0 μL of 50 μM random decamers (Ambion cat #AM5722G) and 4.0 μL of a 1×dNTP mix (Invitrogen cat #10297-018). The plate was sealed with optical sealing tape (Applied Biosystems cat #4360954) and centrifuged for 1.0 min at 1,000×g at RT. Next, the plate was heated for 3.0 min at 70° C. using a 96-well Thermal Cycler GeneAmp PCR System 9700 (Applied Biosystems). The plate was then cooled completely on ice. Next, 3.25 μL of the reaction mix 2 (containing 2 μL of 10× strand buffer, 1.0 μL of MMLV-RT 200 U/μL reverse transcriptase enzyme (Ambion cat #2044), and 0.25 μL of RNase inhibitor 40 U/μL (Ambion cat #AM2682)) were added to each of the wells. Plate was sealed with optical sealing tape and centrifuged for 1.0 min at 1,000×g at RT. Using a 96-well Thermal Cycler, the plate was heated at 42° C. for 60 min proceeded by 95° C. for 10 min. Then, the plates were cooled on ice. The cDNA plates were stored at −20° C. until ready to use for PCR analysis.

qPCR for amplification and quantification of SNCA and GAPDH mRNA expression: cDNA was diluted 5-fold in nuclease free water in a PCR-96-AB-C microplate. 16 μL of Master Mix solution consisting of the following: 10 μL of 2× Taqman Gene Expression Master Mix (Applied Biosystems cat #4369016), 1.0 μL of 20× Taqman primer-probe set (Applied Biosystems), and 5.0 μL of nuclease-free water, was added to each well of a 384-well optical PCR plate (Applied Biosystems cat #4483315). 4.0 μL of diluted cDNA was added to each well of the 384-well optical PCR plate. Plate was sealed with optical sealing tape and centrifuged for 1.0 min at 1,000×g at RT. PCR was performed on the Applied Biosystems 700 HT Fast Real-Time PCR System using the following parameters in standard mode: 50° C. for 2.0 min, 95° C. for 10 min, followed by 40 cycles of 95° C. for 15 secs and 60° C. for 1.0 min.

qRT-PCR primer-probe sets: Primer-probes sets from Applied Biosystems (Thermo Fisher) included the following:
Human alpha synuclein (cat #Hs01103383_ml) FAM labelled
Human PROS1 (cat #HS00165590_ml) FAM labelled
Cyno alpha synuclein (cat #Mf02793033_ml) FAM labelled
Cyno GAPDH (cat #Mf04392546_g1) FAM labelled
Cyno GAPDH (cat #Mf04392546_g1) VIC labelled Primer Limited
Rat alpha synuclein (cat #Rn01425141_ml) FAM labelled
Rat GAPDH (cat #Rn01775763-g1) FAM labelled
Rat GAPDH (cat #4352338E) VIC labelled Primer Limited
Mouse GAPDH (cat #Mm99999915-g1) FAM labelled
Mouse GAPDH (cat #4352339E) VIC labelled Primer Limited.

### Example 5: Analysis of In Vivo Activity and Tolerability of ASO-005459 in Mice

ASO-005459 is a LNA-modified ASO specific for human SNCA. The in vitro results (described above) demonstrate that ASO-005459 is potent and selective for reducing SNCA mRNA in primary neurons. The in vitro results also suggest that that ASO-005459 is well tolerated.

### A53T-PAC Mice

To evaluate whether these results are also true in vivo, 100 μg of ASO-005459 was administered to A53T-PAC mice via ICV injection, and both the tolerability and the SNCA mRNA knockdown (KD) in the hippocampus were assessed at 3 days post injection, as described in Example 4 (above).

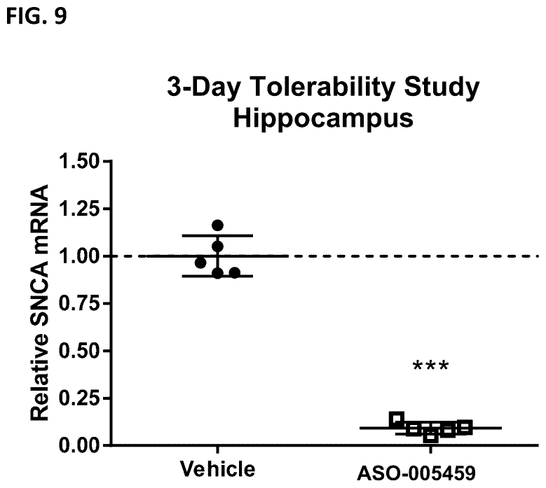

As shown in Table 6 (below), ASO-005459 was well tolerated with an overall mean tolerability score of 1. In addition, ASO-005459 significantly reduced SNCA mRNA levels by >90% in the hippocampus at 3 days post administration. (FIG. 9).

TABLE 6
ASO-005459 tolerability in A53T-PAC mice, 3-day study

| Animal | Hyper- | | Motor | Posture/ | Tremor/ | Total |
|---|---|---|---|---|---|---|
| # | activity | Vigilance | S&C | Breathing | Convulsions | Score |
| 31 | 0 | 2 | 1 | 2 | 0 | 5 |
| 32 | 0 | 0 | 0 | 0 | 0 | 0 |
| 33 | 0 | 0 | 0 | 0 | 0 | 0 |
| 34 | 0 | 0 | 0 | 0 | 0 | 0 |
| 35 | 0 | 0 | 0 | 0 | 0 | 0 |
| mean | | | | | | 1.00 |
| SEM | | | | | | 1.00 |

To evaluate activity in vivo, A53T-PAC mice were dosed with ASO-005459 at 3.13 μg, 12.5 μg, 25 μg, or 50 μg concentration via ICV injection. Tolerability was assessed by measuring body weights at days 7 and 14 post dosing. The expression of SNCA mRNA was assessed at 14 days post dosing, when the animals were sacrificed and their tissues harvested. SNCA mRNA knockdown was measured in three brain regions: Hippocampus, brainstem, and striatum. Brainstem and striatum are two of the regions that are most affected in the brains of MSA and PD patients. In a separate study, C57BL/6 mice were dosed with 100 μg of ASO-005459 and tolerability was assessed by measuring the body weight of the animals at days 7, 14, 21, and 28 post dosing. 100 μg ASO-005459 was dosed ICV in wild-type (WT) C57BL/6 mice, and the body weight and behavior were monitored over a 4-week period. Because ASO-005459 does not target mouse SNCA, reduction in SNCA mRNA expression was not measured in these animals.

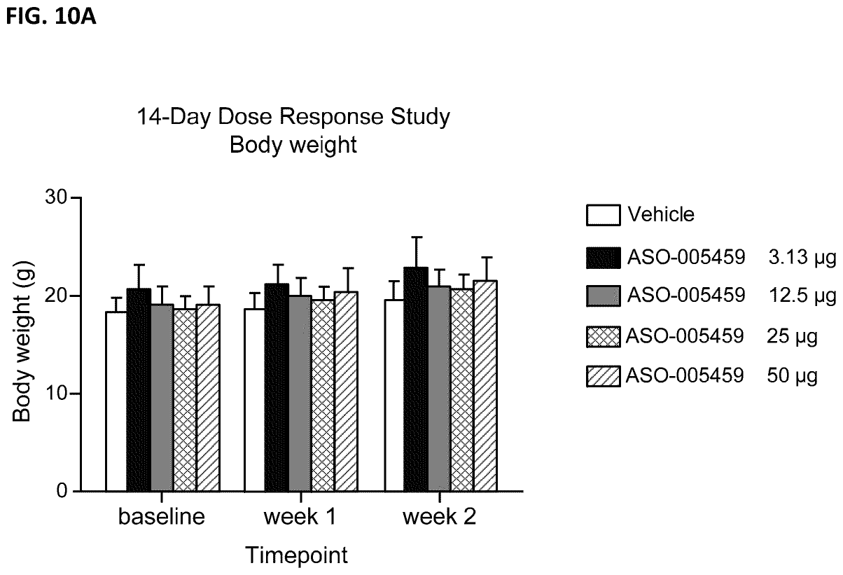

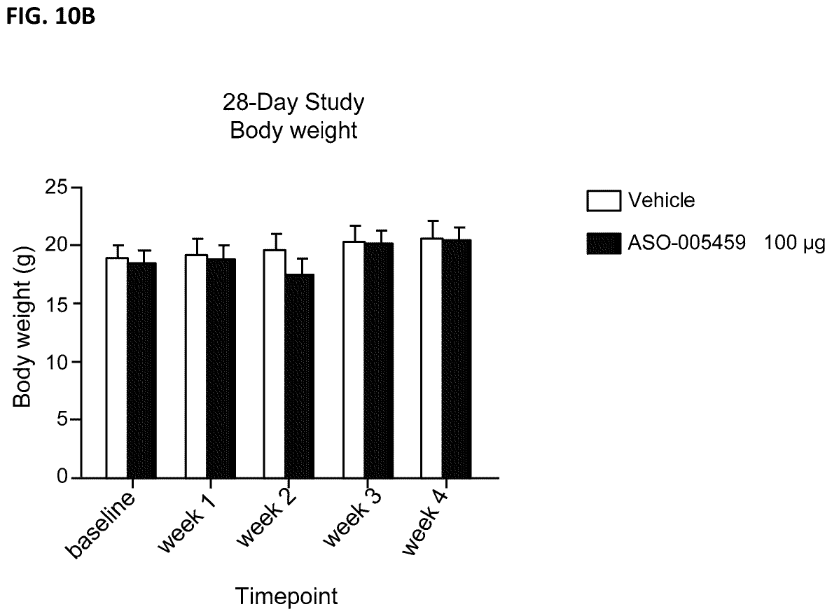

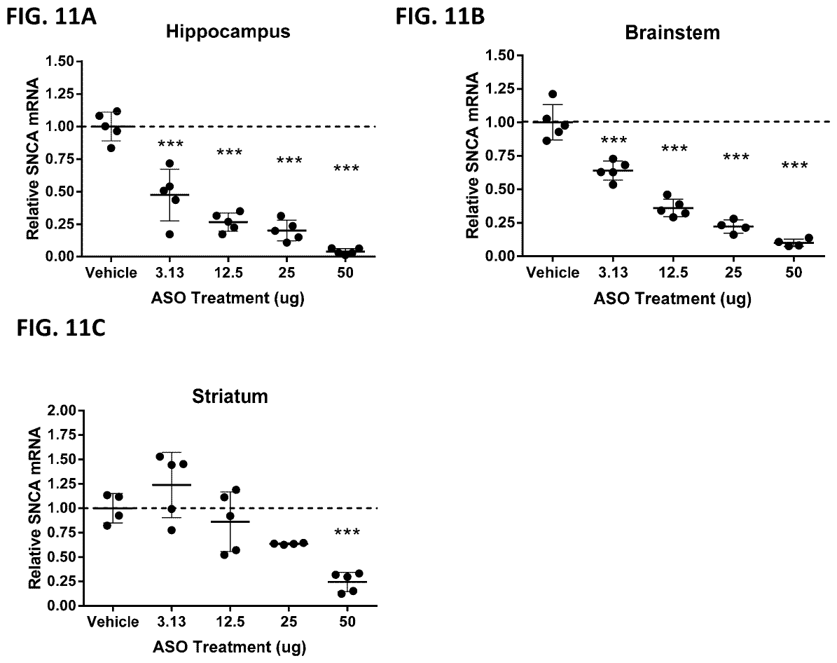

As shown in FIGS. 10A and 10B, there were no significant differences in the body weight of the mice (both A53T-PAC mice and C57BL/6 mice) treated with ASO-005459 (for all concentrations) and those treated with the control vehicle. The animals (C57BL/6) did not exhibit any abnormal behaviors during the course of the experiment. See Table 7 (below). Such results demonstrate that ASO-005459 was well tolerated. Also, in mice treated with ASO-005459, there was a significant and dose-dependent reduction in SNCA mRNA expression in all three brain regions tested. See FIGS. 11A-11C. In the hippocampus, the SNCA mRNA expression was reduced by 53%, 73%, 80%, and 96% for 3.13, 12.5, 25, and 50 μg ASO-005459, respectively. Similar dose-dependent knockdowns were also observed in the brainstem. In the striatum, SNCA mRNA knockdowns were more variable and less robust compared to the other regions (FIGS. 11A-11C): a 75% and 46% knockdown was observed with 50 μg and 25 μg of ASO-005459, respectively. However, with 12.5 μg and 3.13 μg of ASO-005459, there was no significant reduction in SNCA mRNA expression. Possible explanations for the lower activity observed in the striatum could be due to differences in the ASO levels or the kinetics of SNCA mRNA knockdown.

TABLE 7
ASO-005459 tolerability in 28-day study

| | Time- | | | | | Tremor/ | |
|---|---|---|---|---|---|---|---|
| Animal | point | Hyper- | | Motor | Posture/ | Convul- | Total |
| # | (day) | activity | Vigilance | S&C | Breathing | sions | Score |
| 26 | 1 | 0 | 0 | 0 | 0 | 0 | 0 |
| 26 | 28 | 0 | 0 | 0 | 0 | 0 | 0 |
| 27 | 1 | 0 | 0 | 0 | 0 | 0 | 0 |
| 27 | 28 | 0 | 0 | 0 | 0 | 0 | 0 |
| 28 | 1 | 0 | 0 | 0 | 0 | 0 | 0 |
| 28 | 28 | 0 | 0 | 0 | 0 | 0 | 0 |
| 29 | 1 | 0 | 0 | 0 | 0 | 0 | 0 |
| 29 | 28 | 0 | 0 | 0 | 0 | 0 | 0 |
| 30 | 1 | 0 | 0 | 0 | 0 | 0 | 0 |
| 30 | 28 | 0 | 0 | 0 | 0 | 0 | 0 |

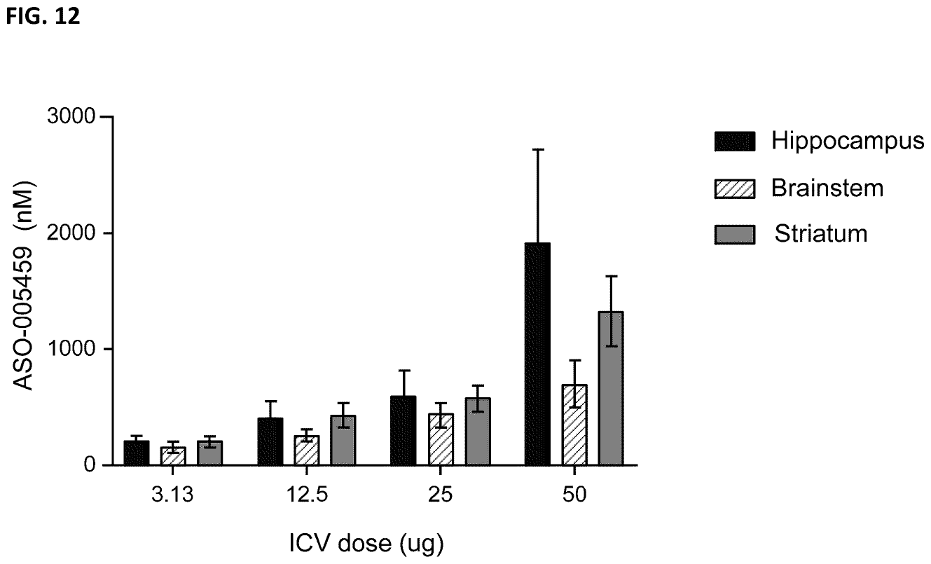

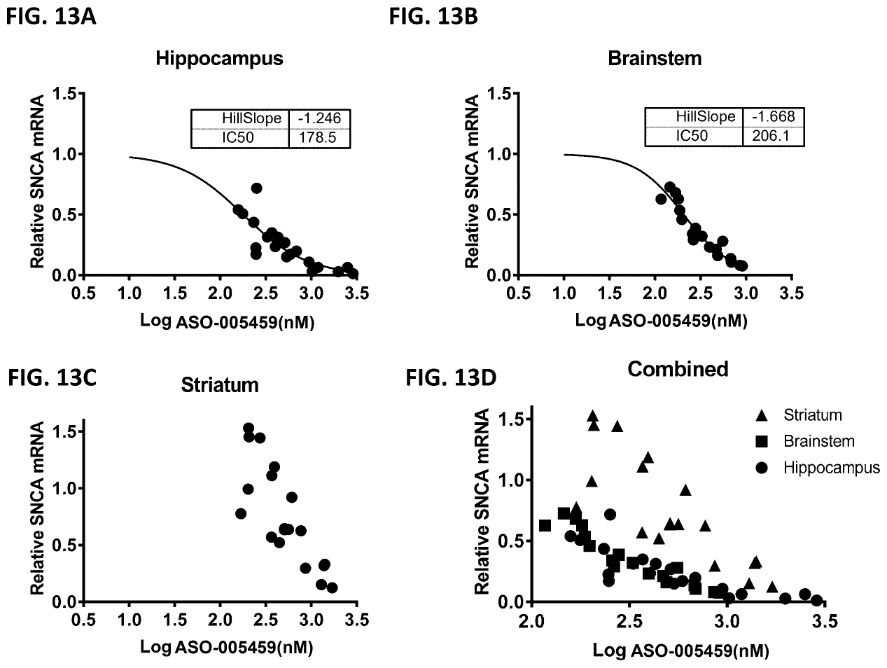

The ASO-005459 levels were measured in all three brain regions of the A53T-PAC mice. As shown in FIG. 12 and Table 8 (below), brain exposures were approximately dose proportional and similar across all three regions, including the striatum. The ASO-005459 exposure-response relationships for the different brain regions are shown in FIGS. 13A-13D. Similar exposure-response relationships were observed in the hippocampus and brainstem with estimated IC₅₀ s of 179 and 206 nM, respectively. In comparison, the exposure response relationship in the striatum was relatively steep suggesting slower kinetics of SNCA mRNA reduction in that region. See FIG. 13C.

TABLE 8
Summary of ASO-005459 brain exposure in
14-day A53T-PAC study

| | Dose | | | Relative |
|---|---|---|---|---|
| | (μg) | Mean (nM) | SD | to hippo |
| Hippocampus | 3.13 | 214 | 43 | |
| | 12.5 | 411 | 140 | |
| | 25 | 600 | 223 | |
| | 50 | 1916 | 807 | |
| Brainstem | 3.13 | 160 | 47 | 0.7 |
| | 12.5 | 266 | 47 | 0.6 |
| | 25 | 440 | 102 | 0.7 |
| | 50 | 707 | 202 | 0.4 |
| Striatum | 3.13 | 212 | 38 | 1.0 |
| | 12.5 | 438 | 103 | 1.1 |
| | 25 | 586 | 109 | 1.0 |
| | 50 | 1332 | 302 | 0.7 |

To provide a more extensive dose-response/time course data, ASO-005459 was again administered (0, 12.5, 25, or 50 μg) directly into the cerebral ventricles of A53T-PAC mice by ICV freehand injection. The animals were sacrificed at 24 hours, 3 days, 4, 8, 12, 16, and 20 weeks post dosing and the SNCA mRNA expression in the brain stem and the striatum was assessed.

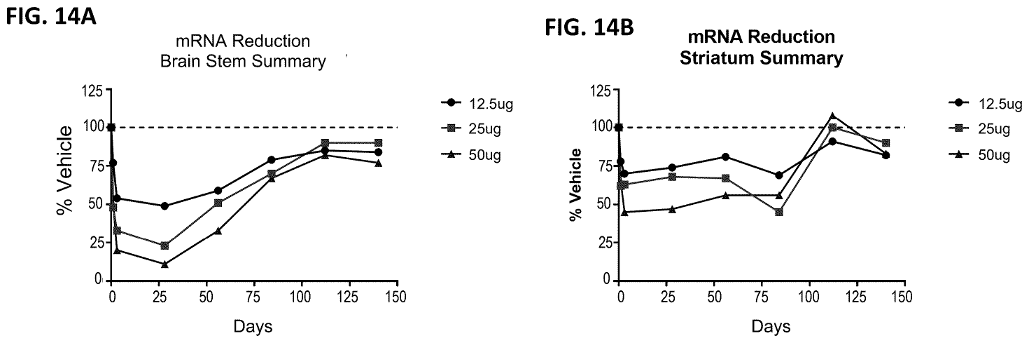

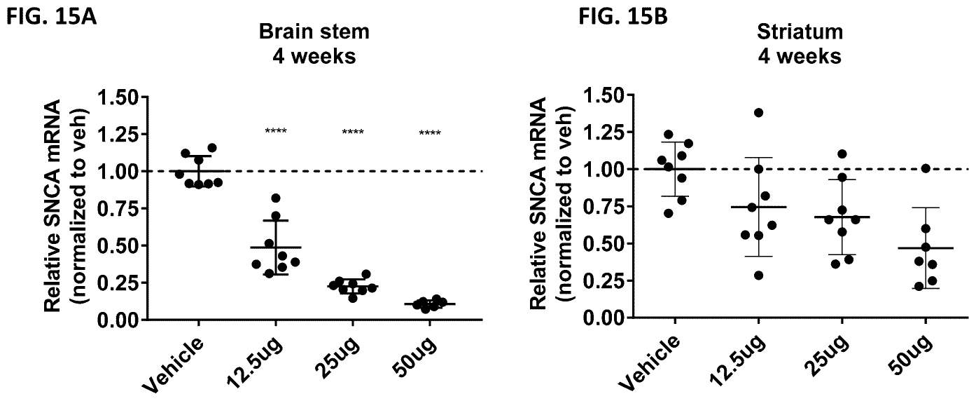

As shown in FIGS. 14A and 14B (and in agreement with the data provided above), administration of ASO-005459 to the A53T-PAC mice resulted in significant reduction of SNCA mRNA expression level (relative to the vehicle control) in both the brain stem and the striatum. The reduction appeared to be both time- and dose-dependent, with peak reduction (˜90%) observed in the brain stem with 50 μg ASO-005459 at about 4 weeks post dosing (FIG. 14A). In the striatum, peak reduction (˜55%) was observed with 50 μg ASO-005459 at about 3 days post dosing (FIG. 14B). SNCA mRNA expression levels remained significantly reduced compared to the vehicle control at 4 weeks post dosing (FIGS. 15A and 15B) and returned to baseline control by about 16 weeks post-dosing (FIGS. 14A and 14B).

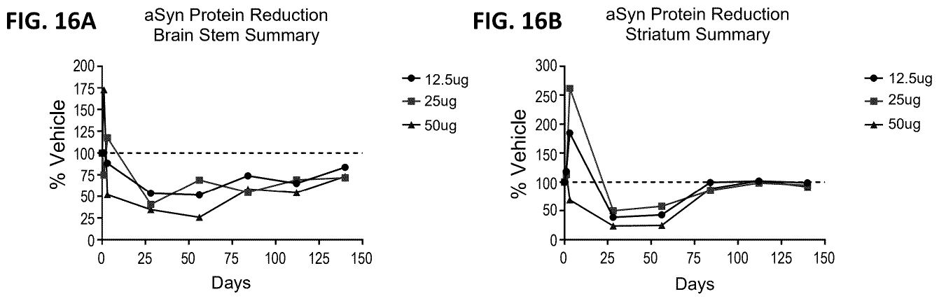

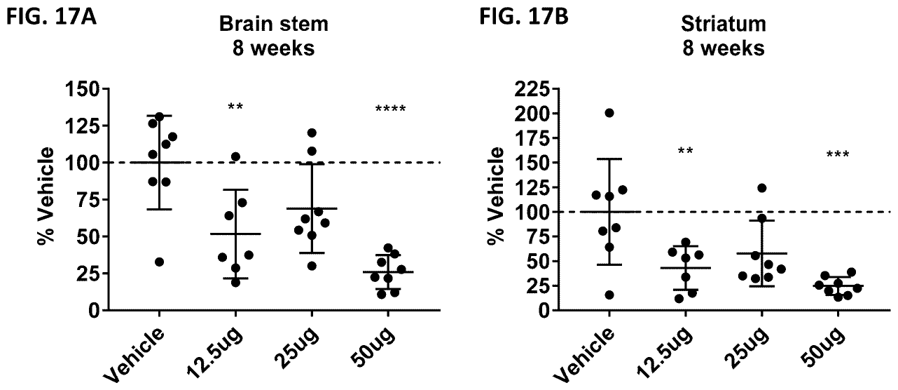

As shown in FIGS. 16A and 16B, administration of ASO-005459 to the animals also resulted in a time- and dose-dependent reduction in the SNCA protein level in both the brain stem and the striatum brain tissue. Peak reduction (˜75%) was observed in the brain stem with a dose of 50 μg at 8 weeks post dosing (FIG. 16A). For the striatum brain tissue, peak reduction (˜75%) was also observed with the 50 μg dose but at 4 weeks post dosing (FIG. 16B). Expression levels for the individual mice at 8 weeks post dosing are provided in FIGS. 17A (brain stem) and 17B (striatum). While SNCA protein level returned close to baseline by about 12 weeks post dosing in the striatum, expression level was still significantly reduced (˜25%) in the brain stem as far out as 16 weeks post-dosing.

### Example 7: Analysis of In Vivo Activity and Tolerability of SNCA-Targeted Antisense Oligonucleotides (ASOs) in Cynomolgus Monkeys

To further evaluate the ASO activity and tolerability in vivo, an intrathecal ported Cynomolgus monkey model (Cyno IT) was developed. This model enables the evaluation of ASO-005459-mediated knockdown of SNCA as well as knockdown of the potential 1-basepair mismatch off-targets PROS1 and IKZF3. The ASO-005459 target sites in SNCA, PROS1, and IKZF3 are completely conserved between human and cyno.

As described above in Example 3, each animal was implanted with an intrathecal cerebrospinal fluid (CSF) catheter entering at the L3 or L4 vertebrae. The ASOs (ASO-005459, ASO-005584, and ASO-005578) were dissolved in saline and administered to the animals, infused over 4.5 min using the IT port (2 animals per dose group). Each of the animals received one of the following: (i) ASO-005578 (4 mg total), (ii) ASO-005584 (4 or 8 mg total), and (iii) ASO-005459 (8 mg total). Animals were then euthanized at various time points post dosing, when the tissues were harvested for analysis of the ASO exposure and activity. Brain regions analyzed included medulla (Med), pons (V-Pons), midbrain (V-MB), cerebellum (CBL), caudate-putamen (left and right) (CauP), hippocampus (left and right) (Hip), frontal cortex (left and right) (FrC), temporal cortex (left and right) (TeC), parietal cortex (left and right) (PaC), occipital cortex (left and right) (Occ), and cortical white matter (WM). Additionally, spinal cord was sampled at the cervical (CSC), thoracic (TSC), and lumbar (LSC) regions. Samples were also collected from liver, kidney, heart, trigeminal nuclei, tibial nerve, and aorta to examine off-target pharmacology in those areas.

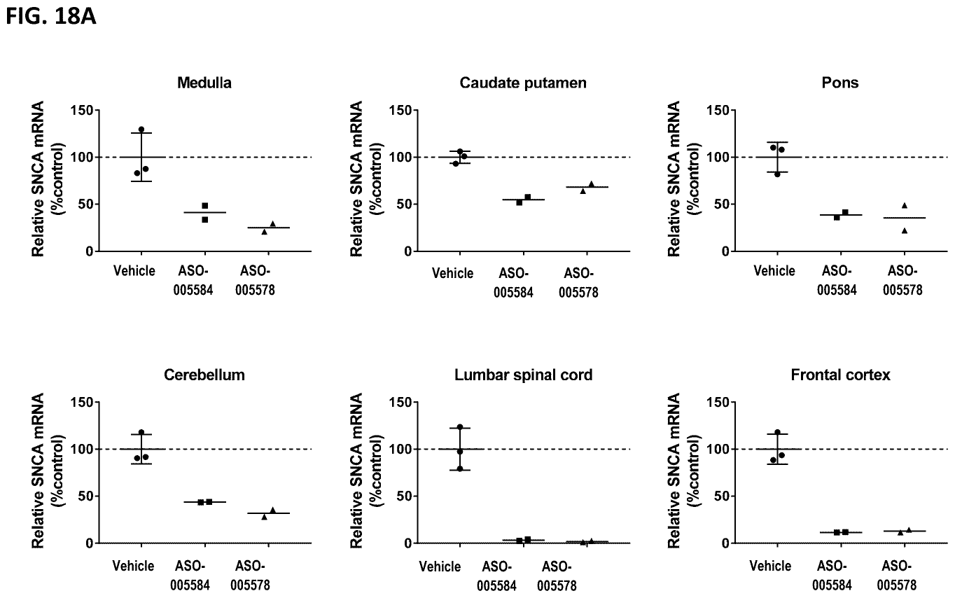

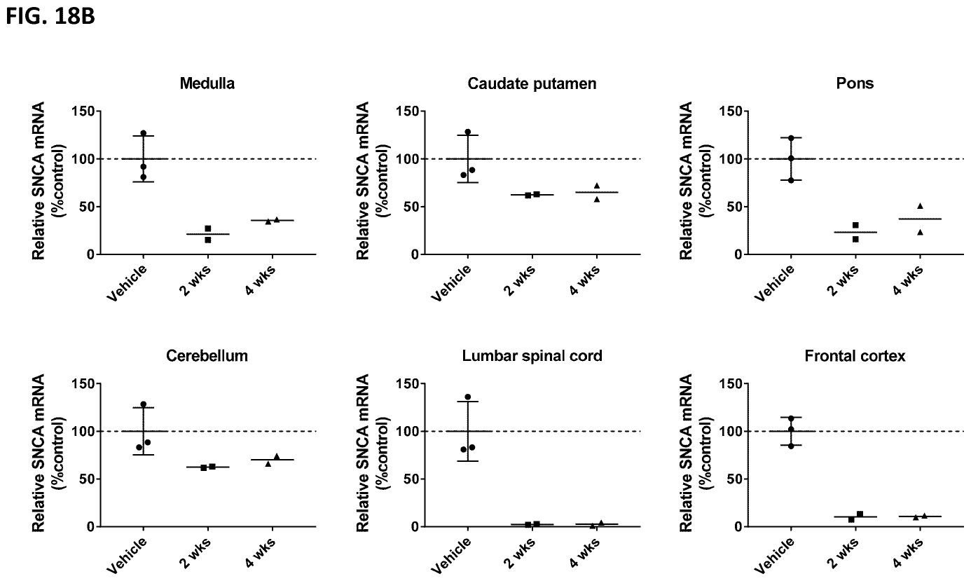

As was observed in mice, the ASOs were well tolerated in cyno with no adverse effects being observed (data not shown). And as shown in FIG. 18A and Table 9 below, the administration of ASO-005578 or ASO-005584 to the animals resulted in significant reduction of SNCA mRNA levels in all brain tissues analyzed. At 2 weeks post dosing, ASO-005578 induced reductions of 75%, 32%, 65%, 69%, 98% and 87% in the medulla, caudate-putamen, pons, cerebellum, lumbar spinal cord and frontal cortex, respectively. ASO-005584 induced reductions of 59%, 45%, 61%, 57%, 97% and 88% in the medulla, caudate-putamen, pons, cerebellum, lumbar spinal cord and frontal cortex, respectively. Similar reductions were observed at the 2-week and 4-week post-dose timepoints following administration of 8 mg ASO-005584 (FIG. 18B). ASO-005459 also exhibited robust knockdown of SNCA mRNA in various regions of the cyno brain (Table 9).

TABLE 9
Effect of ASOs on brain SNCA mRNA levels in cyno brain

| ASO | Dose | Timepoint | | | | | | | | | | | | | | |
|---|---|---|---|---|---|---|---|---|---|---|---|---|---|---|---|---|
| No. | (mg) | (weeks) | Med | CBL | FrC | PaC | CauP | TeC | Occ | Hip | V-MB | V-Pons | CSC | TSC | LSC | WM |
| ASO-005578 | 4 | 2 | 25 | 32 | 13 | 27 | 68 | 24 | 38 | 45 | 27 | 35 | 15 | 7 | 2 | 64 |
| ASO-005584 | 4 | 2 | 41 | 44 | 12 | 34 | 55 | 8 | 38 | 44 | 53 | 39 | 16 | 9 | 3 | 45 |
| | 8 | 2 | 21 | 62 | 10 | 16 | 62 | 6 | 19 | 36 | 34 | 23 | 2 | 2 | 2 | 38 |
| | 8 | 4 | 36 | 70 | 11 | 23 | 65 | 7 | 28 | 48 | 48 | 37 | 5 | 4 | 3 | 78 |
| ASO-005459 | 8 | 24 hours | 174 | 140 | 146 | | 149 | | | | | 137 | | | 73 | |
| | 8 | 24 hours | 129 | 108 | 138 | | 133 | | | | | 145 | | | 90 | |
| | 8 | 3 days | 162 | 69 | 69 | | 112 | | | | | 62 | | | 19 | |
| | 8 | 3 days | 127 | 67 | 72 | | 114 | | | | | 87 | | | 31 | |
| | 2 | 2 | 133 | 140 | 109 | | 161 | | | | | 104 | | | 21 | |
| | 2 | 2 | 117 | 127 | 100 | | 126 | | | | | 180 | | | 111 | |
| | 4 | 2 | 62 | 86 | 22 | | 116 | | | | | 49 | | | 4 | |
| | 4 | 2 | 81 | 72 | 50 | | 145 | | | | | 97 | | | 13 | |
| | 8 | 2 | 31 | 97 | 7 | | 124 | | | | | 66 | | | 14 | |
| | 8 | 2 | 30 | 38 | 15 | 26 | 75 | 12 | 29 | 29 | 86 | 29 | 13 | 6 | 2 | 79 |
| | 8 | 4 | 26 | 50 | 4 | 12 | 53 | 3 | 13 | 9 | 24 | 21 | 4 | 6 | 4 | 87 |
| | 8 | 4 | 38 | 61 | 9 | | 120 | | | | | 30 | | | 10 | |
| | 8 | 8 | 121 | 85 | 40 | | 98 | | | | | 78 | | | 5 | |
| | 8 | 8 | 93 | 52 | 14 | | 61 | | | | | 36 | | | 11 | |
| | 8 | 13 | 30 | 63 | 8 | | 81 | | | | | 27 | | | 1 | |
| | 8 | 13 | 25 | 28 | 8 | | 49 | | | | | 22 | | | 1 | |
| | 8 | 20 | 40 | 38 | 35 | | 76 | | | | | 63 | | | 9 | |
| | 8 | 20 | 22 | 52 | 35 | | 76 | | | | | 26 | | | 11 | |

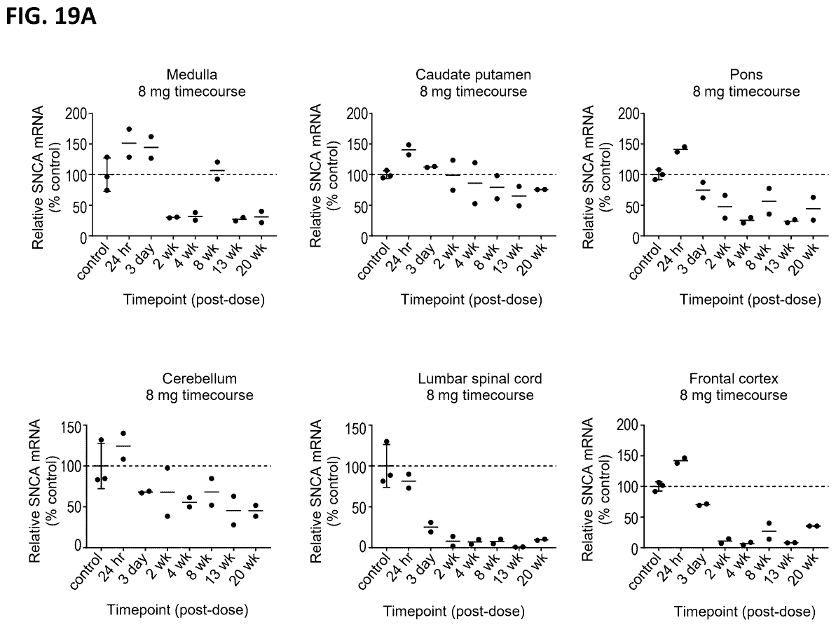

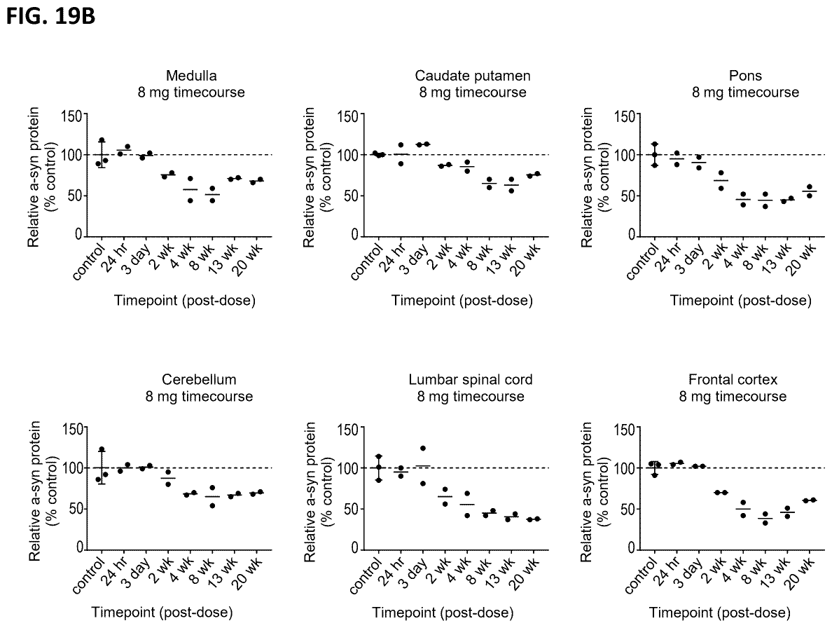

To further characterize the time-dependent reduction of SNCA mRNA described-above, cyno monkeys were dosed with ASO-005459 (8 mg total per animal) and sacrificed at 24 hour, 3 days, 2, 4, 8, 13, or 20 weeks post-dosing to assess the SNCA mRNA expression level in the different tissues. As shown in FIG. 19A, peak reduction was observed between 2 weeks and 13 weeks post-dosing. Peak reductions of 700%, 65%, 7500, 3500, 94% and 99% were observed in the medulla, cerebellum, pons, caudate-putamen, frontal cortex and lumbar spinal cord, respectively. This reduction in SNCA mRNA expression level also correlated with a time-dependent reduction in the SNCA protein expression level (FIG. 19B).

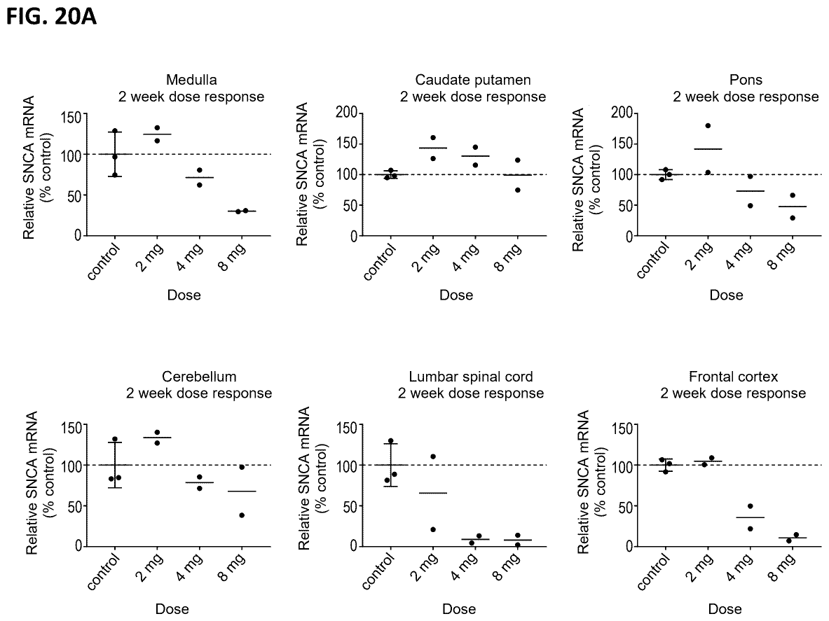

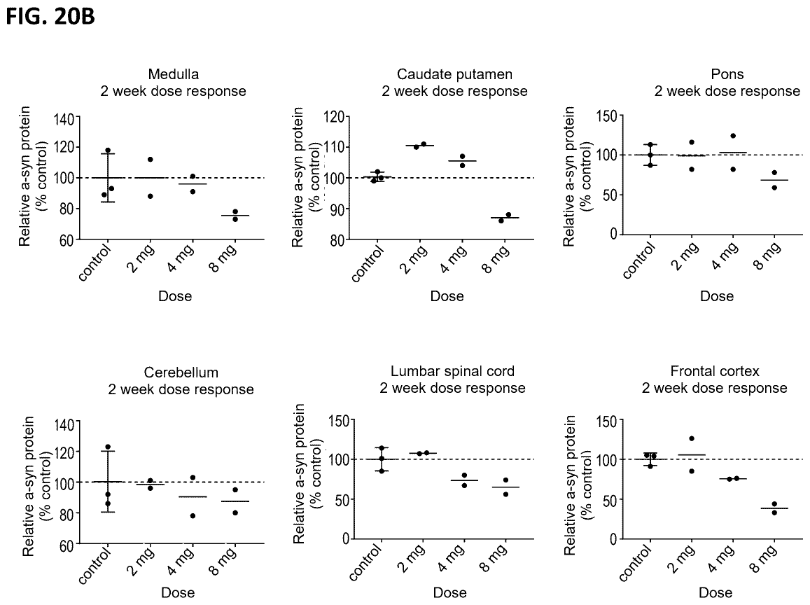

Next, to further assess whether the reduction in expression level in the cyno monkeys was also dependent on the dose, the animals received 2, 4, or 8 mg of ASO-005459 and then, were sacrificed at 2 weeks post-dosing. As shown in FIGS. 20A and 20B, the reduction in both the SNCA mRNA and the SNCA protein expression levels was also dependent on the dose, with the greatest reduction observed with 8 mg of ASO-005459.

The results presented here demonstrate that ASO-005459 is potent and selective for reducing SNCA mRNA and that ASO-005459 is well tolerated in neurons and studies in preclinical species in vivo. Moreover, results from the A53T-PAC neurons confirm that ASO-005459-mediated reductions of mRNA result in reductions of SNCA protein levels in vitro and in vivo. In addition, results in A53T-PAC mice and cyno monkeys demonstrate that ASO-005459 reduces SNCA mRNA and SNCA protein in brain at doses that are well tolerated. Taken together, these findings support the continued development of ASO-005459 as a disease-modifying therapeutic for the treatment of synucleinopathies.

This PCT application claims priority benefit of U.S. Provisional Application No. 62/616,994, filed Jan. 12, 2018, which is incorporated herein by reference in its entirety.

What is claimed:

1. An antisense oligonucleotide comprising a contiguous nucleotide sequence of 10 to 30 nucleotides in length that are complementary to a nucleic acid sequence within an alpha-synuclein (SNCA) transcript, wherein the nucleic acid sequence comprises nucleotides 7592-7637 of SEQ ID NO: 1.

2. The antisense oligonucleotide of claim 1, wherein the nucleic acid sequence comprises nucleotides 7602-7627 of SEQ ID NO: 1.

3. The antisense oligonucleotide of claim 1, wherein the nucleic acid sequence corresponds to nucleotides 7603-7620 of SEQ ID NO: 1.

4. The antisense oligonucleotide of claim 1, wherein the nucleic acid sequence corresponds to nucleotides 7604-7620 of SEQ ID NO: 1.

5. The antisense oligonucleotide of any one of claims 1 to 4, wherein the SNCA transcript comprises SEQ ID NO: 1.

6. The antisense oligonucleotide of any one of claims 1 to 5, wherein the contiguous nucleotide sequence comprises SEQ ID NO: 4 to SEQ ID NO: 21 with one, two, three, or four mismatches.

7. The antisense oligonucleotide of any one of claims 1 to 5, wherein the contiguous nucleotide sequence comprises SEQ ID NO: 4 to SEQ ID NO: 21.

8. The antisense oligonucleotide of any one of claims 1 to 5, wherein the contiguous nucleotide sequence comprises SEQ ID NO: 15 or SEQ ID NO: 7.

9. The antisense oligonucleotide of any one of claims 1 to 8, wherein the antisense oligonucleotide is capable of inhibiting the expression of the human SNCA transcript in a cell which is expressing the human SNCA transcript.

10. The antisense oligonucleotide of any of claims 1 to 9, wherein the contiguous nucleotide sequence comprises at least one nucleotide analogue.

11. The antisense oligonucleotide of any one of claims 1 to 10, which is a gapmer.

12. The antisense oligonucleotide of claim 11, which is an alternating flank gapmer.

13. The antisense oligonucleotide of claim 11 or 12, which comprises the formula of 5′-A-B-C-3′, wherein
(i) region B is a contiguous sequence of at least 6 DNA units, which are capable of recruiting RNase;
(ii) region A is a first wing sequence of 1 to 10 nucleotides, wherein the first wing sequence comprises one or more nucleotide analogues and optionally one or more DNA units and wherein at least one of the nucleotide analogues is located at the 3′ end of A; and
(iii) region C is a second wing sequence of 1 to 10 nucleotides, wherein the second wing sequence comprises one or more nucleotide analogues and optionally one or more DNA units and wherein at least one of the nucleotide analogues is located at the 5′ end of C.

14. The antisense oligonucleotide of claim 13, wherein region A comprises a combination of nucleotide analogues and DNA unit selected from (i) 1-10 nucleotide analogs and 0 DNA unit; (ii) 1-9 nucleotide analogues and 1 DNA unit; (iii) 1-8 nucleotide analogues and 1-2 DNA units; (iv) 1-7 nucleotide analogues and 1-3 DNA units; (v) 1-6 nucleotide analogues and 1-4 DNA units; (vi) 1-5 nucleotide analogues and 1-5 DNA units; (vii) 1-4 nucleotide analogues and 1-6 DNA units; (viii) 1-3 nucleotide analogues and 1-7 DNA units; (ix) 1-2 nucleotide analogues and 1-8 DNA units; and (x) 1 nucleotide analogue and 1-9 DNA units.

15. The antisense oligonucleotide of claim 13 or 14, wherein region C comprises a combination of nucleotide analogues and DNA unit selected from (i) 1-10 nucleotide analogs and 0 DNA unit; (ii) 1-9 nucleotide analogues and 1 DNA unit; (iii) 1-8 nucleotide analogues and 1-2 DNA units; (iv) 1-7 nucleotide analogues and 1-3 DNA units; (v) 1-6 nucleotide analogues and 1-4 DNA units; (vi) 1-5 nucleotide analogues and 1-5 DNA units; (vii) 1-4 nucleotide analogues and 1-6 DNA units; (viii) 1-3 nucleotide analogues and 1-7 DNA units; (ix) 1-2 nucleotide analogues and 1-8 DNA units; and (x) 1 nucleotide analogue and 1-9 DNA units.

16. The antisense oligonucleotide of any one of claims 13 to 15, wherein region A is a first wing design selected from any ASOs in FIGS. 2 and 3 and/or region C is a second wing design selected from any ASOs in FIGS. 2 and 3, wherein the upper letter is a nucleotide analog and the lower letter is a DNA.

17. The antisense oligonucleotide of any one of claims 1 to 16, which comprises at least two, at least three, at least four, at least five, at least six, at least seven, at least eight, at least nine, or at least ten nucleotide analogues.

18. The antisense oligonucleotide of any one of claims 10 to 17, wherein the nucleotide analogue or analogues are one or more sugar modified nucleosides selected from the group consisting of Locked Nucleic Acid (LNA); 2′-O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleoside (cEt), 2′-O-methyl nucleic acid (2′-OMe), 2′-O-methoxyethyl nucleic acid (2′-MOE), and any combination thereof.

19. The antisense oligonucleotide of any one of claims 10 to 17, wherein the nucleotide analogue or analogues comprise a bicyclic sugar.

20. The antisense oligonucleotide of claim 19, wherein the bicyclic sugar comprises cEt, 2′,4′-constrained 2′-O-methoxyethyl (cMOE), LNA, α-L-LNA, β-D-LNA, 2′-O,4′-C-ethylene-bridged nucleic acids (ENA), amino-LNA, oxy-LNA, or thio-LNA.

21. The antisense oligonucleotide of any one of claims 10 to 20, wherein the nucleotide analogue or analogues comprise an LNA.

22. The antisense oligonucleotide of any one of claims 10 to 21, wherein the antisense oligonucleotide comprises one or more 5′ methyl cytosine nucleobases.

23. The antisense oligonucleotide of claim 21, which comprises two to five LNAs on the 5′ region of the antisense oligonucleotide.

24. The antisense oligonucleotide of claim 21, which comprises two to five LNAs on the 3′ region of the antisense oligonucleotide.

25. The antisense oligonucleotide of any one of claims 1 to 24, wherein the antisense oligonucleotide has an in vivo tolerability less than or equal to a total score of 4, wherein the total score is the sum of a unit score of five categories, which are 1) hyperactivity; 2) decreased activity and arousal; 3) motor dysfunction and/or ataxia; 4) abnormal posture and breathing; and 5) tremor and/or convulsions, and wherein the unit score for each category is measured on a scale of 0-4.

26. The antisense oligonucleotide of claim 25, wherein the in vivo tolerability is less than or equal to the total score of 3, the total score of 2, the total score of 1, or the total score of 0.

27. The antisense oligonucleotide of any one of claims 1 to 26, which reduces expression of SNCA mRNA in a cell by at least about 20%, at least about 30%, at least about 40%, at least about 50%, at least about 60%, at least about 70%, at least about 80%, at least about 90%, or about 100% compared to a cell not exposed to the antisense oligonucleotide.

28. The antisense oligonucleotide of any one of claims 1 to 27, which reduces expression of SNCA protein in a cell by at least about 60%, at least about 70%, at least about 80%, at least about 90%, or at least about 95% compared to a cell not exposed to the antisense oligonucleotide.

29. The antisense oligonucleotide of any one claims 1 to 28, which comprises the nucleotides A, T, C, and G and at least one analogue of the nucleotides A, T, C, and G, and has a sequence score greater than or equal to 0.2, wherein the sequence score is calculated by formula I:
# of C nucleotides and analogues thereof—# of G nucleotides and analogues thereof (I)
Total nucleotide length.

30. The antisense oligonucleotide of any one of claims 1 to 29, which has from 10 to 24 nucleotides in length or from 14 to 21 nucleotides in length.

31. The antisense oligonucleotide of any one of claims 1 to 30, which has 14, 15, 16, 17, 18, 19, 20, or 21 nucleotides in length.

32. The antisense oligonucleotide of claim 1 to 31, wherein the nucleotide sequence comprises, consists essentially of, or consists of a sequence selected from the group consisting of SEQ ID NOs: 4 to 21 with a design selected from the group consisting of the designs in FIGS. 2 and 3, wherein the upper case letter is a sugar modified nucleoside and the lower case letter is DNA.

33. The antisense oligonucleotide of claim 32, wherein the nucleotide sequence comprises, consists essentially of, or consists of SEQ ID NO: 15 with the design of ASO-005459 and SEQ ID NO: 7 with the design of ASO-005578 or ASO-005584.

34. The antisense oligonucleotide of any one of claims 1 to 31, wherein the nucleotide sequence comprises, consists essentially of, or consists of a sequence selected from the group consisting of SEQ ID NOs: 4-21 with the corresponding chemical structure in FIGS. 2 and 3.

35. The antisense oligonucleotide of claim 34, wherein the nucleotide sequence comprises, consists essentially of, or consists of ASO-005459, ASO-005578, and ASO-005584.

36. The antisense oligonucleotide of claim 34, wherein the ASO comprises a molecular formula of C₁₇₁H₂₁₄N₅₆O₉₀P₁₆S₁₆.

37. The antisense oligonucleotide of claim 34, wherein the ASO comprises a molecular formula of C₁₈₂H₂₂₉N₅₈O₉₆P₁₇S₁₇.

38. The antisense oligonucleotide of claim 34, wherein the ASO comprises a molecular formula of C₁₈₁H₂₂₇N₅₈O₉₆P₁₇S₁₇.

39. The antisense oligonucleotide of any one of claims 1 to 35, which comprises an internucleoside linkage selected from: a phosphodiester linkage, a phosphotriester linkage, a methylphosphonate linkage, a phosphoramidate linkage, a phosphorothioate linkage, and combinations thereof.

40. The antisense oligonucleotide of claim 39, wherein the internucleoside linkage comprises one or more stereo-defined, modified phosphate linkages.

41. A conjugate comprising the antisense oligonucleotide of any one of claims 1 to 40, wherein the antisense oligonucleotide is covalently attached to at least one non-nucleotide or non-polynucleotide moiety.

42. The conjugate of claim 41, wherein the non-nucleotide or non-polynucleotide moiety comprises a protein, a fatty acid chain, a sugar residue, a glycoprotein, a polymer, or any combinations thereof.

43. A pharmaceutical composition comprising the antisense oligonucleotide of any one claims 1 to 40 or the conjugate of claim 41 or 42 and a pharmaceutically acceptable carrier.

44. The composition of claim 43, which further comprises a therapeutic agent.

45. The composition of claim 44, wherein the therapeutic agent is an alpha-synuclein antagonist.

46. The composition of claim 45, wherein the alpha-synuclein antagonist is an anti-alpha-synuclein antibody or fragment thereof.

47. A kit comprising the antisense oligonucleotide of any one claims 1 to 40, the conjugate of claim 41 or 42, or the composition of any one of claims 43 to 46, and instructions for use.

48. A diagnostic kit comprising the antisense oligonucleotide of any one claims 1 to 40, the conjugate of claim 41 or 42, or the composition of any one of claims 43 to 46, and instructions for use.

49. A method of inhibiting or reducing SNCA protein expression in a cell, the method comprising administering the antisense oligonucleotide of any one claims 1 to 40, or the conjugate of claim 41 or 42, or the composition of any one of claims 43 to 46 to the cell expressing SNCA protein, wherein the SNCA protein expression in the cell is inhibited or reduced after the administration.

50. The method of claim 49, wherein the antisense oligonucleotide inhibits or reduces expression of SNCA mRNA in the cell after the administration.

51. The method of claim 49 or 50, wherein the expression of SNCA mRNA is reduced by at least about 20%, at least about 30%, at least about 40%, at least about 50%, at least about 60%, at least about 70%, at least about 80%, at least about 90%, or about 100% after the administration compared to a cell not exposed to the antisense oligonucleotide.

52. The method of any one of claims 49 to 51, wherein the antisense oligonucleotide reduces expression of SNCA protein in the cell after the administration by at least about 60%, at least about 70%, at least about 80%, or at least about 90% compared to a cell not exposed to the antisense oligonucleotide.

53. The method of any one of claims 49 to 52, wherein the cell is a neuron.

54. A method for treating a synucleinopathy in a subject in need thereof, comprising administering an effective amount of the antisense oligonucleotide of any one claims 1 to 40, the conjugate of claim 41 or 42, or the composition of any one of claims 43 to 46 to the subject.

55. Use of the antisense oligonucleotide of any one claims 1 to 40, the conjugate of claim 41 or 42, or the composition of any one of claims 43 to 46 for the manufacture of a medicament.

56. Use of the antisense oligonucleotide of any one claims 1 to 40, the conjugate of claim 41 or 42, or the composition of any one of claims 43 to 46 for the manufacture of a medicament for the treatment of a synucleinopathy in a subject in need thereof.

57. The antisense oligonucleotide of any one claims 1 to 40, the conjugate of claim 41 or 42, or the composition of any one of claims 43 to 46 for use in therapy.

58. The antisense oligonucleotide of any one claims 1 to 40, the conjugate of claim 41 or 42, or the composition of any one of claims 43 to 46 for use in therapy of a synucleinopathy in a subject in need thereof.

59. The method of claim 54, the use of claim 55 or 56, or the antisense oligonucleotide for use of claim 57 or 58, wherein the synucleinopathy is selected from the group consisting of Parkinson's disease, Parkinson's Disease Dementia (PDD), multiple system atrophy, dementia with Lewy bodies, and any combinations thereof.

60. The method of claim 54 or 59, use of any one of claims 55, 56, and 59, or antisense oligonucleotide for use of any one of claims 57 to 59, wherein the subject is a human.

61. The method of any one of claims 54, 59, and 60, the use of any one of claims 55, 56, 59, and 60, or the antisense oligonucleotide for use of any one of claims 57 to 60, wherein the antisense oligonucleotide, the conjugate, or the composition is administered orally, parenterally, intrathecally, intra-cerebroventricularly, pulmonarily, topically, or intraventricularly.

62. The antisense oligonucleotide of any one claims 10 to 40, the conjugate of claim 41 or 42, the composition of any one of claims 43 to 46, the kit of claim 47 or 48, the method of any one of claims 49 to 54 and 59 to 61, the use of any one of claims 55, 56, and 59 to 61, or the antisense oligonucleotide for use of any one of claims 57 to 61, wherein the nucleotide analog comprises a sugar modified nucleoside.

63. The antisense oligonucleotide, the conjugate, the composition, the kit, the method, the use, or the antisense oligonucleotide for use of claim 62, wherein the sugar modified nucleoside is an affinity enhancing sugar modified nucleoside.


SEQUENCE LISTING (27 sequences)

SEQ ID NO: 1 - DNA, 2400 bp, Homo sapiens
  source 1..2400: mol_type = genomic DNA; note = partial SNCA genomic sequence (i.e., residues 6001 to 8400)
gagatagggacgaggagcacgctgcagggaaagcagcgagcgccgggagaggggcgggcagaagcgctgacaaatcagcggtgggggcggagagccgaggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcaggtaagtacctgtggatctaaacgggcgtctttggaaatcctggagaacgccggatgggagacgaatggtcgtgggcaccgggagggggtggtgctgccatgaggacccgctgggccaggtctctgggaggtgagtacttgtccctttggggagcctaaggaaagagacttgacctggctttcgtcctgcttctgatattcccttctccacaagggctgagagattaggctgcttctccgggatccgcttttccccgggaaacgcgaggatgctccatggagcgtgagcatccaacttttctctcacataaaatctgtctgcccgctctcttggtttttctctgtaaagtaagcaagctgcgtttggcaaataatgaaatggaagtgcaaggaggccaagtcaacaggtggtaacgggttaacaagtgctggcgcggggtccgctagggtggaggctgagaacgccccctcgggtggctggcgcggggttggagacggcccgcgagtgtgagcggcgcctgctcagggtagatagctgagggcgggggtggatgttggatggattagaaccatcacacttgggcctgctgtttgcctgagtttgaaccacaccccgagtgagcagttagttctgttgcctacgcctttccaccatcaacctgttagccttcttctgggattcatgttaaggatacccctgaccctaagcctccagcttccatgcttctaactcatactgttaccctttagaccccgggaatttaaaaaaggggttaatcttttcatgcaactccacttctgaaatgcagtaataacaactcagaggattcatcctaatccgtggttaggtggctagacttttactagccaagatggatgggagatgctaaatttttaatgccagagctaaaaatgtctgctttgtccaatggttaaatgagtgtacacttaaaagagtctcacactttggagggtttctcatgatttttcagtgttttttgtttatttttccccgaaagttctcattcaaagtgtattttatgttttccagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggtaggtaaaccccaaatgtcagtttggtgcttgttcatgagtgatgggttaggataatcaatactctaaatgctggtagttctctctcttgattcatttttgcatcattgcttgtcaaaaaggtggactgagtcagaggtatgtgtaggtaggtgaatgtgaacgtgtgtatttgagctaatagtaaaaaatgcgactgtttgcttttccagatttttaattttgccctaatatttatgactttttaaaaatgaatgtttctgtacctacataattctatttcagagaacagttttaaaaactcatagtcttttaaaaaataatcaagaatattcttaagaatcaaaatcattgatggatctgtgatttcttttaccatcatgaaaaatgtttgtcaattttaatccattctgatttttaaaatatgactttgatatgcccctgtgatgtgtataaagagacctatttgtggccctaaaatggaaagaacagattagtctttgatagagttacttcatgtgatcatttggtctctgtgaacactgaggacagagaaaagtgcttgagggctgctactaatctctcagaaacatttgtatagttcatccatcaaatgacacacatactaaaagaataaagaaattgatgct

SEQ ID NO: 2 - DNA, 3215 bp, Homo sapiens
  source 1..3215: mol_type = other DNA
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 3 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 4 - DNA, 16 bp, synthetic construct
  misc_feature 1..16: note = Antisense Oligonucleotide
  source 1..16: mol_type = other DNA
cctttacaccacactg

SEQ ID NO: 5 - DNA, 18 bp, synthetic construct
  misc_feature 1..18: note = Antisense Oligonucleotide
  source 1..18: mol_type = other DNA
ttcctttacaccacactg

SEQ ID NO: 6 - DNA, 20 bp, synthetic construct
  misc_feature 1..20: note = Antisense Oligonucleotide
  source 1..20: mol_type = other DNA
aattcctttacaccacactg

SEQ ID NO: 7 - DNA, 18 bp, synthetic construct
  misc_feature 1..18: note = Antisense Oligonucleotide
  source 1..18: mol_type = other DNA
attcctttacaccacact

SEQ ID NO: 8 - DNA, 16 bp, synthetic construct
  misc_feature 1..16: note = Antisense Oligonucleotide
  source 1..16: mol_type = other DNA
tcctttacaccacact

SEQ ID NO: 9 - DNA, 19 bp, synthetic construct
  misc_feature 1..19: note = Antisense Oligonucleotide
  source 1..19: mol_type = other DNA
aattcctttacaccacact

SEQ ID NO: 10 - DNA, 20 bp, synthetic construct
  misc_feature 1..20: note = Antisense Oligonucleotide
  source 1..20: mol_type = other DNA
gaattcctttacaccacact

SEQ ID NO: 11 - DNA, 17 bp, synthetic construct
  misc_feature 1..17: note = Antisense Oligonucleotide
  source 1..17: mol_type = other DNA
ttcctttacaccacact

SEQ ID NO: 12 - DNA, 16 bp, synthetic construct
  misc_feature 1..16: note = Antisense Oligonucleotide
  source 1..16: mol_type = other DNA
ttcctttacaccacac

SEQ ID NO: 13 - DNA, 19 bp, synthetic construct
  misc_feature 1..19: note = Antisense Oligonucleotide
  source 1..19: mol_type = other DNA
gaattcctttacaccacac

SEQ ID NO: 14 - DNA, 18 bp, synthetic construct
  misc_feature 1..18: note = Antisense Oligonucleotide
  source 1..18: mol_type = other DNA
aattcctttacaccacac

SEQ ID NO: 15 - DNA, 17 bp, synthetic construct
  misc_feature 1..17: note = Antisense Oligonucleotide
  source 1..17: mol_type = other DNA
attcctttacaccacac

SEQ ID NO: 16 - DNA, 18 bp, synthetic construct
  misc_feature 1..18: note = Antisense Oligonucleotide
  source 1..18: mol_type = other DNA
gaattcctttacaccaca

SEQ ID NO: 17 - DNA, 16 bp, synthetic construct
  misc_feature 1..16: note = Antisense Oligonucleotide
  source 1..16: mol_type = other DNA
attcctttacaccaca

SEQ ID NO: 18 - DNA, 18 bp, synthetic construct
  misc_feature 1..18: note = Antisense Oligonucleotide
  source 1..18: mol_type = other DNA
tgaattcctttacaccac

SEQ ID NO: 19 - DNA, 18 bp, synthetic construct
  misc_feature 1..18: note = Antisense Oligonucleotide
  source 1..18: mol_type = other DNA
atgaattcctttacacca

SEQ ID NO: 20 - DNA, 18 bp, synthetic construct
  misc_feature 1..18: note = Antisense Oligonucleotide
  source 1..18: mol_type = other DNA
aatgaattcctttacacc

SEQ ID NO: 21 - DNA, 17 bp, synthetic construct
  misc_feature 1..17: note = Antisense Oligonucleotide
  source 1..17: mol_type = other DNA
ctaatgaattcctttac

SEQ ID NO: 22 - DNA, 15 bp, synthetic construct
  misc_feature 1..15: note = Antisense Oligonucleotide
  source 1..15: mol_type = other DNA
tctgtcttggctttg

SEQ ID NO: 23 - DNA, 16 bp, synthetic construct
  misc_feature 1..16: note = Antisense Oligonucleotide
  source 1..16: mol_type = other DNA
agaaataagtggtagt

SEQ ID NO: 24 - DNA, 16 bp, synthetic construct
  misc_feature 1..16: note = Antisense Oligonucleotide
  source 1..16: mol_type = other DNA
atttccaaattcactt

SEQ ID NO: 25 - DNA, 20 bp, synthetic construct
  misc_feature 1..20: note = Antisense Oligonucleotide
  source 1..20: mol_type = other DNA
ccaaatcttataataactac

SEQ ID NO: 26 - DNA, 16 bp, synthetic construct
  misc_feature 1..16: note = Antisense Oligonucleotide
  source 1..16: mol_type = other DNA
ggtgaggtttggtaga

SEQ ID NO: 27 - DNA, 18 bp, synthetic construct
  misc_feature 1..18: note = Antisense Oligonucleotide
  source 1..18: mol_type = other DNA
ggtgaggtttggtagaag